(19) United States
(12) Patent Application Publication
(10) Pub. No.: US 2003/0235819 A1
(43) Pub. Date: Dec. 25, 2003

(54) Mutations in the BRCA1 gene

(75) Inventor: Mark B. Rabin, Rockville, MD (US)

Correspondence Address: MORGAN LEWIS & BOCKIUS LLP, 1111 PENNSYLVANIA AVENUE NW, WASHINGTON, DC, 20004, US

(73) Assignee: OncorMed. Inc.

(21) Appl. No.: 09/982,835

(22) Filed: Oct. 22, 2001

Related U.S. Application Data
(62) Division of application No. 09/697,149, filed on Oct. 27, 2000, now abandoned.
Application No. 09/697,149 is a continuation of application No. 09/038,946, filed on Mar. 12, 1998, now abandoned.

(51) Int. Cl. C12Q 1/68; C07H 21/04

(52) U.S. Cl. 435/6; 536/23.2

(57) ABSTRACT

Mutations resulting in stop codons in the BRCA1 gene are described. All of these mutations result in the formation of a truncated BRCA1 protein. Methods for identifying a sequence variation in a BRCA1 polynucleotide sequence are disclosed. The identification process includes allele specific sequence-based assays of known sequence variations. The methods can be used for efficient, and accurate detection of a mutation in a test BRCA1 gene sample for diagnostic and therapeutic purposes.

### FIELD OF THE INVENTION

This invention relates to the cancer suppressor gene BRCA1. More specifically, this invention detects germline mutations of the BRCA1 gene that are associated with breast and ovarian cancer, and somatic cell mutations of the BRCA1 gene indicating the nature of the cancer. Methods and reagents for detecting the presence of these mutations are included.

### COPYRIGHT NOTICE

A portion of the disclosure of this patent document contains material which is subject to copyright protection. The copyright owner has no objection to the facsimile reproduction by anyone of the patent document or the patent disclosure as it appears in the U.S. Patent and Trademark Office patent file or records, but otherwise reserves all copyright rights whatsoever.

### BACKGROUND OF THE INVENTION

BRCA1 is a putative tumor suppressor gene located on chromosome 17. Mutations in the BRCA1 gene are thought to account for roughly 45% of inherited breast cancer and 80-90% of families with increased risk of early onset breast and ovarian cancer (Easton, 1993, et al., American Journal of Human Genetics 52; 678-701). A compilation of the known BRCA1 mutations may be found at the Breast Cancer Information Core world wide web site at http:/Hwww.nhgri.nih.gov/Intramural_research/Lab_transfer/Bic/(BIC) (Friend, S. et al., 1995, Nature Genetics 11: 238). The BRCA1 gene is approximately 100,000 base pairs of genomic DNA encoding the 1836 amino acid BRCA1 protein. The sequence is divided into 24 separate exons. Exons 1 and 4 are noncoding, in that they are not part of the final functional BRCA1 protein product. Each exon consists of 200-400 bp, except for exon 11 which contains about 3600 bp (Weber, B., Science & Medicine (1996). A consensus sequence for the coding region of the human BRCA1 gene, referred to herein as BRCA1 (omi1), was first disclosed in application Ser. No. 08/598,591 (as SEQ ID NO:1, therein), herein incorporated by reference.

Accuracy in detecting mutations in BRCA1 is extremely important, particularly in clinical settings. Direct end-to-end sequencing of the gene potentially provides the most reliable results, given that an accurate reference sequence is available. However, direct sequencing is a cumbersome technique. Detection of one of many known or unknown mutations is further complicated when the gene is large and/or has a complex structure. The human BRCA1 gene, for example, is approximately 100,000 base pairs long and contains 24 exons (Weber, B., Science and Medicine, Scientific American January-February 1996, 12-21). Furthermore, in order to be practical and available to the general population, mutation detection methods must be efficient enough to accommodate a large number of different samples.

A number of techniques that are more rapid but less comprehensive than direct sequencing have been developed for detecting nucleotide sequence variations. These techniques may be used to detect differences between normal and mutant nucleotide sequences. DNA sequence-based allelic discrimination methods include: (1) allele-specific hybridization techniques, which effect detection under high stringent hybridization conditions; (2) single strand conformation polymorphism analysis and heteroduplex analysis which exploit differences in secondary structure of nucleic acid molecules; (3) denaturing gradient gel electrophoresis and constant denaturing gel electrophoresis which detect different alleles based on differences in melting behavior of nucleic acid molecules; (4) restriction enzyme cleavage, which discriminates between alleles based on the presence of absence of corresponding restriction recognition sequences; and (5) chemical or nuclease cleavage which detect base mismatch loci. Other techniques, such as the protein truncation test (Hogervorst F. B., et al., Nat. Genet. June 1995; 10(2):208-212), detect changes in the protein transcripts. For a summary of such techniques, see Marajver & Petty, 1996, Clinics in Lab. Med. 16: 139-167, especially Table 5 at p. 152.

One limitation of all these techniques is that sequence variations are often of unknown clinical significance. Since the triplet code is degenerate, many genetic variants do not alter the amino acid sequence of the resultant protein. Even those sequence alterations that do result in amino acid changes may not have a significant impact on protein function. For example, some amino acid changes will substitute functionally similar amino acids (conservative substitutions) such as one small neutrally charged amino acid for another. Further, some regions of a protein molecule may not be important for protein function. In those regions of the molecule, even large changes the amino acid sequence may be possible. Thus, genetic variants as a whole are often of unknown clinical significance.

If an altered sequence in the coding region of a gene associated with a condition such as cancer is found however, it is important to determine the clinical significance of the variant sequence. Knowing the significance of the sequence variation can be an invaluable tool in determining an appropriate treatment or monitoring regimen. For example, if an individual carries a mutation that interferes with protein function, it may be possible to provide the individual with increased expression of the gene through gene transfer therapy. It has been demonstrated that the gene transfer of the BRCA1 coding sequence into human cancer cells inhibits their growth and reduces tumorigenesis in nude mice. The BRCA1 protein product appears to be a secreted tumor growth inhibitor, making BRCA1 an ideal gene for gene therapy studies. Transduction of only a moderate percentage of tumor cells apparently produces enough growth inhibitor to inhibit all tumor cells. Arteaga, C. L., and J. T. Holt Cancer Research 56: 1098-1103 (1996); Holt, J. T. et al., Nature Genetics 12: 298-302 (1996). The observation of Holt et al. that the BRCA1 growth inhibitor is a secreted protein, also suggests the possibility of tumor suppression by injecting the growth irhibitor into the area of the tumor.

Efforts have independently focused on increasing the efficiency of DNA sequence analyses and increasing the comprehensiveness of the sequence-based techniques. There remains a need, however, for a comprehensive method of detecting mutations in individual gene samples that is both accurate enough to provide a reliable diagnosis to an individual patient and efficient enough to be practical for application to the general population.

Until now, the art has relied upon the occasional occurrence of a previously unreported mutation to increase its base of information for mutation testing in a given gene sequence. To determine the presence or absence of a mutation in a gene from a patient sample, the vast majority of test samples would be subject to complete end-to-end sequencing of the gene. This method is time consuming, often taking six weeks to obtain a result, and is extremely expensive. To ascertain the clinical significance of a previously unidentified sequence variation, those in the art would frequently rely on epidemiological data derived from an analysis of families or populations carrying the newly identified mutation. Other methods of assessing the significance of a newly identified sequence variation would include cloning the altered gene and studying its function in vitro or in vivo or comparing the altered gene sequence with homologous genes in other organisms. Given the realities of many of these genetic diseases, such an analysis comes too late to be of use to the individual bearing the newly identified sequence variation.

There is need in the art, therefore, for improved methods to identify clinically significant mutations. Identification of mutations of the BRCA1 gene would allow better tumor cell analysis for more appropriate therapy, and more widespread diagnostic screening for hereditary cancers than is currently possible, and also permit identification of critical or biologically significant functional areas deduced from the mutational spectrum observed. While mutations occur throughout the BRCA1 gene, there is a need for an assay, test or means for detecting mutations with a high sample number (throughput), sensitivity, accuracy and cost effectiveness.

The present invention addresses these needs and more by providing mutations, molecules, and methods useful for the identification of both known and unknown mutations.

### SUMMARY OF THE INVENTION

It is an object of the invention to provide a method for determining a predisposition or higher susceptibility to cancers in individuals by determining the DNA sequence of the BRCA1 genes in the DNA of a patient specimen and comparing it to a naturally occurring (wild type) BRCA1 gene sequence or any haplotype thereof resulting from polymorphisms to determine whether the sample contains a mutation.

It is a further object of the invention to provide a method of characterizing and classifying a tumor and determining an appropriate therapy dependant upon the type of BRCA1 mutation(s) present.

It is also an object of the present invention to provide a non-chromosomal mutant BRCA1 gene and expressed mutant protein for drug development, gene therapy and other uses to prevent or ameliorate the effects of or resulting from the mutant BRCA1 gene.

It is another object of the present invention to prepare oligonucleotides, or groups of these oligonucleotides, where the oligonucleotide will specifically hybridize to either the wild type BRCA1 gene or a mutant BRCA1 gene to distinguish between the two BRCA1 genes.

It is still another object of the present invention to assay for the presence of a BRCA1 gene encoding a truncated BRCA1 protein by testing for the presence of a truncated BRCA1 protein.

The present invention is based on the discovery of numerous mutations in the published BRCA1 DNA sequence that result in a truncation of the BRCA1 protein. Mutations in the BRCA1 gene that cause truncations are associated with increased susceptibility to, and developmental stage of, different cancers, particularly breast and ovarian cancer. A truncated protein is likely to be non-functional or at least have a different biological activity.

### DETAILED DESCRIPTION OF THE INVENTION

The present invention stems, in part, from the realization that previously unidentified sequence variations as a whole are typically of unknown clinical significance. At this time, epidemiological studies are time-consuming and may be of little use for infrequently identified sequence variations; laboratory studies of protein function are similarly time-consuming and difficult to extrapolate to human disease. However, mutations resulting in a prematurely terminated protein have a high probability of having clinical significance. In part, this is due to the fact that a premature termination of a coding sequence may entirely delete important functional domains, such as zinc fingers, transmembrane domains, phosphorylation sites, glycosylation sites, etc. It is also due, in part, to the fact that the removal of a portion of a protein molecule can significantly disrupt a protein's structure and function, even where no obvious functional domain has been deleted. Thus, the larger the truncation, the greater the likelihood that an important part of the molecule has been removed. Regardless of the cause, a survey of known clinically significant sequence variations (mutations) of the BRCA1 gene reveals that a large percentage of identified mutations are premature truncation mutations of the BRCA1 protein product. Thus, identifying those sequence variations that are premature truncation-causing mutations offers enormous predictive value to the clinician.

The presence of tumor cells with a mutation that inactivates BRCA1 may be clinically significant in determining the tumor stage, likelihood of metastasis, appropriate therapy etc. BRCA1 mutations appear in advanced tumors, several advanced primary cancers and cell lines derived from highly aggressive cancers. Individuals with one of their alleles having a BRCA1 mutation in their germline are at increased susceptibility for cancer, particularly breast and ovarian cancer.

With DNA sequencing technology, genomic DNA has been extracted from samples of whole blood, a cell line or a tumor and the coding regions of the BRCA1 gene were amplified using the polymerase chain reaction (PCR). Each of the coding regions has been sequenced completely and is recited in GENBANK, Accession Number 159546.

A number of mutations have been described in the BRCA1 gene that cause stop codons to be formed, thereby encoding a truncated BRCA1 protein. A list of mutations published to date is in TABLE 1.

The nomenclature of the published literature is inconsistent. See, for example, Beaudet et al, Human Mutations, 2: 245-248 (1993), Antonarakis et al, Human Mutations, 4: 166 (1994), Cotton, Human Mutations, 8: 197-202 (1996), and Beutler et al., Human Mutations, 8: 203-206 (1996). Consequently, the following nomenclature is used to define the mutations and polymorphisms of the present invention. In defining the mutation, the number indicates the nucleotide number corresponding to the BRCA1 gene sequence where the mutation first occurs. For simplified identification purposes, the BRCA1 sequence of SEQ ID NO:1, of U.S. Pat. No. 5,654,155 (GENBANK Accession number 159546) is used. However, the invention is equally applicable to other BRCA1 sequences. Other BRCA1 sequences (haplotypes) that are polymorphisms or genetic variations of BRCA1 may be used, in which case, a corresponding mutation at the corresponding nucleotide number is present.

Haplotypes are distinguished based on the combination of nucleotides present at particular polymorphic loci. Polymorphic nucleotide sites 2201, 2430, 2731, 3232, 3667, 4427, and 4956 usually define a haplotype. Other polymorphic sites of a BRCA1 gene are at Exon 4 (not coding) 49, IVS8-57, 1186, 2196, and 3238. Less common polymorphisms are at 233G>A, 561-34C>T, exon 9-6delT, 710C>T, 1100A>G, 1985G>T, 2202G>A, 2687T>C, 2933A>G, 3263T>C, 4077T>C, 4145A>G, 4193G>A, 4209-141C>A, 4364A>G, 4932T>C, 5106-68G>A, 5106-92G>A, intronic exon17-G>A, 5232G>T, 5272+66A>G, intronic exon18+A>G, 5396+48 12bp insert, 5396+4712bp insert, intronic exon 22+T>C, 5651C>T, 5657G>A, and UGA+36C>G. See, Couch et al., Human Mutations, 8(1):8-18 (1996), where polymorphisms have been reported.

As the BRCA1 gene numbering does not include introns, when the mutation is located within an intron, a nucleotide in the coding sequence +or − a number of nucleotides is given. Insertion mutations are indicated by “ins” and deletion mutations are indicated by “del”. Letters after “ins” or “del” refer to the nucleotide(s) which were inserted or deleted. Alternatively, where several bases have been inserted or deleted, a number may follow “ins” or “del,” indicating the number of affected bases. When the mutation results in one nucleotide being substituted for another, the original nucleotide(s) is placed to the left of the nucleotide number and the nucleotide(s) corresponding to the substituting nucleotide is placed to the right of the nucleotide number. In some cases, the substitution is indicated by both the original and the substituted nucleotides following the nucleotide number, with an “>” between them. In such cases the original nucleotide is to the left of the “>” and the substituted nucleotide is to the right of the “>,” as for example, in the designation 5657G>A.

### TABLE 1

185delAG, 185insA, 188del11, 189insA, 189insTGTC, 192del2, 259insT, 351delA, 448insA, 448delAG, 525insA, 589delCT, 613insT, 633delC, 787insA, 788delA, 794delT, 795delT, 816delGT, 916delTT, 917delTT, 926ins11, 962del4, 1048delA, 1049delG, 1099delCA, 1100delAT, 1103insC, 1129delA, 1135insA, 1191delC, 1201del11, 1205delGA, 1206delA, 1220insC, 1240delC, 1294del40, 1323delG, 1374delG, 138del29, 1395delT, 1406insA, 1411insT, 1438delT, 1459insG, 1479delAG, 1499insA, 1505delG, 1506delA, 1509delA, 1511insC, 151insC, 1559insA, 1611delC, 1623del5, 1670delT, 1675delA, 1701del7, 1768delA, 1832delS, 1942del4, 1996ins4, 2000del4, 2012insT, 2071insA, 2072del4, 2072insG, 2080delA, 2080delA, 2080insA, 2138delA, 2140delC, 2187delA, 2190delA, 2198delCA, 2229delAA, 2274insA, 2294delG, 2295delC, 2307insG, 2312del5, 2313del5, 2314del5, 231delAA, 2325delG, 2329delC, 2329delCA, 2334insCT, 2388delG, 2401delAA, 2415delAG, 2448delT, 2473insA, 2509delAA, 2569delG, 2575delC, 2576delC, 2594delC, 2594insA, 2595delA, 2596delC, 2711delA, 2731insT, 2765delTGC, 2795del4, 2798del4, 2800delAA, 2804delAA, 2809insA, 2819delTT, 2844del4, 2846delTCAA, 2862delTC, 2867insA, 2883del4, 2911delTGGT, 2925del4, 2953delGTA>insC, 2953insCdelGTA, 2954insT, 2982del5, 3039delTT, 3109insAA, 3121 delA, 3124delA, 3135del4, 3166ins5, 3172ins5, 3345delAG, 3345delAG, 3375insGA, 339insA, 3407delAA, 3411delCT, 3449insA, 3450del4, 3476delT, 3596del4, 3599dell, 3600del11, 3604delA, 3668delAGinsT, 3731delA, 3768insA, 3818delA, 3819del5, 3825del8, 3829delT, 3874del4, 3875del4, 3879insT, 3879insT, 3883insA, 3889delAG, 3890delGG, 3896delT, 3938insG, 3960delCA, 3977del4, 3988delAA, 4035delTT, 4050del4, 4091delG, 4154delA, 4184del4, 4239delAG, 4280delTC, 4284delAG, 4601delAA, 4693delAA, 5055delG, 5061delA, 5083del19, 5085del19, 5085del19, 5124delG, 5124delG, 5145del11, 5145del11, 5149del4, 5149del4, 5154del5, 5154del5, 5245delG, 5256delG, 5296del4, 5348delAA, 5382insC, 5389del7, 5404insG, 5438insC, 5439delAA, 5502insT, 5559delG, 5598insGA, 5629delG, 5640delA, 5677insA, A1518T, A2154T, C1599T, C1648G,. C1695T, C1740T, C1806T, C2257G, C2428A, C2457T, C297T, C3042T, C3508G, C3549T, C3726T, C3726T, C3726T, C3726T, C3837T, C3904A, C3960T, C3960T, C3960T, C4086T, C4302T, C4305T, C4341T, C4377T, C4446T, C4808G, C4929T, C5370T, C5622T, C624T, G1081A, G1177A, G1235A, G1371T, G1569T, G2307T, G2508T, G2841T, G3297T, G3759T, G3780T, G3867T, G4740T, G5199T, G5273A, G5292T, G5465A, G546T, G5563A, G5625T, G5630A, IVS12-1643del3835, IVS12-1643del3835, T1411G, T2035A, T3053G, T3358A, T3376G, T3458G, T5298A.

It should be noted that polymorphisms in the coding sequence are known and listed in TABE 2. An example is A180G. In the non-coding regions, a deleted T, 2 base pairs beyond exon 19 (5312+2delT) and a 12 base pair insertion 47 bases beyond exon 20 (5396+47ins12bp) are also known polymorphisms. Other genetic changes which may be polymorphisms or mutations are listed under mutations above. While the present invention references the sequences recited above, it is recognized that polymorphisms, either these recited or others, may be equally used for the purposes of the present invention.

### TABLE 2

del exon 3, del561-702, del561-789, exon 18 delA, 5312+2delT, codong 1749 Pro to Arg, IVS20ins12, dup5396+48, A1100G, G1112C, C1120T, A1186G, A1186G, A120G, M11, D369del, T1256G, G1503A, T151C, A1546G, T1575C, C1605T, G1606A, G1630A, G1639T, T172C, C1735T, A1767C, A180G, 5544delGTT, C1822T, T1831C, T189C, G1985T, exon 9-2A>C, C2053A, G2093C, C212G, C2121T, G2196A, C2198T, C2201T, G2202A, A2286G, C2299T, G233A, G233A, A243G, T2430C, T2434C, G2531C, A2577G, C2596A, C2596A, C2640T, A2646G, T2687C, C2715T, C2731T, C2731T, C2731T, A2933G, T300G, C3030A, T309G, G310A, G310A, G3143A, A3165G, G3202A, A3232G, A3233G, G3238A, G3238A, T3263C, A330G, In6(+3)A>G, A3339G, C3415T, A3446G, T3529C, A3537G, G3543C, C3547T, C3567T, C3567T, A3667G, C3706A, G3720A, G3727A, G3776C, T378G, C3832T, G3867A, T388C, T4077C, A4145G, G4155A, A4158G, G4193A, 4209-141C>A, G4303A, A433G, G4364A, C4380T, C4427T, C4446G, Exon12+6,T>C, G4603T, G4654T, G4719A, G4755A, G5396+48ins12bp, C4801T, C480T, IVS7-3delT, C49T, A4931G, T4932C, A4935G, C4942T, A4956T, A4956G, A5001G, T5002C, T5002C, C5029T, G5040A, G5075A, G5076A, G5112A, G5112A, V1688del, V1688del, G5193A, G5193A, 5194-2A>C, C5214T, G5215A, C5232T, C5242A, C5242A, T5257C, G5263A, IVS18+1G>T, 5272+66A>G, I-18 5272+66G>A, 5272+66G>A, A5277G, 5280delCAG, A5317G, G5332A, A5335G, G5396A, 5396+1 G>A, G5396+1A, 5396+47ins12bp, T5443G, G546A, T5467C, G5482T, T5530A, C5535G, T5542C, T5548G, A5575G, G5586A, 561-34 C>T, G5616A, T5628C, C5651T, 5657G>A, A655G, C676A, G690A, C710T, C710T, G731C, T855G, G876A, G930A, G930C, exon 2-3insAG , IVS20+60ins12, Intron22T>C, T>G ins59bp, exon 9-6delT, G5396+47ins12bp, in18+1G>C, in20-1(G>T), IVS11-2delGT, IVS20ins12, IVS22+5G.A, IVS1-21insAT, IVS11-2A>G, IVS13+1G>T, IVS13+2T>G, IVS13-10C>T, IVS14-2A>G, IVS15+1G>A, IVS16+3G>C, IVS16+6T>C, IVS16-20A>G, IVS16-20A>G, IVS18+6T>G, IVS18-13A>G, IVS2+1G>A, IVS2+IG>T, IVS2-1 delT, IVS2-12C>G, IVS20+60ins12, IVS20-1G>A, IVS22+8T>C, IVS23-10C>A, IVS4-IG>T, IVS5+1G>T, IVS5-11T>G, IVS5-12A>G, IVS6+7G>A, IVS6-2A>T, IVS6-2delA, IVS7-3delT, IVS8+2T>A, IVS8-17G>T, IVS9+3G>A, exon24(+36)C>G, 3′UTR C>G (+36).

It should be noted that not all of the mutations listed in TABLES 1 and 2 are prior art as some of the mutations were published very recently after applicant determined his mutations.

Mutations detected according to the present invention are nonsense and frame shift mutations. Nonsense mutations cause an in-frame stop codon, which results in expression of a to truncated protein of presumably no BRCA1 functional ability or at least a significantly altered BRCA1 biological activity. Frameshift mutations cause an out-of-frame stop codon to be created in the inserted or deleted coding sequence. This formed stop codon may be at the site of mutation or downstream from the mutation. For the example of nonsense mutations, a nucleotide in a codon is mutated to provide a stop codon having a sequence of TAA, TAG or TGA. For the example of a frame shift mutation, 1, 2, 4, 5, or any larger integer not 3 or a multiple of three, nucleotides are inserted into, or deleted from, the BRCA1 gene sequence. Such mutations result in the formation of a stop codon at the codon containing the mutation or within codons downstream from the mutation site, but before the end of the wild type BRCA1 gene.

The presence of one or more of such mutations is expected to be clinically significant as they are capable of producing a truncating BRCA1 mutation. As the effects of truncations are predictably harmful, one has a high degree of certainty that the presence of such a mutation is clinically significant.

For example, truncating mutations 5382insC, 5438insC, and 185delAG are known to be associated with cancer (Abeliovich, et al., Am. J. Hum. Genet., 60:505-514 (1997); Struewing et al., Nat. Genetics, 11:198-200 (1995); Shattuck-Eidens, et al., JAMA 273(7):535-541 (1995). Accordingly, any truncating mutation deleting the same portion or more of the coding sequence is presumed to produce an affected mutant BRCA1 protein and to be associated with cancer or an increased susceptibility to cancer. 5382insC and 5438insC cause frame shifts, producing a stop codon (TGA) at nucleotides 5604-5606. 185delAG causes a frame shift, producing a stop codon (TGA) at nucleotides 234-236. Therefore, for the purposes of the present invention, truncating mutations at this location of a lower nucleotide number are presumed to be harmful or of clinical significance.

Conversely, the clinical significance of many missense mutations is unclear. Even when isolated from a tumor cell, the exact effect on BRCA1 protein must be independently shown. Theoretically, most variations in the BRCA1 sequence will be missense variations and relatively AD fewer will be truncating mutations. At the present time, the rules are unknown for which missense mutations in BRCA1 are predictably harmful or otherwise clinically significant. The BRCA1 gene is simply not sufficiently characterized, and given the history of mutations in other genes and computer programs attempting to find predictability, missense mutations will continue to be unpredictable. Without a certainty of their clinical significance, simply knowing that a missense variation exists may have no value.

In general, truncated proteins are non-functional or of reduced function by lacking proper biological activity. The presence of a BRCA1 gene encoding a truncated protein has a high probably of being clinically significant. This is in part due to important portions of the BRCA1 protein being located in the truncated region and not present in the expressed mutant protein. Important functional domains include, for example, any part of the active site, transmembrane domains, zinc fingers, phosphorylation sites, glycosylation sites, sites interacting with metal ions, coenzymes, cofactors, other ligands or receptors, etc. Furthermore, removal of part of the protein molecule or addition of amino acids to the sequence can significantly disrupt the secondary, tertiary or quaternary (if any) structure of the protein, even when no obvious functional domain has been deleted by the truncation.

For example, the 5382insC mutation is found in breast and ovarian cancers. This single base insertion creates a stop-codon at codon 1829, truncating the protein at a cysteine residue. This truncation deletes less than 2 percent of the coding sequence of BRCA1 . Accordingly, all truncations that delete this region of the BRCA1 gene would also by inference render the truncated BRCA1 protein non-functional for its putative tumor suppressor function. Mutant BRCA1 genes with even smaller portions of the gene being truncated are also known to be associated with cancer. For example, truncating mutations causing a stop codon at nucleotides 5676-5678, truncating 0.5% of the coding sequence, are also known to be associated with breast cancer (5677insA; Shattuck-Eidens et al., JAMA, 273(7):535-541 (1995)). Please note that several mutations in the DNA result in the expression of the same mutant BRCA1 protein because each mutation causes a stop codon to be formed at nucleotide site 5676-5678. This corresponds to codon 1853 (TAC—Tyrosine). Thus, all of these mutations are very likely to cause the same effective result, for example, in terms of susceptibility to cancer or cancer typing. Therefore, detecting these mutations would be diagnostically significant and useful for assessing the susceptibility for cancer. Given these mutations, oligonucleotides complementary to the F regions of these mutations are prepared to assay for the presence of these mutations in a sample.

For the present invention, the truncating mutations are detected in the BRCA1 gene or a fragment thereof which contains a mutation which directly or indirectly causes the formation of an in-frame stop codon (TAA, TAG or TGA) prematurely at any of codons 2 to 1863 A premature in-frame stop codon is one that occurs before the natural stop codon, which does not occur in a wild-type BRCA1 gene and when expressed results in a truncated protein product.

These BRCA1 mutations of the present invention may also be defined as specifically hybridizing to an oligonucleotide probe having the sequence 5′ R1-R2-R3 3′ wherein R1 is an oligonucleotide of at least three nucleotides, R2 is complementary to TAA, TAG or TGA, and R3 is an oligonucleotide of at least three nucleotides, the probe hybridizing to a premature in-frame stop codon on the mutant BRCA1 gene.

Whenever defining an oligonucleotide probe in the present invention, probes capable of potentially hybridizing to the sense strand are referred to. It should be recognized that oligonucleotide probes having a sequence complementary to these oligonucleotide probes may also be used and may specifically hybridize to the anti-sense strand. For diagnostic purposes either may be used, as DNA from biological samples is generally double stranded and contains both the sense and anti-sense strands.

In the present invention, the following BRCA1 mutations of the present invention represent one base changes that result in the formation of an in frame TAA, TAG or TGA. These mutations of the present invention are defined by forming stop codons at specific locations in accordance with TABLE 3. Any expressed protein from BRCA1 genes with these types of mutations should be truncated accordingly. The substituted nucleotide is indicated in lower case letters.

TABLE 3
List of Nonsense Mutations

| Stop Codon Formed | Nucleotide Number | Base Change |
|---|---|---|
| TaA | 127 | T>A |
| TgA | 127 | T>g |
| tAA | 144 | G>T |
| tAA | 147 | G>T |
| tAA | 153 | C>T |
| tAG | 174 | C>T |
| tAA | 177 | A>T |
| TaA | 184 | T>A |
| TgA | 184 | T>g |
| tAG | 186 | G>T |
| TGa | 191 | T>A |
| TGa | 200 | T>A |
| tAG | 204 | G>T |
| TaG | 208 | T>A |
| tAG | 213 | A>T |
| tAA | 216 | G>T |
| tAG | 231 | A>T |
| TGa | 236 | T>A |
| TGa | 251 | C>A |
| tAA | 252 | A>T |
| TGa | 260 | C>A |
| tAA | 267 | A>T |
| tAG | 279 | C>T |
| tAG | 282 | A>T |
| tAA | 285 | A>T |
| TaA | 295 | C>A |
| TgA | 295 | C>g |
| tAG | 297 | C>T |
| TGa | 302 | T>A |
| TaA | 307 | T>A |
| TgA | 307 | T>g |
| TGa | 311 | T>A |
| tAG | 312 | A>T |
| tAA | 327 | A>T |
| tAA | 339 | C>T |
| tAA | 342 | G>T |
| tGA | 351 | A>T |
| tAA | 360 | C>T |
| tAA | 369 | G>T |
| tAG | 372 | G>T |
| TaG | 379 | T>A |
| tAA | 381 | A>T |
| TGa | 392 | T>A |
| tAG | 399 | C>T |
| TaG | 415 | T>A |
| tAG | 417 | G>T |
| TAa | 422 | T>A |
| TAg | 422 | T>G |
| TAa | 434 | T>A |
| TAg | 434 | T>G |
| tAA | 444 | A>T |
| tAG | 447 | A>T |
| tAA | 450 | G>T |
| tAA | 465 | G>T |
| tAA | 474 | A>T |
| tAA | 480 | G>T |
| tAA | 495 | C>T |
| TAa | 509 | C>A |
| TAg | 509 | C>G |
| tGA | 510 | A>T |
| tAA | 522 | A>T |
| tGA | 525 | A>T |
| tAG | 534 | C>T |
| tAA | 540 | G>T |
| tAA | 546 | G>T |
| TaG | 559 | T>A |
| tAG | 561 | C>T |
| tAA | 564 | G>T |
| tAA | 582 | C>T |
| tGA | 597 | G>T |
| tGA | 606 | A>T |
| tAG | 621 | A>T |
| tAG | 624 | C>T |
| tAA | 633 | C>T |
| tAA | 639 | C>T |
| tAG | 642 | A>T |
| TAa | 656 | C>A |
| TAg | 656 | C>G |
| tAA | 660 | G>T |
| TaG | 664 | T>A |
| tGA | 666 | G>T |
| tAA | 681 | G>T |
| tAG | 696 | A>T |
| TAa | 707 | T>A |
| TAg | 707 | T>G |
| TGa | 710 | C>A |
| tGA | 717 | G>T |
| tAA | 723 | C>T |
| tAA | 726 | G>T |
| TaG | 730 | T>A |
| TaA | 733 | T>A |
| TgA | 733 | T>g |
| tAA | 735 | C>T |
| tAA | 747 | C>T |
| tGA | 750 | G>T |
| tAA | 762 | G>T |
| TaG | 772 | T>A |
| tAA | 783 | A>T |
| tAG | 786 | A>T |
| TGa | 797 | T>A |
| tAA | 798 | G>T |
| tAG | 807 | G>T |
| tAA | 828 | G>T |
| tAA | 837 | C>T |
| TaG | 856 | T>A |
| tAG | 867 | G>T |
| tAG | 870 | A>T |
| tAG | 882 | G>T |
| tAA | 894 | G>T |
| tAG | 897 | A>T |
| TAa | 902 | T>A |
| TAg | 902 | T>G |
| tAG | 903 | C>T |
| TaA | 919 | C>A |
| TgA | 919 | C>g |
| TaG | 925 | T>A |
| tAG | 933 | G>T |
| TGa | 941 | T>A |
| TaA | 964 | C>A |
| TgA | 964 | C>g |
| TaA | 967 | T>A |
| TgA | 967 | T>g |
| tAG | 969 | C>T |
| tAG | 975 | G>T |
| TaA | 988 | T>A |
| TgA | 988 | T>g |
| TaA | 991 | T>A |
| TgA | 991 | T>g |
| tAA | 999 | A>T |
| tGA | 1005 | A>T |
| tAA | 1017 | G>T |
| tAG | 1020 | A>T |
| tAA | 1026 | G>T |
| TGa | 1034 | T>A |
| tAA | 1038 | A>T |
| tAA | 1044 | A>T |
| tAG | 1047 | C>T |
| TaA | 1057 | T>A |
| TgA | 1057 | T>g |
| tAA | 1068 | C>T |
| tGA | 1077 | A>T |
| TaG | 1081 | G>A |
| TGa | 1082 | G>A |
| tGA | 1086 | G>T |
| tAG | 1092 | A>T |
| tAA | 1095 | G>T |
| TGa | 1103 | T>A |
| tAA | 1128 | G>T |
| tAA | 1131 | A>T |
| tAG | 1134 | A>T |
| TGa | 1163 | T>A |
| tAG | 1164 | G>T |
| tGA | 1167 | A>T |
| tAA | 1170 | A>T |
| tAA | 1173 | G>T |
| TaG | 1177 | G>A |
| TGa | 1178 | G>A |
| tAG | 1182 | A>T |
| tAG | 1185 | C>T |
| tAA | 1188 | A>T |
| TGa | 1199 | C>A |
| TaA | 1201 | C>A |
| TgA | 1201 | C>g |
| tAG | 1203 | G>T |
| tGA | 1212 | A>T |
| tAA | 1221 | G>T |
| TaG | 1234 | G>A |
| TGa | 1235 | G>A |
| tAG | 1257 | C>T |
| tAA | 1260 | A>T |
| tAG | 1269 | G>T |
| TaG | 1273 | G>A |
| TGa | 1274 | G>A |
| tGA | 1281 | A>T |
| tAA | 1290 | G>T |
| TaA | 1297 | T>A |
| TgA | 1297 | T>g |
| TaA | 1312 | C>A |
| TgA | 1312 | C>g |
| tAG | 1323 | G>T |
| tAA | 1329 | G>T |
| TaA | 1333 | C>A |
| TgA | 1333 | C>g |
| tAA | 1341 | A>T |
| TaG | 1357 | T>A |
| tAG | 1371 | G>T |
| tAA | 1380 | G>T |
| TAa | 1385 | T>A |
| TAg | 1385 | T>G |
| TaA | 1396 | C>A |
| TgA | 1396 | C>g |
| tAG | 1398 | G>T |
| tAA | 1401 | A>T |
| TaA | 1411 | T>A |
| TgA | 1411 | T>g |
| tAG | 1431 | G>T |
| TaA | 1438 | T>A |
| TgA | 1438 | T>g |
| TGa | 1445 | T>A |
| tAA | 1446 | A>T |
| tAA | 1452 | G>T |
| tGA | 1455 | A>T |
| tAA | 1467 | A>T |
| TaA | 1471 | C>A |
| TgA | 1471 | C>g |
| tAG | 1476 | G>T |
| tAA | 1488 | G>T |
| tAA | 1494 | A>T |
| tAA | 1506 | A>T |
| TAa | 1514 | T>A |
| TAg | 1514 | T>G |
| tAG | 1518 | A>T |
| tAG | 1521 | A>T |
| TaA | 1540 | T>A |
| TgA | 1540 | T>g |
| tAA | 1554 | G>T |
| tGA | 1569 | G>T |
| tAG | 1584 | G>T |
| tAG | 1590 | C>T |
| tAA | 1599 | C>T |
| tAG | 1602 | G>T |
| tAA | 1620 | A>T |
| TaA | 1624 | T>A |
| TgA | 1624 | T>g |
| tAG | 1626 | A>T |
| tAA | 1632 | A>T |
| tGA | 1638 | A>T |
| TaA | 1648 | C>A |
| TgA | 1648 | C>g |
| tAG | 1662 | G>T |
| tAG | 1674 | A>T |
| tAA | 1677 | A>T |
| TaG | 1687 | T>A |
| tAA | 1695 | C>T |
| tAG | 1698 | A>T |
| tAA | 1707 | G>T |
| tAG | 1719 | C>T |
| tGA | 1722 | G>T |
| tAA | 1731 | C>T |
| tAG | 1737 | G>T |
| tAG | 1740 | C>T |
| tAA | 1749 | C>T |
| tAG | 1779 | G>T |
| tAA | 1785 | A>T |
| tAA | 1791 | A>T |
| tAG | 1806 | C>T |
| tAG | 1812 | G>T |
| tAA | 1815 | A>T |
| tAA | 1833 | G>T |
| TaA | 1837 | C>A |
| TgA | 1837 | C>g |
| tAA | 1842 | G>T |
| tAA | 1845 | A>T |
| tAA | 1848 | G>T |
| tAA | 1860 | A>T |
| tAA | 1866 | A>T |
| tAA | 1872 | G>T |
| tAA | 1902 | G>T |
| tAA | 1908 | G>T |
| TaA | 1912 | T>A |
| TgA | 1912 | T>g |
| TaA | 1927 | C>A |
| TgA | 1927 | C>g |
| tAA | 1929 | A>T |
| tAA | 1938 | A>T |
| tAG | 1941 | A>T |
| tAG | 1959 | A>T |
| tAA | 1989 | G>T |
| tGA | 2004 | A>T |
| TGa | 2027 | T>A |
| tAA | 2031 | G>T |
| TaG | 2035 | T>A |
| tAA | 2037 | C>T |
| TGa | 2051 | T>A |
| tAA | 2061 | G>T |
| tAG | 2064 | G>T |
| tAG | 2070 | A>T |
| tAA | 2073 | A>T |
| tAA | 2076 | A>T |
| tAG | 2079 | A>T |
| TAa | 2084 | C>A |
| TAg | 2084 | C>G |
| tAA | 2088 | C>T |
| tGA | 2109 | A>T |
| tAA | 2118 | C>T |
| tAA | 2127 | G>T |
| tAA | 2133 | A>T |
| tAA | 2136 | G>T |
| tGA | 2148 | G>T |
| tAG | 2154 | A>T |
| tAG | 2157 | A>T |
| tAG | 2166 | A>T |
| tAA | 2175 | G>T |
| tAG | 2178 | C>T |
| tAA | 2187 | A>T |
| tGA | 2190 | A>T |
| tAG | 2214 | G>T |
| tAG | 2220 | A>T |
| TaA | 2224 | T>A |
| TgA | 2224 | T>g |
| tAG | 2250 | A>T |
| TGa | 2255 | T>A |
| TaA | 2257 | C>A |
| TgA | 2257 | C>g |
| tAA | 2268 | G>T |
| tAA | 2274 | A>T |
| tAA | 2277 | G>T |
| tGA | 2301 | A>T |
| tAA | 2304 | G>T |
| tAA | 2307 | G>T |
| tAA | 2310 | A>T |
| tAA | 2313 | G>T |
| tAG | 2316 | G>T |
| tAA | 2319 | A>T |
| tAA | 2325 | G>T |
| tAA | 2334 | A>T |
| tAA | 2352 | G>T |
| tAA | 2361 | A>T |
| TaA | 2374 | T>A |
| TgA | 2374 | T>g |
| tGA | 2379 | G>T |
| tAA | 2382 | G>T |
| TaG | 2392 | T>A |
| tAA | 2394 | C>T |
| tAA | 2400 | G>T |
| tGA | 2403 | A>T |
| tAG | 2412 | G>T |
| TaA | 2428 | C>A |
| TgA | 2428 | C>g |
| TAa | 2450 | T>A |
| TAg | 2450 | T>G |
| tAG | 2457 | C>T |
| tAA | 2460 | G>T |
| TaG | 2470 | C>A |
| TaA | 2473 | T>A |
| TgA | 2473 | T>g |
| tAA | 2478 | G>T |
| tAG | 2496 | A>T |
| tAA | 2502 | A>T |
| tAA | 2508 | G>T |
| tAA | 2517 | A>T |
| TGa | 2522 | T>A |
| tAG | 2529 | C>T |
| TGa | 2534 | T>A |
| tAA | 2544 | G>T |
| tAG | 2553 | A>T |
| tGA | 2556 | G>T |
| TGa | 2573 | T>A |
| tAA | 2577 | A>T |
| tGA | 2586 | A>T |
| tAA | 2598 | G>T |
| tAG | 2607 | A>T |
| TAa | 2612 | T>A |
| TAg | 2612 | T>G |
| TaG | 2617 | T>A |
| tGA | 2619 | G>T |
| tAA | 2625 | G>T |
| tAA | 2643 | G>T |
| tAA | 2655 | G>T |
| tAA | 2661 | G>T |
| tAA | 2664 | G>T |
| tAA | 2670 | G>T |
| tAG | 2682 | C>T |
| TAa | 2687 | T>A |
| TAg | 2687 | T>G |
| TaG | 2689 | T>A |
| tAG | 2691 | C>T |
| tAG | 2703 | A>T |
| TaA | 2710 | C>A |
| TgA | 2710 | C>g |
| tAG | 2712 | A>T |
| tAG | 2718 | C>T |
| TaA | 2722 | C>A |
| TgA | 2722 | C>g |
| TaA | 2737 | C>A |
| TgA | 2737 | C>g |
| tGA | 2745 | G>T |
| tAA | 2754 | G>T |
| tAG | 2757 | G>T |
| tAA | 2760 | G>T |
| TGa | 2765 | T>A |
| TaA | 2794 | T>A |
| TgA | 2794 | T>g |
| tAG | 2796 | A>T |
| tAA | 2799 | A>T |
| tAA | 2802 | C>T |
| tAA | 2811 | A>T |
| tAA | 2823 | G>T |
| TGa | 2828 | T>A |
| tAA | 2829 | G>T |
| tAA | 2832 | C>T |
| tAG | 2835 | A>T |
| tAA | 2838 | G>T |
| tAA | 2841 | G>T |
| tAA | 2847 | C>T |
| tGA | 2850 | G>T |
| tAG | 2853 | A>T |
| tAG | 2859 | G>T |
| tAG | 2871 | A>T |
| tAG | 2880 | C>T |
| tAG | 2919 | C>T |
| tAA | 2922 | A>T |
| tAG | 2928 | A>T |
| tAA | 2946 | A>T |
| TGa | 2951 | T>A |
| tAA | 2958 | A>T |
| tGA | 2961 | G>T |
| TGa | 2978 | T>A |
| TaA | 2983 | C>A |
| TgA | 2983 | C>g |
| tAG | 2988 | C>T |
| tGA | 2994 | A>T |
| tAA | 3003 | G>T |
| tGA | 3009 | G>T |
| tAA | 3027 | A>T |
| tGA | 3033 | G>T |
| TaA | 3040 | T>A |
| TgA | 3040 | T>g |
| tAA | 3042 | C>T |
| TAa | 3053 | T>A |
| TAg | 3053 | T>G |
| tAG | 3078 | A>T |
| TaA | 3082 | C>A |
| TgA | 3082 | C>g |
| tAA | 3090 | A>T |
| tAA | 3096 | A>T |
| TGa | 3101 | T>A |
| tAG | 3102 | A>T |
| tAA | 3105 | A>T |
| tAG | 3117 | G>T |
| tAA | 3120 | G>T |
| tAG | 3129 | G>T |
| tAA | 3132 | G>T |
| TaA | 3139 | C>A |
| TgA | 3139 | C>g |
| TaA | 3145 | C>A |
| TgA | 3145 | C>g |
| tAA | 3150 | G>T |
| tGA | 3153 | A>T |
| tAA | 3156 | G>T |
| tGA | 3162 | G>T |
| tAG | 3168 | G>T |
| tGA | 3213 | A>T |
| tAA | 3216 | G>T |
| tAA | 3228 | A>T |
| tGA | 3231 | G>T |
| TaA | 3241 | C>A |
| TgA | 3241 | C>g |
| tAA | 3255 | G>T |
| tAA | 3276 | G>T |
| tAA | 3297 | G>T |
| tAA | 3315 | G>T |
| tAA | 3324 | C>T |
| tAA | 3330 | G>T |
| tGA | 3339 | A>T |
| tGA | 3345 | A>T |
| tAA | 3354 | A>T |
| TaG | 3358 | T>A |
| tGA | 3372 | A>T |
| TaA | 3376 | T>A |
| TgA | 3376 | T>g |
| TaG | 3385 | T>A |
| tAA | 3387 | C>T |
| tAG | 3393 | G>T |
| TAa | 3401 | T>A |
| TAg | 3401 | T>G |
| tAA | 3402 | A>T |
| tAA | 3405 | C>T |
| tGA | 3417 | G>T |
| TGa | 3428 | T>A |
| tAG | 3429 | A>T |
| tAA | 3438 | G>T |
| tAA | 3444 | A>T |
| tAG | 3447 | A>T |
| tAA | 3450 | C>T |
| tAA | 3453 | G>T |
| TAa | 3458 | T>A |
| TAg | 3458 | T>G |
| tAA | 3459 | G>T |
| tAA | 3462 | G>T |
| tAG | 3471 | C>T |
| TAa | 3500 | T>A |
| TAg | 3500 | T>G |
| TaA | 3508 | C>A |
| TgA | 3508 | C>g |
| TaA | 3517 | T>A |
| TgA | 3517 | T>g |
| tAA | 3519 | G>T |
| tAG | 3522 | C>T |
| tGA | 3531 | G>T |
| tAG | 3549 | C>T |
| TGa | 3557 | T>A |
| tAG | 3561 | G>T |
| TaA | 3580 | T>A |
| TgA | 3580 | T>g |
| tAA | 3591 | G>T |
| tAG | 3597 | A>T |
| tAA | 3600 | G>T |
| tAA | 3618 | G>T |
| tAG | 3630 | A>T |
| tAA | 3633 | G>T |
| tAA | 3654 | A>T |
| tAG | 3663 | C>T |
| tGA | 3666 | A>T |
| tGA | 3669 | G>T |
| tAG | 3672 | G>T |
| TaG | 3712 | T>A |
| tAG | 3717 | C>T |
| TAa | 3725 | C>A |
| TAg | 3725 | C>G |
| tGA | 3726 | C>T |
| tGA | 3729 | A>T |
| tAG | 3738 | A>T |
| tAA | 3741 | A>T |
| TaA | 3745 | T>A |
| TgA | 3745 | T>g |
| tAG | 3747 | G>T |
| TaA | 3754 | C>A |
| TgA | 3754 | C>g |
| tAA | 3756 | G>T |
| tAG | 3759 | G>T |
| TaA | 3766 | T>A |
| TgA | 3766 | T>g |
| tAG | 3774 | G>T |
| tAA | 3780 | G>T |
| tAG | 3783 | G>T |
| TGa | 3794 | C>A |
| tAA | 3798 | C>T |
| TaG | 3805 | T>A |
| TaA | 3808 | T>A |
| TgA | 3808 | T>g |
| tAA | 3816 | A>T |
| tAG | 3837 | C>T |
| tAG | 3867 | G>T |
| TGa | 3872 | T>A |
| tAG | 3879 | A>T |
| tAG | 3888 | G>T |
| tAG | 3891 | G>T |
| TaA | 3898 | T>A |
| TgA | 3898 | T>g |
| TaA | 3901 | T>A |
| TgA | 3901 | T>g |
| TaA | 3904 | C>A |
| TgA | 3904 | C>g |
| TaG | 3907 | T>A |
| tAG | 3909 | A>T |
| TaA | 3919 | T>A |
| TgA | 3919 | T>g |
| TGa | 3929 | C>A |
| tAG | 3936 | C>T |
| TaG | 3946 | T>A |
| tAG | 3951 | A>T |
| tAG | 3960 | C>T |
| tAA | 3963 | G>T |
| tAG | 3978 | G>T |
| tAA | 3981 | G>T |
| tAA | 3987 | A>T |
| TGa | 3992 | T>A |
| TaG | 4003 | T>A |
| TaA | 4012 | C>A |
| TgA | 4012 | C>g |
| tAG | 4014 | C>T |
| TGa | 4019 | C>A |
| tAA | 4023 | G>T |
| TaG | 4027 | T>A |
| tAA | 4029 | G>T |
| TaG | 4036 | T>A |
| tAG | 4056 | C>T |
| TaG | 4069 | T>A |
| tAA | 4083 | A>T |
| tAA | 4086 | C>T |
| tAG | 4098 | C>T |
| tAA | 4104 | G>T |
| tAG | 4110 | C>T |
| tGA | 4113 | G>T |
| tAG | 4131 | A>T |
| tAA | 4134 | G>T |
| TaG | 4138 | T>A |
| TaA | 4144 | C>A |
| TgA | 4144 | C>g |
| tAA | 4152 | G>T |
| tAA | 4155 | G>T |
| tGA | 4158 | A>T |
| tGA | 4161 | G>T |
| TaG | 4171 | T>A |
| tAA | 4173 | G>T |
| tAA | 4176 | G>T |
| tAA | 4185 | C>T |
| tAA | 4188 | G>T |
| tAG | 4191 | G>T |
| tAA | 4194 | C>T |
| TaA | 4207 | C>A |
| TgA | 4207 | C>g |
| TaA | 4213 | T>A |
| TgA | 4213 | T>g |
| tAA | 4218 | G>T |
| TGa | 4235 | T>A |
| tAG | 4236 | G>T |
| tAA | 4242 | G>T |
| tAA | 4257 | G>T |
| TGa | 4265 | C>A |
| TaA | 4267 | C>A |
| TgA | 4267 | C>g |
| tAG | 4281 | C>T |
| TaA | 4294 | T>A |
| TgA | 4294 | T>g |
| tAG | 4302 | C>T |
| tAG | 4305 | C>T |
| tAA | 4320 | C>T |
| tAG | 4335 | A>T |
| tAG | 4341 | C>T |
| tAG | 4344 | C>T |
| tAA | 4347 | G>T |
| tAA | 4356 | G>T |
| tAA | 4362 | G>T |
| TaA | 4372 | T>A |
| TgA | 4372 | T>g |
| tAA | 4374 | G>T |
| tAG | 4377 | C>T |
| tAG | 4389 | C>T |
| TAa | 4406 | C>A |
| TAg | 4406 | C>G |
| tAG | 4437 | G>T |
| tGA | 4446 | C>T |
| tAA | 4455 | G>T |
| tAA | 4458 | C>T |
| TaA | 4468 | C>A |
| TgA | 4468 | C>g |
| tAA | 4470 | G>T |
| tAA | 4473 | A>T |
| TaA | 4483 | T>A |
| TgA | 4483 | T>g |
| TaA | 4489 | C>A |
| TgA | 4489 | C>g |
| tAG | 4491 | C>T |
| tAA | 4494 | A>T |
| tAA | 4503 | G>T |
| TAa | 4508 | C>A |
| TAg | 4508 | C>G |
| tAG | 4518 | C>T |
| tAA | 4527 | G>T |
| tAG | 4545 | A>T |
| tAG | 4551 | G>T |
| tAA | 4578 | A>T |
| tAA | 4584 | A>T |
| tAA | 4587 | G>T |
| tGA | 4593 | G>T |
| tAA | 4599 | G>T |
| TaA | 4606 | C>A |
| TgA | 4606 | C>g |
| tAA | 4617 | A>T |
| TGa | 4622 | C>A |
| TaA | 4627 | C>A |
| TgA | 4627 | C>g |
| TaA | 4630 | T>A |
| TgA | 4630 | T>g |
| TaG | 4642 | G>A |
| TGa | 4643 | G>A |
| TAa | 4646 | C>A |
| TAg | 4646 | C>G |
| TGa | 4658 | C>A |
| tAG | 4671 | C>T |
| tGA | 4677 | A>T |
| TAa | 4685 | C>A |
| TAg | 4685 | C>G |
| tAA | 4692 | C>T |
| tAG | 4695 | G>T |
| tAG | 4698 | G>T |
| tAG | 4707 | A>T |
| tAG | 4722 | G>T |
| tAG | 4725 | G>T |
| tAA | 4728 | C>T |
| tAG | 4731 | C>T |
| tAA | 4737 | G>T |
| tAG | 4740 | G>T |
| TaG | 4759 | T>A |
| tAA | 4764 | G>T |
| TAa | 4775 | C>A |
| TAg | 4775 | C>G |
| TaG | 4777 | T>A |
| tAA | 4785 | C>T |
| tAG | 4794 | G>T |
| tGA | 4797 | G>T |
| TAa | 4808 | C>A |
| TAg | 4808 | C>G |
| tAA | 4812 | G>T |
| tGA | 4818 | G>T |
| tAA | 4845 | G>T |
| tAA | 4860 | G>T |
| tGA | 4866 | A>T |
| tAG | 4875 | G>T |
| TaA | 4879 | C>A |
| TgA | 4879 | C>g |
| TaA | 4906 | C>A |
| TgA | 4906 | C>g |
| TaG | 4918 | T>A |
| tAA | 4920 | A>T |
| tAA | 4929 | C>T |
| TaG | 4933 | T>A |
| tAA | 4935 | A>T |
| tAA | 4944 | G>T |
| tAG | 4953 | C>T |
| TAa | 4994 | T>A |
| TAg | 4994 | T>G |
| tAA | 5004 | G>T |
| tAA | 5007 | G>T |
| tAG | 5022 | G>T |
| tAG | 5025 | A>T |
| tAA | 5031 | G>T |
| TaG | 5035 | T>A |
| TaA | 5044 | C>A |
| TgA | 5044 | C>g |
| tAA | 5049 | G>T |
| tAA | 5061 | A>T |
| tGA | 5064 | A>T |
| tAA | 5097 | G>T |
| tAA | 5100 | G>T |
| TAa | 5117 | C>A |
| TAg | 5117 | C>G |
| tAG | 5118 | A>T |
| tGA | 5127 | A>T |
| tAA | 5130 | A>T |
| TaA | 5146 | T>A |
| TgA | 5146 | T>g |
| tAA | 5163 | G>T |
| tAG | 5166 | G>T |
| tAA | 5187 | A>T |
| tAG | 5199 | G>T |
| TGa | 5210 | T>A |
| tAA | 5211 | G>T |
| tAA | 5223 | A>T |
| TAa | 5228 | T>A |
| TAg | 5228 | T>G |
| tGA | 5235 | G>T |
| tGA | 5244 | G>T |
| tGA | 5247 | G>T |
| tAA | 5250 | A>T |
| TaG | 5254 | G>A |
| TGa | 5255 | G>A |
| TAa | 5267 | T>A |
| TAg | 5267 | T>G |
| TaG | 5272 | G>A |
| TGa | 5273 | G>A |
| tAG | 5280 | C>T |
| tAA | 5289 | A>T |
| tAA | 5292 | G>T |
| tGA | 5295 | A>T |
| tAA | 5298 | A>T |
| tAG | 5310 | G>T |
| tAA | 5322 | G>T |
| tGA | 5328 | A>T |
| tGA | 5331 | G>T |
| tGA | 5346 | G>T |
| tGA | 5349 | A>T |
| tAA | 5358 | C>T |
| tAG | 5367 | A>T |
| tGA | 5370 | C>T |
| tGA | 5376 | A>T |
| tAA | 5379 | G>T |
| tAG | 5385 | C>T |
| tGA | 5391 | A>T |
| tAG | 5394 | A>T |
| tAA | 5412 | G>T |
| TGa | 5420 | T>A |
| TGa | 5423 | C>A |
| TAa | 5426 | T>A |
| TAg | 5426 | T>G |
| tAA | 5454 | C>T |
| tAA | 5460 | G>T |
| TaG | 5464 | G>A |
| TGa | 5465 | G>A |
| tAG | 5472 | C>T |
| TGa | 5480 | T>A |
| tAG | 5496 | A>T |
| tAG | 5499 | G>T |
| TaA | 5506 | C>A |
| TgA | 5506 | C>g |
| TaA | 5509 | C>A |
| TgA | 5509 | C>g |
| tAG | 5550 | C>T |
| TaG | 5563 | G>A |
| TGa | 5564 | G>A |
| tAG | 5568 | G>T |
| tAG | 5595 | C>T |
| TGa | 5603 | T>A |
| tAG | 5604 | G>T |
| tGA | 5622 | C>T |
| tAG | 5625 | G>T |
| TaG | 5629 | G>A |
| TGa | 5630 | G>A |
| TaG | 5635 | T>A |
| TAa | 5654 | C>A |
| TAg | 5654 | C>G |
| tAG | 5655 | C>T |
| TGa | 5660 | C>A |
| tAG | 5661 | C>T |
| tAG | 5664 | G>T |
| TAa | 5678 | C>A |
| TAg | 5678 | C>G |
| tAG | 5688 | C>T |
| TAa | 5708 | C>A |
| TAg | 5708 | C>G |
| TaA | 5710 | G>A |

The genes of the present invention containing nonsense mutations are also defined as BRCA1 genes having the sequence 5′ R1-R2-R3 3′; where R1 is the wild type BRCA1 DNA sequence from codon 1 to X−1; R2 is TAA, TAG or TGA; R3 is the wild type BRCA1 DNA sequence from codon X+1 to 1862; and where X=2 to 1861.

The genes of the present invention containing nonsense mutations are also defined by being capable of specifically hybridizing to an oligonucleotide probe having at least 12 nucleotides in length and having the sequence 5′ R1-R2-R3 3′; where R1 contains at its 3′ end three nucleotides complementary to codon X−1 of the wild-type BRCA1 gene; R2=a sequence complementary to TAG, TGA or TAA; R3 contains at its 5′ end three nucleotides complementary to codon X+1 of the wild type BRCA1 gene; where X=2 to 1862 Other oligonucleotide probes complementary to these probes are also acceptable, hybridizing to the antisense strand. The oligonucleotide probe is unable to specifically hybridize to the wild-type BRCA1 gene with the same binding affinity as to the mutant BRCA1 gene.

The present invention also involves frame shift mutations involving insertions or deletions of 1, 2, 4, 5, 7, 8, or any other number which is not 3 or a multiple of 3, nucleotides. Single base deletions of the present invention form one or more stop codons as indicated in the following TABLE 4. The formed in-frame stop codon and the location are provided. Any expressed protein from BRCA1 genes with these types of mutations should be truncated accordingly. It should be recognized that the present invention includes deletions of 3n+1 bases, where n is an integer greater than zero and less than 1862. These larger deletions mutations have stop codons at nucleotide numbers corresponding to the listed stop codon at the nucleotide number listed. The corresponding nucleotide numbers of the stop codons will be 3n nucleotides smaller than those listed.

TABLE 4
Single Base Deletions

| Codon Formed | Nucleotide Number |
|---|---|
| TAG | 183-185 |
| TGA | 207-209 |
| TGA | 264-266 |
| TGA | 249-251 |
| TGA | 258-260 |
| TGA | 309-311 |
| TAA | 321-323 |
| TGA | 378-380 |
| TAA | 471-473 |
| TAG | 420-422 |
| TAA | 432-434 |
| TGA | 603-605 |
| TAA | 507-509 |
| TGA | 612-614 |
| TAA | 816-818 |
| TAA | 654-656 |
| TGA | 708-710 |
| TGA | 855-857 |
| TGA | 1008-1010 |
| TAA | 918-920 |
| TAG | 1014-1016 |
| TAG | 1056-1058 |
| TGA | 1032-1034 |
| TAG | 1137-1139 |
| TGA | 1101-1103 |
| TGA | 1143-1145 |
| TAA | 1236-1238 |
| TGA | 1176-1178 |
| TAG | 1200-1202 |
| TGA | 1233-1235 |
| TAA | 1242-1244 |
| TAG | 1296-1298 |
| TAG | 1344-1346 |
| TAA | 1332-1334 |
| TAA | 1365-1367 |
| TAG | 1374-1376 |
| TAG | 1404-1406 |
| TAG | 1395-1397 |
| TAA | 1437-1439 |
| TAG | 1473-1475 |
| TGA | 1443-1445 |
| TAG | 1470-1472 |
| TAA | 1539-1541 |
| TAA | 1548-1550 |
| TAA | 1560-1562 |
| TAG | 1566-1568 |
| TAA | 1593-1595 |
| TAA | 1623-1625 |
| TGA | 1710-1712 |
| TAG | 1647-1649 |
| TAA | 1713-1715 |
| TGA | 1752-1754 |
| TGA | 1755-1757 |
| TAG | 1830-1832 |
| TAA | 1878-1880 |
| TAA | 1890-1892 |
| TAA | 1911-1913 |
| TGA | 1950-1952 |
| TAA | 1926-1928 |
| TAG | 1992-1994 |
| TAG | 1995-1997 |
| TAA | 2010-2012 |
| TAA | 2067-2069 |
| TGA | 2025-2027 |
| TAA | 2067-2069 |
| TGA | 2217-2219 |
| TAA | 2082-2084 |
| TGA | 2217-2219 |
| TAA | 2223-2225 |
| TAG | 2322-2324 |
| TAA | 2256-2258 |
| TAG | 2322-2324 |
| TAA | 2373-2375 |
| TAG | 2409-2411 |
| TAG | 2490-2492 |
| TAG | 2448-2450 |
| TAG | 2490-2492 |
| TGA | 2523-2525 |
| TAA | 2559-2561 |
| TAG | 2652-2654 |
| TAA | 2793-2795 |
| TAA | 2709-2711 |
| TAA | 2793-2795 |
| TAA | 2736-2738 |
| TAA | 2793-2795 |
| TAG | 3114-3116 |
| TGA | 2949-2951 |
| TAG | 3114-3116 |
| TGA | 3099-3101 |
| TAG | 3114-3116 |
| TGA | 3186-3188 |
| TAA | 3138-3140 |
| TGA | 3186-3188 |
| TAG | 3258-3260 |
| TAA | 3240-3242 |
| TAG | 3258-3260 |
| TAG | 3300-3302 |
| TAG | 3333-3335 |
| TGA | 3357-3359 |
| TAG | 3375-3377 |
| TAA | 3441-3443 |
| TAA | 3399-3401 |
| TAA | 3441-3443 |
| TGA | 3426-3428 |
| TAA | 3441-3443 |
| TAG | 3465-3467 |
| TAG | 3456-3458 |
| TAG | 3465-3467 |
| TGA | 3501-3503 |
| TAG | 3516-3518 |
| TAG | 3507-3509 |
| TAG | 3516-3518 |
| TAG | 3579-3581 |
| TAA | 3594-3596 |
| TAG | 3744-3746 |
| TAA | 3819-3821 |
| TAG | 3753-3755 |
| TAA | 3819-3821 |
| TGA | 3906-3908 |
| TAA | 3918-3920 |
| TAA | 3939-3941 |
| TGA | 3927-3929 |
| TAA | 3939-3941 |
| TGA | 4035-4037 |
| TGA | 4017-4019 |
| TGA | 4035-4037 |
| TGA | 4068-4070 |
| TGA | 4089-4091 |
| TGA | 4122-4124 |
| TAG | 4212-4214 |
| TAG | 4143-4145 |
| TAG | 4212-4214 |
| TAA | 4206-4208 |
| TAG | 4212-4214 |
| TAA | 4293-4295 |
| TAG | 4266-4268 |
| TAA | 4293-4295 |
| TGA | 4329-4331 |
| TAA | 4332-4334 |
| TAG | 4359-4361 |
| TAG | 4371-4373 |
| TAA | 4416-4418 |
| TAA | 4482-4484 |
| TAG | 4467-4469 |
| TAA | 4482-4484 |
| TAA | 4512-4514 |
| TAG | 4629-4631 |
| TGA | 4758-4760 |
| TAA | 4644-4646 |
| TGA | 4758-4760 |
| TAG | 4791-4793 |
| TGA | 4917-4919 |
| TAG | 4878-4880 |
| TGA | 4917-4919 |
| TAA | 4905-4907 |
| TGA | 4917-4919 |
| TGA | 4932-4934 |
| TGA | 5013-5015 |
| TAA | 4992-4994 |
| TGA | 5013-5015 |
| TGA | 5034-5036 |
| TGA | 5088-5090 |
| TAA | 5043-5045 |
| TGA | 5088-5090 |
| TAA | 5145-5147 |
| TAA | 5115-5117 |
| TAA | 5145-5147 |
| TAA | 5154-5156 |
| TGA | 5184-5186 |
| TGA | 5220-5222 |
| TAG | 5232-5234 |
| TAG | 5256-5258 |
| TGA | 5274-5276 |
| TGA | 5304-5306 |
| TAG | 5409-5411 |
| TGA | 5493-5495 |
| TAG | 5424-5426 |
| TGA | 5463-5465 |
| TGA | 5616-5618 |
| TGA | 5562-5564 |
| TGA | 5616-5618 |
| TAG | 5643-5645 |
| TGA | 5679-5681 |

Two base deletions of the present invention form stop codons as indicated in the following TABLE 5. The formed in-frame stop codon and its location are provided. Any expressed protein from BRCA1 genes with these types of mutations should be truncated accordingly. It should be recognized that the present invention includes deletions of 3n+2 bases, where n is an integer greater than zero and less than 1862. These larger deletions mutations have stop codons at nucleotide numbers corresponding to the listed stop codon at the nucleotide number listed. The corresponding nucleotide numbers of the stop codons will be 3n nucleotides smaller than those listed.

TABLE 5
Two Base Deletions

| Codon Formed | Nucleotide Number |
|---|---|
| TGA | 141-143 |
| TAA | 162-164 |
| TGA | 234-236 |
| TGA | 183-185 |
| TGA | 234-236 |
| TAA | 309-311 |
| TGA | 249-251 |
| TGA | 309-311 |
| TGA | 315-317 |
| TAG | 354-356 |
| TGA | 366-368 |
| TGA | 402-404 |
| TAA | 378-380 |
| TGA | 402-404 |
| TAA | 432-434 |
| TGA | 414-416 |
| TAA | 432-434 |
| TAA | 453-455 |
| TGA | 462-464 |
| TGA | 477-479 |
| TGA | 537-539 |
| TAG | 507-509 |
| TGA | 537-539 |
| TAA | 588-590 |
| TGA | 657-659 |
| TGA | 669-671 |
| TGA | 678-680 |
| TAA | 690-692 |
| TAA | 693-695 |
| TGA | 759-761 |
| TAG | 705-707 |
| TGA | 759-761 |
| TAG | 708-710 |
| TGA | 759-761 |
| TGA | 795-797 |
| TGA | 771-773 |
| TGA | 795-797 |
| TGA | 804-806 |
| TGA | 825-827 |
| TAA | 843-845 |
| TAA | 846-848 |
| TGA | 849-851 |
| TGA | 864-866 |
| TAA | 855-857 |
| TGA | 864-866 |
| TGA | 879-881 |
| TAG | 906-908 |
| TAA | 900-902 |
| TAG | 906-908 |
| TGA | 972-974 |
| TAA | 918-920 |
| TGA | 972-974 |
| TAA | 996-998 |
| TGA | 1023-1025 |
| TGA | 1032-1034 |
| TAA | 1035-1037 |
| TAA | 1071-1073 |
| TAA | 1089-1091 |
| TGA | 1101-1103 |
| TGA | 1104-1106 |
| TAG | 1107-1109 |
| TGA | 1149-1151 |
| TGA | 1161-1163 |
| TAA | 1179-1181 |
| TGA | 1176-1178 |
| TAA | 1179-1181 |
| TAG | 1209-1211 |
| TGA | 1200-1202 |
| TAG | 1209-1211 |
| TGA | 1218-1220 |
| TAG | 1245-1247 |
| TAA | 1263-1265 |
| TGA | 1266-1268 |
| TGA | 1284-1286 |
| TAG | 1278-1280 |
| TGA | 1284-1286 |
| TGA | 1287-1289 |
| TGA | 1302-1304 |
| TGA | 1305-1307 |
| TGA | 1314-1316 |
| TGA | 1326-1328 |
| TGA | 1347-1349 |
| TAA | 1332-1334 |
| TGA | 1347-1349 |
| TGA | 1368-1370 |
| TGA | 1356-1358 |
| TGA | 1368-1370 |
| TGA | 1377-1379 |
| TGA | 1419-1421 |
| TGA | 1395-1397 |
| TGA | 1419-1421 |
| TGA | 1428-1430 |
| TGA | 1443-1445 |
| TGA | 1449-1451 |
| TAA | 1479-1481 |
| TAA | 1464-1466 |
| TAA | 1479-1481 |
| TGA | 1485-1487 |
| TGA | 1551-1553 |
| TAG | 1512-1514 |
| TGA | 1551-1553 |
| TAG | 1539-1541 |
| TGA | 1551-1553 |
| TGA | 1581-1583 |
| TAA | 1617-1619 |
| TAA | 1629-1631 |
| TAA | 1623-1625 |
| TAA | 1629-1631 |
| TGA | 1659-1661 |
| TGA | 1704-1706 |
| TAA | 1725-1727 |
| TAA | 1764-1766 |
| TAG | 1767-1769 |
| TGA | 1776-1778 |
| TAA | 1782-1784 |
| TGA | 1794-1796 |
| TGA | 1809-1811 |
| TAA | 1821-1823 |
| TGA | 1869-1871 |
| TAA | 1857-1859 |
| TGA | 1869-1871 |
| TAA | 1935-1937 |
| TAA | 1911-1913 |
| TAA | 1935-1937 |
| TAA | 1926-1928 |
| TAA | 1935-1937 |
| TAG | 1944-1946 |
| TGA | 1986-1988 |
| TAG | 2001-2003 |
| TAA | 2019-2021 |
| TGA | 2028-2030 |
| TGA | 2040-2042 |
| TAG | 2043-2045 |
| TAG | 2052-2054 |
| TGA | 2058-2060 |
| TAA | 2130-2132 |
| TAA | 2082-2084 |
| TAA | 2130-2132 |
| TAA | 2160-2162 |
| TGA | 2172-2174 |
| TAA | 2184-2186 |
| TGA | 2193-2195 |
| TGA | 2199-2201 |
| TAA | 2247-2249 |
| TGA | 2265-2267 |
| TAA | 2256-2258 |
| TGA | 2265-2267 |
| TAA | 2271-2273 |
| TAG | 2289-2291 |
| TAA | 2331-2333 |
| TAA | 2340-2342 |
| TAA | 2343-2345 |
| TGA | 2349-2351 |
| TGA | 2397-2399 |
| TAG | 2373-2375 |
| TGA | 2397-2399 |
| TAG | 2415-2417 |
| TGA | 2442-2444 |
| TAG | 2481-2483 |
| TAG | 2448-2450 |
| TAG | 2481-2483 |
| TAA | 2514-2516 |
| TGA | 2541-2543 |
| TAA | 2580-2582 |
| TAA | 2574-2576 |
| TAA | 2580-2582 |
| TAG | 2583-2585 |
| TGA | 2589-2591 |
| TAA | 2604-2606 |
| TGA | 2622-2624 |
| TAA | 2628-2630 |
| TGA | 2667-2669 |
| TGA | 2673-2675 |
| TGA | 2820-2822 |
| TAA | 2700-2702 |
| TGA | 2820-2822 |
| TAA | 2709-2711 |
| TGA | 2820-2822 |
| TAA | 2736-2738 |
| TGA | 2820-2822 |
| TAA | 2793-2795 |
| TGA | 2820-2822 |
| TGA | 2826-2828 |
| TGA | 2856-2858 |
| TAA | 2862-2864 |
| TAA | 2886-2888 |
| TAA | 2925-2927 |
| TGA | 2934-2936 |
| TAA | 2937-2939 |
| TAG | 2949-2951 |
| TAG | 2967-2969 |
| TAA | 3024-3026 |
| TAG | 2991-2993 |
| TAA | 3024-3026 |
| TAA | 3087-3089 |
| TAG | 3051-3053 |
| TAA | 3087-3089 |
| TAA | 3093-3095 |
| TGA | 3099-3101 |
| TGA | 3126-3128 |
| TGA | 3147-3149 |
| TGA | 3165-3167 |
| TAG | 3195-3197 |
| TAA | 3201-3203 |
| TAA | 3204-3206 |
| TAG | 3210-3212 |
| TAA | 3225-3227 |
| TAA | 3249-3251 |
| TAG | 3240-3242 |
| TAA | 3249-3251 |
| TGA | 3252-3254 |
| TAA | 3270-3272 |
| TAG | 3264-3266 |
| TAA | 3270-3272 |
| TGA | 3273-3275 |
| TAA | 3291-3293 |
| TAG | 3285-3287 |
| TAA | 3291-3293 |
| TGA | 3294-3296 |
| TGA | 3309-3311 |
| TAG | 3306-3308 |
| TGA | 3309-3311 |
| TGA | 3312-3314 |
| TAG | 3336-3338 |
| TAG | 3369-3371 |
| TAA | 3357-3359 |
| TAG | 3369-3371 |
| TGA | 3390-3392 |
| TAA | 3399-3401 |
| TAA | 3420-3422 |
| TGA | 3426-3428 |
| TGA | 3435-3437 |
| TAA | 3456-3458 |
| TAA | 3477-3479 |
| TAA | 3510-3512 |
| TGA | 3507-3509 |
| TAA | 3510-3512 |
| TAG | 3534-3536 |
| TGA | 3516-3518 |
| TAG | 3534-3536 |
| TGA | 3558-3560 |
| TGA | 3567-3569 |
| TGA | 3570-3572 |
| TGA | 3582-3584 |
| TGA | 3579-3581 |
| TGA | 3582-3584 |
| TGA | 3588-3590 |
| TAG | 3606-3608 |
| TGA | 3615-3617 |
| TGA | 3621-3623 |
| TAA | 3627-3629 |
| TAG | 3648-3650 |
| TAG | 3675-3677 |
| TAG | 3687-3689 |
| TAG | 3768-3770 |
| TAG | 3723-3725 |
| TAG | 3768-3770 |
| TGA | 3744-3746 |
| TAG | 3768-3770 |
| TGA | 3753-3755 |
| TAG | 3768-3770 |
| TGA | 3771-3773 |
| TGA | 3777-3779 |
| TAA | 3813-3815 |
| TAG | 3843-3845 |
| TAG | 3849-3851 |
| TAA | 3876-3878 |
| TAG | 3912-3914 |
| TAA | 3906-3908 |
| TAG | 3912-3914 |
| TGA | 3921-3923 |
| TAA | 3918-3920 |
| TGA | 3921-3923 |
| TAA | 3930-3932 |
| TAG | 3927-3929 |
| TAA | 3930-3932 |
| TAG | 3972-3974 |
| TGA | 3975-3977 |
| TAG | 3996-3998 |
| TGA | 4020-4022 |
| TAG | 4017-4019 |
| TGA | 4020-4022 |
| TGA | 4101-4103 |
| TGA | 4026-4028 |
| TGA | 4101-4103 |
| TAA | 4080-4082 |
| TGA | 4101-4103 |
| TGA | 4125-4127 |
| TGA | 4146-4148 |
| TGA | 4143-4145 |
| TGA | 4146-4148 |
| TGA | 4149-4151 |
| TAA | 4179-4181 |
| TGA | 4170-4172 |
| TAA | 4179-4181 |
| TGA | 4215-4217 |
| TAA | 4206-4208 |
| TGA | 4215-4217 |
| TGA | 4233-4235 |
| TGA | 4239-4241 |
| TGA | 4254-4256 |
| TGA | 4284-4286 |
| TAA | 4323-4325 |
| TGA | 4353-4355 |
| TAA | 4395-4397 |
| TGA | 4371-4373 |
| TAA | 4395-4397 |
| TGA | 4419-4421 |
| TGA | 4434-4436 |
| TAG | 4497-4499 |
| TGA | 4467-4469 |
| TAG | 4497-4499 |
| TGA | 4500-4502 |
| TGA | 4539-4541 |
| TGA | 4548-4550 |
| TAG | 4563-4565 |
| TAA | 4575-4577 |
| TAA | 4581-4583 |
| TAA | 4614-4616 |
| TGA | 4632-4634 |
| TGA | 4629-4631 |
| TGA | 4632-4634 |
| TAG | 4635-4637 |
| TAG | 4674-4676 |
| TGA | 4641-4643 |
| TAG | 4674-4676 |
| TAA | 4704-4706 |
| TGA | 4713-4715 |
| TGA | 4833-4835 |
| TGA | 4836-4838 |
| TGA | 4842-4844 |
| TGA | 4848-4850 |
| TGA | 4857-4859 |
| TGA | 4977-4979 |
| TAA | 4917-4919 |
| TGA | 4977-4979 |
| TAA | 4932-4934 |
| TGA | 4977-4979 |
| TAA | 4992-4994 |
| TAA | 5148-5150 |
| TAA | 5115-5117 |
| TAA | 5148-5150 |
| TGA | 5160-5162 |
| TGA | 5196-5198 |
| TGA | 5208-5210 |
| TAG | 5259-5261 |
| TAA | 5286-5288 |
| TGA | 5307-5309 |
| TGA | 5313-5315 |
| TGA | 5319-5321 |
| TGA | 5601-5603 |
| TAG | 5400-5402 |
| TGA | 5601-5603 |
| TGA | 5421-5423 |
| TGA | 5601-5603 |
| TAG | 5424-5426 |
| TGA | 5601-5603 |
| TGA | 5634-5636 |
| TAA | 5652-5654 |
| TGA | 5658-5660 |
| TAG | 5706-5708 |

Deletion mutations in the BRCA1 gene containing a truncating mutation may also be defined as having the sequence 5′ R1-R2 3′; where R1 is the wild type BRCA1 DNA sequence from nucleotide number 120 to X; R2 contains the wild type BRCA1 DNA sequence from nucleotide number X+Y+1 to 5571, where Y=3n+1 or 3n+2 where n is an integer of zero or greater; and where X=123 to 5707.

Alternatively, the mutations may be defined as being specifically hybridizable to an oligonucleotide probe being at least 12 nucleotides in length and having the sequence 5′ R1-R2 3′; where R1 contains at its 3′ end three nucleotides complementary to nucleotide numbers X-2, X-1 and X the wild-type BRCA1 gene; R2 contains at its 5′ end three nucleotides complementary to nucleotide numbers X+Y+1, X+Y+2, and X+Y+3 of the wild type BRCA1 DNA sequence; where Y=3n+1 or 3n+2 where n is an integer of zero or greater; and where X=122 to 5706.

Single base insertions of the present invention form stop codons as indicated in the following TABLE 6. The formed in-frame stop codon and the location are provided. Any expressed protein from BRCA1 genes with these types of mutations should be truncated accordingly. It should be recognized that the present invention includes insertions of 3n+1 bases, where n is an integer greater than zero and less than 1861. These larger insertions mutations have stop codons at nucleotide numbers corresponding to the listed stop codon at the nucleotide number listed. The corresponding nucleotide numbers of the stop codons will be 3n nucleotides larger than those listed.

TABLE 6
Single Base Insertions

| Codon Formed | Nucleotide Number |
|---|---|
| TGA | 144-146 |
| TGA | 123-125 |
| TGA | 144-146 |
| TAA | 165-167 |
| TGA | 144-146 |
| TAA | 165-167 |
| TGA | 147-149 |
| TAA | 165-167 |
| TAA | 156-158 |
| TAA | 165-167 |
| TGA | 237-239 |
| TAA | 165-167 |
| TGA | 237-239 |
| TAA | 177-179 |
| TGA | 237-239 |
| TGA | 186-188 |
| TGA | 237-239 |
| TAG | 189-191 |
| TGA | 237-239 |
| TGA | 189-191 |
| TGA | 237-239 |
| TAG | 198-200 |
| TGA | 237-239 |
| TGA | 198-200 |
| TGA | 237-239 |
| TGA | 204-206 |
| TGA | 237-239 |
| TAA | 213-215 |
| TGA | 237-239 |
| TGA | 216-218 |
| TGA | 237-239 |
| TAA | 231-233 |
| TGA | 237-239 |
| TAG | 234-236 |
| TGA | 237-239 |
| TGA | 234-236 |
| TGA | 237-239 |
| TAA | 312-314 |
| TGA | 237-239 |
| TAA | 312-314 |
| TAG | 249-251 |
| TAA | 312-314 |
| TGA | 249-251 |
| TAA | 312-314 |
| TAA | 252-254 |
| TAA | 312-314 |
| TAG | 258-260 |
| TAA | 312-314 |
| TGA | 258-260 |
| TAA | 312-314 |
| TAA | 267-269 |
| TAA | 312-314 |
| TAA | 276-278 |
| TAA | 312-314 |
| TAA | 282-284 |
| TAA | 312-314 |
| TAA | 285-287 |
| TAA | 312-314 |
| TAG | 300-302 |
| TAA | 312-314 |
| TGA | 300-302 |
| TAA | 312-314 |
| TAG | 309-311 |
| TAA | 312-314 |
| TGA | 309-311 |
| TAA | 312-314 |
| TGA | 318-320 |
| TAA | 312-314 |
| TGA | 318-320 |
| TAA | 315-317 |
| TGA | 318-320 |
| TAG | 357-359 |
| TGA | 318-320 |
| TAG | 357-359 |
| TAA | 327-329 |
| TAG | 357-359 |
| TAG | 330-332 |
| TAG | 357-359 |
| TAG | 333-335 |
| TAG | 357-359 |
| TGA | 342-344 |
| TAG | 357-359 |
| TAG | 345-347 |
| TAG | 357-359 |
| TAG | 351-353 |
| TAG | 357-359 |
| TGA | 369-371 |
| TAG | 357-359 |
| TGA | 369-371 |
| TGA | 405-407 |
| TGA | 369-371 |
| TGA | 405-407 |
| TGA | 372-374 |
| TGA | 405-407 |
| TAA | 381-383 |
| TGA | 405-407 |
| TAG | 390-392 |
| TGA | 405-407 |
| TGA | 390-392 |
| TGA | 405-407 |
| TAA | 435-437 |
| TGA | 405-407 |
| TAA | 435-437 |
| TGA | 417-419 |
| TAA | 435-437 |
| TAA | 420-422 |
| TAA | 435-437 |
| TGA | 420-422 |
| TAA | 435-437 |
| TAA | 420-422 |
| TAA | 435-437 |
| TAG | 420-422 |
| TAA | 435-437 |
| TAA | 426-428 |
| TAA | 435-437 |
| TAG | 429-431 |
| TAA | 435-437 |
| TAA | 432-434 |
| TAA | 435-437 |
| TGA | 432-434 |
| TAA | 435-437 |
| TAA | 432-434 |
| TAA | 435-437 |
| TAG | 432-434 |
| TAA | 435-437 |
| TAA | 456-458 |
| TAA | 435-437 |
| TAA | 456-458 |
| TAA | 444-446 |
| TAA | 456-458 |
| TAA | 447-449 |
| TAA | 456-458 |
| TGA | 450-452 |
| TAA | 456-458 |
| TAA | 453-455 |
| TAA | 456-458 |
| TGA | 465-467 |
| TAA | 456-458 |
| TGA | 465-467 |
| TGA | 480-482 |
| TGA | 465-467 |
| TGA | 480-482 |
| TAA | 474-476 |
| TGA | 480-482 |
| TGA | 477-479 |
| TGA | 480-482 |
| TGA | 540-542 |
| TGA | 480-482 |
| TGA | 540-542 |
| TAG | 498-500 |
| TGA | 540-542 |
| TAA | 507-509 |
| TGA | 540-542 |
| TGA | 507-509 |
| TGA | 540-542 |
| TAA | 507-509 |
| TGA | 540-542 |
| TAG | 507-509 |
| TGA | 540-542 |
| TAG | 510-512 |
| TGA | 540-542 |
| TAA | 513-515 |
| TGA | 540-542 |
| TAA | 522-524 |
| TGA | 540-542 |
| TAG | 525-527 |
| TGA | 540-542 |
| TAG | 537-539 |
| TGA | 540-542 |
| TAA | 591-593 |
| TGA | 540-542 |
| TAA | 591-593 |
| TGA | 546-548 |
| TAA | 591-593 |
| TAA | 549-551 |
| TAA | 591-593 |
| TGA | 564-566 |
| TAA | 591-593 |
| TAG | 570-572 |
| TAA | 591-593 |
| TAG | 576-578 |
| TAA | 591-593 |
| TGA | 660-662 |
| TAA | 591-593 |
| TGA | 660-662 |
| TAG | 606-608 |
| TGA | 660-662 |
| TAG | 615-617 |
| TGA | 660-662 |
| TAA | 621-623 |
| TGA | 660-662 |
| TAA | 642-644 |
| TGA | 660-662 |
| TAA | 654-656 |
| TGA | 660-662 |
| TGA | 654-656 |
| TGA | 660-662 |
| TAA | 654-656 |
| TGA | 660-662 |
| TAG | 654-656 |
| TGA | 660-662 |
| TGA | 672-674 |
| TGA | 660-662 |
| TGA | 672-674 |
| TGA | 681-683 |
| TGA | 672-674 |
| TGA | 681-683 |
| TAA | 693-695 |
| TGA | 681-683 |
| TAA | 693-695 |
| TGA | 684-686 |
| TAA | 693-695 |
| TAA | 696-698 |
| TAA | 693-695 |
| TAA | 696-698 |
| TGA | 762-764 |
| TAA | 696-698 |
| TGA | 762-764 |
| TAA | 705-707 |
| TGA | 762-764 |
| TGA | 705-707 |
| TGA | 762-764 |
| TAA | 705-707 |
| TGA | 762-764 |
| TAG | 705-707 |
| TGA | 762-764 |
| TAG | 708-710 |
| TGA | 762-764 |
| TGA | 708-710 |
| TGA | 762-764 |
| TAG | 711-713 |
| TGA | 762-764 |
| TGA | 720-722 |
| TGA | 762-764 |
| TGA | 726-728 |
| TGA | 762-764 |
| TAG | 756-758 |
| TGA | 762-764 |
| TGA | 759-761 |
| TGA | 762-764 |
| TGA | 798-800 |
| TGA | 762-764 |
| TGA | 798-800 |
| TAG | 768-770 |
| TGA | 798-800 |
| TGA | 774-776 |
| TGA | 798-800 |
| TAA | 783-785 |
| TGA | 798-800 |
| TAA | 786-788 |
| TGA | 798-800 |
| TAG | 795-797 |
| TGA | 798-800 |
| TGA | 795-797 |
| TGA | 798-800 |
| TGA | 807-809 |
| TGA | 798-800 |
| TGA | 807-809 |
| TGA | 828-830 |
| TGA | 807-809 |
| TGA | 828-830 |
| TGA | 813-815 |
| TGA | 828-830 |
| TAA | 822-824 |
| TGA | 828-830 |
| TAA | 846-848 |
| TGA | 828-830 |
| TAA | 846-848 |
| TAG | 843-845 |
| TAA | 846-848 |
| TAA | 849-851 |
| TAA | 846-848 |
| TAA | 849-851 |
| TGA | 852-854 |
| TAA | 849-851 |
| TGA | 852-854 |
| TGA | 867-869 |
| TGA | 852-854 |
| TGA | 867-869 |
| TAA | 858-860 |
| TGA | 867-869 |
| TGA | 882-884 |
| TGA | 867-869 |
| TGA | 882-884 |
| TAA | 870-872 |
| TGA | 882-884 |
| TAG | 909-911 |
| TGA | 882-884 |
| TAG | 909-911 |
| TAG | 885-887 |
| TAG | 909-911 |
| TGA | 894-896 |
| TAG | 909-911 |
| TAA | 897-899 |
| TAG | 909-911 |
| TAA | 900-902 |
| TAG | 909-911 |
| TGA | 900-902 |
| TAG | 909-911 |
| TAA | 900-902 |
| TAG | 909-911 |
| TAG | 900-902 |
| TAG | 909-911 |
| TGA | 975-977 |
| TAG | 909-911 |
| TGA | 975-977 |
| TAA | 921-923 |
| TGA | 975-977 |
| TGA | 933-935 |
| TGA | 975-977 |
| TAG | 939-941 |
| TGA | 975-977 |
| TGA | 939-941 |
| TGA | 975-977 |
| TAA | 948-950 |
| TGA | 975-977 |
| TAG | 960-962 |
| TGA | 975-977 |
| TAA | 999-1001 |
| TGA | 975-977 |
| TAA | 999-1001 |
| TAA | 978-980 |
| TAA | 999-1001 |
| TAG | 981-983 |
| TAA | 999-1001 |
| TAG | 984-986 |
| TAA | 999-1001 |
| TGA | 1026-1028 |
| TAA | 999-1001 |
| TGA | 1026-1028 |
| TGA | 1002-1004 |
| TGA | 1026-1028 |
| TAG | 1005-1007 |
| TGA | 1026-1028 |
| TAA | 1011-1013 |
| TGA | 1026-1028 |
| TGA | 1017-1019 |
| TGA | 1026-1028 |
| TAA | 1020-1022 |
| TGA | 1026-1028 |
| TAA | 1035-1037 |
| TGA | 1026-1028 |
| TAA | 1035-1037 |
| TAG | 1032-1034 |
| TAA | 1035-1037 |
| TGA | 1032-1034 |
| TAA | 1035-1037 |
| TAA | 1038-1040 |
| TAA | 1035-1037 |
| TAA | 1038-1040 |
| TAA | 1074-1076 |
| TAA | 1038-1040 |
| TAA | 1074-1076 |
| TAG | 1041-1043 |
| TAA | 1074-1076 |
| TAA | 1044-1046 |
| TAA | 1074-1076 |
| TAG | 1062-1064 |
| TAA | 1074-1076 |
| TAG | 1065-1067 |
| TAA | 1074-1076 |
| TAA | 1092-1094 |
| TAA | 1074-1076 |
| TAA | 1092-1094 |
| TAG | 1077-1079 |
| TAA | 1092-1094 |
| TAG | 1080-1082 |
| TAA | 1092-1094 |
| TGA | 1080-1082 |
| TAA | 1092-1094 |
| TAG | 1089-1091 |
| TAA | 1092-1094 |
| TAA | 1104-1106 |
| TAA | 1092-1094 |
| TAA | 1104-1106 |
| TGA | 1095-1097 |
| TAA | 1104-1106 |
| TAG | 1101-1103 |
| TAA | 1104-1106 |
| TGA | 1101-1103 |
| TAA | 1104-1106 |
| TGA | 1107-1109 |
| TAA | 1104-1106 |
| TGA | 1107-1109 |
| TAG | 1110-1112 |
| TGA | 1107-1109 |
| TAG | 1110-1112 |
| TGA | 1152-1154 |
| TAG | 1110-1112 |
| TGA | 1152-1154 |
| TAG | 1122-1124 |
| TGA | 1152-1154 |
| TGA | 1128-1130 |
| TGA | 1152-1154 |
| TAA | 1131-1133 |
| TGA | 1152-1154 |
| TAA | 1134-1136 |
| TGA | 1152-1154 |
| TGA | 1140-1142 |
| TGA | 1152-1154 |
| TAA | 1146-1148 |
| TGA | 1152-1154 |
| TGA | 1164-1166 |
| TGA | 1152-1154 |
| TGA | 1164-1166 |
| TAG | 1161-1163 |
| TGA | 1164-1166 |
| TGA | 1161-1163 |
| TGA | 1164-1166 |
| TAA | 1182-1184 |
| TGA | 1164-1166 |
| TAA | 1182-1184 |
| TAG | 1167-1169 |
| TAA | 1182-1184 |
| TAA | 1170-1172 |
| TAA | 1182-1184 |
| TGA | 1173-1175 |
| TAA | 1182-1184 |
| TAG | 1176-1178 |
| TAA | 1182-1184 |
| TGA | 1176-1178 |
| TAA | 1182-1184 |
| TAA | 1179-1181 |
| TAA | 1182-1184 |
| TAG | 1212-1214 |
| TAA | 1182-1184 |
| TAG | 1212-1214 |
| TAA | 1188-1190 |
| TAG | 1212-1214 |
| TAG | 1197-1199 |
| TAG | 1212-1214 |
| TGA | 1197-1199 |
| TAG | 1212-1214 |
| TGA | 1203-1205 |
| TAG | 1212-1214 |
| TAA | 1206-1208 |
| TAG | 1212-1214 |
| TGA | 1221-1223 |
| TAG | 1212-1214 |
| TGA | 1221-1223 |
| TGA | 1215-1217 |
| TGA | 1221-1223 |
| TAG | 1248-1250 |
| TGA | 1221-1223 |
| TAG | 1248-1250 |
| TGA | 1224-1226 |
| TAG | 1248-1250 |
| TAG | 1233-1235 |
| TAG | 1248-1250 |
| TGA | 1233-1235 |
| TAG | 1248-1250 |
| TAA | 1245-1247 |
| TAG | 1248-1250 |
| TAA | 1266-1268 |
| TAG | 1248-1250 |
| TAA | 1266-1268 |
| TAG | 1251-1253 |
| TAA | 1266-1268 |
| TAA | 1260-1262 |
| TAA | 1266-1268 |
| TGA | 1269-1271 |
| TAA | 1266-1268 |
| TGA | 1269-1271 |
| TGA | 1287-1289 |
| TGA | 1269-1271 |
| TGA | 1287-1289 |
| TAG | 1272-1274 |
| TGA | 1287-1289 |
| TGA | 1272-1274 |
| TGA | 1287-1289 |
| TAG | 1281-1283 |
| TGA | 1287-1289 |
| TAG | 1284-1286 |
| TGA | 1287-1289 |
| TGA | 1290-1292 |
| TGA | 1287-1289 |
| TGA | 1290-1292 |
| TGA | 1305-1307 |
| TGA | 1290-1292 |
| TGA | 1305-1307 |
| TGA | 1308-1310 |
| TGA | 1305-1307 |
| TGA | 1308-1310 |
| TGA | 1317-1319 |
| TGA | 1308-1310 |
| TGA | 1317-1319 |
| TGA | 1329-1331 |
| TGA | 1317-1319 |
| TGA | 1329-1331 |
| TGA | 1323-1325 |
| TGA | 1329-1331 |
| TGA | 1350-1352 |
| TGA | 1329-1331 |
| TGA | 1350-1352 |
| TAA | 1335-1337 |
| TGA | 1350-1352 |
| TAA | 1341-1343 |
| TGA | 1350-1352 |
| TGA | 1371-1373 |
| TGA | 1350-1352 |
| TGA | 1371-1373 |
| TGA | 1359-1361 |
| TGA | 1371-1373 |
| TAA | 1368-1370 |
| TGA | 1371-1373 |
| TGA | 1380-1382 |
| TGA | 1371-1373 |
| TGA | 1380-1382 |
| TGA | 1377-1379 |
| TGA | 1380-1382 |
| TGA | 1422-1424 |
| TGA | 1380-1382 |
| TGA | 1422-1424 |
| TAA | 1383-1385 |
| TGA | 1422-1424 |
| TGA | 1383-1385 |
| TGA | 1422-1424 |
| TAA | 1383-1385 |
| TGA | 1422-1424 |
| TAG | 1383-1385 |
| TGA | 1422-1424 |
| TGA | 1398-1400 |
| TGA | 1422-1424 |
| TAA | 1401-1403 |
| TGA | 1422-1424 |
| TGA | 1407-1409 |
| TGA | 1422-1424 |
| TAG | 1419-1421 |
| TGA | 1422-1424 |
| TGA | 1431-1433 |
| TGA | 1422-1424 |
| TGA | 1431-1433 |
| TAA | 1446-1448 |
| TGA | 1431-1433 |
| TAA | 1446-1448 |
| TAG | 1443-1445 |
| TAA | 1446-1448 |
| TGA | 1443-1445 |
| TAA | 1446-1448 |
| TGA | 1452-1454 |
| TAA | 1446-1448 |
| TGA | 1452-1454 |
| TAG | 1449-1451 |
| TGA | 1452-1454 |
| TAA | 1482-1484 |
| TGA | 1452-1454 |
| TAA | 1482-1484 |
| TAG | 1455-1457 |
| TAA | 1482-1484 |
| TAA | 1467-1469 |
| TAA | 1482-1484 |
| TGA | 1476-1478 |
| TAA | 1482-1484 |
| TAG | 1479-1481 |
| TAA | 1482-1484 |
| TGA | 1488-1490 |
| TAA | 1482-1484 |
| TGA | 1488-1490 |
| TGA | 1554-1556 |
| TGA | 1488-1490 |
| TGA | 1554-1556 |
| TGA | 1491-1493 |
| TGA | 1554-1556 |
| TAA | 1494-1496 |
| TGA | 1554-1556 |
| TAA | 1506-1508 |
| TGA | 1554-1556 |
| TAA | 1512-1514 |
| TGA | 1554-1556 |
| TGA | 1512-1514 |
| TGA | 1554-1556 |
| TAA | 1512-1514 |
| TGA | 1554-1556 |
| TAG | 1512-1514 |
| TGA | 1554-1556 |
| TAA | 1518-1520 |
| TGA | 1554-1556 |
| TAA | 1521-1523 |
| TGA | 1554-1556 |
| TAG | 1527-1529 |
| TGA | 1554-1556 |
| TAA | 1536-1538 |
| TGA | 1554-1556 |
| TAG | 1542-1544 |
| TGA | 1554-1556 |
| TGA | 1584-1586 |
| TGA | 1554-1556 |
| TGA | 1584-1586 |
| TAA | 1557-1559 |
| TGA | 1584-1586 |
| TAA | 1620-1622 |
| TGA | 1584-1586 |
| TAA | 1620-1622 |
| TGA | 1602-1604 |
| TAA | 1620-1622 |
| TAA | 1617-1619 |
| TAA | 1620-1622 |
| TAA | 1632-1634 |
| TAA | 1620-1622 |
| TAA | 1632-1634 |
| TAA | 1626-1628 |
| TAA | 1632-1634 |
| TGA | 1662-1664 |
| TAA | 1632-1634 |
| TGA | 1662-1664 |
| TAG | 1635-1637 |
| TGA | 1662-1664 |
| TAG | 1638-1640 |
| TGA | 1662-1664 |
| TGA | 1707-1709 |
| TGA | 1662-1664 |
| TGA | 1707-1709 |
| TGA | 1665-1667 |
| TGA | 1707-1709 |
| TAA | 1674-1676 |
| TGA | 1707-1709 |
| TAA | 1677-1679 |
| TGA | 1707-1709 |
| TGA | 1683-1685 |
| TGA | 1707-1709 |
| TAA | 1698-1700 |
| TGA | 1707-1709 |
| TAA | 1728-1730 |
| TGA | 1707-1709 |
| TAA | 1728-1730 |
| TAA | 1716-1718 |
| TAA | 1728-1730 |
| TAA | 1767-1769 |
| TAA | 1728-1730 |
| TAA | 1767-1769 |
| TGA | 1737-1739 |
| TAA | 1767-1769 |
| TAA | 1743-1745 |
| TAA | 1767-1769 |
| TAA | 1758-1760 |
| TAA | 1767-1769 |
| TAG | 1770-1772 |
| TAA | 1767-1769 |
| TAG | 1770-1772 |
| TGA | 1779-1781 |
| TAG | 1770-1772 |
| TGA | 1779-1781 |
| TAA | 1785-1787 |
| TGA | 1779-1781 |
| TAA | 1785-1787 |
| TAA | 1782-1784 |
| TAA | 1785-1787 |
| TGA | 1797-1799 |
| TAA | 1785-1787 |
| TGA | 1797-1799 |
| TAA | 1791-1793 |
| TGA | 1797-1799 |
| TGA | 1812-1814 |
| TGA | 1797-1799 |
| TGA | 1812-1814 |
| TAA | 1809-1811 |
| TGA | 1812-1814 |
| TAA | 1824-1826 |
| TGA | 1812-1814 |
| TAA | 1824-1826 |
| TAA | 1815-1817 |
| TAA | 1824-1826 |
| TAA | 1818-1820 |
| TAA | 1824-1826 |
| TGA | 1872-1874 |
| TAA | 1824-1826 |
| TGA | 1872-1874 |
| TGA | 1833-1835 |
| TGA | 1872-1874 |
| TGA | 1842-1844 |
| TGA | 1872-1874 |
| TAA | 1845-1847 |
| TGA | 1872-1874 |
| TGA | 1848-1850 |
| TGA | 1872-1874 |
| TAA | 1860-1862 |
| TGA | 1872-1874 |
| TAA | 1866-1868 |
| TGA | 1872-1874 |
| TAA | 1938-1940 |
| TGA | 1872-1874 |
| TAA | 1938-1940 |
| TAG | 1881-1883 |
| TAA | 1938-1940 |
| TAG | 1884-1886 |
| TAA | 1938-1940 |
| TAG | 1887-1889 |
| TAA | 1938-1940 |
| TAG | 1893-1895 |
| TAA | 1938-1940 |
| TAA | 1896-1898 |
| TAA | 1938-1940 |
| TGA | 1902-1904 |
| TAA | 1938-1940 |
| TGA | 1908-1910 |
| TAA | 1938-1940 |
| TAA | 1914-1916 |
| TAA | 1938-1940 |
| TAA | 1923-1925 |
| TAA | 1938-1940 |
| TAA | 1929-1931 |
| TAA | 1938-1940 |
| TAG | 1947-1949 |
| TAA | 1938-1940 |
| TAG | 1947-1949 |
| TAA | 1941-1943 |
| TAG | 1947-1949 |
| TAA | 1944-1946 |
| TAG | 1947-1949 |
| TGA | 1989-1991 |
| TAG | 1947-1949 |
| TGA | 1989-1991 |
| TAG | 1953-1955 |
| TGA | 1989-1991 |
| TAG | 1956-1958 |
| TGA | 1989-1991 |
| TAA | 1959-1961 |
| TGA | 1989-1991 |
| TAG | 1971-1973 |
| TGA | 1989-1991 |
| TAG | 2004-2006 |
| TGA | 1989-1991 |
| TAG | 2004-2006 |
| TAG | 2001-2003 |
| TAG | 2004-2006 |
| TAA | 2022-2024 |
| TAG | 2004-2006 |
| TAA | 2022-2024 |
| TAA | 2007-2009 |
| TAA | 2022-2024 |
| TAG | 2013-2015 |
| TAA | 2022-2024 |
| TGA | 2031-2033 |
| TAA | 2022-2024 |
| TGA | 2031-2033 |
| TAG | 2025-2027 |
| TGA | 2031-2033 |
| TGA | 2025-2027 |
| TGA | 2031-2033 |
| TGA | 2043-2045 |
| TGA | 2031-2033 |
| TGA | 2043-2045 |
| TAG | 2046-2048 |
| TGA | 2043-2045 |
| TAG | 2046-2048 |
| TAG | 2055-2057 |
| TAG | 2046-2048 |
| TAG | 2055-2057 |
| TAG | 2049-2051 |
| TAG | 2055-2057 |
| TGA | 2049-2051 |
| TAG | 2055-2057 |
| TGA | 2061-2063 |
| TAG | 2055-2057 |
| TGA | 2061-2063 |
| TAG | 2058-2060 |
| TGA | 2061-2063 |
| TAA | 2133-2135 |
| TGA | 2061-2063 |
| TAA | 2133-2135 |
| TGA | 2064-2066 |
| TAA | 2133-2135 |
| TAA | 2070-2072 |
| TAA | 2133-2135 |
| TAA | 2073-2075 |
| TAA | 2133-2135 |
| TAA | 2076-2078 |
| TAA | 2133-2135 |
| TAA | 2079-2081 |
| TAA | 2133-2135 |
| TAA | 2082-2084 |
| TAA | 2133-2135 |
| TGA | 2082-2084 |
| TAA | 2133-2135 |
| TAA | 2082-2084 |
| TAA | 2133-2135 |
| TAG | 2082-2084 |
| TAA | 2133-2135 |
| TAA | 2085-2087 |
| TAA | 2133-2135 |
| TAG | 2100-2102 |
| TAA | 2133-2135 |
| TAG | 2106-2108 |
| TAA | 2133-2135 |
| TAG | 2109-2111 |
| TAA | 2133-2135 |
| TAA | 2112-2114 |
| TAA | 2133-2135 |
| TGA | 2127-2129 |
| TAA | 2133-2135 |
| TAA | 2163-2165 |
| TAA | 2133-2135 |
| TAA | 2163-2165 |
| TGA | 2136-2138 |
| TAA | 2163-2165 |
| TAA | 2154-2156 |
| TAA | 2163-2165 |
| TAA | 2157-2159 |
| TAA | 2163-2165 |
| TAG | 2160-2162 |
| TAA | 2163-2165 |
| TGA | 2175-2177 |
| TAA | 2163-2165 |
| TGA | 2175-2177 |
| TAA | 2166-2168 |
| TGA | 2175-2177 |
| TAA | 2172-2174 |
| TGA | 2175-2177 |
| TAA | 2187-2189 |
| TGA | 2175-2177 |
| TAA | 2187-2189 |
| TAG | 2184-2186 |
| TAA | 2187-2189 |
| TGA | 2196-2198 |
| TAA | 2187-2189 |
| TGA | 2196-2198 |
| TAG | 2190-2192 |
| TGA | 2196-2198 |
| TGA | 2202-2204 |
| TGA | 2196-2198 |
| TGA | 2202-2204 |
| TAG | 2199-2201 |
| TGA | 2202-2204 |
| TAA | 2250-2252 |
| TGA | 2202-2204 |
| TAA | 2250-2252 |
| TGA | 2214-2216 |
| TAA | 2250-2252 |
| TAA | 2220-2222 |
| TAA | 2250-2252 |
| TAA | 2229-2231 |
| TAA | 2250-2252 |
| TGA | 2268-2270 |
| TAA | 2250-2252 |
| TGA | 2268-2270 |
| TAG | 2253-2255 |
| TGA | 2268-2270 |
| TGA | 2253-2255 |
| TGA | 2268-2270 |
| TAA | 2259-2261 |
| TGA | 2268-2270 |
| TAG | 2265-2267 |
| TGA | 2268-2270 |
| TAA | 2274-2276 |
| TGA | 2268-2270 |
| TAA | 2274-2276 |
| TAG | 2292-2294 |
| TAA | 2274-2276 |
| TAG | 2292-2294 |
| TGA | 2277-2279 |
| TAG | 2292-2294 |
| TAA | 2286-2288 |
| TAG | 2292-2294 |
| TAA | 2334-2336 |
| TAG | 2292-2294 |
| TAA | 2334-2336 |
| TAG | 2301-2303 |
| TAA | 2334-2336 |
| TGA | 2304-2306 |
| TAA | 2334-2336 |
| TGA | 2307-2309 |
| TAA | 2334-2336 |
| TAA | 2310-2312 |
| TAA | 2334-2336 |
| TGA | 2313-2315 |
| TAA | 2334-2336 |
| TGA | 2316-2318 |
| TAA | 2334-2336 |
| TAA | 2319-2321 |
| TAA | 2334-2336 |
| TGA | 2325-2327 |
| TAA | 2334-2336 |
| TAA | 2343-2345 |
| TAA | 2334-2336 |
| TAA | 2343-2345 |
| TAA | 2346-2348 |
| TAA | 2343-2345 |
| TAA | 2346-2348 |
| TGA | 2352-2354 |
| TAA | 2346-2348 |
| TGA | 2352-2354 |
| TGA | 2400-2402 |
| TGA | 2352-2354 |
| TGA | 2400-2402 |
| TGA | 2355-2357 |
| TGA | 2400-2402 |
| TAA | 2361-2363 |
| TGA | 2400-2402 |
| TGA | 2364-2366 |
| TGA | 2400-2402 |
| TAG | 2376-2378 |
| TGA | 2400-2402 |
| TGA | 2382-2384 |
| TGA | 2400-2402 |
| TAG | 2385-2387 |
| TGA | 2400-2402 |
| TAG | 2418-2420 |
| TGA | 2400-2402 |
| TAG | 2418-2420 |
| TAG | 2403-2405 |
| TAG | 2418-2420 |
| TGA | 2412-2414 |
| TAG | 2418-2420 |
| TAG | 2415-2417 |
| TAG | 2418-2420 |
| TGA | 2445-2447 |
| TAG | 2418-2420 |
| TGA | 2445-2447 |
| TAG | 2421-2423 |
| TGA | 2445-2447 |
| TAG | 2484-2486 |
| TGA | 2445-2447 |
| TAG | 2484-2486 |
| TAA | 2448-2450 |
| TAG | 2484-2486 |
| TGA | 2448-2450 |
| TAG | 2484-2486 |
| TAA | 2448-2450 |
| TAG | 2484-2486 |
| TAG | 2448-2450 |
| TAG | 2484-2486 |
| TGA | 2460-2462 |
| TAG | 2484-2486 |
| TAG | 2463-2465 |
| TAG | 2484-2486 |
| TGA | 2478-2480 |
| TAG | 2484-2486 |
| TAA | 2517-2519 |
| TAG | 2484-2486 |
| TAA | 2517-2519 |
| TAA | 2496-2498 |
| TAA | 2517-2519 |
| TAA | 2502-2504 |
| TAA | 2517-2519 |
| TGA | 2508-2510 |
| TAA | 2517-2519 |
| TAA | 2514-2516 |
| TAA | 2517-2519 |
| TGA | 2544-2546 |
| TAA | 2517-2519 |
| TGA | 2544-2546 |
| TAG | 2520-2522 |
| TGA | 2544-2546 |
| TGA | 2520-2522 |
| TGA | 2544-2546 |
| TAG | 2526-2528 |
| TGA | 2544-2546 |
| TAG | 2532-2534 |
| TGA | 2544-2546 |
| TGA | 2532-2534 |
| TGA | 2544-2546 |
| TAA | 2583-2585 |
| TGA | 2544-2546 |
| TAA | 2583-2585 |
| TAA | 2547-2549 |
| TAA | 2583-2585 |
| TAA | 2553-2555 |
| TAA | 2583-2585 |
| TAG | 2571-2573 |
| TAA | 2583-2585 |
| TGA | 2571-2573 |
| TAA | 2583-2585 |
| TAA | 2577-2579 |
| TAA | 2583-2585 |
| TGA | 2580-2582 |
| TAA | 2583-2585 |
| TAG | 2586-2588 |
| TAA | 2583-2585 |
| TAG | 2586-2588 |
| TGA | 2592-2594 |
| TAG | 2586-2588 |
| TGA | 2592-2594 |
| TAA | 2589-2591 |
| TGA | 2592-2594 |
| TAA | 2607-2609 |
| TGA | 2592-2594 |
| TAA | 2607-2609 |
| TGA | 2598-2600 |
| TAA | 2607-2609 |
| TGA | 2625-2627 |
| TAA | 2607-2609 |
| TGA | 2625-2627 |
| TAA | 2610-2612 |
| TGA | 2625-2627 |
| TGA | 2610-2612 |
| TGA | 2625-2627 |
| TAA | 2610-2612 |
| TGA | 2625-2627 |
| TAG | 2610-2612 |
| TGA | 2625-2627 |
| TAA | 2631-2633 |
| TGA | 2625-2627 |
| TAA | 2631-2633 |
| TGA | 2670-2672 |
| TAA | 2631-2633 |
| TGA | 2670-2672 |
| TAG | 2637-2639 |
| TGA | 2670-2672 |
| TGA | 2643-2645 |
| TGA | 2670-2672 |
| TAG | 2649-2651 |
| TGA | 2670-2672 |
| TGA | 2655-2657 |
| TGA | 2670-2672 |
| TGA | 2661-2663 |
| TGA | 2670-2672 |
| TGA | 2664-2666 |
| TGA | 2670-2672 |
| TAG | 2667-2669 |
| TGA | 2670-2672 |
| TGA | 2676-2678 |
| TGA | 2670-2672 |
| TGA | 2676-2678 |
| TGA | 2823-2825 |
| TGA | 2676-2678 |
| TGA | 2823-2825 |
| TAA | 2685-2687 |
| TGA | 2823-2825 |
| TGA | 2685-2687 |
| TGA | 2823-2825 |
| TAA | 2685-2687 |
| TGA | 2823-2825 |
| TAG | 2685-2687 |
| TGA | 2823-2825 |
| TAA | 2694-2696 |
| TGA | 2823-2825 |
| TAA | 2703-2705 |
| TGA | 2823-2825 |
| TAA | 2712-2714 |
| TGA | 2823-2825 |
| TAA | 2739-2741 |
| TGA | 2823-2825 |
| TAA | 2748-2750 |
| TGA | 2823-2825 |
| TGA | 2754-2756 |
| TGA | 2823-2825 |
| TGA | 2757-2759 |
| TGA | 2823-2825 |
| TGA | 2760-2762 |
| TGA | 2823-2825 |
| TAG | 2763-2765 |
| TGA | 2823-2825 |
| TGA | 2763-2765 |
| TGA | 2823-2825 |
| TAA | 2796-2798 |
| TGA | 2823-2825 |
| TAA | 2799-2801 |
| TGA | 2823-2825 |
| TAG | 2805-2807 |
| TGA | 2823-2825 |
| TAA | 2811-2813 |
| TGA | 2823-2825 |
| TGA | 2829-2831 |
| TGA | 2823-2825 |
| TGA | 2829-2831 |
| TAG | 2826-2828 |
| TGA | 2829-2831 |
| TGA | 2826-2828 |
| TGA | 2829-2831 |
| TGA | 2859-2861 |
| TGA | 2829-2831 |
| TGA | 2859-2861 |
| TAA | 2835-2837 |
| TGA | 2859-2861 |
| TGA | 2838-2840 |
| TGA | 2859-2861 |
| TGA | 2841-2843 |
| TGA | 2859-2861 |
| TAA | 2844-2846 |
| TGA | 2859-2861 |
| TAA | 2853-2855 |
| TGA | 2859-2861 |
| TAA | 2856-2858 |
| TGA | 2859-2861 |
| TAA | 2865-2867 |
| TGA | 2859-2861 |
| TAA | 2865-2867 |
| TAA | 2889-2891 |
| TAA | 2865-2867 |
| TAA | 2889-2891 |
| TAA | 2871-2873 |
| TAA | 2889-2891 |
| TAA | 2928-2930 |
| TAA | 2889-2891 |
| TAA | 2928-2930 |
| TAA | 2922-2924 |
| TAA | 2928-2930 |
| TGA | 2925-2927 |
| TAA | 2928-2930 |
| TGA | 2937-2939 |
| TAA | 2928-2930 |
| TGA | 2937-2939 |
| TAA | 2940-2942 |
| TGA | 2937-2939 |
| TAA | 2940-2942 |
| TAG | 2952-2954 |
| TAA | 2940-2942 |
| TAG | 2952-2954 |
| TAA | 2946-2948 |
| TAG | 2952-2954 |
| TAG | 2949-2951 |
| TAG | 2952-2954 |
| TGA | 2949-2951 |
| TAG | 2952-2954 |
| TAG | 2970-2972 |
| TAG | 2952-2954 |
| TAG | 2970-2972 |
| TAA | 2958-2960 |
| TAG | 2970-2972 |
| TAA | 3027-3029 |
| TAG | 2970-2972 |
| TAA | 3027-3029 |
| TAG | 2976-2978 |
| TAA | 3027-3029 |
| TGA | 2976-2978 |
| TAA | 3027-3029 |
| TAG | 2994-2996 |
| TAA | 3027-3029 |
| TAA | 3000-3002 |
| TAA | 3027-3029 |
| TGA | 3003-3005 |
| TAA | 3027-3029 |
| TAA | 3024-3026 |
| TAA | 3027-3029 |
| TAA | 3090-3092 |
| TAA | 3027-3029 |
| TAA | 3090-3092 |
| TAA | 3045-3047 |
| TAA | 3090-3092 |
| TAA | 3051-3053 |
| TAA | 3090-3092 |
| TGA | 3051-3053 |
| TAA | 3090-3092 |
| TAA | 3051-3053 |
| TAA | 3090-3092 |
| TAG | 3051-3053 |
| TAA | 3090-3092 |
| TAA | 3078-3080 |
| TAA | 3090-3092 |
| TAA | 3096-3098 |
| TAA | 3090-3092 |
| TAA | 3096-3098 |
| TAA | 3102-3104 |
| TAA | 3096-3098 |
| TAA | 3102-3104 |
| TAG | 3099-3101 |
| TAA | 3102-3104 |
| TGA | 3099-3101 |
| TAA | 3102-3104 |
| TGA | 3129-3131 |
| TAA | 3102-3104 |
| TGA | 3129-3131 |
| TAA | 3105-3107 |
| TGA | 3129-3131 |
| TAA | 3108-3110 |
| TGA | 3129-3131 |
| TGA | 3117-3119 |
| TGA | 3129-3131 |
| TGA | 3120-3122 |
| TGA | 3129-3131 |
| TAA | 3123-3125 |
| TGA | 3129-3131 |
| TGA | 3150-3152 |
| TGA | 3129-3131 |
| TGA | 3150-3152 |
| TGA | 3132-3134 |
| TGA | 3150-3152 |
| TGA | 3168-3170 |
| TGA | 3150-3152 |
| TGA | 3168-3170 |
| TAG | 3153-3155 |
| TGA | 3168-3170 |
| TGA | 3156-3158 |
| TGA | 3168-3170 |
| TAA | 3165-3167 |
| TGA | 3168-3170 |
| TAG | 3198-3200 |
| TGA | 3168-3170 |
| TAG | 3198-3200 |
| TAA | 3171-3173 |
| TAG | 3198-3200 |
| TAG | 3180-3182 |
| TAG | 3198-3200 |
| TAG | 3189-3191 |
| TAG | 3198-3200 |
| TAA | 3204-3206 |
| TAG | 3198-3200 |
| TAA | 3204-3206 |
| TAA | 3207-3209 |
| TAA | 3204-3206 |
| TAA | 3207-3209 |
| TAG | 3213-3215 |
| TAA | 3207-3209 |
| TAG | 3213-3215 |
| TAA | 3228-3230 |
| TAG | 3213-3215 |
| TAA | 3228-3230 |
| TGA | 3216-3218 |
| TAA | 3228-3230 |
| TAA | 3219-3221 |
| TAA | 3228-3230 |
| TAA | 3252-3254 |
| TAA | 3228-3230 |
| TAA | 3252-3254 |
| TAG | 3237-3239 |
| TAA | 3252-3254 |
| TAG | 3243-3245 |
| TAA | 3252-3254 |
| TAA | 3246-3248 |
| TAA | 3252-3254 |
| TGA | 3255-3257 |
| TAA | 3252-3254 |
| TGA | 3255-3257 |
| TAA | 3273-3275 |
| TGA | 3255-3257 |
| TAA | 3273-3275 |
| TAG | 3267-3269 |
| TAA | 3273-3275 |
| TGA | 3276-3278 |
| TAA | 3273-3275 |
| TGA | 3276-3278 |
| TAA | 3294-3296 |
| TGA | 3276-3278 |
| TAA | 3294-3296 |
| TAG | 3288-3290 |
| TAA | 3294-3296 |
| TGA | 3297-3299 |
| TAA | 3294-3296 |
| TGA | 3297-3299 |
| TGA | 3312-3314 |
| TGA | 3297-3299 |
| TGA | 3312-3314 |
| TAG | 3309-3311 |
| TGA | 3312-3314 |
| TGA | 3315-3317 |
| TGA | 3312-3314 |
| TGA | 3315-3317 |
| TAG | 3339-3341 |
| TGA | 3315-3317 |
| TAG | 3339-3341 |
| TAA | 3318-3320 |
| TAG | 3339-3341 |
| TGA | 3330-3332 |
| TAG | 3339-3341 |
| TAG | 3372-3374 |
| TAG | 3339-3341 |
| TAG | 3372-3374 |
| TAA | 3342-3344 |
| TAG | 3372-3374 |
| TAG | 3345-3347 |
| TAG | 3372-3374 |
| TAA | 3354-3356 |
| TAG | 3372-3374 |
| TAA | 3360-3362 |
| TAG | 3372-3374 |
| TGA | 3393-3395 |
| TAG | 3372-3374 |
| TGA | 3393-3395 |
| TAA | 3402-3404 |
| TGA | 3393-3395 |
| TAA | 3402-3404 |
| TAA | 3399-3401 |
| TAA | 3402-3404 |
| TGA | 3399-3401 |
| TAA | 3402-3404 |
| TAA | 3399-3401 |
| TAA | 3402-3404 |
| TAG | 3399-3401 |
| TAA | 3402-3404 |
| TAA | 3423-3425 |
| TAA | 3402-3404 |
| TAA | 3423-3425 |
| TAG | 3408-3410 |
| TAA | 3423-3425 |
| TAG | 3420-3422 |
| TAA | 3423-3425 |
| TAA | 3429-3431 |
| TAA | 3423-3425 |
| TAA | 3429-3431 |
| TAG | 3426-3428 |
| TAA | 3429-3431 |
| TGA | 3426-3428 |
| TAA | 3429-3431 |
| TGA | 3438-3440 |
| TAA | 3429-3431 |
| TGA | 3438-3440 |
| TGA | 3459-3461 |
| TGA | 3438-3440 |
| TGA | 3459-3461 |
| TAA | 3444-3446 |
| TGA | 3459-3461 |
| TAA | 3447-3449 |
| TGA | 3459-3461 |
| TGA | 3453-3455 |
| TGA | 3459-3461 |
| TAA | 3456-3458 |
| TGA | 3459-3461 |
| TGA | 3456-3458 |
| TGA | 3459-3461 |
| TAA | 3456-3458 |
| TGA | 3459-3461 |
| TAG | 3456-3458 |
| TGA | 3459-3461 |
| TAA | 3480-3482 |
| TGA | 3459-3461 |
| TAA | 3480-3482 |
| TGA | 3462-3464 |
| TAA | 3480-3482 |
| TAA | 3513-3515 |
| TAA | 3480-3482 |
| TAA | 3513-3515 |
| TGA | 3486-3488 |
| TAA | 3513-3515 |
| TAA | 3498-3500 |
| TAA | 3513-3515 |
| TGA | 3498-3500 |
| TAA | 3513-3515 |
| TAA | 3498-3500 |
| TAA | 3513-3515 |
| TAG | 3498-3500 |
| TAA | 3513-3515 |
| TGA | 3510-3512 |
| TAA | 3513-3515 |
| TAG | 3537-3539 |
| TAA | 3513-3515 |
| TAG | 3537-3539 |
| TGA | 3519-3521 |
| TAG | 3537-3539 |
| TAG | 3534-3536 |
| TAG | 3537-3539 |
| TGA | 3561-3563 |
| TAG | 3537-3539 |
| TGA | 3561-3563 |
| TAG | 3555-3557 |
| TGA | 3561-3563 |
| TGA | 3555-3557 |
| TGA | 3561-3563 |
| TGA | 3570-3572 |
| TGA | 3561-3563 |
| TGA | 3570-3572 |
| TGA | 3573-3575 |
| TGA | 3570-3572 |
| TGA | 3573-3575 |
| TGA | 3585-3587 |
| TGA | 3573-3575 |
| TGA | 3585-3587 |
| TGA | 3582-3584 |
| TGA | 3585-3587 |
| TGA | 3591-3593 |
| TGA | 3585-3587 |
| TGA | 3591-3593 |
| TAG | 3609-3611 |
| TGA | 3591-3593 |
| TAG | 3609-3611 |
| TAA | 3597-3599 |
| TAG | 3609-3611 |
| TGA | 3600-3602 |
| TAG | 3609-3611 |
| TGA | 3603-3605 |
| TAG | 3609-3611 |
| TGA | 3618-3620 |
| TAG | 3609-3611 |
| TGA | 3618-3620 |
| TGA | 3624-3626 |
| TGA | 3618-3620 |
| TGA | 3624-3626 |
| TAA | 3621-3623 |
| TGA | 3624-3626 |
| TAA | 3630-3632 |
| TGA | 3624-3626 |
| TAA | 3630-3632 |
| TAG | 3651-3653 |
| TAA | 3630-3632 |
| TAG | 3651-3653 |
| TGA | 3633-3635 |
| TAG | 3651-3653 |
| TAG | 3636-3638 |
| TAG | 3651-3653 |
| TAG | 3678-3680 |
| TAG | 3651-3653 |
| TAG | 3678-3680 |
| TAA | 3654-3656 |
| TAG | 3678-3680 |
| TAG | 3657-3659 |
| TAG | 3678-3680 |
| TAG | 3666-3668 |
| TAG | 3678-3680 |
| TGA | 3672-3674 |
| TAG | 3678-3680 |
| TAG | 3690-3692 |
| TAG | 3678-3680 |
| TAG | 3690-3692 |
| TAG | 3681-3683 |
| TAG | 3690-3692 |
| TAG | 3684-3686 |
| TAG | 3690-3692 |
| TAG | 3771-3773 |
| TAG | 3690-3692 |
| TAG | 3771-3773 |
| TAA | 3723-3725 |
| TAG | 3771-3773 |
| TGA | 3723-3725 |
| TAG | 3771-3773 |
| TAA | 3723-3725 |
| TAG | 3771-3773 |
| TAG | 3723-3725 |
| TAG | 3771-3773 |
| TAG | 3729-3731 |
| TAG | 3771-3773 |
| TAA | 3738-3740 |
| TAG | 3771-3773 |
| TAA | 3741-3743 |
| TAG | 3771-3773 |
| TGA | 3747-3749 |
| TAG | 3771-3773 |
| TGA | 3756-3758 |
| TAG | 3771-3773 |
| TGA | 3759-3761 |
| TAG | 3771-3773 |
| TAA | 3762-3764 |
| TAG | 3771-3773 |
| TGA | 3774-3776 |
| TAG | 3771-3773 |
| TGA | 3774-3776 |
| TGA | 3780-3782 |
| TGA | 3774-3776 |
| TGA | 3780-3782 |
| TGA | 3777-3779 |
| TGA | 3780-3782 |
| TAA | 3816-3818 |
| TGA | 3780-3782 |
| TAA | 3816-3818 |
| TGA | 3783-3785 |
| TAA | 3816-3818 |
| TAG | 3792-3794 |
| TAA | 3816-3818 |
| TGA | 3792-3794 |
| TAA | 3816-3818 |
| TAG | 3846-3848 |
| TAA | 3816-3818 |
| TAG | 3846-3848 |
| TAA | 3822-3824 |
| TAG | 3846-3848 |
| TAA | 3825-3827 |
| TAG | 3846-3848 |
| TAG | 3852-3854 |
| TAG | 3846-3848 |
| TAG | 3852-3854 |
| TAA | 3879-3881 |
| TAG | 3852-3854 |
| TAA | 3879-3881 |
| TGA | 3867-3869 |
| TAA | 3879-3881 |
| TAG | 3870-3872 |
| TAA | 3879-3881 |
| TGA | 3870-3872 |
| TAA | 3879-3881 |
| TAG | 3915-3917 |
| TAA | 3879-3881 |
| TAG | 3915-3917 |
| TAA | 3882-3884 |
| TAG | 3915-3917 |
| TGA | 3888-3890 |
| TAG | 3915-3917 |
| TGA | 3891-3893 |
| TAG | 3915-3917 |
| TAA | 3894-3896 |
| TAG | 3915-3917 |
| TAA | 3909-3911 |
| TAG | 3915-3917 |
| TAA | 3912-3914 |
| TAG | 3915-3917 |
| TGA | 3924-3926 |
| TAG | 3915-3917 |
| TGA | 3924-3926 |
| TAA | 3921-3923 |
| TGA | 3924-3926 |
| TAA | 3933-3935 |
| TGA | 3924-3926 |
| TAA | 3933-3935 |
| TAG | 3927-3929 |
| TAA | 3933-3935 |
| TGA | 3927-3929 |
| TAA | 3933-3935 |
| TAG | 3930-3932 |
| TAA | 3933-3935 |
| TAG | 3975-3977 |
| TAA | 3933-3935 |
| TAG | 3975-3977 |
| TAA | 3951-3953 |
| TAG | 3975-3977 |
| TGA | 3963-3965 |
| TAG | 3975-3977 |
| TGA | 3978-3980 |
| TAG | 3975-3977 |
| TGA | 3978-3980 |
| TAG | 3999-4001 |
| TGA | 3978-3980 |
| TAG | 3999-4001 |
| TGA | 3981-3983 |
| TAG | 3999-4001 |
| TAA | 3987-3989 |
| TAG | 3999-4001 |
| TAG | 3990-3992 |
| TAG | 3999-4001 |
| TGA | 3990-3992 |
| TAG | 3999-4001 |
| TGA | 4023-4025 |
| TAG | 3999-4001 |
| TGA | 4023-4025 |
| TAG | 4017-4019 |
| TGA | 4023-4025 |
| TGA | 4017-4019 |
| TGA | 4023-4025 |
| TAG | 4020-4022 |
| TGA | 4023-4025 |
| TGA | 4104-4106 |
| TGA | 4023-4025 |
| TGA | 4104-4106 |
| TGA | 4029-4031 |
| TGA | 4104-4106 |
| TGA | 4032-4034 |
| TGA | 4104-4106 |
| TAA | 4044-4046 |
| TGA | 4104-4106 |
| TAA | 4050-4052 |
| TGA | 4104-4106 |
| TGA | 4059-4061 |
| TGA | 4104-4106 |
| TAA | 4083-4085 |
| TGA | 4104-4106 |
| TAG | 4092-4094 |
| TGA | 4104-4106 |
| TGA | 4128-4130 |
| TGA | 4104-4106 |
| TGA | 4128-4130 |
| TAG | 4107-4109 |
| TGA | 4128-4130 |
| TAG | 4125-4127 |
| TGA | 4128-4130 |
| TGA | 4149-4151 |
| TGA | 4128-4130 |
| TGA | 4149-4151 |
| TAA | 4131-4133 |
| TGA | 4149-4151 |
| TGA | 4134-4136 |
| TGA | 4149-4151 |
| TGA | 4146-4148 |
| TGA | 4149-4151 |
| TGA | 4152-4154 |
| TGA | 4149-4151 |
| TGA | 4152-4154 |
| TAA | 4182-4184 |
| TGA | 4152-4154 |
| TAA | 4182-4184 |
| TGA | 4155-4157 |
| TAA | 4182-4184 |
| TAG | 4158-4160 |
| TAA | 4182-4184 |
| TGA | 4173-4175 |
| TAA | 4182-4184 |
| TGA | 4176-4178 |
| TAA | 4182-4184 |
| TAA | 4179-4181 |
| TAA | 4182-4184 |
| TGA | 4218-4220 |
| TAA | 4182-4184 |
| TGA | 4218-4220 |
| TGA | 4188-4190 |
| TGA | 4218-4220 |
| TGA | 4191-4193 |
| TGA | 4218-4220 |
| TAG | 4197-4199 |
| TGA | 4218-4220 |
| TGA | 4203-4205 |
| TGA | 4218-4220 |
| TAA | 4209-4211 |
| TGA | 4218-4220 |
| TGA | 4236-4238 |
| TGA | 4218-4220 |
| TGA | 4236-4238 |
| TAG | 4233-4235 |
| TGA | 4236-4238 |
| TGA | 4233-4235 |
| TGA | 4236-4238 |
| TGA | 4242-4244 |
| TGA | 4236-4238 |
| TGA | 4242-4244 |
| TAG | 4239-4241 |
| TGA | 4242-4244 |
| TGA | 4257-4259 |
| TGA | 4242-4244 |
| TGA | 4257-4259 |
| TAG | 4248-4250 |
| TGA | 4257-4259 |
| TGA | 4287-4289 |
| TGA | 4257-4259 |
| TGA | 4287-4289 |
| TGA | 4260-4262 |
| TGA | 4287-4289 |
| TAG | 4263-4265 |
| TGA | 4287-4289 |
| TGA | 4263-4265 |
| TGA | 4287-4289 |
| TAG | 4284-4286 |
| TGA | 4287-4289 |
| TAA | 4326-4328 |
| TGA | 4287-4289 |
| TAA | 4326-4328 |
| TAG | 4308-4310 |
| TAA | 4326-4328 |
| TGA | 4311-4313 |
| TAA | 4326-4328 |
| TGA | 4356-4358 |
| TAA | 4326-4328 |
| TGA | 4356-4358 |
| TAA | 4335-4337 |
| TGA | 4356-4358 |
| TGA | 4347-4349 |
| TGA | 4356-4358 |
| TAA | 4398-4400 |
| TGA | 4356-4358 |
| TAA | 4398-4400 |
| TGA | 4362-4364 |
| TAA | 4398-4400 |
| TGA | 4374-4376 |
| TAA | 4398-4400 |
| TAG | 4386-4388 |
| TAA | 4398-4400 |
| TGA | 4422-4424 |
| TAA | 4398-4400 |
| TGA | 4422-4424 |
| TAG | 4401-4403 |
| TGA | 4422-4424 |
| TAA | 4404-4406 |
| TGA | 4422-4424 |
| TGA | 4404-4406 |
| TGA | 4422-4424 |
| TAA | 4404-4406 |
| TGA | 4422-4424 |
| TAG | 4404-4406 |
| TGA | 4422-4424 |
| TAG | 4419-4421 |
| TGA | 4422-4424 |
| TGA | 4437-4439 |
| TGA | 4422-4424 |
| TGA | 4437-4439 |
| TAG | 4500-4502 |
| TGA | 4437-4439 |
| TAG | 4500-4502 |
| TGA | 4440-4442 |
| TAG | 4500-4502 |
| TAA | 4449-4451 |
| TAG | 4500-4502 |
| TGA | 4455-4457 |
| TAG | 4500-4502 |
| TAG | 4461-4463 |
| TAG | 4500-4502 |
| TGA | 4470-4472 |
| TAG | 4500-4502 |
| TAA | 4473-4475 |
| TAG | 4500-4502 |
| TAA | 4494-4496 |
| TAG | 4500-4502 |
| TAG | 4497-4499 |
| TAG | 4500-4502 |
| TGA | 4503-4505 |
| TAG | 4500-4502 |
| TGA | 4503-4505 |
| TGA | 4542-4544 |
| TGA | 4503-4505 |
| TGA | 4542-4544 |
| TAA | 4506-4508 |
| TGA | 4542-4544 |
| TGA | 4506-4508 |
| TGA | 4542-4544 |
| TAA | 4506-4508 |
| TGA | 4542-4544 |
| TAG | 4506-4508 |
| TGA | 4542-4544 |
| TAG | 4515-4517 |
| TGA | 4542-4544 |
| TAA | 4521-4523 |
| TGA | 4542-4544 |
| TGA | 4527-4529 |
| TGA | 4542-4544 |
| TGA | 4551-4553 |
| TGA | 4542-4544 |
| TGA | 4551-4553 |
| TAA | 4545-4547 |
| TGA | 4551-4553 |
| TAG | 4566-4568 |
| TGA | 4551-4553 |
| TAG | 4566-4568 |
| TGA | 4563-4565 |
| TAG | 4566-4568 |
| TAA | 4578-4580 |
| TAG | 4566-4568 |
| TAA | 4578-4580 |
| TAG | 4575-4577 |
| TAA | 4578-4580 |
| TAA | 4584-4586 |
| TAA | 4578-4580 |
| TAA | 4584-4586 |
| TAA | 4581-4583 |
| TAA | 4584-4586 |
| TAA | 4617-4619 |
| TAA | 4584-4586 |
| TAA | 4617-4619 |
| TGA | 4587-4589 |
| TAA | 4617-4619 |
| TGA | 4599-4601 |
| TAA | 4617-4619 |
| TAG | 4602-4604 |
| TAA | 4617-4619 |
| TGA | 4635-4637 |
| TAA | 4617-4619 |
| TGA | 4635-4637 |
| TAG | 4620-4622 |
| TGA | 4635-4637 |
| TGA | 4620-4622 |
| TGA | 4635-4637 |
| TGA | 4632-4634 |
| TGA | 4635-4637 |
| TAG | 4638-4640 |
| TGA | 4635-4637 |
| TAG | 4638-4640 |
| TAG | 4677-4679 |
| TAG | 4638-4640 |
| TAG | 4677-4679 |
| TAG | 4641-4643 |
| TAG | 4677-4679 |
| TGA | 4641-4643 |
| TAG | 4677-4679 |
| TAA | 4644-4646 |
| TAG | 4677-4679 |
| TGA | 4644-4646 |
| TAG | 4677-4679 |
| TAA | 4644-4646 |
| TAG | 4677-4679 |
| TAG | 4644-4646 |
| TAG | 4677-4679 |
| TAG | 4653-4655 |
| TAG | 4677-4679 |
| TAG | 4656-4658 |
| TAG | 4677-4679 |
| TGA | 4656-4658 |
| TAG | 4677-4679 |
| TAG | 4665-4667 |
| TAG | 4677-4679 |
| TAA | 4674-4676 |
| TAG | 4677-4679 |
| TAA | 4707-4709 |
| TAG | 4677-4679 |
| TAA | 4707-4709 |
| TAA | 4680-4682 |
| TAA | 4707-4709 |
| TAA | 4683-4685 |
| TAA | 4707-4709 |
| TGA | 4683-4685 |
| TAA | 4707-4709 |
| TAA | 4683-4685 |
| TAA | 4707-4709 |
| TAG | 4683-4685 |
| TAA | 4707-4709 |
| TGA | 4695-4697 |
| TAA | 4707-4709 |
| TGA | 4698-4700 |
| TAA | 4707-4709 |
| TGA | 4716-4718 |
| TAA | 4707-4709 |
| TGA | 4716-4718 |
| TGA | 4836-4838 |
| TGA | 4716-4718 |
| TGA | 4836-4838 |
| TGA | 4722-4724 |
| TGA | 4836-4838 |
| TGA | 4725-4727 |
| TGA | 4836-4838 |
| TGA | 4737-4739 |
| TGA | 4836-4838 |
| TGA | 4740-4742 |
| TGA | 4836-4838 |
| TGA | 4755-4757 |
| TGA | 4836-4838 |
| TGA | 4764-4766 |
| TGA | 4836-4838 |
| TAA | 4773-4775 |
| TGA | 4836-4838 |
| TGA | 4773-4775 |
| TGA | 4836-4838 |
| TAA | 4773-4775 |
| TGA | 4836-4838 |
| TAG | 4773-4775 |
| TGA | 4836-4838 |
| TAG | 4782-4784 |
| TGA | 4836-4838 |
| TGA | 4788-4790 |
| TGA | 4836-4838 |
| TGA | 4794-4796 |
| TGA | 4836-4838 |
| TAA | 4806-4808 |
| TGA | 4836-4838 |
| TGA | 4806-4808 |
| TGA | 4836-4838 |
| TAA | 4806-4808 |
| TGA | 4836-4838 |
| TAG | 4806-4808 |
| TGA | 4836-4838 |
| TGA | 4812-4814 |
| TGA | 4836-4838 |
| TAG | 4824-4826 |
| TGA | 4836-4838 |
| TGA | 4839-4841 |
| TGA | 4836-4838 |
| TGA | 4839-4841 |
| TGA | 4845-4847 |
| TGA | 4839-4841 |
| TGA | 4845-4847 |
| TGA | 4851-4853 |
| TGA | 4845-4847 |
| TGA | 4851-4853 |
| TGA | 4860-4862 |
| TGA | 4851-4853 |
| TGA | 4860-4862 |
| TGA | 4980-4982 |
| TGA | 4860-4862 |
| TGA | 4980-4982 |
| TGA | 4863-4865 |
| TGA | 4980-4982 |
| TAG | 4866-4868 |
| TGA | 4980-4982 |
| TGA | 4875-4877 |
| TGA | 4980-4982 |
| TAA | 4893-4895 |
| TGA | 4980-4982 |
| TAA | 4920-4922 |
| TGA | 4980-4982 |
| TAA | 4935-4937 |
| TGA | 4980-4982 |
| TGA | 4944-4946 |
| TGA | 4980-4982 |
| TAA | 4995-4997 |
| TGA | 4980-4982 |
| TAA | 4995-4997 |
| TAA | 4992-4994 |
| TAA | 4995-4997 |
| TGA | 4992-4994 |
| TAA | 4995-4997 |
| TAA | 4992-4994 |
| TAA | 4995-4997 |
| TAG | 4992-4994 |
| TAA | 4995-4997 |
| TAA | 5151-5153 |
| TAA | 4995-4997 |
| TAA | 5151-5153 |
| TGA | 5004-5006 |
| TAA | 5151-5153 |
| TGA | 5007-5009 |
| TAA | 5151-5153 |
| TAG | 5010-5012 |
| TAA | 5151-5153 |
| TAG | 5016-5018 |
| TAA | 5151-5153 |
| TAG | 5019-5021 |
| TAA | 5151-5153 |
| TGA | 5022-5024 |
| TAA | 5151-5153 |
| TAA | 5025-5027 |
| TAA | 5151-5153 |
| TGA | 5031-5033 |
| TAA | 5151-5153 |
| TGA | 5049-5051 |
| TAA | 5151-5153 |
| TAG | 5052-5054 |
| TAA | 5151-5153 |
| TAA | 5058-5060 |
| TAA | 5151-5153 |
| TAA | 5061-5063 |
| TAA | 5151-5153 |
| TAG | 5064-5066 |
| TAA | 5151-5153 |
| TGA | 5097-5099 |
| TAA | 5151-5153 |
| TGA | 5100-5102 |
| TAA | 5151-5153 |
| TAA | 5115-5117 |
| TAA | 5151-5153 |
| TGA | 5115-5117 |
| TAA | 5151-5153 |
| TAA | 5115-5117 |
| TAA | 5151-5153 |
| TAG | 5115-5117 |
| TAA | 5151-5153 |
| TAA | 5118-5120 |
| TAA | 5151-5153 |
| TAG | 5127-5129 |
| TAA | 5151-5153 |
| TAA | 5130-5132 |
| TAA | 5151-5153 |
| TGA | 5163-5165 |
| TAA | 5151-5153 |
| TGA | 5163-5165 |
| TGA | 5199-5201 |
| TGA | 5163-5165 |
| TGA | 5199-5201 |
| TGA | 5166-5168 |
| TGA | 5199-5201 |
| TAA | 5187-5189 |
| TGA | 5199-5201 |
| TGA | 5193-5195 |
| TGA | 5199-5201 |
| TGA | 5211-5213 |
| TGA | 5199-5201 |
| TGA | 5211-5213 |
| TAG | 5208-5210 |
| TGA | 5211-5213 |
| TGA | 5208-5210 |
| TGA | 5211-5213 |
| TAG | 5262-5264 |
| TGA | 5211-5213 |
| TAG | 5262-5264 |
| TAA | 5223-5225 |
| TAG | 5262-5264 |
| TAA | 5226-5228 |
| TAG | 5262-5264 |
| TGA | 5226-5228 |
| TAG | 5262-5264 |
| TAA | 5226-5228 |
| TAG | 5262-5264 |
| TAG | 5226-5228 |
| TAG | 5262-5264 |
| TAA | 5250-5252 |
| TAG | 5262-5264 |
| TAG | 5253-5255 |
| TAG | 5262-5264 |
| TGA | 5253-5255 |
| TAG | 5262-5264 |
| TAA | 5289-5291 |
| TAG | 5262-5264 |
| TAA | 5289-5291 |
| TAA | 5265-5267 |
| TAA | 5289-5291 |
| TGA | 5265-5267 |
| TAA | 5289-5291 |
| TAA | 5265-5267 |
| TAA | 5289-5291 |
| TAG | 5265-5267 |
| TAA | 5289-5291 |
| TAG | 5271-5273 |
| TAA | 5289-5291 |
| TGA | 5271-5273 |
| TAA | 5289-5291 |
| TGA | 5310-5312 |
| TAA | 5289-5291 |
| TGA | 5310-5312 |
| TGA | 5292-5294 |
| TGA | 5310-5312 |
| TAG | 5295-5297 |
| TGA | 5310-5312 |
| TAA | 5298-5300 |
| TGA | 5310-5312 |
| TAA | 5307-5309 |
| TGA | 5310-5312 |
| TGA | 5316-5318 |
| TGA | 5310-5312 |
| TGA | 5316-5318 |
| TGA | 5322-5324 |
| TGA | 5316-5318 |
| TGA | 5322-5324 |
| TGA | 5604-5606 |
| TGA | 5322-5324 |
| TGA | 5604-5606 |
| TAG | 5328-5330 |
| TGA | 5604-5606 |
| TGA | 5334-5336 |
| TGA | 5604-5606 |
| TAA | 5343-5345 |
| TGA | 5604-5606 |
| TAG | 5349-5351 |
| TGA | 5604-5606 |
| TAA | 5352-5354 |
| TGA | 5604-5606 |
| TAA | 5367-5369 |
| TGA | 5604-5606 |
| TAG | 5376-5378 |
| TGA | 5604-5606 |
| TGA | 5379-5381 |
| TGA | 5604-5606 |
| TGA | 5388-5390 |
| TGA | 5604-5606 |
| TAG | 5391-5393 |
| TGA | 5604-5606 |
| TAA | 5394-5396 |
| TGA | 5604-5606 |
| TAG | 5403-5405 |
| TGA | 5604-5606 |
| TGA | 5412-5414 |
| TGA | 5604-5606 |
| TAG | 5418-5420 |
| TGA | 5604-5606 |
| TGA | 5418-5420 |
| TGA | 5604-5606 |
| TAG | 5421-5423 |
| TGA | 5604-5606 |
| TGA | 5421-5423 |
| TGA | 5604-5606 |
| TAA | 5424-5426 |
| TGA | 5604-5606 |
| TGA | 5424-5426 |
| TGA | 5604-5606 |
| TAA | 5424-5426 |
| TGA | 5604-5606 |
| TAG | 5424-5426 |
| TGA | 5604-5606 |
| TAA | 5439-5441 |
| TGA | 5604-5606 |
| TGA | 5451-5453 |
| TGA | 5604-5606 |
| TGA | 5460-5462 |
| TGA | 5604-5606 |
| TAG | 5463-5465 |
| TGA | 5604-5606 |
| TGA | 5463-5465 |
| TGA | 5604-5606 |
| TAG | 5478-5480 |
| TGA | 5604-5606 |
| TGA | 5478-5480 |
| TGA | 5604-5606 |
| TAA | 5496-5498 |
| TGA | 5604-5606 |
| TGA | 5499-5501 |
| TGA | 5604-5606 |
| TGA | 5556-5558 |
| TGA | 5604-5606 |
| TAG | 5562-5564 |
| TGA | 5604-5606 |
| TGA | 5562-5564 |
| TGA | 5604-5606 |
| TGA | 5568-5570 |
| TGA | 5604-5606 |
| TGA | 5571-5573 |
| TGA | 5604-5606 |
| TAA | 5574-5576 |
| TGA | 5604-5606 |
| TAG | 5601-5603 |
| TGA | 5604-5606 |
| TGA | 5601-5603 |
| TGA | 5604-5606 |
| TGA | 5625-5627 |
| TGA | 5628-5630 |
| TGA | 5637-5639 |
| TAG | 5640-5642 |
| TAG | 5652-5654 |
| TGA | 5658-5660 |
| TGA | 5664-5666 |
| TGA | 5670-5672 |
| TAG | 5676-5678 |
| TAG | 5700-5702 |
| TAG | 5706-5708 |

Two base insertions of the present invention form stop codons as indicated in the following TABLE 7. The formed in-frame stop codon and the location are provided. Any expressed protein from BRCA1 genes with these types of mutations should be truncated accordingly. It should be recognized that the present invention includes insertions of 3n+2 bases, where n is an integer greater than zero and less than 1861. These larger insertions mutations have stop codons at nucleotide numbers corresponding to the listed stop codon at the nucleotide number listed. The corresponding nucleotide numbers of the stop codons will be 3n nucleotides larger than those listed.

TABLE 7
Two Base Insertions

| Codon Formed | Nucleotide Number |
|---|---|
| TAG | 123-125 |
| TAA | 126-128 |
| TAG | 126-128 |
| TGA | 126-128 |
| TAA | 129-131 |
| TAG | 129-131 |
| TGA | 129-131 |
| TAG | 132-134 |
| TAG | 141-143 |
| TAG | 144-146 |
| TAG | 147-149 |
| TAG | 150-152 |
| TAA | 156-158 |
| TAG | 159-161 |
| TAA | 162-164 |
| TAA | 165-167 |
| TAG | 168-170 |
| TAA | 171-173 |
| TAA | 177-179 |
| TGA | 177-179 |
| TAA | 180-182 |
| TAA | 183-185 |
| TAG | 183-185 |
| TGA | 183-185 |
| TAG | 186-188 |
| TAA | 189-191 |
| TAG | 189-191 |
| TGA | 189-191 |
| TAA | 195-197 |
| TAA | 198-200 |
| TAG | 198-200 |
| TGA | 198-200 |
| TAG | 204-206 |
| TAA | 207-209 |
| TAG | 207-209 |
| TGA | 207-209 |
| TAA | 210-212 |
| TGA | 210-212 |
| TAA | 213-215 |
| TAG | 216-218 |
| TAG | 222-224 |
| TAA | 225-227 |
| TAG | 225-227 |
| TGA | 225-227 |
| TAA | 228-230 |
| TAA | 231-233 |
| TAA | 234-236 |
| TAG | 234-236 |
| TGA | 234-236 |
| TAG | 237-239 |
| TAA | 243-245 |
| TAA | 246-248 |
| TAG | 246-248 |
| TGA | 246-248 |
| TAA | 249-251 |
| TAG | 249-251 |
| TGA | 249-251 |
| TAA | 252-254 |
| TAA | 255-257 |
| TAG | 255-257 |
| TGA | 255-257 |
| TAA | 258-260 |
| TAG | 258-260 |
| TGA | 258-260 |
| TAA | 261-263 |
| TAA | 267-269 |
| TGA | 267-269 |
| TAA | 276-278 |
| TAA | 282-284 |
| TGA | 282-284 |
| TAA | 285-287 |
| TGA | 285-287 |
| TAG | 288-290 |
| TAA | 294-296 |
| TAG | 294-296 |
| TGA | 294-296 |
| TAA | 300-302 |
| TAG | 300-302 |
| TGA | 300-302 |
| TAA | 306-308 |
| TAG | 306-308 |
| TGA | 306-308 |
| TAA | 309-311 |
| TAG | 309-311 |
| TGA | 309-311 |
| TAA | 312-314 |
| TAA | 315-317 |
| TGA | 315-317 |
| TAG | 318-320 |
| TAA | 321-323 |
| TAA | 324-326 |
| TAA | 327-329 |
| TAA | 330-332 |
| TAA | 333-335 |
| TGA | 333-335 |
| TAG | 342-344 |
| TAA | 345-347 |
| TAA | 348-350 |
| TAA | 351-353 |
| TGA | 351-353 |
| TAA | 354-356 |
| TAG | 354-356 |
| TGA | 354-356 |
| TAA | 357-359 |
| TAG | 366-368 |
| TAG | 369-371 |
| TAG | 372-374 |
| TAA | 378-380 |
| TAG | 378-380 |
| TGA | 378-380 |
| TAA | 381-383 |
| TGA | 381-383 |
| TAA | 384-386 |
| TAA | 387-389 |
| TAA | 390-392 |
| TAG | 390-392 |
| TGA | 390-392 |
| TAG | 393-395 |
| TAA | 396-398 |
| TAG | 396-398 |
| TGA | 396-398 |
| TAG | 405-407 |
| TAA | 408-410 |
| TAG | 411-413 |
| TAA | 414-416 |
| TAG | 414-416 |
| TGA | 414-416 |
| TAG | 417-419 |
| TAA | 420-422 |
| TAG | 420-422 |
| TGA | 420-422 |
| TAG | 423-425 |
| TAA | 426-428 |
| TAA | 429-431 |
| TAA | 432-434 |
| TAG | 432-434 |
| TGA | 432-434 |
| TAA | 435-437 |
| TAA | 438-440 |
| TAG | 438-440 |
| TGA | 438-440 |
| TAG | 441-443 |
| TAA | 444-446 |
| TAA | 447-449 |
| TAG | 450-452 |
| TAA | 453-455 |
| TAA | 456-458 |
| TAA | 459-461 |
| TAG | 459-461 |
| TGA | 459-461 |
| TAG | 465-467 |
| TAA | 474-476 |
| TAG | 477-479 |
| TAG | 480-482 |
| TAG | 483-485 |
| TAA | 486-488 |
| TAG | 486-488 |
| TGA | 486-488 |
| TAA | 489-491 |
| TAA | 492-494 |
| TAA | 498-500 |
| TAA | 501-503 |
| TAG | 504-506 |
| TAA | 507-509 |
| TAG | 507-509 |
| TGA | 507-509 |
| TAA | 510-512 |
| TAA | 513-515 |
| TAG | 519-521 |
| TAA | 522-524 |
| TAA | 525-527 |
| TAA | 537-539 |
| TGA | 537-539 |
| TAG | 540-542 |
| TAG | 546-548 |
| TAA | 549-551 |
| TAA | 555-557 |
| TAG | 555-557 |
| TGA | 555-557 |
| TAA | 558-560 |
| TAG | 558-560 |
| TGA | 558-560 |
| TAG | 564-566 |
| TAA | 567-569 |
| TAA | 570-572 |
| TAA | 576-578 |
| TAG | 579-581 |
| TAA | 588-590 |
| TAG | 588-590 |
| TGA | 588-590 |
| TAA | 591-593 |
| TAG | 597-599 |
| TAA | 600-602 |
| TAG | 603-605 |
| TAA | 606-608 |
| TGA | 606-608 |
| TAA | 609-611 |
| TAA | 615-617 |
| TGA | 615-617 |
| TAA | 618-620 |
| TGA | 618-620 |
| TAA | 621-623 |
| TAA | 630-632 |
| TGA | 630-632 |
| TAA | 642-644 |
| TAA | 645-647 |
| TGA | 645-647 |
| TAA | 648-650 |
| TAG | 648-650 |
| TGA | 648-650 |
| TAG | 651-653 |
| TAA | 654-656 |
| TAG | 654-656 |
| TGA | 654-656 |
| TAA | 657-659 |
| TAG | 660-662 |
| TAA | 663-665 |
| TAG | 663-665 |
| TGA | 663-665 |
| TAG | 666-668 |
| TAA | 669-671 |
| TAG | 669-671 |
| TGA | 669-671 |
| TAG | 672-674 |
| TAA | 675-677 |
| TAG | 675-677 |
| TGA | 675-677 |
| TAA | 678-680 |
| TAG | 678-680 |
| TGA | 678-680 |
| TAG | 681-683 |
| TAG | 684-686 |
| TAA | 687-689 |
| TAG | 690-692 |
| TAA | 693-695 |
| TAA | 696-698 |
| TAG | 699-701 |
| TAA | 702-704 |
| TAA | 705-707 |
| TAG | 705-707 |
| TGA | 705-707 |
| TAA | 708-710 |
| TAG | 708-710 |
| TGA | 708-710 |
| TAA | 711-713 |
| TAG | 714-716 |
| TAG | 717-719 |
| TAG | 720-722 |
| TAG | 726-728 |
| TAA | 729-731 |
| TAG | 729-731 |
| TGA | 729-731 |
| TAA | 732-734 |
| TAG | 732-734 |
| TGA | 732-734 |
| TAA | 738-740 |
| TAA | 741-743 |
| TAG | 750-752 |
| TAA | 753-755 |
| TAA | 756-758 |
| TAG | 759-761 |
| TAG | 762-764 |
| TAA | 765-767 |
| TAA | 768-770 |
| TAA | 771-773 |
| TAG | 771-773 |
| TGA | 771-773 |
| TAG | 774-776 |
| TAA | 777-779 |
| TAG | 777-779 |
| TGA | 777-779 |
| TAG | 780-782 |
| TAA | 783-785 |
| TAA | 786-788 |
| TAG | 789-791 |
| TAG | 792-794 |
| TAA | 795-797 |
| TAG | 795-797 |
| TGA | 795-797 |
| TAG | 798-800 |
| TAA | 801-803 |
| TAG | 801-803 |
| TGA | 801-803 |
| TAA | 804-806 |
| TAG | 804-806 |
| TGA | 804-806 |
| TAG | 807-809 |
| TAA | 810-812 |
| TGA | 810-812 |
| TAG | 813-815 |
| TAG | 816-818 |
| TAA | 819-821 |
| TAA | 822-824 |
| TAA | 825-827 |
| TAG | 828-830 |
| TAA | 843-845 |
| TAA | 846-848 |
| TAA | 849-851 |
| TAG | 852-854 |
| TAA | 855-857 |
| TAG | 855-857 |
| TGA | 855-857 |
| TAA | 858-860 |
| TGA | 858-860 |
| TAA | 861-863 |
| TAA | 864-866 |
| TAG | 867-869 |
| TAA | 870-872 |
| TGA | 870-872 |
| TAG | 876-878 |
| TAG | 879-881 |
| TAG | 882-884 |
| TAA | 885-887 |
| TGA | 885-887 |
| TAG | 894-896 |
| TAA | 897-899 |
| TAA | 900-902 |
| TAG | 900-902 |
| TGA | 900-902 |
| TAG | 906-908 |
| TAA | 909-911 |
| TAA | 912-914 |
| TAG | 912-914 |
| TGA | 912-914 |
| TAG | 915-917 |
| TAA | 918-920 |
| TAG | 918-920 |
| TGA | 918-920 |
| TAA | 921-923 |
| TAA | 924-926 |
| TAG | 924-926 |
| TGA | 924-926 |
| TAG | 930-932 |
| TAG | 933-935 |
| TAA | 939-941 |
| TAG | 939-941 |
| TGA | 939-941 |
| TAG | 942-944 |
| TAA | 945-947 |
| TAA | 948-950 |
| TAA | 951-953 |
| TAG | 957-959 |
| TAA | 960-962 |
| TAA | 963-965 |
| TAG | 963-965 |
| TGA | 963-965 |
| TAA | 966-968 |
| TAG | 966-968 |
| TGA | 966-968 |
| TAG | 975-977 |
| TAA | 978-980 |
| TGA | 978-980 |
| TAA | 981-983 |
| TAA | 984-986 |
| TAA | 987-989 |
| TAG | 987-989 |
| TGA | 987-989 |
| TAA | 990-992 |
| TAG | 990-992 |
| TGA | 990-992 |
| TAA | 996-998 |
| TAA | 999-1001 |
| TAG | 1002-1004 |
| TAA | 1005-1007 |
| TAA | 1008-1010 |
| TAA | 1011-1013 |
| TGA | 1011-1013 |
| TAG | 1014-1016 |
| TAG | 1017-1019 |
| TAA | 1020-1022 |
| TAG | 1023-1025 |
| TAG | 1026-1028 |
| TAA | 1029-1031 |
| TAG | 1029-1031 |
| TGA | 1029-1031 |
| TAA | 1032-1034 |
| TAG | 1032-1034 |
| TGA | 1032-1034 |
| TAA | 1035-1037 |
| TAA | 1038-1040 |
| TAA | 1041-1043 |
| TAA | 1044-1046 |
| TAG | 1053-1055 |
| TAA | 1056-1058 |
| TAG | 1056-1058 |
| TGA | 1056-1058 |
| TAG | 1059-1061 |
| TAA | 1062-1064 |
| TAA | 1065-1067 |
| TGA | 1065-1067 |
| TAA | 1074-1076 |
| TAA | 1077-1079 |
| TAA | 1080-1082 |
| TAG | 1080-1082 |
| TGA | 1080-1082 |
| TAG | 1083-1085 |
| TAG | 1086-1088 |
| TAA | 1089-1091 |
| TAA | 1092-1094 |
| TAG | 1095-1097 |
| TAA | 1098-1100 |
| TAA | 1101-1103 |
| TAG | 1101-1103 |
| TGA | 1101-1103 |
| TAA | 1104-1106 |
| TAG | 1107-1109 |
| TAA | 1110-1112 |
| TAA | 1116-1118 |
| TGA | 1116-1118 |
| TAA | 1122-1124 |
| TAA | 1125-1127 |
| TAG | 1128-1130 |
| TAA | 1131-1133 |
| TAA | 1134-1136 |
| TAG | 1137-1139 |
| TAG | 1140-1142 |
| TAA | 1146-1148 |
| TGA | 1146-1148 |
| TAG | 1149-1151 |
| TAG | 1152-1154 |
| TAA | 1161-1163 |
| TAG | 1161-1163 |
| TGA | 1161-1163 |
| TAG | 1164-1166 |
| TAA | 1167-1169 |
| TGA | 1167-1169 |
| TAA | 1170-1172 |
| TAG | 1173-1175 |
| TAA | 1176-1178 |
| TAG | 1176-1178 |
| TGA | 1176-1178 |
| TAA | 1179-1181 |
| TGA | 1179-1181 |
| TAA | 1182-1184 |
| TAA | 1188-1190 |
| TGA | 1188-1190 |
| TAA | 1197-1199 |
| TAG | 1197-1199 |
| TGA | 1197-1199 |
| TAA | 1200-1202 |
| TAG | 1200-1202 |
| TGA | 1200-1202 |
| TAG | 1203-1205 |
| TAA | 1206-1208 |
| TGA | 1206-1208 |
| TAA | 1212-1214 |
| TAG | 1215-1217 |
| TAA | 1218-1220 |
| TAG | 1221-1223 |
| TAG | 1224-1226 |
| TAG | 1227-1229 |
| TAA | 1233-1235 |
| TAG | 1233-1235 |
| TGA | 1233-1235 |
| TAA | 1236-1238 |
| TGA | 1236-1238 |
| TAA | 1239-1241 |
| TAA | 1245-1247 |
| TAA | 1248-1250 |
| TAA | 1251-1253 |
| TAA | 1254-1256 |
| TAA | 1260-1262 |
| TGA | 1260-1262 |
| TAG | 1263-1265 |
| TAA | 1266-1268 |
| TAG | 1269-1271 |
| TAA | 1272-1274 |
| TAG | 1272-1274 |
| TGA | 1272-1274 |
| TAA | 1275-1277 |
| TAG | 1275-1277 |
| TGA | 1275-1277 |
| TAA | 1278-1280 |
| TAG | 1278-1280 |
| TGA | 1278-1280 |
| TAA | 1281-1283 |
| TAA | 1284-1286 |
| TAG | 1287-1289 |
| TAG | 1290-1292 |
| TAA | 1296-1298 |
| TAG | 1296-1298 |
| TGA | 1296-1298 |
| TAG | 1299-1301 |
| TAA | 1302-1304 |
| TAG | 1302-1304 |
| TGA | 1302-1304 |
| TAG | 1305-1307 |
| TAG | 1308-1310 |
| TAA | 1311-1313 |
| TAG | 1311-1313 |
| TGA | 1311-1313 |
| TAG | 1317-1319 |
| TAG | 1320-1322 |
| TAG | 1323-1325 |
| TAA | 1326-1328 |
| TAG | 1326-1328 |
| TGA | 1326-1328 |
| TAG | 1329-1331 |
| TAA | 1332-1334 |
| TAG | 1332-1334 |
| TGA | 1332-1334 |
| TAA | 1335-1337 |
| TAG | 1338-1340 |
| TAA | 1341-1343 |
| TAG | 1344-1346 |
| TAG | 1347-1349 |
| TAG | 1350-1352 |
| TAG | 1353-1355 |
| TAA | 1356-1358 |
| TAG | 1356-1358 |
| TGA | 1356-1358 |
| TAG | 1359-1361 |
| TAG | 1362-1364 |
| TAA | 1368-1370 |
| TAG | 1371-1373 |
| TAG | 1374-1376 |
| TAG | 1377-1379 |
| TAG | 1380-1382 |
| TAA | 1383-1385 |
| TAG | 1383-1385 |
| TGA | 1383-1385 |
| TAA | 1386-1388 |
| TAG | 1386-1388 |
| TGA | 1386-1388 |
| TAG | 1389-1391 |
| TAA | 1392-1394 |
| TAG | 1392-1394 |
| TGA | 1392-1394 |
| TAA | 1395-1397 |
| TAG | 1395-1397 |
| TGA | 1395-1397 |
| TAG | 1398-1400 |
| TAA | 1401-1403 |
| TGA | 1401-1403 |
| TAA | 1404-1406 |
| TAG | 1407-1409 |
| TAA | 1410-1412 |
| TAG | 1410-1412 |
| TGA | 1410-1412 |
| TAG | 1416-1418 |
| TAA | 1419-1421 |
| TAG | 1422-1424 |
| TAG | 1431-1433 |
| TAG | 1434-1436 |
| TAA | 1437-1439 |
| TAG | 1437-1439 |
| TGA | 1437-1439 |
| TAA | 1440-1442 |
| TAA | 1443-1445 |
| TAG | 1443-1445 |
| TGA | 1443-1445 |
| TAA | 1446-1448 |
| TAA | 1449-1451 |
| TAG | 1452-1454 |
| TAA | 1455-1457 |
| TAG | 1458-1460 |
| TAA | 1464-1466 |
| TAG | 1464-1466 |
| TGA | 1464-1466 |
| TAA | 1467-1469 |
| TAA | 1470-1472 |
| TAG | 1470-1472 |
| TGA | 1470-1472 |
| TAG | 1473-1475 |
| TAG | 1476-1478 |
| TAA | 1479-1481 |
| TGA | 1479-1481 |
| TAA | 1482-1484 |
| TAA | 1485-1487 |
| TAG | 1488-1490 |
| TAG | 1491-1493 |
| TAA | 1494-1496 |
| TAA | 1497-1499 |
| TAA | 1500-1502 |
| TAG | 1500-1502 |
| TGA | 1500-1502 |
| TAG | 1503-1505 |
| TAA | 1506-1508 |
| TGA | 1506-1508 |
| TAA | 1509-1511 |
| TAA | 1512-1514 |
| TAG | 1512-1514 |
| TGA | 1512-1514 |
| TAA | 1518-1520 |
| TGA | 1518-1520 |
| TAA | 1521-1523 |
| TGA | 1521-1523 |
| TAG | 1524-1526 |
| TAA | 1527-1529 |
| TAA | 1536-1538 |
| TAA | 1539-1541 |
| TAG | 1539-1541 |
| TGA | 1539-1541 |
| TAA | 1542-1544 |
| TAG | 1548-1550 |
| TAA | 1551-1553 |
| TAG | 1554-1556 |
| TAA | 1557-1559 |
| TAA | 1563-1565 |
| TAA | 1566-1568 |
| TAG | 1569-1571 |
| TAG | 1572-1574 |
| TAA | 1575-1577 |
| TAG | 1575-1577 |
| TGA | 1575-1577 |
| TAG | 1578-1580 |
| TAA | 1581-1583 |
| TAG | 1584-1586 |
| TAA | 1593-1595 |
| TGA | 1593-1595 |
| TAA | 1596-1598 |
| TAG | 1602-1604 |
| TAA | 1614-1616 |
| TAA | 1617-1619 |
| TAA | 1620-1622 |
| TAA | 1623-1625 |
| TAG | 1623-1625 |
| TGA | 1623-1625 |
| TAA | 1626-1628 |
| TAA | 1632-1634 |
| TAA | 1635-1637 |
| TAA | 1638-1640 |
| TGA | 1638-1640 |
| TAA | 1644-1646 |
| TAA | 1647-1649 |
| TAG | 1647-1649 |
| TGA | 1647-1649 |
| TAG | 1650-1652 |
| TAG | 1662-1664 |
| TAG | 1665-1667 |
| TAA | 1668-1670 |
| TAG | 1668-1670 |
| TGA | 1668-1670 |
| TAA | 1671-1673 |
| TAA | 1674-1676 |
| TAA | 1677-1679 |
| TGA | 1677-1679 |
| TAG | 1680-1682 |
| TAG | 1683-1685 |
| TAA | 1686-1688 |
| TAG | 1686-1688 |
| TGA | 1686-1688 |
| TAG | 1689-1691 |
| TAG | 1692-1694 |
| TAA | 1698-1700 |
| TAA | 1701-1703 |
| TGA | 1701-1703 |
| TAG | 1707-1709 |
| TAA | 1710-1712 |
| TAA | 1713-1715 |
| TGA | 1713-1715 |
| TAA | 1716-1718 |
| TAG | 1722-1724 |
| TAA | 1725-1727 |
| TAA | 1728-1730 |
| TAA | 1734-1736 |
| TAG | 1737-1739 |
| TAA | 1743-1745 |
| TGA | 1743-1745 |
| TAG | 1746-1748 |
| TAG | 1752-1754 |
| TAA | 1755-1757 |
| TGA | 1755-1757 |
| TAA | 1758-1760 |
| TGA | 1758-1760 |
| TAA | 1761-1763 |
| TAA | 1764-1766 |
| TAA | 1767-1769 |
| TAA | 1770-1772 |
| TAG | 1773-1775 |
| TAG | 1779-1781 |
| TAA | 1782-1784 |
| TGA | 1782-1784 |
| TAA | 1785-1787 |
| TAA | 1788-1790 |
| TAA | 1791-1793 |
| TAG | 1794-1796 |
| TAG | 1797-1799 |
| TAA | 1800-1802 |
| TAG | 1800-1802 |
| TGA | 1800-1802 |
| TAA | 1803-1805 |
| TAA | 1809-1811 |
| TGA | 1809-1811 |
| TAG | 1812-1814 |
| TAA | 1815-1817 |
| TGA | 1815-1817 |
| TAA | 1818-1820 |
| TAA | 1824-1826 |
| TAA | 1830-1832 |
| TAG | 1833-1835 |
| TAA | 1836-1838 |
| TAG | 1836-1838 |
| TGA | 1836-1838 |
| TAG | 1842-1844 |
| TAA | 1845-1847 |
| TAG | 1848-1850 |
| TAA | 1851-1853 |
| TAG | 1851-1853 |
| TGA | 1851-1853 |
| TAG | 1854-1856 |
| TAA | 1857-1859 |
| TAG | 1857-1859 |
| TGA | 1857-1859 |
| TAA | 1860-1862 |
| TAA | 1863-1865 |
| TAA | 1866-1868 |
| TGA | 1866-1868 |
| TAG | 1869-1871 |
| TAG | 1872-1874 |
| TAA | 1878-1880 |
| TAA | 1881-1883 |
| TAA | 1884-1886 |
| TAA | 1887-1889 |
| TAA | 1890-1892 |
| TAA | 1893-1895 |
| TAA | 1896-1898 |
| TAA | 1899-1901 |
| TAG | 1902-1904 |
| TAG | 1908-1910 |
| TAA | 1911-1913 |
| TAG | 1911-1913 |
| TGA | 1911-1913 |
| TAA | 1914-1916 |
| TAA | 1917-1919 |
| TAA | 1923-1925 |
| TAA | 1926-1928 |
| TAG | 1926-1928 |
| TGA | 1926-1928 |
| TAA | 1929-1931 |
| TAG | 1932-1934 |
| TAA | 1938-1940 |
| TAA | 1941-1943 |
| TAA | 1944-1946 |
| TGA | 1944-1946 |
| TAA | 1947-1949 |
| TGA | 1953-1955 |
| TAA | 1953-1955 |
| TGA | 1953-1955 |
| TAA | 1956-1958 |
| TGA | 1956-1958 |
| TAA | 1959-1961 |
| TGA | 1959-1961 |
| TAA | 1962-1964 |
| TAG | 1962-1964 |
| TGA | 1962-1964 |
| TAA | 1965-1967 |
| TAG | 1965-1967 |
| TGA | 1965-1967 |
| TAA | 1968-1970 |
| TAA | 1971-1973 |
| TAA | 1977-1979 |
| TAG | 1983-1985 |
| TAG | 1989-1991 |
| TAG | 1995-1997 |
| TAG | 1998-2000 |
| TAA | 2001-2003 |
| TAA | 2004-2006 |
| TAA | 2007-2009 |
| TAA | 2013-2015 |
| TAA | 2022-2024 |
| TAA | 2025-2027 |
| TAG | 2025-2027 |
| TGA | 2025-2027 |
| TAA | 2028-2030 |
| TAG | 2031-2033 |
| TAA | 2034-2036 |
| TAG | 2034-2036 |
| TGA | 2034-2036 |
| TAA | 2040-2042 |
| TAG | 2043-2045 |
| TAA | 2046-2048 |
| TAA | 2049-2051 |
| TAG | 2049-2051 |
| TGA | 2049-2051 |
| TAA | 2052-2054 |
| TAG | 2052-2054 |
| TGA | 2052-2054 |
| TAA | 2055-2057 |
| TAA | 2058-2060 |
| TAG | 2061-2063 |
| TAG | 2064-2066 |
| TAA | 2067-2069 |
| TGA | 2067-2069 |
| TAA | 2070-2072 |
| TAA | 2073-2075 |
| TGA | 2073-2075 |
| TAA | 2076-2078 |
| TAA | 2079-2081 |
| TAA | 2082-2084 |
| TAG | 2082-2084 |
| TGA | 2082-2084 |
| TAA | 2085-2087 |
| TAA | 2091-2093 |
| TAG | 2097-2099 |
| TAA | 2100-2102 |
| TAA | 2106-2108 |
| TAA | 2109-2111 |
| TAA | 2112-2114 |
| TAA | 2124-2126 |
| TAG | 2127-2129 |
| TAG | 2130-2132 |
| TAA | 2133-2135 |
| TAG | 2136-2138 |
| TAG | 2142-2144 |
| TAA | 2145-2147 |
| TAG | 2148-2150 |
| TAG | 2151-2153 |
| TAA | 2154-2156 |
| TAA | 2157-2159 |
| TGA | 2157-2159 |
| TAA | 2160-2162 |
| TGA | 2160-2162 |
| TAA | 2163-2165 |
| TAA | 2166-2168 |
| TAA | 2172-2174 |
| TAG | 2175-2177 |
| TAA | 2181-2183 |
| TGA | 2181-2183 |
| TAA | 2184-2186 |
| TAA | 2187-2189 |
| TAA | 2190-2192 |
| TAG | 2196-2198 |
| TAA | 2199-2201 |
| TAG | 2202-2204 |
| TAA | 2205-2207 |
| TAA | 2208-2210 |
| TAG | 2208-2210 |
| TGA | 2208-2210 |
| TAG | 2214-2216 |
| TAA | 2220-2222 |
| TGA | 2220-2222 |
| TAA | 2223-2225 |
| TAG | 2223-2225 |
| TGA | 2223-2225 |
| TAA | 2226-2228 |
| TAA | 2229-2231 |
| TAG | 2232-2234 |
| TAG | 2238-2240 |
| TAA | 2241-2243 |
| TAG | 2241-2243 |
| TGA | 2241-2243 |
| TAA | 2244-2246 |
| TAG | 2244-2246 |
| TGA | 2244-2246 |
| TAA | 2247-2249 |
| TAA | 2250-2252 |
| TAA | 2253-2255 |
| TAG | 2253-2255 |
| TGA | 2253-2255 |
| TAA | 2256-2258 |
| TAG | 2256-2258 |
| TGA | 2256-2258 |
| TAA | 2259-2261 |
| TAA | 2262-2264 |
| TAA | 2265-2267 |
| TAG | 2268-2270 |
| TAA | 2274-2276 |
| TAG | 2277-2279 |
| TAA | 2280-2282 |
| TAG | 2280-2282 |
| TGA | 2280-2282 |
| TAG | 2283-2285 |
| TAA | 2286-2288 |
| TAA | 2292-2294 |
| TAA | 2301-2303 |
| TAG | 2304-2306 |
| TAG | 2307-2309 |
| TAA | 2310-2312 |
| TAG | 2313-2315 |
| TAG | 2316-2318 |
| TAA | 2319-2321 |
| TGA | 2319-2321 |
| TAG | 2325-2327 |
| TAA | 2328-2330 |
| TAG | 2331-2333 |
| TAA | 2334-2336 |
| TAG | 2337-2339 |
| TAA | 2340-2342 |
| TAG | 2340-2342 |
| TGA | 2340-2342 |
| TAA | 2343-2345 |
| TAA | 2346-2348 |
| TAG | 2349-2351 |
| TAG | 2352-2354 |
| TAG | 2355-2357 |
| TAA | 2361-2363 |
| TAG | 2364-2366 |
| TAA | 2370-2372 |
| TAA | 2373-2375 |
| TAG | 2373-2375 |
| TGA | 2373-2375 |
| TAA | 2376-2378 |
| TAG | 2379-2381 |
| TAG | 2382-2384 |
| TAA | 2385-2387 |
| TAG | 2388-2390 |
| TAA | 2391-2393 |
| TAG | 2391-2393 |
| TGA | 2391-2393 |
| TAA | 2397-2399 |
| TAG | 2400-2402 |
| TAA | 2403-2405 |
| TAA | 2406-2408 |
| TAG | 2406-2408 |
| TGA | 2406-2408 |
| TAG | 2409-2411 |
| TAG | 2412-2414 |
| TAA | 2415-2417 |
| TGA | 2415-2417 |
| TAA | 2418-2420 |
| TAA | 2421-2423 |
| TAA | 2424-2426 |
| TAA | 2427-2429 |
| TAG | 2427-2429 |
| TGA | 2427-2429 |
| TAG | 2433-2435 |
| TAG | 2439-2441 |
| TAA | 2442-2444 |
| TAG | 2445-2447 |
| TAA | 2448-2450 |
| TAG | 2448-2450 |
| TGA | 2448-2450 |
| TAG | 2451-2453 |
| TAA | 2454-2456 |
| TAG | 2460-2462 |
| TAA | 2463-2465 |
| TAA | 2466-2468 |
| TAA | 2469-2471 |
| TAG | 2469-2471 |
| TGA | 2469-2471 |
| TAA | 2472-2474 |
| TAG | 2472-2474 |
| TGA | 2472-2474 |
| TAG | 2478-2480 |
| TAG | 2481-2483 |
| TAA | 2484-2486 |
| TAA | 2487-2489 |
| TAG | 2493-2495 |
| TAA | 2496-2498 |
| TGA | 2496-2498 |
| TAG | 2499-2501 |
| TAA | 2502-2504 |
| TAA | 2505-2507 |
| TAG | 2508-2510 |
| TAA | 2514-2516 |
| TAA | 2517-2519 |
| TAA | 2520-2522 |
| TAG | 2520-2522 |
| TGA | 2520-2522 |
| TAG | 2523-2525 |
| TAA | 2526-2528 |
| TGA | 2526-2528 |
| TAA | 2532-2534 |
| TAG | 2532-2534 |
| TGA | 2532-2534 |
| TAG | 2535-2537 |
| TAG | 2538-2540 |
| TAA | 2541-2543 |
| TAG | 2541-2543 |
| TGA | 2541-2543 |
| TAG | 2544-2546 |
| TAA | 2547-2549 |
| TAA | 2553-2555 |
| TAG | 2556-2558 |
| TAA | 2562-2564 |
| TAG | 2568-2570 |
| TAA | 2571-2573 |
| TAG | 2571-2573 |
| TGA | 2571-2573 |
| TAA | 2574-2576 |
| TAG | 2574-2576 |
| TGA | 2574-2576 |
| TAA | 2577-2579 |
| TAG | 2580-2582 |
| TAA | 2583-2585 |
| TAA | 2586-2588 |
| TAA | 2589-2591 |
| TAG | 2592-2594 |
| TAA | 2595-2597 |
| TAG | 2598-2600 |
| TAG | 2601-2603 |
| TAA | 2604-2606 |
| TAG | 2604-2606 |
| TGA | 2604-2606 |
| TAA | 2607-2609 |
| TAA | 2610-2612 |
| TAG | 2610-2612 |
| TGA | 2610-2612 |
| TAA | 2616-2618 |
| TAG | 2616-2618 |
| TGA | 2616-2618 |
| TAG | 2619-2621 |
| TAG | 2625-2627 |
| TAG | 2628-2630 |
| TAA | 2631-2633 |
| TAA | 2637-2639 |
| TAG | 2643-2645 |
| TAA | 2646-2648 |
| TAA | 2649-2651 |
| TAA | 2652-2654 |
| TAG | 2655-2657 |
| TAA | 2658-2660 |
| TAG | 2661-2663 |
| TAG | 2664-2666 |
| TAA | 2667-2669 |
| TAG | 2670-2672 |
| TAG | 2676-2678 |
| TAG | 2679-2681 |
| TAA | 2685-2687 |
| TAG | 2685-2687 |
| TGA | 2685-2687 |
| TAA | 2688-2690 |
| TAG | 2688-2690 |
| TGA | 2688-2690 |
| TAA | 2694-2696 |
| TGA | 2694-2696 |
| TAA | 2697-2699 |
| TAA | 2700-2702 |
| TAG | 2700-2702 |
| TGA | 2700-2702 |
| TAA | 2703-2705 |
| TAG | 2706-2708 |
| TAA | 2709-2711 |
| TAG | 2709-2711 |
| TGA | 2709-2711 |
| TAA | 2712-2714 |
| TAA | 2721-2723 |
| TAG | 2721-2723 |
| TGA | 2721-2723 |
| TAA | 2724-2726 |
| TAG | 2724-2726 |
| TGA | 2724-2726 |
| TAG | 2727-2729 |
| TAA | 2733-2735 |
| TAG | 2733-2735 |
| TGA | 2733-2735 |
| TAA | 2736-2738 |
| TAG | 2736-2738 |
| TGA | 2736-2738 |
| TAA | 2739-2741 |
| TAG | 2745-2747 |
| TAA | 2748-2750 |
| TAG | 2751-2753 |
| TAG | 2754-2756 |
| TAG | 2757-2759 |
| TAG | 2760-2762 |
| TAA | 2763-2765 |
| TAG | 2763-2765 |
| TGA | 2763-2765 |
| TAG | 2766-2768 |
| TAA | 2769-2771 |
| TAA | 2772-2774 |
| TAG | 2772-2774 |
| TGA | 2772-2774 |
| TAA | 2775-2777 |
| TAG | 2775-2777 |
| TGA | 2775-2777 |
| TAG | 2778-2780 |
| TAA | 2784-2786 |
| TAG | 2784-2786 |
| TGA | 2784-2786 |
| TAG | 2787-2789 |
| TAA | 2790-2792 |
| TAG | 2790-2792 |
| TGA | 2790-2792 |
| TAA | 2793-2795 |
| TAG | 2793-2795 |
| TGA | 2793-2795 |
| TAA | 2796-2798 |
| TAA | 2799-2801 |
| TGA | 2799-2801 |
| TAA | 2805-2807 |
| TAA | 2811-2813 |
| TAG | 2814-2816 |
| TAA | 2817-2819 |
| TAA | 2820-2822 |
| TAG | 2820-2822 |
| TGA | 2820-2822 |
| TAG | 2823-2825 |
| TAA | 2826-2828 |
| TAG | 2826-2828 |
| TGA | 2826-2828 |
| TAG | 2829-2831 |
| TAA | 2835-2837 |
| TAG | 2838-2840 |
| TAG | 2841-2843 |
| TAA | 2844-2846 |
| TAG | 2850-2852 |
| TAA | 2853-2855 |
| TAA | 2856-2858 |
| TGA | 2856-2858 |
| TAG | 2859-2861 |
| TAA | 2862-2864 |
| TAG | 2862-2864 |
| TGA | 2862-2864 |
| TAA | 2865-2867 |
| TAA | 2868-2870 |
| TAA | 2871-2873 |
| TAG | 2877-2879 |
| TAA | 2883-2885 |
| TGA | 2883-2885 |
| TAG | 2886-2888 |
| TAA | 2889-2891 |
| TAA | 2892-2894 |
| TAA | 2895-2897 |
| TAG | 2898-2900 |
| TAG | 2901-2903 |
| TAA | 2904-2906 |
| TAG | 2904-2906 |
| TGA | 2904-2906 |
| TAG | 2910-2912 |
| TAG | 2913-2915 |
| TAG | 2916-2918 |
| TAA | 2922-2924 |
| TGA | 2922-2924 |
| TAG | 2925-2927 |
| TAA | 2928-2930 |
| TAG | 2934-2936 |
| TAG | 2937-2939 |
| TAA | 2940-2942 |
| TAG | 2943-2945 |
| TAA | 2946-2948 |
| TAA | 2949-2951 |
| TAG | 2949-2951 |
| TGA | 2949-2951 |
| TAA | 2952-2954 |
| TAA | 2955-2957 |
| TAA | 2958-2960 |
| TAG | 2961-2963 |
| TAG | 2964-2966 |
| TAA | 2967-2969 |
| TAG | 2967-2969 |
| TGA | 2967-2969 |
| TAA | 2970-2972 |
| TAA | 2973-2975 |
| TAG | 2973-2975 |
| TGA | 2973-2975 |
| TAA | 2976-2978 |
| TAG | 2976-2978 |
| TGA | 2976-2978 |
| TAA | 2982-2984 |
| TAG | 2982-2984 |
| TGA | 2982-2984 |
| TAA | 2985-2987 |
| TAG | 2985-2987 |
| TGA | 2985-2987 |
| TAA | 2991-2993 |
| TAG | 2991-2993 |
| TGA | 2991-2993 |
| TAA | 2994-2996 |
| TAG | 2997-2999 |
| TAA | 3000-3002 |
| TAG | 3003-3005 |
| TAA | 3006-3008 |
| TAG | 3009-3011 |
| TAA | 3015-3017 |
| TAA | 3018-3020 |
| TAA | 3024-3026 |
| TAA | 3027-3029 |
| TAG | 3033-3035 |
| TAA | 3039-3041 |
| TAG | 3039-3041 |
| TGA | 3039-3041 |
| TAA | 3045-3047 |
| TAA | 3051-3053 |
| TAG | 3051-3053 |
| TGA | 3051-3053 |
| TAA | 3057-3059 |
| TAA | 3069-3071 |
| TAG | 3069-3071 |
| TGA | 3069-3071 |
| TAA | 3075-3077 |
| TAA | 3078-3080 |
| TAA | 3081-3083 |
| TAG | 3081-3083 |
| TGA | 3081-3083 |
| TAA | 3084-3086 |
| TAG | 3084-3086 |
| TGA | 3084-3086 |
| TAG | 3087-3089 |
| TAA | 3090-3092 |
| TAA | 3093-3095 |
| TAA | 3096-3098 |
| TAA | 3099-3101 |
| TAG | 3099-3101 |
| TGA | 3099-3101 |
| TAA | 3102-3104 |
| TAA | 3105-3107 |
| TGA | 3105-3107 |
| TAA | 3108-3110 |
| TAG | 3117-3119 |
| TAG | 3120-3122 |
| TAA | 3123-3125 |
| TAA | 3126-3128 |
| TAG | 3126-3128 |
| TGA | 3126-3128 |
| TAG | 3129-3131 |
| TAG | 3132-3134 |
| TAA | 3138-3140 |
| TAG | 3138-3140 |
| TGA | 3138-3140 |
| TAA | 3141-3143 |
| TAA | 3144-3146 |
| TAG | 3144-3146 |
| TGA | 3144-3146 |
| TAG | 3150-3152 |
| TAA | 3153-3155 |
| TAG | 3156-3158 |
| TAA | 3159-3161 |
| TAG | 3162-3164 |
| TAA | 3165-3167 |
| TAG | 3168-3170 |
| TAA | 3171-3173 |
| TGA | 3171-3173 |
| TAA | 3174-3176 |
| TAA | 3180-3182 |
| TAA | 3183-3185 |
| TAG | 3186-3188 |
| TAA | 3189-3191 |
| TGA | 3189-3191 |
| TAA | 3192-3194 |
| TAA | 3195-3197 |
| TAA | 3198-3200 |
| TAA | 3204-3206 |
| TAA | 3207-3209 |
| TAA | 3210-3212 |
| TAA | 3213-3215 |
| TAG | 3216-3218 |
| TAA | 3219-3221 |
| TAG | 3222-3224 |
| TAA | 3225-3227 |
| TAG | 3225-3227 |
| TGA | 3225-3227 |
| TAA | 3228-3230 |
| TAG | 3231-3233 |
| TAG | 3234-3236 |
| TAA | 3237-3239 |
| TAA | 3240-3242 |
| TAG | 3240-3242 |
| TGA | 3240-3242 |
| TAA | 3243-3245 |
| TAA | 3246-3248 |
| TAA | 3249-3251 |
| TAA | 3252-3254 |
| TAG | 3255-3257 |
| TAG | 3258-3260 |
| TAG | 3261-3263 |
| TAA | 3264-3266 |
| TAG | 3264-3266 |
| TGA | 3264-3266 |
| TAA | 3267-3269 |
| TAA | 3270-3272 |
| TAA | 3273-3275 |
| TAG | 3276-3278 |
| TAG | 3279-3281 |
| TAG | 3282-3284 |
| TAA | 3285-3287 |
| TAG | 3285-3287 |
| TGA | 3285-3287 |
| TAA | 3288-3290 |
| TAA | 3291-3293 |
| TAA | 3294-3296 |
| TAG | 3297-3299 |
| TAA | 3300-3302 |
| TAG | 3303-3305 |
| TAA | 3306-3308 |
| TAG | 3306-3308 |
| TGA | 3306-3308 |
| TAA | 3309-3311 |
| TAG | 3312-3314 |
| TAG | 3315-3317 |
| TAA | 3318-3320 |
| TAA | 3321-3323 |
| TAG | 3327-3329 |
| TAG | 3330-3332 |
| TAG | 3336-3338 |
| TAA | 3339-3341 |
| TAA | 3342-3344 |
| TAA | 3345-3347 |
| TAG | 3348-3350 |
| TAA | 3354-3356 |
| TAA | 3357-3359 |
| TAG | 3357-3359 |
| TGA | 3357-3359 |
| TAA | 3360-3362 |
| TGA | 3360-3362 |
| TAG | 3363-3365 |
| TAA | 3366-3368 |
| TAA | 3372-3374 |
| TAA | 3375-3377 |
| TAG | 3375-3377 |
| TGA | 3375-3377 |
| TAG | 3378-3380 |
| TAG | 3381-3383 |
| TAA | 3384-3386 |
| TAG | 3384-3386 |
| TGA | 3384-3386 |
| TAG | 3393-3395 |
| TAG | 3396-3398 |
| TAA | 3399-3401 |
| TAG | 3399-3401 |
| TGA | 3399-3401 |
| TAA | 3402-3404 |
| TAA | 3408-3410 |
| TAG | 3417-3419 |
| TAA | 3420-3422 |
| TAA | 3423-3425 |
| TAA | 3426-3428 |
| TAG | 3426-3428 |
| TGA | 3426-3428 |
| TAA | 3429-3431 |
| TAG | 3438-3440 |
| TAA | 3441-3443 |
| TAA | 3444-3446 |
| TAA | 3447-3449 |
| TAG | 3453-3455 |
| TAA | 3456-3458 |
| TAG | 3456-3458 |
| TGA | 3456-3458 |
| TAG | 3459-3461 |
| TAG | 3462-3464 |
| TAG | 3465-3467 |
| TAG | 3468-3470 |
| TAA | 3474-3476 |
| TGA | 3474-3476 |
| TAG | 3477-3479 |
| TAA | 3480-3482 |
| TAA | 3483-3485 |
| TAG | 3486-3488 |
| TAA | 3489-3491 |
| TAG | 3489-3491 |
| TGA | 3489-3491 |
| TAA | 3492-3494 |
| TAG | 3492-3494 |
| TGA | 3492-3494 |
| TAA | 3498-3500 |
| TAG | 3498-3500 |
| TGA | 3498-3500 |
| TAA | 3504-3506 |
| TGA | 3504-3506 |
| TAA | 3507-3509 |
| TAG | 3507-3509 |
| TGA | 3507-3509 |
| TAG | 3510-3512 |
| TAA | 3513-3515 |
| TAA | 3516-3518 |
| TAG | 3516-3518 |
| TGA | 3516-3518 |
| TAG | 3519-3521 |
| TAA | 3528-3530 |
| TAG | 3531-3533 |
| TAA | 3534-3536 |
| TAA | 3537-3539 |
| TAG | 3543-3545 |
| TAA | 3546-3548 |
| TAG | 3546-3548 |
| TGA | 3546-3548 |
| TAG | 3552-3554 |
| TAA | 3555-3557 |
| TAG | 3555-3557 |
| TGA | 3555-3557 |
| TAA | 3558-3560 |
| TAG | 3558-3560 |
| TGA | 3558-3560 |
| TAG | 3561-3563 |
| TAA | 3564-3566 |
| TGA | 3564-3566 |
| TAG | 3570-3572 |
| TAG | 3573-3575 |
| TAA | 3579-3581 |
| TAG | 3579-3581 |
| TGA | 3579-3581 |
| TAG | 3582-3584 |
| TAG | 3585-3587 |
| TAG | 3588-3590 |
| TAG | 3591-3593 |
| TAA | 3594-3596 |
| TAA | 3597-3599 |
| TAG | 3600-3602 |
| TAG | 3603-3605 |
| TAA | 3606-3608 |
| TAA | 3609-3611 |
| TAA | 3612-3614 |
| TAG | 3612-3614 |
| TGA | 3612-3614 |
| TAG | 3615-3617 |
| TAG | 3618-3620 |
| TAA | 3621-3623 |
| TAG | 3624-3626 |
| TAA | 3627-3629 |
| TAA | 3630-3632 |
| TAG | 3633-3635 |
| TAA | 3636-3638 |
| TAA | 3639-3641 |
| TAG | 3639-3641 |
| TGA | 3639-3641 |
| TAG | 3642-3644 |
| TAG | 3645-3647 |
| TAA | 3648-3650 |
| TAG | 3648-3650 |
| TGA | 3648-3650 |
| TAA | 3651-3653 |
| TAA | 3654-3656 |
| TAA | 3657-3659 |
| TAG | 3660-3662 |
| TAA | 3666-3668 |
| TGA | 3666-3668 |
| TAG | 3669-3671 |
| TAG | 3672-3674 |
| TAA | 3678-3680 |
| TAA | 3681-3683 |
| TAA | 3684-3686 |
| TGA | 3684-3686 |
| TAA | 3690-3692 |
| TAA | 3696-3698 |
| TAG | 3696-3698 |
| TGA | 3696-3698 |
| TAA | 3699-3701 |
| TAA | 3705-3707 |
| TAA | 3711-3713 |
| TAG | 3711-3713 |
| TGA | 3711-3713 |
| TAG | 3714-3716 |
| TAG | 3720-3722 |
| TAA | 3723-3725 |
| TAG | 3723-3725 |
| TGA | 3723-3725 |
| TAA | 3729-3731 |
| TAG | 3732-3734 |
| TAG | 3735-3737 |
| TAA | 3738-3740 |
| TAA | 3741-3743 |
| TGA | 3741-3743 |
| TAA | 3744-3746 |
| TAG | 3744-3746 |
| TGA | 3744-3746 |
| TAG | 3747-3749 |
| TAA | 3750-3752 |
| TAG | 3750-3752 |
| TGA | 3750-3752 |
| TAA | 3753-3755 |
| TAG | 3753-3755 |
| TGA | 3753-3755 |
| TAG | 3756-3758 |
| TAG | 3759-3761 |
| TAA | 3762-3764 |
| TGA | 3762-3764 |
| TAA | 3765-3767 |
| TAG | 3765-3767 |
| TGA | 3765-3767 |
| TAA | 3768-3770 |
| TAG | 3768-3770 |
| TGA | 3768-3770 |
| TAA | 3771-3773 |
| TAG | 3774-3776 |
| TAG | 3777-3779 |
| TAG | 3780-3782 |
| TAG | 3783-3785 |
| TAA | 3792-3794 |
| TAG | 3792-3794 |
| TGA | 3792-3794 |
| TAA | 3795-3797 |
| TAG | 3795-3797 |
| TGA | 3795-3797 |
| TAA | 3804-3806 |
| TAG | 3804-3806 |
| TGA | 3804-3806 |
| TAA | 3807-3809 |
| TAG | 3807-3809 |
| TGA | 3807-3809 |
| TAA | 3810-3812 |
| TAG | 3810-3812 |
| TGA | 3810-3812 |
| TAG | 3813-3815 |
| TAA | 3816-3818 |
| TAG | 3819-3821 |
| TAA | 3822-3824 |
| TAA | 3825-3827 |
| TAA | 3828-3830 |
| TAA | 3834-3836 |
| TAG | 3834-3836 |
| TGA | 3834-3836 |
| TAA | 3840-3842 |
| TAG | 3840-3842 |
| TGA | 3840-3842 |
| TAA | 3843-3845 |
| TAA | 3846-3848 |
| TAA | 3852-3854 |
| TAA | 3855-3857 |
| TAG | 3858-3860 |
| TAG | 3861-3863 |
| TAA | 3864-3866 |
| TAG | 3867-3869 |
| TAA | 3870-3872 |
| TAG | 3870-3872 |
| TGA | 3870-3872 |
| TAA | 3876-3878 |
| TAG | 3876-3878 |
| TGA | 3876-3878 |
| TAA | 3879-3881 |
| TAA | 3882-3884 |
| TGA | 3882-3884 |
| TAA | 3885-3887 |
| TAG | 3888-3890 |
| TAG | 3891-3893 |
| TAA | 3894-3896 |
| TGA | 3894-3896 |
| TAA | 3897-3899 |
| TAG | 3897-3899 |
| TGA | 3897-3899 |
| TAA | 3900-3902 |
| TAG | 3900-3902 |
| TGA | 3900-3902 |
| TAA | 3903-3905 |
| TAG | 3903-3905 |
| TGA | 3903-3905 |
| TAA | 3906-3908 |
| TAG | 3906-3908 |
| TGA | 3906-3908 |
| TAA | 3909-3911 |
| TGA | 3909-3911 |
| TAA | 3912-3914 |
| TGA | 3912-3914 |
| TAA | 3915-3917 |
| TAA | 3918-3920 |
| TAG | 3918-3920 |
| TGA | 3918-3920 |
| TAA | 3921-3923 |
| TAG | 3924-3926 |
| TAA | 3927-3929 |
| TAG | 3927-3929 |
| TGA | 3927-3929 |
| TAA | 3930-3932 |
| TAA | 3933-3935 |
| TAG | 3939-3941 |
| TAA | 3942-3944 |
| TAA | 3945-3947 |
| TAG | 3945-3947 |
| TGA | 3945-3947 |
| TAG | 3948-3950 |
| TAA | 3951-3953 |
| TAG | 3954-3956 |
| TAA | 3957-3959 |
| TAG | 3957-3959 |
| TGA | 3957-3959 |
| TAG | 3963-3965 |
| TAA | 3975-3977 |
| TAG | 3978-3980 |
| TAG | 3981-3983 |
| TAA | 3984-3986 |
| TAA | 3987-3989 |
| TAA | 3990-3992 |
| TAG | 3990-3992 |
| TGA | 3990-3992 |
| TAA | 3993-3995 |
| TAG | 3993-3995 |
| TGA | 3993-3995 |
| TAG | 3996-3998 |
| TAA | 3999-4001 |
| TAA | 4002-4004 |
| TAG | 4002-4004 |
| TGA | 4002-4004 |
| TAA | 4005-4007 |
| TAG | 4005-4007 |
| TGA | 4005-4007 |
| TAA | 4008-4010 |
| TAG | 4008-4010 |
| TGA | 4008-4010 |
| TAA | 4011-4013 |
| TAG | 4011-4013 |
| TGA | 4011-4013 |
| TAA | 4017-4019 |
| TAG | 4017-4019 |
| TGA | 4017-4019 |
| TAA | 4020-4022 |
| TAG | 4023-4025 |
| TAA | 4026-4028 |
| TAG | 4026-4028 |
| TGA | 4026-4028 |
| TAG | 4029-4031 |
| TAG | 4032-4034 |
| TAA | 4035-4037 |
| TAG | 4035-4037 |
| TGA | 4035-4037 |
| TAA | 4038-4040 |
| TGA | 4038-4040 |
| TAG | 4041-4043 |
| TAA | 4044-4046 |
| TAA | 4047-4049 |
| TAA | 4050-4052 |
| TAA | 4053-4055 |
| TAG | 4059-4061 |
| TAA | 4065-4067 |
| TAG | 4065-4067 |
| TGA | 4065-4067 |
| TAA | 4068-4070 |
| TAG | 4068-4070 |
| TGA | 4068-4070 |
| TAA | 4071-4073 |
| TGA | 4071-4073 |
| TAG | 4074-4076 |
| TAA | 4077-4079 |
| TAG | 4077-4079 |
| TGA | 4077-4079 |
| TAA | 4080-4082 |
| TAG | 4080-4082 |
| TGA | 4080-4082 |
| TAA | 4083-4085 |
| TAA | 4089-4091 |
| TAA | 4092-4094 |
| TGA | 4092-4094 |
| TAA | 4101-4103 |
| TAG | 4101-4103 |
| TGA | 4101-4103 |
| TAG | 4104-4106 |
| TAA | 4107-4109 |
| TAG | 4113-4115 |
| TAG | 4116-4118 |
| TAG | 4119-4121 |
| TAA | 4125-4127 |
| TGA | 4125-4127 |
| TAG | 4128-4130 |
| TAA | 4131-4133 |
| TAG | 4134-4136 |
| TAA | 4137-4139 |
| TAG | 4137-4139 |
| TGA | 4137-4139 |
| TAG | 4140-4142 |
| TAA | 4143-4145 |
| TAG | 4143-4145 |
| TGA | 4143-4145 |
| TAG | 4146-4148 |
| TAG | 4149-4151 |
| TAG | 4152-4154 |
| TAG | 4155-4157 |
| TAA | 4158-4160 |
| TAG | 4161-4163 |
| TAA | 4164-4166 |
| TAG | 4167-4169 |
| TAA | 4170-4172 |
| TAG | 4170-4172 |
| TGA | 4170-4172 |
| TAG | 4173-4175 |
| TAG | 4176-4178 |
| TAA | 4179-4181 |
| TAA | 4182-4184 |
| TAG | 4188-4190 |
| TAG | 4191-4193 |
| TAA | 4197-4199 |
| TAA | 4200-4202 |
| TAG | 4203-4205 |
| TAA | 4206-4208 |
| TAG | 4206-4208 |
| TGA | 4206-4208 |
| TAA | 4209-4211 |
| TAA | 4212-4214 |
| TAG | 4212-4214 |
| TGA | 4212-4214 |
| TAG | 4215-4217 |
| TAG | 4218-4220 |
| TAG | 4221-4223 |
| TAG | 4224-4226 |
| TAA | 4227-4229 |
| TAG | 4227-4229 |
| TGA | 4227-4229 |
| TAG | 4230-4232 |
| TAA | 4233-4235 |
| TAG | 4233-4235 |
| TGA | 4233-4235 |
| TAG | 4236-4238 |
| TAA | 4239-4241 |
| TGA | 4239-4241 |
| TAG | 4242-4244 |
| TAA | 4245-4247 |
| TAA | 4248-4250 |
| TAG | 4251-4253 |
| TAA | 4254-4256 |
| TAG | 4254-4256 |
| TGA | 4254-4256 |
| TAG | 4257-4259 |
| TAG | 4260-4262 |
| TAA | 4263-4265 |
| TAG | 4263-4265 |
| TGA | 4263-4265 |
| TAA | 4266-4268 |
| TAG | 4266-4268 |
| TGA | 4266-4268 |
| TAG | 4269-4271 |
| TAA | 4275-4277 |
| TAG | 4275-4277 |
| TGA | 4275-4277 |
| TAA | 4278-4280 |
| TAG | 4278-4280 |
| TGA | 4278-4280 |
| TAA | 4284-4286 |
| TGA | 4284-4286 |
| TAG | 4287-4289 |
| TAA | 4290-4292 |
| TAA | 4293-4295 |
| TAG | 4293-4295 |
| TGA | 4293-4295 |
| TAA | 4296-4298 |
| TAA | 4299-4301 |
| TAA | 4308-4310 |
| TGA | 4308-4310 |
| TAG | 4311-4313 |
| TAA | 4314-4316 |
| TAA | 4317-4319 |
| TAA | 4326-4328 |
| TAA | 4332-4334 |
| TGA | 4332-4334 |
| TAA | 4335-4337 |
| TAG | 4347-4349 |
| TAA | 4350-4352 |
| TAG | 4353-4355 |
| TAG | 4356-4358 |
| TAG | 4362-4364 |
| TAG | 4365-4367 |
| TAG | 4368-4370 |
| TAA | 4371-4373 |
| TAG | 4371-4373 |
| TGA | 4371-4373 |
| TAG | 4374-4376 |
| TAG | 4383-4385 |
| TAA | 4386-4388 |
| TGA | 4386-4388 |
| TAA | 4395-4397 |
| TAG | 4395-4397 |
| TGA | 4395-4397 |
| TAA | 4398-4400 |
| TAA | 4401-4403 |
| TAA | 4404-4406 |
| TAG | 4404-4406 |
| TGA | 4404-4406 |
| TAA | 4410-4412 |
| TAG | 4410-4412 |
| TGA | 4410-4412 |
| TAA | 4413-4415 |
| TAA | 4416-4418 |
| TAA | 4419-4421 |
| TAG | 4422-4424 |
| TAA | 4425-4427 |
| TAG | 4425-4427 |
| TGA | 4425-4427 |
| TAA | 4428-4430 |
| TAG | 4428-4430 |
| TGA | 4428-4430 |
| TAG | 4431-4433 |
| TAG | 4437-4439 |
| TAG | 4440-4442 |
| TAA | 4449-4451 |
| TAG | 4455-4457 |
| TAA | 4461-4463 |
| TAA | 4464-4466 |
| TAA | 4467-4469 |
| TAG | 4467-4469 |
| TGA | 4467-4469 |
| TAG | 4470-4472 |
| TAA | 4473-4475 |
| TAG | 4476-4478 |
| TAG | 4479-4481 |
| TAA | 4482-4484 |
| TAG | 4482-4484 |
| TGA | 4482-4484 |
| TAA | 4485-4487 |
| TAA | 4488-4490 |
| TAG | 4488-4490 |
| TGA | 4488-4490 |
| TAA | 4494-4496 |
| TGA | 4494-4496 |
| TAA | 4497-4499 |
| TAA | 4500-4502 |
| TAG | 4503-4505 |
| TAA | 4506-4508 |
| TAG | 4506-4508 |
| TGA | 4506-4508 |
| TAA | 4512-4514 |
| TAA | 4515-4517 |
| TAA | 4521-4523 |
| TGA | 4521-4523 |
| TAG | 4527-4529 |
| TAG | 4530-4532 |
| TAA | 4536-4538 |
| TAG | 4536-4538 |
| TGA | 4536-4538 |
| TAG | 4539-4541 |
| TAG | 4542-4544 |
| TAA | 4545-4547 |
| TAA | 4548-4550 |
| TAG | 4548-4550 |
| TGA | 4548-4550 |
| TAG | 4551-4553 |
| TAG | 4554-4556 |
| TAA | 4557-4559 |
| TAG | 4557-4559 |
| TGA | 4557-4559 |
| TAG | 4560-4562 |
| TAG | 4563-4565 |
| TAA | 4566-4568 |
| TAA | 4569-4571 |
| TAG | 4569-4571 |
| TGA | 4569-4571 |
| TAA | 4572-4574 |
| TAA | 4575-4577 |
| TAA | 4578-4580 |
| TAA | 4581-4583 |
| TAA | 4584-4586 |
| TAG | 4587-4589 |
| TAG | 4593-4595 |
| TAG | 4596-4598 |
| TAG | 4599-4601 |
| TAA | 4602-4604 |
| TAA | 4605-4607 |
| TAG | 4605-4607 |
| TGA | 4605-4607 |
| TAA | 4608-4610 |
| TAG | 4608-4610 |
| TGA | 4608-4610 |
| TAA | 4614-4616 |
| TAG | 4614-4616 |
| TGA | 4614-4616 |
| TAA | 4617-4619 |
| TAA | 4620-4622 |
| TAG | 4620-4622 |
| TGA | 4620-4622 |
| TAA | 4626-4628 |
| TAG | 4626-4628 |
| TGA | 4626-4628 |
| TAA | 4629-4631 |
| TAG | 4629-4631 |
| TGA | 4629-4631 |
| TAG | 4632-4634 |
| TAG | 4635-4637 |
| TAA | 4638-4640 |
| TAA | 4641-4643 |
| TAG | 4641-4643 |
| TGA | 4641-4643 |
| TAA | 4644-4646 |
| TAG | 4644-4646 |
| TGA | 4644-4646 |
| TAA | 4647-4649 |
| TAA | 4653-4655 |
| TAA | 4656-4658 |
| TAG | 4656-4658 |
| TGA | 4656-4658 |
| TAA | 4659-4661 |
| TAG | 4659-4661 |
| TGA | 4659-4661 |
| TAG | 4662-4664 |
| TAA | 4665-4667 |
| TGA | 4665-4667 |
| TAA | 4674-4676 |
| TGA | 4674-4676 |
| TAA | 4677-4679 |
| TAA | 4680-4682 |
| TAA | 4683-4685 |
| TAG | 4683-4685 |
| TGA | 4683-4685 |
| TAA | 4689-4691 |
| TAG | 4689-4691 |
| TGA | 4689-4691 |
| TAG | 4695-4697 |
| TAG | 4698-4700 |
| TAA | 4704-4706 |
| TAA | 4707-4709 |
| TAG | 4710-4712 |
| TAG | 4713-4715 |
| TAG | 4716-4718 |
| TAG | 4719-4721 |
| TAG | 4722-4724 |
| TAG | 4725-4727 |
| TAG | 4737-4739 |
| TAG | 4740-4742 |
| TAA | 4743-4745 |
| TAG | 4743-4745 |
| TGA | 4743-4745 |
| TAG | 4746-4748 |
| TAG | 4755-4757 |
| TAA | 4758-4760 |
| TAG | 4758-4760 |
| TGA | 4758-4760 |
| TAA | 4761-4763 |
| TGA | 4761-4763 |
| TAG | 4764-4766 |
| TAA | 4767-4769 |
| TAA | 4770-4772 |
| TAG | 4770-4772 |
| TGA | 4770-4772 |
| TAA | 4773-4775 |
| TAG | 4773-4775 |
| TGA | 4773-4775 |
| TAA | 4776-4778 |
| TAG | 4776-4778 |
| TGA | 4776-4778 |
| TAA | 4782-4784 |
| TAG | 4788-4790 |
| TAG | 4794-4796 |
| TAG | 4797-4799 |
| TAA | 4800-4802 |
| TAA | 4806-4808 |
| TAG | 4806-4808 |
| TGA | 4806-4808 |
| TAG | 4812-4814 |
| TAA | 4815-4817 |
| TAG | 4815-4817 |
| TGA | 4815-4817 |
| TAG | 4818-4820 |
| TAA | 4821-4823 |
| TAA | 4824-4826 |
| TAA | 4830-4832 |
| TAG | 4830-4832 |
| TGA | 4830-4832 |
| TAA | 4833-4835 |
| TAG | 4833-4835 |
| TGA | 4833-4835 |
| TAG | 4836-4838 |
| TAG | 4839-4841 |
| TAG | 4845-4847 |
| TAA | 4848-4850 |
| TAG | 4848-4850 |
| TGA | 4848-4850 |
| TAG | 4851-4853 |
| TAA | 4857-4859 |
| TAG | 4857-4859 |
| TGA | 4857-4859 |
| TAG | 4860-4862 |
| TAG | 4863-4865 |
| TAA | 4866-4868 |
| TAG | 4869-4871 |
| TAG | 4875-4877 |
| TAA | 4878-4880 |
| TAG | 4878-4880 |
| TGA | 4878-4880 |
| TAG | 4881-4883 |
| TAG | 4887-4889 |
| TAG | 4890-4892 |
| TAA | 4893-4895 |
| TAA | 4896-4898 |
| TAA | 4902-4904 |
| TAG | 4902-4904 |
| TGA | 4902-4904 |
| TAA | 4905-4907 |
| TAG | 4905-4907 |
| TGA | 4905-4907 |
| TAA | 4908-4910 |
| TAA | 4911-4913 |
| TAG | 4911-4913 |
| TGA | 4911-4913 |
| TAG | 4914-4916 |
| TAA | 4917-4919 |
| TAG | 4917-4919 |
| TGA | 4917-4919 |
| TAA | 4920-4922 |
| TGA | 4920-4922 |
| TAG | 4923-4925 |
| TAA | 4932-4934 |
| TAG | 4932-4934 |
| TGA | 4932-4934 |
| TAA | 4935-4937 |
| TGA | 4935-4937 |
| TAG | 4938-4940 |
| TAG | 4941-4943 |
| TAG | 4944-4946 |
| TAA | 4947-4949 |
| TAG | 4947-4949 |
| TGA | 4947-4949 |
| TAG | 4950-4952 |
| TAG | 4956-4958 |
| TAG | 4962-4964 |
| TAG | 4965-4967 |
| TAG | 4968-4970 |
| TAA | 4974-4976 |
| TAA | 4977-4979 |
| TAG | 4980-4982 |
| TAA | 4983-4985 |
| TAG | 4986-4988 |
| TAG | 4989-4991 |
| TAA | 4992-4994 |
| TAG | 4992-4994 |
| TGA | 4992-4994 |
| TAA | 4995-4997 |
| TAG | 4998-5000 |
| TAA | 5001-5003 |
| TAG | 5004-5006 |
| TAG | 5007-5009 |
| TAA | 5010-5012 |
| TAG | 5013-5015 |
| TAA | 5016-5018 |
| TGA | 5016-5018 |
| TAA | 5019-5021 |
| TAG | 5022-5024 |
| TAA | 5025-5027 |
| TGA | 5025-5027 |
| TAG | 5031-5033 |
| TAA | 5034-5036 |
| TAG | 5034-5036 |
| TGA | 5034-5036 |
| TAA | 5037-5039 |
| TGA | 5037-5039 |
| TAG | 5040-5042 |
| TAA | 5043-5045 |
| TAG | 5043-5045 |
| TGA | 5043-5045 |
| TAA | 5046-5048 |
| TAG | 5049-5051 |
| TAA | 5052-5054 |
| TAG | 5055-5057 |
| TAA | 5058-5060 |
| TAA | 5061-5063 |
| TAA | 5064-5066 |
| TAA | 5067-5069 |
| TAA | 5070-5072 |
| TAG | 5070-5072 |
| TGA | 5070-5072 |
| TAA | 5073-5075 |
| TAG | 5076-5078 |
| TGA | 5091-5093 |

Insertion mutant BRCA1 genes containing a truncating mutation may also be defined as having the sequence 5′ R1-R2-R3 3′; where R1 is the wild type BRCA1 DNA sequence from nucleotide number 120 to X; R2 is 3n+1 or 3n+2 nucleotides of any sequence where n is an integer of zero or greater; R3 contains the wild type BRCA1 DNA sequence of nucleotide number X+1 to 5711, and where X=123 to 5707.

Alternatively, an insertion mutant BRCA1 gene or fragment thereof containing a truncating mutation is capable of specifically hybridizing to an oligonucleotide probe being at least 9 nucleotides in length and having the sequence 5′ R1-R2-R3 3′; where R1 contains at its 3′ end three nucleotides complementary to nucleotide numbers X-2, X-1 and X of the wild-type BRCA1 gene; R2=an oligonucleotide having Y nucleotides of any sequence; R3 contains at its 5′ end three nucleotides complementary to nucleotide numbers X+1, X+2 and X+3 of the wild type BRCA1 gene; where Y is 3n+1 or 3n+2 where n is an integer of zero to 1861, and where X=122 to 5707.

It should be recognized that for TABLES 4-7, each line indicating a stop codon represents several different mutations, each one of which creates the same truncating stop codon. For example, in TABLE 4, the line indicated by TGA stop codon at 207-209 represents twenty-four different mutations, 185delA, 186delG, 187delA, 188delG, 189delT, 190delG, 191 delT, 192delC, 193delC, 194delC, 195delA, 196delT, 197delC, 198delT, 199delG, 200delT, 201delC, 202delT, 203delG, 204delG, 205delA, 206delG, 207delT, 208delT. The corresponding mutations for each line in each table are easily determined by anyone of very modest skill in the art knowing only the BRCA1 sequence such as given SEQ ID NO:1 and TABLES 4-7.

A substantially complete listing of all of the mutations, their sites etc. of the present invention is described in Appendix A, B, C, D and E. Likewise, the choice of mutations and corresponding oligonucleotides may be chosen from and determined by the list in Appendix A-E.

An alternative method for defining the mutations of the present invention is by their nucleotide numbers. Mutant BRCA1 genes having nonsense mutations may be described as having the nucleotide sequence R4-R5-R6, where R4 is nucleotide numbers 120 to 3×of the BRCA1 gene; R5 is TAG, TAA or TGA; and R6 is nucleotide numbers 3X+4 to 5711 of the BRCA1 gene; where X is 41 to 1903.

Mutant BRCA1 genes having deletion mutations may be described as having the nucleotide sequence R4-R5, where R4 is nucleotide numbers 120 to Y of the BRCA1 gene; and R5 is nucleotide numbers Y+Z+1 to 5711 of the BRCA1 gene; where Y is 124 to 5707, and Z is 3n+1 or 3n+2 where N is an integer of zero or greater.

Mutant BRCA1 genes having insertion mutations may be described as having the nucleotide sequence R4-R5-R6, where R4 is nucleotide numbers 120 to Y of the BRCA1 gene; R5 is 3n+1 or 3n+2 nucleotides of any sequence where n is an integer of zero or greater; and R6 is nucleotides Y+1 to 5711; wherein Y is from 122 to 5707.

While the present invention encompasses genes with numerous mutations in the BRCA1 gene, applicant reserves the right to lessen the scope and number of mutations to be included in the present invention.

It should be recognized that mutations causing truncations which form a smaller protein molecule than mutations causing truncations of known mutations associated with cancer are expected to also be associated with cancer. Removing additional amino acids from a non-function protein is also believed to result in a non-functional protein.

Useful oligonucleotides according to the present invention are those which will specifically hybridize to BRCA1 sequences in the region of the mutations. The oligonucleotides of the present invention are preferably “biologically active” with respect to structural attributes, such as the capacity of a nucleic acid to hybridize to another nucleic acid molecule or to be used by a polymerase as a primer. Alternatively, such attributes may be catalytic, and thus involve the capacity of the agent to mediate a chemical reaction or response. Typically these oligonucleotides are about 13 to 27 nucleotides in length (longer for large insertions) and have the nucleotide sequence corresponding to the region of the mutations at their respective nucleotide locations on the BRCA1 sequence. Such molecules can be labeled, according to any technique known in the art, such as with radiolabels, fluorescent labels, enzymatic labels, sequence tags, biotin, other ligands, etc.

According to another aspect of the invention, the oligonucleotides contain one or more of the specific mutations constituting DNA probes. Generally it is preferred for each DNA probe to encompass only one mutation. Such molecules may be labeled and can be used as allele-specific oligonucleotide probes to detect the mutation of interest.

Alternatively, the oligonucleotide may be one primer of a PCR primer pair, which upon annealing, will amplify a product. In the situation wherein the target DNA sample does not contain a sequence complementary to the oligonucleotide, annealing does not occur, and thus amplification of a product does not occur.

Polynucleotide-containing biological samples, such as blood, can be tested to determine whether the BRCA1 gene contains one of the specific mutations listed above. To amplify the BRCA1 gene, one may use PCR using primers which hybridize to the ends of the exons or to the introns flanking the exons. To detect mutations in the introns, primers amplifying the introns, especially the regions adjacent to the exons (particularly the splice site regions), may be used. Examples of suitable primers are given in Friedman et al., Nat. Genetics, 8:399-404 (1994).

Amplification may also be performed by a number of other techniques, such as by cloning the gene or gene fragments, and linking the BRCA1 gene or fragments thereof in the sample to a vector. “Shot gun” cloning is particularly preferred. For the purposes of this to application, a vector may be any polynucleotide containing system which induces replication such as a plasmid, cosmid, virus, transposon, or portions thereof.

In one embodiment of the invention, the BRCA1 gene or A DNA fragment complementary to its coding sequence is ligated to a vector which is placed inside a suitable host cell or other system for replicating the vector. After replication, the BRCA1 gene or its fragments are then separated from the vector, e.g. by restriction endonuclease digestion, to amplify the copy number of BRCA1 in a particular preparation.

Probes are synthesized to specifically hybridize to any of the list of mutations in TABLES 3-7. On each side of the mutation, the probe overlaps at least 3 nucleotides so that the probes specifically hybridize to a DNA with the mutation. Likewise for probes specific to the mutation site with a sequence complementary for the wild type DNA sequence. By using either or both (if the sample is heterozygous) of these probes which differentially hybridize to mutant and wild-type BRCA1 sequences, one can determine the presence or absence of a mutant BRCA1 gene. Probes which hybridize to the complementary strand of the target DNA may also be used in the same manner.

A pair of isolated allele specific oligonucleotide probes are provided for the mutation 185delAG.

| | | |
|---|---|---|
| wild-type | 5′-AAT CTT AGA GTG TCC CA-3′, | SEQ ID NO:3 |
| mutant | 5′-ATC TTA GTG TCC CAC CT-3′, | SEQ ID NO:4 |

SEQ ID NO:3 preferentially may be hybridized to a target BRCA1 sequence under conditions where this probe anneals with a wild type BRCA1 gene or gene fragments, whereas SEQ ID NO:4 preferentially may be hybridized to a target BRCA1 sequence under conditions where this probe anneals with BRCA1 gene or gene fragments containing the 185delAG mutation.

A pair of isolated allele specific oligonucleotide probes are provided for the mutation 1136insA.

| | | |
|---|---|---|
| wild-type | 5′-CAG AAA AAA AGG TAG AT-3′, | SEQ ID NO:5 |
| mutant | 5′-CAG AAA AAA AAG GTA GA-3′, | SEQ ID NO:6 |

SEQ ID NO:5 preferentially may be hybridized to a target BRCA1 sequence under conditions where this probe anneals with a wild type BRCA1 gene or gene fragments, whereas SEQ ID NO:6 preferentially may be hybridized to a target BRCA1 sequence under conditions where this probe anneals with BRCA1 gene or gene fragments containing the 1136insA mutation.

A pair of isolated allele specific oligonucleotide probes are provided for the mutation 5382insC.

| | | |
|---|---|---|
| wild-type | 5′-AGA GAA TCC CAG GAC AG-3′, | SEQ ID NO:7 |
| mutant | 5′-AGA GAA TCC CCA GGA CA-3′, | SEQ ID NO:8 |

SEQ ID NO:9 preferentially may be hybridized to a target BRCA1 sequence under conditions where this probe anneals with a wild type BRCA1 gene or gene fragments, whereas SEQ ID NO:10 preferentially may be hybridized to a target BRCA1 sequence under conditions where this probe anneals with BRCA1 gene or gene fragments containing the 5382insC mutation.

A pair of isolated allele specific oligonucleotide probes are provided for the mutation C4446T.

| | |
|---|---|
| wild-type | |
| 5′-AGG ACC TGC GAA ATC CA-3′, | SEQ ID NO:9 |
| mutant | |
| 5′-AGG ACC TGT GAA ATC CA-3′, | SEQ ID NO:10 |

SEQ ID NO:11 preferentially may be hybridized to a target BRCA1 sequence under conditions where this probe anneals with a wild type BRCA1 gene or gene fragments, whereas SEQ ID NO:12 preferentially may be hybridized to a target BRCA1 sequence under conditions where this probe anneals with BRCA1 gene or gene fragments containing the C4446T mutation. Comparable probes can be prepared for each mutation of the present invention.

These allele specific oligonucleotides are useful in diagnosis of a subject at risk of having cancer. The allele specific oligonucleotides hybridize with a target polynucleotide sequence containing the mutations listed in TABLES 3-7. The probes having a sequence to naturally occurring (wild-type) BRCA1 hybridize preferentially to the wild type sequence and are useful, for example, as controls. The probes complementary to the sequences containing the mutations listed in TABLES 3-7 are designed to hybridize preferentially to the sequences carrying the specified mutant sequence.

The primers of the invention embrace oligonucleotides of sufficient length and appropriate sequence so as to provide initiation of polymerization on a significant number of nucleic acids in the mutated locus. Examples of preferred sequences for the primers of the present invention are given in the references cited above.

Environmental conditions conducive to synthesis of extension products include the presence of nucleoside triphosphates, an agent for polymerization, such as DNA polymerase, and suitable conditions such as temperature, ion composition, ionic strength and pH. The primer is preferably single stranded for maximum efficiency in amplification, but may be double stranded. If double stranded, the primer is preferably first treated to separate its strands before being used to prepare extension products. The primer must be sufficiently long to specifically prime the synthesis of extension products in the presence of the inducing agent for polymerization. The exact length of primer will depend on many factors, including temperature, buffer, and nucleotide composition. The oligonucleotide primer typically contains 13-20 or more nucleotides, although it may contain fewer nucleotides.

Primers of the invention are designed to be “substantially” complementary to each strand of the genomic locus to be amplified. This means that the primers must be sufficiently complementary to hybridize with their respective strands under conditions which allow the agent for polymerization to perform a polymerase-mediated primer extension reaction. In other words, the primers should have sufficient complementarity with the 5′ and 3′ sequences flanking the mutation to hybridize therewith and permit amplification of the genomic locus. “Substantially” the same as it refers to oligonucleotide sequences which have the functional ability to hybridize or anneal with sufficiently stringent conditions to generate sufficient specificity to distinguish between the presence or absence of the mutation. This is measurable by the temperature of melting being sufficiently different to permit easy identification of whether the oligonucleotide is binding to the normal or mutant BRCA1 gene sequence. Conventional stringency conditions are described, for example, by Sambrook et al, Molecular Cloning, a laboratory Manual, 2nd Edition, Cold Spring Harbor Press, Cold Spring Harbor, N.Y. (1989), more recent editions and Haymes, et al., Nucleic Acid Hybridization, A Practical Approach, IRL Press, Washington, D.C. (1985).

Oligonucleotide primers of the invention are employed in the amplification process, which is an enzymatic chain reaction that preferably produces exponential quantities of mutated locus relative to the number of reaction steps involved. Typically, one primer is complementary to the negative (−) strand of the mutated locus and the other is complementary to the positive (+) strand. Annealing the primers to denatured nucleic acid is generally followed by extension with an enzyme, such as the large fragment of DNA polymerase I (Klenow) and nucleotides, and results in newly synthesized + and − strands containing the target mutated locus sequence. Because these newly synthesized sequences are also templates, repeated cycles of denaturing, primer annealing, and extension results in exponential production of the region (i.e., the target mutated locus sequence) defined by the primers. The product of the chain reaction is a discreet nucleic acid duplex with termini corresponding to the ends of the specific primers employed.

The oligonucleotide primers of the invention may be prepared using any suitable method, such as conventional phosphotriester and phosphodiester methods or automated embodiments thereof. In one such automated embodiment, diethylphosphoramidites are used as starting materials and may be synthesized as described by Beaucage, et al., Tetrahedron Letters, 22:1859-1862, (1981). One method for synthesizing oligonucleotides on a modified solid support is described in U.S. Pat. No. 4,458,066.

Any nucleic acid specimen, in purified or non-purified form, can be utilized as the is starting nucleic acid or acids, providing it contains, or is suspected of containing, the specific nucleic acid sequence containing the mutated locus. Thus, the process may amplify, for example, DNA or RNA, including messenger RNA, wherein DNA or RNA may be single stranded or double stranded. In the event that RNA is to be used as a template, enzymes, and/or conditions optimal for reverse transcribing the template to DNA would preferably be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The specific nucleic acid sequence to be amplified, i.e., the mutated locus, may be a fraction of a larger molecule or can. be present initially as a discrete molecule, so that the specific sequence constitutes the entire nucleic acid. It is not necessary that the sequence to be amplified be present initially in a pure form; it may be a minor fraction of a complex mixture, such as contained in whole human DNA.

DNA utilized herein may be extracted from a body sample, such as blood, tissue material and the like by a variety of techniques such as that described by Maniatis, et. al. in Molecular Cloning: A Laboratory Manual, Cold Spring Harbor, N.Y., p. 280-281, 1982). If the extracted sample is impure, it may be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

The deoxyribonucleotide triphosphates dATP, dCTP, dGTP, and dTTP are added to the synthesis mixture, either separately or together with the primers, in adequate amounts and the resulting solution is heated to about 90′-100° C. from about 1 to 10 minutes, preferably from 1 to 4 minutes. This is sufficient to denature any double strands. After this heating period, the solution is allowed to cool at a rate which is preferable for the primer hybridization. To the cooled mixture is added an appropriate agent for effecting the primer extension reaction (called herein agent for polymerization), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C. Thermostable DNA polymerases, such as Taq polymerase, may function at a higher temperature.

The agent for polymerization may bge any compound or system which will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (i e., those enzymes which perform primer extension after being subjected to temperatures sufficiently elevated to cause denaturation), such as Taq polymerase. The suitable enzyme will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths.

The newly synthesized strand and its complementary nucleic acid strand will form a double-stranded molecule under hybridizing conditions described above and this hybrid is used in subsequent steps of the process. In the next step, the newly synthesized double-stranded molecule is subjected to denaturing conditions using any of the procedures described above to provide single-stranded molecules.

The steps of denaturing, annealing, and extension product synthesis can be repeated as often as needed to amplify the target nucleic acid sequence to the extent necessary for detection. The amount of the specific nucleic acid sequence produced will accumulate in an exponential fashion (PCR. A Practical Approach, ILR Press, Eds. M. J. McPherson, P. Quirke, and G. R; Taylor, 1992).

The amplification products may be detected by Southern blot analysis using non-isotopic detection methods. In such a process, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the probe, or will be able to ascertain such, using routine experimentation. In the preferred embodiment, the amplification products are determinable by separating the mixture on an agarose gel containing ethidium bromide which causes DNA to be fluorescent.

Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et.al., Bio/Technology, 3:1008-1012, (1985)), allele-specific oligonucleotide (ASO) probe analysis (Conner, et. al., Proc. Natl. Acad. Sci. U.S.A., 80:278, (1983)), oligonucleotide ligation assays (OLAs) (Landgren, et. al., Science, 241:1007, (1988)), and the like. Molecular techniques for DNA analysis have been reviewed (Landgren, et. al., Science, 242:229-237, (1988)).

Preferably, the method of amplifying is by PCR, as described herein and as is commonly used by those of ordinary skill in the art. Alternative methods of amplification have been described and can also be employed as long as the BRCA1 locus amplified by PCR using primers of the invention is similarly amplified by the alternative means. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is 10⁸ to 10⁹ fold. The QB replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labeled halves of a sequence of interest which are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA. nucleotides to geometrically amplify targeted sequences. A 2-base gap separates the oligonucleotide probe pairs, and the RCR fills and joins the gap, mimicking normal DNA repair. Nucleic acid amplification by strand displacement activation (SDA) typically utilizes a short primer containing a recognition site for Hinc II with short overhang on the 5′ end which binds to target DNA. A DNA polymerase fills in the part of the primer opposite the overhang with sulfur-containing adenine analogs. Hinc II is added but only cuts the unmodified DNA strand. A DNA polymerase that lacks 5′ exonuclease activity enters at the cite of the nick and begins to polymerize, displacing the initial primer strand downstream and building a new one which serves as more primer. SDA produces greater than 10⁷-fold amplification in 2 hours at 37° C. Unlike PCR and LCR, SDA does not require instrumented Temperature cycling. Another modification of the PCR is the TAQMAN amplification (PERKIN ELMER) where an oligonucleotide is labeled with a fluorescent and a quencher. This oligonucleotide anneals to the target between the primers so that when one primer is extended, the 5′ nuclease activity of Taq cleaves of the fluorescent label which is then qualitatively detected and quantitatively determined to correspond to the copy number of amplification. Although PCR is the preferred method of amplification in the invention, other methods such as the above can also be used to amplify the BRCA1 locus in accordance with the present invention.

To sequence the coding region of the BRCA1 gene, each exon is amplified separately using a pair of PCR primers and the resulting PCR products are sequenced in the forward and reverse directions. Any combination of the primers mentioned above which encompass the entire BRCA1 coding region may be used.

An alternative method for determining whether a truncating mutation is present is the Protein Truncation Assay (PTA). Protein truncation assay enables us to identify three types mutations in a truncated BRCA1 protein: nonsense mutation, frame shift mutation, and splice-site mutations. Nonsense mutations (see TABLE 3) result when a single base change in a codon creates a signal to terminate the production of the protein. These signals or stop codons come in three types: TGA, TAA, TAG. Frame shift mutations (see TABLES 4-7) occur when bases are added or deleted from the normal sequence. Thus, disrupting the reading frame of the protein and causing a stop codon downstream from the alteration. Splice-site mutations occurring at the intron/exon boundaries have the potential of causing the deletion of an entire exon. Examples of protein truncation assays for BRCA1 is mentioned in Furnari et al, Proceedings of the National Academy of Sciences U.S.A., 96: p. 12479-12484 (11-1997) and Tashiro et al, Cancer Research, 57: 3935-3940 (1997).

Preferably, the Polymerase Chain Reaction (PCR) is performed to amplify the BRCA1 gene copy number. The amplified BRCA1 gene is transcribed and translated in vitro. Detection of truncated proteins is made possible by the use of polyacrylamide gel electrophoresis. The migration of the mutant band on the gel allows for size targeting of the alteration; thus reducing confirmatory sequencing to a minimum.

In another embodiment of the invention, a method is provided for diagnosing a subject having a predisposition or higher susceptibility to cancer, or other pathology associated with BRCA1 mutations, comprising sequencing a target nucleic acid of a sample from a subject by dideoxy sequencing following amplification of the target nucleic acid. In such an embodiment, one does not even need to use any of the oligonucleotides, either primers or probes as described herein. The BRCA1 gene, or fragments thereof, may be directly cloned and then sequenced. (such as by dideoxy methods) to determine the presence of absence of a mutation. In such a situation, one need only compare the sequence obtained to a naturally occurring (wild type) BRCA1 gene, or a portion thereof.

In another embodiment of the invention a method is provided for diagnosing a subject having a predisposition or higher susceptibility to cancer comprising contacting a target nucleic acid of a sample from a subject with a reagent that detects the presence of one of the mutations of the present invention and detecting the mutation.

In yet another embodiment of the invention, a method is provided for determining whether either gene therapy or protein therapy (with normal BRCA1 protein) is appropriate for the prevention or treatment of cancers and other BRCA1 related syndromes. For this method, BRCA1 mutations are assayed for in a biological sample for BRCA1 mutations. When present, the use of gene therapy or protein therapy to prevent cancer in the individual is appropriate. Likewise when BRCA1 mutations are found in tumor cells from a patient, gene therapy or protein therapy is appropriate for that individual.

In another embodiment of the invention, a method and reagents are provided for repairing the gene mutation in at least some cells by applying an oligomer comprising the sequence of the wild-type probes to repair the individual's genome by triple strand hybridization. See U.S. Pat. Nos. 5,650,316 and 5,624,803 for example. This is a form of gene therapy to correct the defect in either apparently normal tissue or in an active tumor. Gene repair may also be performed on excised tumor cells which may be helpful in determining the preferred therapy to be used, particularly the reagents used for gene therapy. Other forms of gene therapy, such as providing a complete copy of a normal BRCA1 gene may also be used. Some gene therapy techniques specific to BRCA1 are discussed in Furnari et al, Proceedings of the National Academy of Sciences. U.S.A., 96: p. 12479-12484 (11-1997).

Since the method of the present invention may be applied to detect a mutant BRCA1 gene in a fetus, therapeutic or preventative measures may be possible. Screening of eggs or sperm from heterozygous individuals may permit one to selectively conceive a zygote without the mutant BRCA1 gene since only one half of the sperm or eggs will contain the mutation.

In another embodiment of the invention a method is provided for characterizing a tumor. Histologic type, morphologic grade, differences between inherited and sporadic cancer appear to be distinguished. One method comprises sequencing the target nucleic acid isolated from the tumor or other biological sample to determine if the mutation is present. Sanger, et al., J. Mol. Biol. 142:1617 (1980).

Characterizing a tumor as having originated from an inherited gene, a known or suspected cause, or a sporadic cancer gene may be clinically significant as the prevalence of bilateral breast cancer is higher in individuals with a known mutation in a tumor suppressor gene than in sporadic cases. Weber, Scientific American, January-February p. 12-21 (1996). The tumor may be classified based on tissue taken from the tumor itself or from a non-tumor site which contains DNA.

Yet another embodiment of the present invention is an isolated mutant BRCA1 DNA sequence which may be the entire sequence, an intron, an exon thereof or a fragment or combination thereof. The BRCA1 DNA may be hybridized to an oligonucleotide probe, primer or polynucleotide and still be considered “isolated”. The DNA sequence must contain at least one mutation from the list provided in TABLES 3-7. Preferably, the isolated DNA sequence contains a sequence complementary to at least one of the oligonucleotides complementary to the mutations listed in TABLES 3-7. However, the DNA sequence may contain the DNA sequence of these oligonucleotides. This sequence alone has usefulness or after cloning and expression to determine suitable treatments to prevent formation of a tumor, prevent transmission of the mutant gene to offspring or to decide other prophylactic, diagnostic and treatment protocols. The isolated DNA sequence may also be used for drug design by protein replacement, protein mimetics, screening known and unknown compounds, anti-idiotype antibodies to the BRCA1 active site, for the preparation of an immunogen or vaccine and determining appropriate gene therapy to counter the pathology associated with the mutant BRCA1 gene. For diagnostic purposes, knowing the mutant BRCA1 sequence for comparison purposes is the critical step in diagnosis.

Another method comprises contacting a target nucleic acid of a sample from a subject with a reagent that detects the presence of the mutation and detecting the mutation. A number of hybridization methods are well known to those skilled in the art. Many of them are useful in carrying out the invention.

The materials for use in the method of the invention are also ideally suited for the preparation of a diagnostic kit. Such a kit may comprise a carrier means being compartmentalized to receive in close confinement one or more container means such as vials, tubes, and the like, each of the container means comprising one or more of the separate elements to be used in the method. For example, one of the container means may comprise means for amplifying BRCA1 DNA, said means comprising the necessary enzyme(s) and oligonucleotide primers for amplifying said target DNA from the subject. Another container may contain oligonucleotide probes for detecting the presence or absence of a mutation.

The oligonucleotide primers include primers having a sequences referenced above or primer sequences substantially complementary or substantially homologous thereto. Other primers flanking the BRCA1 locus or a region containing one of the mutation sites may be used. The target flanking 5′ and 3′ polynucleotide sequence include other oligonucleotide primers for amplifying the BRCA1 locus will be known or readily ascertainable to those of skill in the art. See the GENBANK sequences mentioned above where flanking sequences are given.

Oligonucleotide probes including probes having substantially the sequence complementary to the mutations listed in TABLES 3-7 or complementary sequences are useful. Other oligonucleotide probes which hybridize to one or more of the BRCA1 mutation sites and sequences substantially complementary or homologous thereto may be used. Other oligonucleotide probes for detecting the mutations will be known or readily ascertainable to those of skill in the art.

The following definitions are provided for the purpose of understanding this invention.

The term “primer” as used herein refers to a sequence comprising two or more deoxyribonucleotides or ribonucleotides, preferably more than three, and more preferably more than eight and most preferably at least 20 nucleotides of the BRCA1 gene wherein the sequence corresponds to a sequence flanking one of the mutations or wild type sequences of BRCA1 corresponding to the mutation sites. Primers may be used to initiate DNA synthesis via the PCR. Oligonucleotides of the present invention can be used for primer hybridization and others will be known or readily ascertainable to those of skill in the art.

The term “substantially complementary to” or “substantially the sequence” refers to sequences which hybridize to the sequences provided under stringent conditions and/or sequences having sufficient homology with, such that the allele specific oligonucleotides of the invention hybridize to the sequence.

“Isolated” as used herein refers to being substantially free of other proteins, lipids, carbohydrates or other materials with which they may be associated. It also refers to being substantially free of polynucleic acids being covalently bound thereto. A DNA may be hybridized to another DNA and still be considered “isolated”, such as being hybridized to a solid phase bound or labeled oligonucleotide probe. Such association is typically either in cellular material or in a synthesis medium.

“Biological sample” refers to a polynucleotide containing sample originally from a biological source. The sample may be from a living, dead, paraffin-embedded tumor specimen or even archeological source from a variety of tissues and cells. Examples include: body fluid [blood (leukocytes), urine (epithelial cells), saliva, cervical and vaginal secretions, milk . . . ] skin, hair roots/follicle, mucus membrane (e.g. buccal or tongue cell scrapings), cervicovaginal cells (from PAP smear, etc.) internal tissue (normal or tumor), chorionic villus tissue, amniotic cells, placental cells, fetal cells, cord blood, sperm or egg.

“Coding sequence” or “DNA coding sequence” refers to those portions of a gene which, taken together, code for a peptide (protein), or for which the nucleic acid itself has function. The DNA coding sequence generally encodes the “complete” protein which is one which has the same biological activity as the naturally occurring BRCA1 protein.

A “target polynucleotide” refers to the nucleotide sequence of interest e.g., the BRCA1 encoding polynucleotide. The nucleotides may be deoxyribonucleotides, ribonucleotides, acyclic derivatives and other functional equivalents such as spacer molecules (inosine, the sugar moiety without a base, etc.) and other molecules which are incorporated by a RNA polymerase, a DNA polymerase or a reverse transcriptase.

“Consensus” means the most commonly occurring in the population.

As used herein, a nucleic acid molecule is the “complement” of another nucleic acid molecule if it exhibits complete complementarity. As used herein, molecules are said to exhibit a “complete complementarity” when every nucleotide of one of the molecules is complementary to a nucleotide of the other. Two molecules are said to be “substantially complementary” if they can hybridize to one another with sufficient stability to permit them to remain annealed to one another under at least conventional “high-stringency” conditions. Similarly, the molecules are said to be “partially complementary” if they can hybridize to one another with sufficient stability to permit them to remain annealed to one another under conventional “low-stringency” conditions. Conventional stringency conditions are described, for example, by Sambrook, J., et al., (In: Molecular Cloning, a Laboratory Manual, 2nd Edition, Cold Spring Harbor Press, Cold Spring Harbor, N.Y. (1989)), and by Haymes, B. D., et al. (In: Nucleic Acid Hybridization, A Practical Approach, IRL Press, Washington, D.C. (1985)), both herein incorporated by reference).

As used herein, an oligonucleotide is said to be capable of “specifically hybridizing” to a complementary target polynucleotide if the two molecules are capable of forming an anti-parallel, double-stranded nucleic acid structure under stringent hybridization conditions, whereas the oligonucleotide is substantially unable to form such a structure when incubated under the same conditions with a target polynucleotide to which the oligonucleotide is not substantially complementary.

“Sequence variation” as used herein refers to any difference in nucleotide sequence between two different oligonucleotide or polynucleotide sequences.

“Polymorphism” as used herein refers to a sequence variation in a gene which is not necessarily associated with pathology.

“Mutation” as used herein refers to an altered genetic sequence which results in the gene coding for a non-functioning protein or a protein with substantially reduced or altered function. Generally, a deleterious mutation is associated with pathology or the potential for pathology. The mutations in the present invention usually involve non-sense and frame shift mutations which cause a truncated (and presumably non-functional) protein to be formed. These truncations are at the terminus of the protein rather than a deletion of one or more amino acids in an internal, non-terminal region of the BRCA1 protein.

The “mutation site” is the location of the added, deleted or substituted bases in the wild-type or consensus BRCA1 DNA sequence which describes the mutant BRCA1 DNA sequence.

“Predetermined sequence variation” as used herein refers to a nucleotide sequence that is designed to be different than the corresponding sequence in a reference nucleotide sequence. A predetermined sequence variation can be a known mutation in a BRCA1 gene.

“BRCA1 gene” refers the published gene sequences, such as those appearing in the GENBANK database under Accession Number, 159546, 2489823, and Y08757. Other different sequences which include polymorphisms and genetic alterations, particularly those which don't cause an amino acid change or which are naturally occurring (wild types), which are not associated with pathology are also considered the BRCA1 gene. The corresponding nucleotides would then be used even if the nucleotide number differs. Generally, the sense strand is referred to. The BRCA1 gene may be in fragments. “Fragments” are segments of the BRCA1 gene, generally about 15 or more nucleotides in length, usually a few hundred or more nucleotides in length and potentially containing the particular mutation site of interest. The complementary strand to the sense strand of the BRCA1 gene (the so-called antisense strand) is also considered the “BRCA1 gene”. While the BRCA1 gene discussed herein is the human BRCA1 gene, the corresponding assays and reagents for the gene in other animals may also be used. The BRCA1 gene includes the coding sequences, non-coding sequences (e.g. introns) and regulatory regions affecting gene expression.

“Allele specific detection assay” as used herein refers to an assay to detect the presence or absence of a predetermined sequence variation in a test polynucleotide or oligonucleotide by annealing the test polynucleotide or oligonucleotide with a polynucleotide or oligonucleotide of predetermined sequence such that differential DNA sequence based techniques or DNA amplification methods discriminate between normal and mutant. Allele Specific Oligonucleotide hybridization is sometimes referred to ASO or the ASO method.

“Sequence variation locating assay” as used herein refers to an assay that detects a sequence variation in a test polynucleotide or oligonucleotide and localizes the position of the sequence variation to a sub-region of the test polynucleotide, without necessarily determining the precise base change or position of the sequence variation.

“Targeted confirmatory sequencing” as used herein refers to sequencing a polynucleotide in the region wherein a sequence variation has been located by a sequence variation locating assay in order to determine the precise base change and/or position of the sequence variation.

“Probe” includes any oligonucleotide which hybridizes to a BRCA1 or mutant BRCA1 sequence. The probe may be labeled (directly or indirectly) or it may act as a primer such as a PCR primer.

“Cancer”, “tumor” and “neoplasm” are used interchangeably to refer to certain abnormal cells. The terms are not meant to denote a stage of malignancy.

The invention in several of its embodiments includes:

Detection of Predetermined Sequence Variations

Stage I analysis is used to determine the presence or absence of a predetermined nucleotide sequence variation; preferably a known mutation or set of known mutations in the test gene. In accordance with the invention, such predetermined sequence variations are preferably detected by allele specific hybridization, a sequence-dependent-based technique which permits discrimination between normal and mutant alleles. An allele specific assay is dependent on the differential ability of mismatched nucleotide sequences (e.g., normal:mutant) to hybridize with each other, as compared with matching (e.g., normal:normal or mutant:mutant) sequences.

Detection of Predetermined Sequence Variations Using Allele Specific Hybridization

A variety of methods well-known in the art can be used for detection of predetermined sequence variations by allele specific hybridization. Preferably, the test gene is probed with allele specific oligonucleotides (ASOs); and each ASO contains the sequence of a known mutation. ASO analysis detects specific sequence variations in a target polynucleotide fragment by testing the ability of a specific oligonucleotide probe to hybridize to the target polynucleotide fragment. Preferably, the oligonucleotide contains the mutant sequence (or its complement). The presence of a sequence variation in the target sequence is indicated by hybridization between the oligonucleotide probe and the target fragment under conditions in which an oligonucleotide probe containing a normal sequence does not hybridize to the target fragment. A lack of hybridization between the sequence variant (e.g., mutant) oligonucleotide probe and the target polynucleotide fragment indicates the absence of the specific sequence variation (e.g., mutation) in the target fragment. In a preferred embodiment, the test samples are probed in a standard dot blot format. Each region within the test gene that contains the sequence corresponding to the ASO is individually applied to a solid surface, for example, as an individual dot on a membrane. Each individual region can be produced, for example, as a separate PCR amplification product using methods well-known in the art (see, for example, the experimental embodiment set forth in Mullis, U.S. Pat. No. 4,683,202). The use of such a dot blot format is described in detail in the Examples below, detailing the Stage I analysis of the human BRCA1 gene to detect the presence or absence of different known mutations using corresponding ASOs.

Membrane-based formats that can be used as alternatives to the dot blot format for performing ASO analysis include, but are not limited to, reverse dot blot, MAD (multiplex amplification assay), and multiplex allele-specific diagnostic assay (MASDA).

In a reverse dot blot format, oligonucleotide or polynucleotide probes having known sequence are immobilized on the solid surface, and are subsequently hybridized with the labeled test polynucleotide sample. In this situation, the primers may be labeled or the NTPs maybe labeled prior to amplification to prepare a labeled test polynucleotide sample. Alternatively, the test polynucleotide sample may be labeled subsequent to isolation and/or synthesis.

In a multiplex format, individual samples contain multiple target sequences within the test gene, instead of just a single target sequence. For example, multiple PCR products each containing at least one of the ASO target sequences are applied within the same sample dot. Multiple PCR products can be produced simultaneously in a single amplification reaction using the methods of Caskey et al., U.S. Pat. No. 5,582,989. The same blot, therefore, can be probed by each ASO whose corresponding sequence is represented in the sample dots.

A MASDA format expands the level of complexity of the multiplex format by using multiple ASOs to probe each blot (containing dots with multiple target sequences). This procedure is described in detail in U.S. Pat. No. 5,589,330 by A. P. Shuber, and in Michalowsky et al., American Journal of Human Genetics, 59(4): A272, poster 1573 (October 1996), each of which is incorporated herein by reference in its entirety. First, hybridization between the multiple ASO probe and immobilized sample is detected. This method relies on the prediction that the presence of a mutation among the multiple target sequences in a given dot is sufficiently rare that any positive hybridization signal results from a single ASO within the probe mixture hybridizing with the corresponding mutant target. The hybridizing ASO is then identified by isolating it from the site of hybridization and determining its nucleotide sequence.

Suitable materials that can be used in the dot blot, reverse dot blot, multiplex, and MASDA formats are well-known in the art and include, but are not limited to nylon and nitrocellulose membranes.

When the target sequences are produced by PCR amplification, the starting material can be chromosomal DNA in which case the DNA is directly amplified. Alternatively, the starting material can be mRNA, in which case the mRNA is preferably first reversed transcribed into cDNA and then amplified according to the well known technique of RT-PCR (see, for example, to U.S. Pat. No. 5,561,058 by Gelfand et al.).

The methods described above are suitable for moderate screening of a limited number of sequence variations. However, with the need in molecular diagnosis for rapid, cost effective large scale screening, technologies have developed that integrate the basic concept of ASO, but far exceed the capacity for mutation detection and sample number. These alternative methods to the ones described above include, but are not limited to, large scale chip array sequence-based techniques. The use of large scale arrays allows for the rapid analysis of many sequence variants. A review of the differences in the application and development of chip arrays is covered by Southern, Trends In Genetics, 12: 110-115 (March 1996) and Cheng et al., Molecular Diagnosis, 1:183-200 (Sept. 1996). Several approaches exist involving the manufacture of chip arrays. Differences include, but not restricted to: type of solid support to attach the immobilized oligonucleotides, labeling techniques for identification of variants and changes in the hybridization of the target polynucleotide to the probe.

A promising methodology for large scale analysis on “DNA chips” is described in detail in Hacia et al., Nature Genetics, 14:441-447 (1996), which is hereby incorporated by reference in its entirety. As described in Hacia et al., high density arrays of over 96,000 oligonucleotides, each 20 nucleotides in length, are immobilized to a single glass or silicon chip using light directed chemical synthesis. Contingent on the number and design of the oligonucleotide probe, potentially every base in a sequence can be interrogated for alterations. Oligonucleotides applied to the chip, therefore, can contain sequence variations that are not yet known to occur in the population, or they can be limited to mutations that are known to occur in the population.

Prior to hybridization with oligonucleotide probes on the chip, the test sample is preferably isolated, amplified and labeled (e.g. fluorescent markers) by means well known to those skilled in the art. The test polynucleotide sample is then hybridized to the immobilized oligonucleotides. The intensity of hybridization of the target polynucleotide to the immobilized probe is quantitated and compared to a reference sequence. The resulting genetic information can be used in molecular diagnosis.

A common, but not limiting, utility of the “DNA chip” in molecular diagnosis is screening for known mutations. However, this may impose a limitation to the technique by only looking at mutations that have been described in the field. The present invention allows allele specific hybridization analysis be performed with a far greater number of mutations than 1b previously available. In accordance with the present invention, DNA chips may be constructed with any number of ASO's specific for any number of mutations of the present invention. Such DNA chips may include hundreds, thousands, or more different ASO's, optionally enabling the screening for all possible mutations of the present invention in a single DNA chip. Preferably, DNA chips of the present invention contain about 10 to about 1000, about 100 to about 1,000, about 1,000 to about 10,000, about 10,000 to about 10,0000, or even greater than 100,000 allele specific oligonucleotides specific for the mutations of the present invention. Additionally, such a DNA chip may optionally contain ASO's specific for those missense mutations for which a clinical significance has been established and/or ASO's specific for wild-type BRCA1 DNA sequences. Thus, the efficiency and comprehensiveness of large scale ASO analysis will be broadened, reducing the need for cumbersome end-to-end sequence analysis, not only with known mutations but in a comprehensive manner all mutations which might occur as predicted by the principles accepted, and the cost and time associated with these cumbersome tests will be decreased.

### EXAMPLE

Genomic DNA (at least about 100 ng) is isolated from white blood cells of a subject with a family history of various cancer. Genomic DNA (at least about 100 ng) is also isolated from a wide variety of fresh tumor cells from biopsy, frozen tumor tissue previously surgically removed and tumor cell lines. Dideoxy sequence analysis is performed following polymerase chain reaction amplification of the BRCA1 gene. The primers are the same as used in the references above.

Each segment of the BRCA1 gene is subjected to direct dideoxy sequence analysis by asymmetric amplification using the polymerase chain reaction (PCR) to generate a single stranded product amplified from this DNA sample. Shuldiner, et al., Handbook of Techniques in Endocrine Research, p. 457-486, DePablo, F., Scanes, C., eds., Academic Press, Inc., 1993. Fluorescent dye is attached for automated sequencing using the TAQ DYE TERMINATOR KIT (PERKIN-ELMER cat# 401628). DNA sequencing is performed in both forward and reverse directions on an APPLIED BIOSYSTEMS, INC. (ABI) automated sequencer (Model 377). The software used for analysis of the resulting data is ASEQUENCE NAVIGATOR@ purchased through ABI.

The methods of the invention, which can be used to detect sequence variations in any polynucleotide sample, are demonstrated in the Example set forth in this section, for the purpose of illustration, for one gene in particular, namely, the human BRCA1 gene. The BRCA1 coding sequence is approximately 5592 base pairs encoding the 403 amino acids BRCA1 protein.

Designing an Allele Specific Oligonucleotide (ASO) Probe

An allele specific oligonucleotide probe is a short, single stranded polynucleotide that is engineered to hybridize exactly to a target sequence under a given set of conditions. Routinely, ASO probes are designed to contain sequences identical to the normal allele and sequence variation respectively. Hybridization of the probe to the target allows for the discrimination of a variant sample. Under stringent conditions, a probe with a variation as simple as a single-base pair will not hybridize to a normal sequence due to a destabilizing effect of the normal-mutant duplex (Ikuta, S. et al, Nucleic Acids Research, 15: 797-811 (1987). For use in this invention, probes are used to discriminate between a wild-type or normal sequence from one that is mutated. Each probe pair contains a polynucleotide sequence that encompassed an area that would identify a selected mutation of the BRCA1 gene.

The design of an ASO hybridization probe must meet two basic requirements. (Current Protocols in Human Genetics, 9.4, (1995)). First, probes that are used together in the same pool should be around the same length. Although the standard length of a probe is optimally about 17 base pairs, the range can be as short as about 13 or as long as 30 or more. If the mutation contains a long insertion, a longer probe may be desirable. Second, the mismatched region should not be placed at the end of the probe, but approximately in the middle of the sequence. In addition, the placement of a mismatch, in the case of a longer probe, should not be at the end, but at a position that allows strong hybridization and stabilization of the polynucleotide strand. In order to minimize the effects of variations in base composition of the probes, tetramethylammonium chloride may be used as in the ASO hybrid's buffer (Shuber, U.S. Pat. No. 5,633,134). Conventionally, ASO probes are synthesized on a DNA synthesizer. They can be labeled with isotopic or non-isotopic detection agents using means familiar to those of skill in the art. The process outlined in this application for making and using probes can be applicable for other gene sequences.

Protein Truncation Assay

PCR Amplification

BRCA1 is first amplified by PCR from a patient sample using one or more primer sets. A single set of primers may be used to amplify the entire BRCA1 gene or multiple sets of primers may be used. Preferably one need not use a separate set of primers for each exon because the protein expression products are so small that detecting a truncation will be difficult.

Using the primer sets referenced above in a reaction containing Ex Taq Buffer 10×5.0 mL, dNTP's 2.5 mM 4.0 mL, Forward primer 10 mM 1.0 mL, Reverse primer 10 mM 1.0 mL, TaKaRa Ex Taq (Oncor RR001B) 2.5 U 1.0 mL, Template DNA 100 ng/mL 1.0 mL and OmniPure dH2O to 50 mL final volume. One DNA control (placental DNA), one positive control reaction and three no template control reactions are included for each sample batch.

PCR for the BRCA1 gene is performed using the following thermocycling conditions (4 linked programs):

| Temperature | Time | #of Cycles | |
|---|---|---|---|
| 94° C. | 5 min. | 1 | |
| 94° C. 55° C. 72° C. | 30 sec. 1 min. 3 min. | 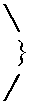 | 35 |
| 72° C. | 5 min. | 1 | |
| 4° C. | hold | 1 | |

5 m\μL of the PCR product is placed on a 2% agarose gel. On the same gel a DNA 100 BP LADDER (Gibco BRL 15628-019) and a low DNA MASS LADDER (Gibco BRL 10068-013) is placed to verify product size.

The resulting product is analyzed according to the following rules: l)Each patient sample must show a band of the correct size. If a patient sample demonstrates smearing or multiple bands, the PCR reaction needs to be repeated (no more than three times) until a clean, single band is detected. If no PCR product is visible or if only a weak band is visible, but the placental DNA sample worked well, the sample is reamplified with twice as much template. The volume of the reaction is adjusted appropriately.

All “No template” (2-3) reactions must not show amplification products of any size. If any one shows any contamination (i.e. specific amplification product), all PCR products should be thrown away and the entire PCR set-up should be repeated after appropriate PCR decontamination procedures have been taken.

The intensity of the patient sample PCR product is compared with that of the DNA 100 bp ladder. The optimum amount of PCR product on the gel should be 50-100 ng. If less than this is present or if the intensity of the patient sample is less than half the intensity of the placental control sample, repeat the PCR reaction until sufficient quantity is obtained. If no PCR product is visible or if only a weak band is visible, but the placental DNA sample worked well, the patient sample is reamplified with twice as much template DNA.

The PCR product is precipitated by adding the following to each tube: 3M sodium acetate 30 μL, 20 mg/mL glycogen 2 μL, dH2O 178 μL, and PCR product 90 μL. The reaction is mixed by inverting the tubes 4-6 times. 600 μL of 100% ethanol (200 proof) is added to each tube and the reaction can be placed at −20° C. overnight. The following day, samples are allowed to equilibrate to room temperature before proceeding. The tubes are centrifuged at 13,000 rpm for 15 minutes at room temperature in an IEC microcentrifuge. The supernatant is removed leaving a pellet. 1 mL cold 70% ethanol is added to each tube and centrifuged at 13,000 rpm for 5 minutes at room temperature in the IEC microcentrifuge. The supernatant is again removed leaving the pellet. The tubes are dried by vacuum for 10-15 minutes until no ethanol remains in the tube. Redissolve the pellet in 10 μL of dH2O.

1 mL of the PCR product is electrophoresed on a 2% TAE agarose gel, in parallel with a DNA 100 bp ladder and a DNA mass ladder to verify product size and amount of product. The mass of the 1 μL purified PCR product is estimated using the DNA mass ladder as a reference. The band equals one-tenth of the total quantity of purified PCR product. The amount required for the lysate reaction is between 500-750 ng. For example, if the band is the intensity of the 100 ng marker, then the amount needed for the lysate reaction is 5.0-7.5 μL of purified PCR product. If there is less than 500 ng total, the PCR must be repeated.

In vitro transcription/translation

This procedure is performed using the TnT Coupled Reticulocyte Lysate system from Promega L4610. Each kit contains the reagents necessary for 80 25 μL translation reactions. This kit allows the synthesis of the specified protein from PCR product.

The TnT Rabbit Reticulocyte Lysate is thawed in gloved hands and immediately place on ice. The TnT T7 RNA Polymerase and RNase Inhibitor (Boehringer Mannheim 799025) are placed on ice at all times. Solutions are mixed in the following order in a 0.5 mL microcentrifuge tube. Rabbit Reticulocyte Lysate 12.5 μL, TnT Reaction buffer 1.0 μL, TnT T7 RNA Polymerase 0.5 μL, Amino Acid Mixture minus Met (1 mM) 0.5 μL, RNase inhibitor 0.5 μL, per 15.0 ml sample.

The following are added to a clean labeled 0.5 mL microcentrifuge tube for each reaction: master mix 15.0 μL, 35S methionine (Amersham SJ1015) (1 mCi/100 μL) 2.0 μL, DNA template (500-750 ng) per μL, sdH2O to 25 μL final volume. The reactions are placed in the 30° C. water bath for 1.5 hours.

Gel Preparation, Run and Handling

Pre-run the gel (15% Tris-glycine Ready gel (15 well) BioRad 161-0938 or (10 well) BioRad 161-0908) in a Mini Protean II Gel apparatus (BioRad 165-2941) for 15 minutes to equilibrate the gel with the SDS from the running buffer (10×Tris/Glycine/SDS, BioRad 161-0732). Load samples and check the buffer volume during the course of the run to assure that nothing has leaked out. Decreased buffer volume in the middle chamber will interfere with the current. Monitor the progress of the gel running to prevent the lower marker (blue band) from running off the gel. The average running time is 3 hours.

The gel is rinsed with dH2O after fixing the gel to remove excess salicylic acid and oriented. A mixture of 5.26 mL of beta-mercaptoethanol (BioRad 161-0710) and 94.74 mL of Laemelli sample buffer (BioRad 161-0737) was prepared. The 35S ladder is added as follows in a labeled, 0.5 mL microcentrifuge tube: 7 kD marker in 5 μL, 11 kD marker in 5 μL, 25 kD marker in 2 μL, 74 kD marker in 2 μL. A second labeled tube completes the 35S ladder with: combined lysate 14 μL, sample buffer 32 μL, 100 μM IAA 12 μL, sdH2O 22 mL with a total of 20 μL per gel.

In a 0.5 μL microcentrifuge tube, the following is added:

2 μL lysate reaction, 8 μL sample buffer, 4 μL 100 mM IAA, 6 μL sdH2O, to a total load volume of 20 μL. The samples are heated at 95° C. for 3 minutes and then placed on ice for 2 minutes before loading gel. For every gel run, the following controls are included:

a. 10 μL of Broad Range Prestained SDS-PAGE standard (BioRad 161-0318) heated to 37° C. to dissolve any precipitated material.

b. 35S ladder

c. one negative control per sample

d. one positive control specific to the fragment under analysis

During the first 20 minutes, the samples are run at 10 mA (20 mA for two gels). This allows the protein to migrate through the resolving gel at a slower rate. After 20 minutes, the current is increased to 20 mA (40 mA for two gels) for the remainder of the gel run. The buffer volume is checked during the course of the run to assure that nothing has leaked out. Decreased buffer volume in the middle chamber will interfere with the current. The average running time is 3 hours. The lower marker on the polypeptide standard is 7.1 kD (blue band) and should not run off the gel.

After running the gel, the plastic adhesive is removed from the back of the ready gel and gently peel off the top plate. The gel will remain attached to the back plate. The gel is placed in fixative solution of Isopropanol 250 mL, sdH2O 650 mL and acetic acid 100 mL in a volume sufficient to cover gel for 15 minutes with swirling the gel occasionally by hand or on a rotating plate. The gel is then placed in a fluorogenic agent of salicylic acid 160 g and sdH2O to 1 L final volume, pH adjusted to pH 6.0, in a volume sufficient to cover gel for 15 minutes with swirling the gel occasionally by hand or on a rotating plate. The gel is then rinsed with sdH2O to remove any excess salicylic acid.

The gel is dried by sandwiching it between two sheets of cellophane and drying it in a gel dryer (BioRad 165-1771) for 1.5-2.5 hours. The dried gel is removed, placed in a film cassette (Fisher IB1502350) taped to a piece of film (Kodak BioMax MR Sigma 870-1302) over the dried gel(s), oriented with Identi-kit tape (Diversified Biotech ID-100) and exposed for 3-5 XD hours at −80° C. The film may be exposed overnight at −20° C. or over the weekend at room temperature if needed.

Patient samples are compared to the normal genomic DNA fragments. Truncated proteins are possible at any point along the sequence. Therefore, a shift in bands located in any patient sample is an indication of a mutation. There are areas in the sequence where a mutation can occur that are difficult to detect because of the small molecular weight protein formed by the truncation or because a stop signal occurs at the end of the sequence. These mutations are scanned for as follows. The base pairs at the 5′ end of BRCA1 are sequenced. This assures the ability to detect, by means of sequencing, proteins 10 kD or less and the ability to detect, by means of protein truncation, anything greater than 10 kD. A mutation causing a stop codon to occur at the end of the BRCA1 gene is identified by sequencing the final 257 base pairs.

The truncated BRCA1 protein should be of equal intensity on the gel as the normal BRCA1 protein. When a potential truncated band is identified, the protein must be sized using the 35S radiolabeled marker. For example, if a band appears to be in the area of the 50 kD marker, then the range of inspection for the mutation is between 40-50 kD. The position of the stop signal does not always indicate the position where the mutation occurred. Using the following conversion factor: 270 bp=10 kD, the molecular weight can be converted into base pairs.

Detailed Method for the Detection of Sequence Variations in Polynucleotides

Isolation of Genomic DNA

White blood cells are collected from the patients and genomic DNA is extracted from the white blood cells according to well-known methods (Sambrook, et al., Molecular Cloning, A Laboratory Manual, 2nd Ed., 1989, Cold Spring Harbor Laboratory Press, at 9.16-9.19). Genomic DNA is similarly extracted from a wide variety of fresh tumor cells from biopsy, frozen tumor tissue previously surgically removed and tumor cell lines.

PCR Amplification for Sequencing

The genomic DNA is used as a template to amplify a DNA fragment encompassing the site of the mutation to be tested. The 25 μl PCR reaction contains the following components: 1 μl template (100 ng/ ml) DNA, 2.5 μl 10×PCR Buffer (PERKIN-ELMER), 1.5 μl dNTP (2 mM each dATP, dCTP, dGTP, dTTP), 1.5 μl Forward Primer (10 mM), 1.5 μl Reverse Primer (10 mM), 0.5 μl (2.5 U total) AMPLITAQ GOLD™ TAQ DNA POLYMERASE or AMPLITAQ7 TAQ DNA POLYMERASE (PERKIN-ELMER), 1.0 to 5.0 μl (25 mM) MgCl₂ (depending on the primer) and distilled water (dH₂O) up to 25 μl. All reagents for each exon except the genomic DNA can be combined in a master mix and aliquoted into the reaction tubes as a pooled mixture.

For each exon analyzed, the following control PCRs are set up:

(1) “Negative” DNA control (100 ng placental DNA (SIGMA CHEMICAL CO., St. Louis, Mo.)

(2) Three “no template” controls

PCR for all exons is performed using the following thermocycling conditions:

| Temperature | Time | | Number of Cycles |
|---|---|---|---|
| 90° C. | 5 min. (AMPLITAQ) | | 1 |
| or | 10 min. (GOLD) | | |
| 95° C. 55° C. 72° C. | 30 sec. 30 sec. 1 min. | 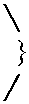 | 30 cycles |
| 72° C. | 5 min. | | 1 |
| 4° C. | hold | | 1 |

Quality Control Agarose Gel of PCR Amplification:

The quality of the PCR products is examined prior to further analysis by electrophoresing an aliquot of each PCR reaction sample on an agarose gel. 5 μl of each PCR reaction is run on an agarose gel along side a DNA 100 BP DNA LADDER (Gibco BRL cat# 15628-019). The electrophoresed PCR products are analyzed according to the following criteria:

Each patient sample must show a single band of the size corresponding the number of base pairs expected from the length of the PCR product from the forward primer to the reverse primer. If a patient sample demonstrates smearing or multiple bands, the PCR reaction must be repeated until a clean, single band is detected. If no PCR product is visible or if only a weak band is visible, but the control reactions with placental DNA template produced a robust band, the patient sample should be re-amplified with 2×as much template DNA.

All three “no template” reactions must show no amplification products. Any PCR product present in these reactions is the result of contamination. If any one of the “no template” reactions shows contamination, all PCR products should be discarded and the entire PCR set of reactions should be repeated after the appropriate PCR decontamination procedures have been taken.

The optimum amount of PCR product on the gel should be between 50 and 100 ng, which can be determined by comparing the intensity of the patient sample PCR products with that of the DNA ladder. If the patient sample PCR products contain less than 50 to 100 ng, the PCR reaction should be repeated until sufficient quantity is obtained.

DNA Sequencing

For DNA sequencing, double stranded PCR products are labeled with four different fluorescent dyes, one specific for each nucleotide, in a cycle sequencing reaction. With Dye Terminator Chemistry, when one of these nucleotides is incorporated into the elongating sequence it causes a termination at that point. Over the course of the cycle sequencing reaction, the dye-labeled nucleotides are incorporated along the length of the PCR product generating many different length fragments.

The dye-labeled PCR products will separate according to size when electrophoresed through a polyacrylamide gel. At the lower portion of the gel on an ABI automated sequencer, the fragments pass through a region where a laser beam continuously scans across the gel. The laser excites the fluorescent dyes attached to the fragments causing the emission of light at a specific wavelength for each dye. Either a photomultiplier tube (PMT) detects the fluorescent light and converts is into an electrical signal (ABI 373) or the light is collected and separated according to wavelength by a spectrograph onto a cooled, charge coupled device (CCD) camera (ABI 377). In either case the data collection software will collect the signals and store them for subsequent sequence analysis.

PCR products are first purified for sequencing using a QIAQUICK-SPIN PCR PURIFICATION KIT (QIAGEN #28104). The purified PCR products are labeled by adding primers, fluorescently tagged dNTPs and Taq Polymerase FS in an ABI Prism Dye Terminator Cycle Sequencing Kit (PERKIN ELMER/ABI catalog #02154) in a PERKIN ELMER GENEAMP 9600 thermocycler.

The amounts of each component are:

| | |
|---|---|
| For Samples | For Controls |

| Reagent | Volume | Reagent | Volume |
|---|---|---|---|
| Dye mix | 8.0 μL | PGEM | 2.0 μL |
| Primer (1.6 mM) | 2.0 μL | M13 | 2.0 μL |
| PCR product | 2.0 μL | Dye mix | 8.0 μL |
| sdH2O | 8.0 μL | sdH2O | 8.0 μL |

The thermocycling conditions are:

| Temperature | Time | # of Cycles | |
|---|---|---|---|
| 96° C. 50° C. 60° C. | 15 sec. 5 sec. 4 min. | 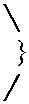 | 25 |
| 4° C. | hold | | 1 |

The product is then loaded into a gel and placed into an ABI DNA Sequencer (Models 373A & 377) and run. The sequence obtained is analyzed by comparison to the wild type (reference) sequence using SEQUENCE NAVIGATOR software. When a sequence does not align, it indicates a possible mutation. The DNA sequence is determined in both the forward and reverse directions. All results are provided to a second reader for review.

Heterozygous/homozygous point mutations and polymorphisms must be seen in both strands. Frame shift mutations will be seen in both strands and must have clear double peaks in frame shift regions to be so identified.

PCR Amplification for ASO

The genomic DNA is used as a template to amplify a separate DNA fragment encompassing the site of the mutation to be tested. The 50 ml PCR reaction contains the following components: 1 ml template (100 ng/ ml) DNA, 5.0 ml 10×PCR Buffer (PERKIN-ELMER), 2.5 ml dNTP (2 mM each dATP, dCTP, dGTP, dTTP), 2.5 ml Forward Primer (10 mM), 2.5 ml Reverse Primer (10 mM), 0.5 ml (2.5 U total) AMPLITAQ7 TAQ DNA POLYMERASE or AMPLITAQ GOLD™ DNA POLYMERASE (PERKIN-ELMER), 1.0 to 5.0 ml (25 mM) MgCl₂ (depending on the primer) and distilled water (dH₂O) up to 50 ml. All reagents for each exon except the genomic DNA can be combined in a master mix and aliquoted into the reaction tubes as a pooled mixture.

For each exon analyzed, the following control PCRs are set up:

(1) “Negative” DNA control (100 ng placental DNA (SIGMA CHEMICAL CO., St. Louis, Mo.)

(2) Three “no template” controls

PCR for all exons is performed using the following thermocycling conditions:

| Temperature | Time | | Number of Cycles |
|---|---|---|---|
| 95° C. | 5 min. (AMPLITAQ) | | 1 |
| | or 10 min. (GOLD) | | |
| 95° C. 55° C. 72° C. | 30 sec. 30 sec. 1 min. | 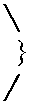 | 30 cycles |
| 72° C. | 5 min. | | 1 |
| 4° C. | hold | | 1 |

The quality control agarose gel of PCR amplification is performed as above.

Binding PCR Products to Nylon Membrane

The PCR products are denatured no more than 30 minutes prior to binding the PCR products to the nylon membrane. To denature the PCR products, the remaining PCR reaction (45 ml) and the appropriate positive control mutant gene amplification product are diluted to 200 ml final volume with PCR Diluent Solution (500 mM NaOH, 2.0 M NaCl, 25 mM EDTA) and mixed thoroughly. The mixture is heated to 95° C. for 5 minutes, and immediately placed on ice and held on ice until loaded onto dot blotter, as described below.

The PCR products are bound to 9 cm by 13 cm nylon ZETA PROBE BLOTTING MEMBRANE (BIO-RAD, Hercules, Calif., catalog number 162-0153) using a BIO-RAD dot blotter apparatus. Forceps and gloves are used at all times throughout the ASO analysis to manipulate the membrane, with care taken never to touch the surface of the membrane with bare hands or latex gloves.

Pieces of 3 MM filter paper [WHATMAN7, Clifton, N.J.] and nylon membrane are pre-wet in 10×SSC prepared fresh from 20×SSC buffer stock. The vacuum apparatus is rinsed thoroughly with dH₂O prior to assembly with the membrane. 100 ml of each denatured PCR product is added to the wells of the blotting apparatus. Each row of the blotting apparatus contains a set of reactions for a single exon to be tested, including a placental DNA (negative) control, a synthetic oligonucleotide with the desired mutation or a PCR product from a known mutant sample (positive control), and three no template DNA controls.

After applying PCR products, the nylon filter is placed DNA side up on a piece of 3 MM filter paper saturated with denaturing solution (1.5 M NaCl, 0.5 M NaOH) for 5 minutes. The membrane is transferred to a piece of 3MM filter paper saturated with neutralizing solution (1 M Tris-HCl, pH 8, 1.5 M NaCl) for 5 minutes. The neutralized membrane is then transferred to a dry 3 MM filter DNA side up, and exposed to ultraviolet light (STRALINKER, STRATAGENE, La Jolla, Calif.) for exactly 45 seconds the fix the DNA to the membrane. This UV crosslinking should be performed within 30 min. of the denaturation/neutralization steps. The nylon membrane is then cut into strips such that each strip contains a single row of blots of one set of reactions for a single exon.

Hybridizing Labeled Oligonucleotides to the Nylon Membrane

Prehybridization

The strip is prehybridized at 52° C. incubation using the HYBAID7 (SAVANT INSTRUMENTS, INC., Holbrook, N.Y.) hybridization oven. 2×SSC (15 to 20 ml) is preheated to 52° C. in a water bath. For each nylon strip, a single piece of nylon mesh cut slightly larger than the nylon membrane strip (approximately 1″×5″) is pre-wet with 2×SSC. Each single nylon membrane is removed from the prehybridization solution and placed on top of the nylon mesh. The membrane/mesh “sandwich” is then transferred onto a piece of Parafilm™. The membrane/mesh sandwich is rolled lengthwise and placed into an appropriate HYBAID7 bottle, such that the rotary action of the HYBAID7 apparatus caused the membrane to unroll. The bottle is capped and gently rolled to cause the membrane/mesh to unroll and to evenly distribute the 2×SSC, making sure that no air bubbles formed between the membrane and mesh or between the mesh and the side of the bottle. The 2×SSC is discarded and replaced with 5 ml TMAC Hybridization Solution, which contains 3 M TMAC (tetramethyl ammoniumchloride—SIGMA T-3411), 100 mM Na₃PO₄ (pH 6.8), 1 mM EDTA, 5× Denhardt's (1% Ficoll, 1% polyvinylpyrrolidone, 1% BSA (fraction V)), 0.6% SDS, and 100 mg/ml Herring Sperm DNA. The filter strips are prehybridized at 52° C. with medium rotation (approx. 8.5 setting on the HYBAID7 speed control) for at least one hour. Prehybridization can also be performed overnight.

Labeling Oligonucleotides

The DNA sequences of the numerous oligonucleotide probes are used to detect the BRCA1 mutation. For each mutation, a mutant and a normal oligonucleotide must be labeled. While only five pairs of oligonucleotide probes are listed below, corresponding oligonucleotides for each mutation may be prepared and used in a similar manner.

| | |
|---|---|
| mutation 185delAG. | |
| wild-type | |
| 5′-AAT CTT AGA GTG TCC CA-3′, | SEQ ID NO:3 |
| mutant | |
| 5′-ATC TTA GTG TCC CAC CT-3′, | SEQ ID NO:4 |
| mutation 1136insA. | |
| wild-type | |
| 5′-CAG AAA AAA AGG TAG AT-3′, | SEQ ID NO:5 |
| mutant | |
| 5′-CAG AAA AAA AAG GTA GA-3′, | SEQ ID NO:6 |
| mutation 5383insC. | |
| wild-type | |
| 5′-AGA GAA TCC CAG GAC AG-3′, | SEQ ID NO:7 |
| mutant | |
| 5′-AGA GAA TCC CCA GGA CA-3′, | SEQ ID NO:8 |
| mutationC4446T. | |
| wild-type | |
| 5′-AGG ACC TGC GAA ATC CA-3′, | SEQ ID NO:9 |
| mutant | |
| 5′-AGG ACC TGT GAA ATC CA-3′, | SEQ ID NO:10 |

Each labeling reaction contains 2 ml 5×Kinase buffer (or 1 ml of 10×Kinase buffer), 5 ml gamma-ATP ³²P (not more than one week old), 1 μl T4 polynucleotide kinase, 3 μl oligonucleotide (20 mM stock), sterile H₂O to 10 μl final volume if necessary. The reactions are incubated at 37° C. for 30 minutes, then at 65° C. for 10 minutes to heat inactivate the kinase. The kinase reaction is diluted with an equal volume (10 μl) of sterile dH₂O (distilled water).

The oligonucleotides are purified on STE MICRO SELECT-D, G-25 spin columns (catalog no. 5303-356769), according to the manufacturer's instructions. The 20 μl synthetic oligonucleotide eluate is diluted with 80 μl dH₂O (final volume=100 μl). The amount of radioactivity in the oligonucleotide sample is determined by measuring the radioactive counts per minute (cpm). The total radioactivity must be at least 2 million cpm. For any samples containing less than 2 million cpm total, the labeling reaction is repeated.

Hybridization with Mutant Oligonucleotides

Approximately 2-5 million cpm of the labeled mutant oligonucleotide probe is diluted into 5 ml of TMAC hybridization solution, containing 40 μl of 20 mM stock of unlabeled normal oligonucleotide. The probe mix is preheated to 52° C. in the hybridization oven. The pre-hybridization solution is removed from each bottle and replaced with the probe mix. The filter is hybridized for 1 hour at 52° C. with moderate agitation. Following hybridization, the probe mix is decanted into a storage tube and stored at −20° C. The filter is rinsed by adding approximately 20 ml of 2×SSC+0.1% SDS at room temperature and rolling the capped bottle gently for approximately 30 seconds and pouring off the rinse. The filter is then washed with 2×SSC+0. 1% SDS at room temperature for 20 to 30 minutes, with shaking.

The membrane is removed from the wash and placed on a dry piece of 3MM WHATMAN filter paper then wrapped in one layer of plastic wrap, placed on the autoradiography film, and exposed for about five hours depending upon a survey meter indicating the level of radioactivity. The film is developed in an automatic film processor.

Control Hybridization with Normal Oligonucleotides

The purpose of this step is to ensure that the PCR products are transferred efficiently to the nylon membrane.

Following hybridization with the mutant oligonucleotide, as described in the Examples above, each nylon membrane is washed in 2×SSC, 0.1% SDS for 20 minutes at 65° C. to melt off the mutant oligonucleotide probes. The nylon strips are then prehybridized together in 40 ml of TMAC hybridization solution for at least 1 hour at 52° C. in a shaking water bath. 2-5 million counts of each of the normal labeled oligonucleotide probes plus 40 μl of 20 mM stock of unlabeled normal oligonucleotide are added directly to the container containing the nylon membranes and the prehybridization solution. The filter and probes are hybridized at 52° C. with shaking for at least 1 hour. Hybridization can be performed overnight, if necessary. The hybridization solution is poured off, and the nylon membrane is rinsed in 2×SSC, 0. 1% SDS for 1 minute with gentle swirling by hand. The rinse is poured off and the membrane is washed in 2×SSC, 0.1% SDS at room temperature for 20 minutes with shaking.

The nylon membrane is removed placed on a dry piece of 3 MM WHATMAN filter paper. The nylon membrane is then wrapped in one layer of plastic wrap and placed on autoradiography film, and exposure is for at least 1 hour.

For each sample, adequate transfer to the membrane is indicated by a strong autoradiographic hybridization signal. For each sample, an absent or weak signal when hybridized with its normal oligonucleotide, indicates an unsuccessful transfer of PCR product, and it is a false negative. The ASO analysis must be repeated for any sample that did not successfully transfer to the nylon membrane.

Interpreting Results

After hybridizing with mutant oligonucleotides, the results for each exon are interpreted as follows:

TABLE 4A

| Result | | | | | Interpretation | Action |
|---|---|---|---|---|---|---|
| 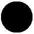 | 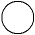 | 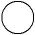 | | 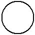 | All quality controls indicate assay is successful | Record results, dark circles are mutation positive, and all others are negative |
| (+ | (−) | NT | NT | NT | Assay not specific, | Rewash |
| | | | | | mutant oligonucleotide | membrane 30 |
| | | | | | hybridizing to normal | minutes longer |
| | | | | | DNA. | at appropriate |
| (+) | (−) | NT | NT | NT | | temperature |
| | | | | | Mutant oligonucleotide | and re-expose. |
| | | | | | probe is either washed | Rehybridize |
| | | | | | off or did not | with remaining |
| | | | | | label well enough, | oligonucleo- |
| | | | | | or PCR product is not | signal, perform |
| | | | | | transferred to | tide labeled |
| | | | | | membrane | If still no |
| | | | | | efficiently. | normal |
| | | | | | | oligonucleo- |
| | | | | | | tide hyb. as |
| | | | | | | per the Exam- |
| | | | | | | ples to test |
| | | | | | | transfer of PCR |
| | | | | | | to membrane. |
| (+) | (−) | NT | NT | NT | | |
| | | | | | Positive and negative | Perform |
| | | | | | controls indicate assay | standard clean |
| | | | | | is successful, but PCR | up procedures |
| (+) | (−) | NT | NT | NT | is contaminated. | for PCR |
| | | | | | | contamination. |

After hybridization with normal oligonucleotides, interpret the results as follows:

TABLE 4B

| | | | | | | Results indicate transfer | Record results. |
|---|---|---|---|---|---|---|---|
| | | | | | | of PCR products to | |
| | | | | | | membrane is successful. | |
| (+) | (−) | NT | NT | NT | | | |
| 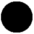 | | 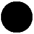 | | 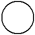 | | Results indicate transfer of patient sample #1 is inefficient. May get false negative from this sample | This sample will have to be transferred to another membrane and the assay repeated |
| (+) | (−) | #1 | NT | NT | NT | | |

The sample #1 should be lighter than the controls. Patient samples containing a mutation are generally heterozygous and will hybridize to both the normal and mutant oligonucleotide probes.

Data on Specific Mutations

The following specific mutations are examples of those which are presumed to be clinically significant for typing current cancer cells or a germ line mutation increasing the susceptibility to a tumor. A few of these mutations were also found by others as stated in TABLE 1 above.

TABLE 8
List of Nonsense Mutations

T127A, T127g, G144T, G147T, C153T, C174T, A177T, T184A, T184g, G186T, T191A,
T200A, G204T, T208A, A213T, G216T, A231T, T236A, C251A, A252T, C260A, A267T,
C279T, A282T, A285T, C295A, C295g, C297T, T302A, T307A, T307g, T311A, A312T,
A327T, C339T, G342T, A351T, C360T, G369T, G372T, T379A, A381T, T392A, C399T,
T415A, G417T, T422A, T422G, T434A, T434G, A444T, A447T, G450T, G465T, A474T,
G480T, C495T, C509A, C509G, A510T, A522T, A525T, C534T, G540T, G546T, T559A,
C561T, G564T, C582T, G597T, A606T, A621T, C624T, C633T, C639T, A642T, C656A,
C656G, G660T, T664A, G666T, G681T, A696T, T707A, T707G, C710A, G717T, C723T,
G726T, T730A, T733A, T733g, C735T, C747T, G750T, G762T, T772A, A783T, A786T,
T797A, G798T, G807T, G828T, C837T, T856A, G867T, A870T, G882T, G894T, A897T,
T902A, T902G, C903T, C919A, C919g, T925A, G933T, T941A, C964A, C964g, T967A,
T967g, C969T, G975T, T988A, T988g, T991A, T991g, A999T, A1005T, G1017T, A1020T,
G1026T, T1034A, A1038T, A1044T, C1047T, T1057A, T1057g, C1068T, A1077T, G1081A,
G1082A, G1086T, A1092T, G1095T, T1103A, G1128T, A1131T, A1134T, T1163A, G1164T,
A1167T, A1170T, G1173T, G1177A, G1178A, A1182T, C1185T, A1188T, C1199A, C1201A,
C1201g, G1203T, A1212T, G1221T, G1234A, G1235A, C1257T, A1260T, G1269T, G1273A,
G1274A, A1281T, G1290T, T1297A, T1297g, C1312A, C1312g, G1323T, G1329T, C1333A,
C1333g, A1341T, T1357A, G1371T, G1380T, T1385A, T1385G, C1396A, C1396g, G1398T,
A1401T, T1411A, T1411g, G1431T, T1438A, T1438g, T1445A, A1446T, G1452T, A1455T,
A1467T, C1471A, C1471g, G1476T, G1488T, A1494T, A1506T, T1514A, T1514G, A1518T,
A1521T, T1540A, T1540g, G1554T, G1569T, G1584T, C1590T, C1599T, G1602T, A1620T,
T1624A, T1624g, A1626T, A1632T, A1638T, C1648A, C1648g, G1662T, A1674T, A1677T,
T1687A, C1695T, A1698T, G1707T, C1719T, G1722T, C1731T, G1737T, C1740T, C1749T,
G1779T, A1785T, A1791T, C1806T, G1812T, A1815T, G1833T, C1837A, C1837g, G1842T,
A1845T, G1848T, A1860T, A1866T, G1872T, G1902T, G1908T, T1912A, T1912g, C1927A,
C1927g, A1929T, A1938T, A1941T, A1959T, G1989T, A2004T, T2027A, G2031T, T2035A,
C2037T, T2051A, G2061T, G2064T, A2070T, A2073T, A2076T, A2079T, C2084A, C2084G,
C2088T, A2109T, C2118T, G2127T, A2133T, G2136T, G2148T, A2154T, A2157T, A2166T,
G2175T, C2178T, A2187T, A2190T, G2214T, A2220T, T2224A, T2224g, A2250T, T2255A,
C2257A, C2257g, G2268T, A2274T, G2277T, A2301T, G2304T, G2307T, A2310T, G2313T,
G2316T, A2319T, G2325T, A2334T, G2352T, A2361T, T2374A, T2374g, G2379T, G2382T,
T2392A, C2394T, G2400T, A2403T, G2412T, C2428A, C2428g, T2450A, T2450G, C2457T,
G2460T, C2470A, T2473A, T2473g, G2478T, A2496T, A2502T, G2508T, A2517T, T2522A,
C2529T, T2534A, G2544T, A2553T, G2556T, T2573A, A2577T, A2586T, G2598T, A2607T,
T2612A, T2612G, T2617A, G2619T, G2625T, G2643T, G2655T, G2661T, G2664T, G2670T,
C2682T, T2687A, T2687G, T2689A, C2691T, A2703T, C2710A, C2710g, A2712T, C2718T,
C2722A, C2722g, C2737A, C2737g, G2745T, G2754T, G2757T, G2760T, T2765A, T2794A,
T2794g, A2796T, A2799T, C2802T, A2811T, G2823T, T2828A, G2829T, C2832T, A2835T,
G2838T, G2841T, C2847T, G2850T, A2853T, G2859T, A2871T, C2880T, C2919T, A2922T,
A2928T, A2946T, T2951A, A2958T, G2961T, T2978A, C2983A, C2983g, C2988T, A2994T,
G3003T, G3009T, A3027T, G3033T, T3040A, T3040g, C3042T, T3053A, T3053G, A3078T,
C3082A, C3082g, A3090T, A3096T, T3101A, A3102T, A3105T, G3117T, G3120T, G3129T,
G3132T, C3139A, C3139g, C3145A, C3145g, G3150T, A3153T, G3156T, G3162T, G3168T,
A3213T, G3216T, A3228T, G3231T, C3241A, C3241g, G3255T, G3276T, G3297T, G3315T,
C3324T, G3330T, A3339T, A3345T, A3354T, T3358A, A3372T, T3376A, T3376g, T3385A,
C3387T, G3393T, T3401A, T3401G, A3402T, C3405T, G3417T, T3428A, A3429T, G3438T,
A3444T, A3447T, C3450T, G3453T, T3458A, T3458G, G3459T, G3462T, C3471T, T3500A,
T3500G, C3508A, C3508g, T3517A, T3517g, G3519T, C3522T, G3531T, C3549T, T3557A,
G3561T, T3580A, T3580g, G3591T, A3597T, G3600T, G3618T, A3630T, G3633T, A3654T,
C3663T, A3666T, G3669T, G3672T, T3712A, C3717T, C3725A, C3725G, C3726T, A3729T,
A3738T, A3741T, T3745A, T3745g, G3747T, C3754A, C3754g, G3756T, G3759T, T3766A,
T3766g, G3774T, G3780T, G3783T, C3794A, C3798T, T3805A, T3808A, T3808g, A3816T,
C3837T, G3867T, T3872A, A3879T, G3888T, G3891T, T3898A, T3898g, T3901A, T3901g,
C3904A, C3904g, T3907A, A3909T, T3919A, T3919g, C3929A, C3936T, T3946A, A3951T,
C3960T, G3963T, G3978T, G3981T, A3987T, T3992A, T4003A, C4012A, C4012g, C4014T,
C4019A, G4023T, T4027A, G4029T, T4036A, C4056T, T4069A, A4083T, C4086T, C4098T,
G4104T, C4110T, G4113T, A4131T, G4134T, T4138A, C4144A, C4144g, G4152T, G4155T,
A4158T, G4161T, T4171A, G4173T, G4176T, C4185T, G4188T, G4191T, C4194T, C4207A,
C4207g, T4213A, T4213g, G4218T, T4235A, G4236T, G4242T, G4257T, C4265A, C4267A,
C4267g, C4281T, T4294A, T4294g, C4302T, C4305T, C4320T, A4335T, C4341T, C4344T,
G4347T, G4356T, G4362T, T4372A, T4372g, G4374T, C4377T, C4389T, C4406A, C4406G,
G4437T, C4446T, G4455T, C4458T, C4468A, C4468g, G4470T, A4473T, T4483A, T4483g,
C4489A, C4489g, C4491T, A4494T, G4503T, C4508A, C4508G, C4518T, G4527T, A4545T,
G4551T, A4578T, A4584T, G4587T, G4593T, G4599T, C4606A, C4606g, A4617T, C4622A,
C4627A, C4627g, T4630A, T4630g, G4642A, G4643A, C4646A, C4646G, C4658A, C4671T,
A4677T, C4685A, C4685G, C4692T, G4695T, G4698T, A4707T, G4722T, G4725T, C4728T,
C4731T, G4737T, G4740T, T4759A, G4764T, C4775A, C4775G, T4777A, C4785T, G4794T,
G4797T, C4808A, C4808G, G4812T, G4818T, G4845T, G4860T, A4866T, G4875T, C4879A,
C4879g, C4906A, C4906g, T4918A, A4920T, C4929T, T4933A, A4935T, G4944T, C4953T,
T4994A, T4994G, G5004T, G5007T, G5022T, A5025T, G5031T, T5035A, C5044A, C5044g,
G5049T, A5061T, A5064T, G5097T, G5100T, C5117A, C5117G, A5118T, A5127T, A5130T,
T5146A, T5146g, G5163T, G5166T, A5187T, G5199T, T5210A, G5211T, A5223T, T5228A,
T5228G, G5235T, G5244T, G5247T, A5250T, G5254A, G5255A, T5267A, T5267G, G5272A,
G5273A, C5280T, A5289T, G5292T, A5295T, A5298T, G5310T, G5322T, A5328T, G5331T,
G5346T, A5349T, C5358T, A5367T, C5370T, A5376T, G5379T, C5385T, A5391T, A5394T,
G5412T, T5420A, C5423A, T5426A, T5426G, C5454T, G5460T, G5464A, G5465A, C5472T,
T5480A, A5496T, G5499T, C5506A, C5506g, C5509A, C5509g, C5550T, G5563A, G5564A,
G5568T, C5595T, T5603A, G5604T, C5622T, G5625T, G5629A, G5630A, T5635A, C5654A,
C5654G, C5655T, C5660A, C5661T, G5664T, C5678A, C5678G, C5688T, C5708A, C5708G,
G5710A.

TABLE 9
List of One Base deletions

124delA, 125delT, 126delT, 127delT, 128delA, 129delT, 130delC, 131delT, 132delG, 133delC,
134delT, 135delC, 136delT, 137delT, 138delC, 139delG, 140delC, 141delG, 142delT, 143delT,
144delG, 145delA, 146delA, 147delG, 148delA, 149delA, 150delG, 151delT, 152delA, 153delC,
154delA, 155delA, 156delA, 157delA, 158delT, 159delG, 160delT, 161delC, 162delA, 163delT,
164delT, 165delA, 166delA, 167delT, 168delG, 169delC, 170delT, 171delA, 172delT, 173delG,
174delC, 175delA, 176delG, 177delA, 178delA, 179delA, 180delA, 181delT, 182delC, 183delT,
184delT, 185delA, 186delG, 187delA, 188delG, 189delT, 190delG, 191delT, 192delC, 193delC,
194delC, 195delA, 196delT, 197delC, 198delT, 199delG, 200delT, 201delC, 202delT, 203delG,
204delG, 205delA, 206delG, 207delT, 208delT, 209delG, 210delA, 211delT, 212delC, 213delA,
214delA, 215delG, 216delG, 217delA, 218delA, 219delC, 220delC, 221delT, 222delG, 223delT,
224delC, 225delT, 226delC, 227delC, 228delA, 229delC, 230delA, 231delA, 232delA, 233delG,
234delT, 235delG, 236delT, 237delG, 238delA, 239delC, 240delC, 241delA, 242delC, 243delA,
244delT, 245delA, 246delT, 247delT, 248delT, 249delT, 250delG, 251delC, 252delA, 253delA,
254delA, 255delT, 256delT, 257delT, 258delT, 259delG, 260delC, 261delA, 262delT, 263delG,
264delC, 265delT, 266delG, 267delA, 268delA, 269delA, 270delC, 271delT, 272delT, 273delC,
274delT, 275delC, 276delA, 277delA, 278delC, 279delC, 280delA, 281delG, 282delA, 283delA,
284delG, 285delA, 286delA, 287delA, 288delG, 289delG, 290delG, 291delC, 292delC, 293delT,
294delT, 295delC, 296delA, 297delC, 298delA, 299delG, 300delT, 301delG, 302delT, 303delC,
304delC, 305delT, 306delT, 307delT, 308delA, 309delT, 310delG, 311delT, 312delA, 313delA,
314delG, 315delA, 316delA, 317delT, 318delG, 319delA, 320delT, 321delA, 322delT, 323delA,
324delA, 325delC, 326delC, 327delA, 328delA, 329delA, 330delA, 331delG, 332delG,
333delA, 334delG, 335delC, 336delC, 337delT, 338delA, 339delC, 340delA, 341delA, 342delG,
343delA, 344delA, 345delA, 346delG, 347delT, 348delA, 349delC, 350delG, 351delA, 352delG,
353delA, 354delT, 355delT, 356delT, 357delA, 358delG, 359delT, 360delC, 361delA, 362delA,
363delC, 364delT, 365delT, 366delG, 367delT, 368delT, 369delG, 370delA, 371delA, 372delG,
373delA, 374delG, 375delC, 376delT, 377delA, 378delT, 379delT, 380delG, 381delA, 382delA,
383delA, 384delA, 385delT, 386delC, 387delA, 388delT, 389delT, 390delT, 391delG, 392delT,
393delG, 394delC, 395delT, 396delT, 397delT, 398delT, 399delC, 400delA, 401delG, 402delC,
403delT, 404delT, 405delG, 406delA, 407delC, 408delA, 409delC, 410delA, 411delG, 412delG,
413delT, 414delT, 415delT, 416delG, 417delG, 418delA, 419delG, 420delT, 421delA, 422delT,
423delG, 424delC, 425delA, 426delA, 427delA, 428delC, 429delA, 430delG, 431delC, 432delT,
433delA, 434delT, 435delA, 436delA, 437delT, 438delT, 439delT, 440delT, 441delG, 442delC,
443delA, 444delA, 445delA, 446delA, 447delA, 448delA, 449delG, 450delG, 451delA,
452delA, 453delA, 454delA, 455delT, 456delA, 457delA, 458delC, 459delT, 460delC, 461delT,
462delC, 463delC, 464delT, 465delG, 466delA, 467delA, 468delC, 469delA, 470delT, 471delC,
472delT, 473delA, 474delA, 475delA, 476delA, 477delG, 478delA, 479delT, 480delG, 481delA,
482delA, 483delG, 484delT, 485delT, 486delT, 487delC, 488delT, 489delA, 490delT, 491delC,
492delA, 493delT, 494delC, 495delC, 496delA, 497delA, 498delA, 499delG, 500delT, 501delA,
502delT, 503delG, 504delG, 505delG, 506delC, 507delT, 508delA, 509delC, 510delA, 511delG,
512delA, 513delA, 514delA, 515delC, 516delC, 517delG, 518delT, 519delG, 520delC, 521delC,
522delA, 523delA, 524delA, 525delA, 526delG, 527delA, 528delC, 529delT, 530delT, 531delC,
532delT, 533delA, 534delC, 535delA, 536delG, 537delA, 538delG, 539delT, 540delG, 541delA,
542delA, 543delC, 544delC, 545delC, 546delG, 547delA, 548delA, 549delA, 550delA, 551delT,
552delC, 553delC, 554delT, 555delT, 556delC, 557delC, 558delT, 559delT, 560delG, 561delC,
562delA, 563delG, 564delG, 565delA, 566delA, 567delA, 568delC, 569delC, 570delA,
571delG, 572delT, 573delC, 574delT, 575delC, 576delA, 577delG, 578delT, 579delG, 580delT,
581delC, 582delC, 583delA, 584delA, 585delC, 586delT, 587delC, 588delT, 589delC, 590delT,
591delA, 592delA, 593delC, 594delC, 595delT, 596delT, 597delG, 598delG, 599delA, 600delA,
601delC, 602delT, 603delG, 604delT, 605delG, 606delA, 607delG, 608delA, 609delA, 610delC,
611delT, 612delC, 613delT, 614delG, 615delA, 616delG, 617delG, 618delA, 619delC, 620delA,
621delA, 622delA, 623delG, 624delC, 625delA, 626delG, 627delC, 628delG, 629delG,
630delA, 631delT, 632delA, 633delC, 634delA, 635delA, 636delC, 637delC, 638delT, 639delC,
640delA, 641delA, 642delA, 643delA, 644delG, 645delA, 646delC, 647delG, 648delT, 649delC,
650delT, 651delG, 652delT, 653delC, 654delT, 655delA, 656delC, 657delA, 658delT, 659delT,
660delG, 661delA, 662delA, 663delT, 664delT, 665delG, 666delG, 667delG, 668delA, 669delT,
670delC, 671delT, 672delG, 673delA, 674delT, 675delT, 676delC, 677delT, 678delT, 679delC,
680delT, 681delG, 682delA, 683delA, 684delG, 685delA, 686delT, 687delA, 688delC, 689delC,
690delG, 691delT, 692delT, 693delA, 694delA, 695delT, 696delA, 697delA, 698delG, 699delG,
700delC, 701delA, 702delA, 703delC, 704delT, 705delT, 706delA, 707delT, 708delT, 709delG,
710delC, 711delA, 712delG, 713delT, 714delG, 715delT, 716delG, 717delG, 718delG, 719delA,
720delG, 721delA, 722delT, 723delC, 724delA, 725delA, 726delG, 727delA, 728delA, 729delT,
730delT, 731delG, 732delT, 733delT, 734delA, 735delC, 736delA, 737delA, 738delA, 739delT,
740delC, 741delA, 742delC, 743delC, 744delC, 745delC, 746delT, 747delC, 748delA, 749delA,
750delG, 751delG, 752delA, 753delA, 754delC, 755delC, 756delA, 757delG, 758delG,
759delG, 760delA, 761delT, 762delG, 763delA, 764delA, 765delA, 766delT, 767delC, 768delA,
769delG, 770delT, 771delT, 772delT, 773delG, 774delG, 775delA, 776delT, 777delT, 778delC,
779delT, 780delG, 781delC, 782delA, 783delA, 784delA, 785delA, 786delA, 787delA, 788delG,
789delG, 790delC, 791delT, 792delG, 793delC, 794delT, 795delT, 796delG, 797delT, 798delG,
799delA, 800delA, 801delT, 802delT, 803delT, 804delT, 805delC, 806delT, 807delG, 808delA,
809delG, 810delA, 811delC, 812delG, 813delG, 814delA, 815delT, 816delG, 817delT, 818delA,
819delA, 820delC, 821delA, 822delA, 823delA, 824delT, 825delA, 826delC, 827delT, 828delG,
829delA, 830delA, 831delC, 832delA, 833delT, 834delC, 835delA, 836delT, 837delC, 838delA,
839delA, 840delC, 841delC, 842delC, 843delA, 844delG, 845delT, 846delA, 847delA, 848delT,
849delA, 850delA, 851delT, 852delG, 853delA, 854delT, 855delT, 856delT, 857delG, 858delA,
859delA, 860delC, 861delA, 862delC, 863delC, 864delA, 865delC, 866delT, 867delG, 868delA,
869delG, 870delA, 871delA, 872delG, 873delC, 874delG, 875delT, 876delG, 877delC, 878delA,
879delG, 880delC, 881delT, 882delG, 883delA, 884delG, 885delA, 886delG, 887delG, 888delC,
889delA, 890delT, 891delC, 892delC, 893delA, 894delG, 895delA, 896delA, 897delA, 898delA,
899delG, 900delT, 901delA, 902delT, 903delC, 904delA, 905delG, 906delG, 907delG, 908delT,
909delA, 910delG, 911delT, 912delT, 913delC, 914delT, 915delG, 916delT, 917delT, 918delT,
919delC, 920delA, 921delA, 922delA, 923delC, 924delT, 925delT, 926delG, 927delC, 928delA,
929delT, 930delG, 931delT, 932delG, 933delG, 934delA, 935delG, 936delC, 937delC, 938delA,
939delT, 940delG, 941delT, 942delG, 943delG, 944delC, 945delA, 946delC, 947delA, 948delA,
949delA, 950delT, 951delA, 952delC, 953delT, 954delC, 955delA, 956delT, 957delG, 958delC,
959delC, 960delA, 961delG, 962delC, 963delT, 964delC, 965delA, 966delT, 967delT, 968delA,
969delC, 970delA, 971delG, 972delC, 973delA, 974delT, 975delG, 976delA, 977delG, 978delA,
979delA, 980delC, 981delA, 982delG, 983delC, 984delA, 985delG, 986delT, 987delT, 988delT,
989delA, 990delT, 991delT, 992delA, 993delC, 994delT, 995delC, 996delA, 997delC, 998delT,
999delA, 1000delA, 1001delA, 1002delG, 1003delA, 1004delC, 1005delA, 1006delG,
1007delA, 1008delA, 1009delT, 1010delG, 1011delA, 1012delA, 1013delT, 1014delG,
1015delT, 1016delA, 1017delG, 1018delA, 1019delA, 1020delA, 1021delA, 1022delG,
1023delG, 1024delC, 1025delT, 1026delG, 1027delA, 1028delA, 1029delT, 1030delT,
1031delC, 1032delT, 1033delG, 1034delT, 1035delA, 1036delA, 1037delT, 1038delA,
1039delA, 1040delA, 1041delA, 1042delG, 1043delC, 1044delA, 1045delA, 1046delA,
1047delC, 1048delA, 1049delG, 1050delC, 1051delC, 1052delT, 1053delG, 1054delG,
1055delC, 1056delT, 1057delT, 1058delA, 1059delG, 1060delC, 1061delA, 1062delA,
1063delG, 1064delG, 1065delA, 1066delG, 1067delC, 1068delC, 1069delA, 1070delA,
1071delC, 1072delA, 1073delT, 1074delA, 1075delA, 1076delC, 1077delA, 1078delG,
1079delA, 1080delT, 1081delG, 1082delG, 1083delG, 1084delC, 1085delT, 1086delG,
1087delG, 1088delA, 1089delA, 1090delG, 1091delT, 1092delA, 1093delA, 1094delG,
1095delG, 1096delA, 1097delA, 1098delA, 1099delC, 1100delA, 1101delT, 1102delG,
1103delT, 1104delA, 1105delA, 1106delT, 1107delG, 1108delA, 1109delT, 1110delA,
1111delG, 1112delG, 1113delC, 1114delG, 1115delG, 1116delA, 1117delC, 1118delT,
1119delC, 1120delC, 1121delC, 1122delA, 1123delG, 1124delC, 1125delA, 1126delC,
1127delA, 1128delG, 1129delA, 1130delA, 1131delA, 1132delA, 1133delA, 1134delA,
1135delA, 1136delG, 1137delG, 1138delT, 1139delA, 1140delG, 1141delA, 1142delT,
1143delC, 1144delT, 1145delG, 1146delA, 1147delA, 1148delT, 1149delG, 1150delC,
1151delT, 1152delG, 1153delA, 1154delT, 1155delC, 1156delC, 1157delC, 1158delC,
1159delT, 1160delG, 1161delT, 1162delG, 1163delT, 1164delG, 1165delA, 1166delG,
1167delA, 1168delG, 1169delA, 1170delA, 1171delA, 1172delA, 1173delG, 1174delA,
1175delA, 1176delT, 1177delG, 1178delG, 1179delA, 1180delA, 1181delT, 1182delA,
1183delA, 1184delG, 1185delC, 1186delA, 1187delG, 1188delA, 1189delA, 1190delA,
1191delC, 1192delT, 1193delG, 1194delC, 1195delC, 1196delA, 1197delT, 1198delG,
1199delC, 1200delT, 1201delC, 1202delA, 1203delG, 1204delA, 1205delG, 1206delA,
1207delA, 1208delT, 1209delC, 1210delC, 1211delT, 1212delA, 1213delG, 1214delA,
1215delG, 1216delA, 1217delT, 1218delA, 1219delC, 1220delT, 1221delG, 1222delA,
1223delA, 1224delG, 1225delA, 1226delT, 1227delG, 1228delT, 1229delT, 1230delC,
1231delC, 1232delT, 1233delT, 1234delG, 1235delG, 1236delA, 1237delT, 1238delA,
1239delA, 1240delC, 1241delA, 1242delC, 1243delT, 1244delA, 1245delA, 1246delA,
1247delT, 1248delA, 1249delG, 1250delC, 1251delA, 1252delG, 1253delC, 1254delA,
1255delT, 1256delT, 1257delC, 1258delA, 1259delG, 1260delA, 1261delA, 1262delA,
1263delG, 1264delT, 1265delT, 1266delA, 1267delA, 1268delT, 1269delG, 1270delA,
1271delG, 1272delT, 1273delG, 1274delG, 1275delT, 1276delT, 1277delT, 1278delT,
1279delC, 1280delC, 1281delA, 1282delG, 1283delA, 1284delA, 1285delG, 1286delT,
1287delG, 1288delA, 1289delT, 1290delG, 1291delA, 1292delA, 1293delC, 1294delT,
1295delG, 1296delT, 1297delT, 1298delA, 1299delG, 1300delG, 1301delT, 1302delT,
1303delC, 1304delT, 1305delG, 1306delA, 1307delT, 1308delG, 1309delA, 1310delC,
1311delT, 1312delC, 1313delA, 1314delC, 1315delA, 1316delT, 1317delG, 1318delA,
1319delT, 1320delG, 1321delG, 1322delG, 1323delG, 1324delA, 1325delG, 1326delT,
1327delC, 1328delT, 1329delG, 1330delA, 1331delA, 1332delT, 1333delC, 1334delA,
1335delA, 1336delA, 1337delT, 1338delG, 1339delC, 1340delC, 1341delA, 1342delA,
1343delA, 1344delG, 1345delT, 1346delA, 1347delG, 1348delC, 1349delT, 1350delG,
1351delA, 1352delT, 1353delG, 1354delT, 1355delA, 1356delT, 1357delT, 1358delG,
1359delG, 1360delA, 1361delC, 1362delG, 1363delT, 1364delT, 1365delC, 1366delT,
1367delA, 1368delA, 1369delA, 1370delT, 1371delG, 1372delA, 1373delG, 1374delG,
1375delT, 1376delA, 1377delG, 1378delA, 1379delT, 1380delG, 1381delA, 1382delA,
1383delT, 1384delA, 1385delT, 1386delT, 1387delC, 1388delT, 1389delG, 1390delG,
1391delT, 1392delT, 1393delC, 1394delT, 1395delT, 1396delC, 1397delA, 1398delG,
1399delA, 1400delG, 1401delA, 1402delA, 1403delA, 1404delA, 1405delT, 1406delA,
1407delG, 1408delA, 1409delC, 1410delT, 1411delT, 1412delA, 1413delC, 1414delT,
1415delG, 1416delG, 1417delC, 1418delC, 1419delA, 1420delG, 1421delT, 1422delG,
1423delA, 1424delT, 1425delC, 1426delC, 1427delT, 1428delC, 1429delA, 1430delT,
1431delG, 1432delA, 1433delG, 1434delG, 1435delC, 1436delT, 1437delT, 1438delT,
1439delA, 1440delA, 1441delT, 1442delA, 1443delT, 1444delG, 1445delT, 1446delA,
1447delA, 1448delA, 1449delA, 1450delG, 1451delT, 1452delG, 1453delA, 1454delA,
1455delA, 1456delG, 1457delA, 1458delG, 1459delT, 1460delT, 1461delC, 1462delA,
1463delC, 1464delT, 1465delC, 1466delC, 1467delA, 1468delA, 1469delA, 1470delT,
1471delC, 1472delA, 1473delG, 1474delT, 1475delA, 1476delG, 1477delA, 1478delG,
1479delA, 1480delG, 1481delT, 1482delA, 1483delA, 1484delT, 1485delA, 1486delT,
1487delT, 1488delG, 1489delA, 1490delA, 1491delG, 1492delA, 1493delC, 1494delA,
1495delA, 1496delA, 1497delA, 1498delT, 1499delA, 1500delT, 1501delT, 1502delT,
1503delG, 1504delG, 1505delG, 1506delA, 1507delA, 1508delA, 1509delA, 1510delC,
1511delC, 1512delT, 1513delA, 1514delT, 1515delC, 1516delG, 1517delG, 1518delA,
1519delA, 1520delG, 1521delA, 1522delA, 1523delG, 1524delG, 1525delC, 1526delA,
1527delA, 1528delG, 1529delC, 1530delC, 1531delT, 1532delC, 1533delC, 1534delC,
1535delC, 1536delA, 1537delA, 1538delC, 1539delT, 1540delT, 1541delA, 1542delA,
1543delG, 1544delC, 1545delC, 1546delA, 1547delT, 1548delG, 1549delT, 1550delA,
1551delA, 1552delC, 1553delT, 1554delG, 1555delA, 1556delA, 1557delA, 1558delA,
1559delT, 1560delC, 1561delT, 1562delA, 1563delA, 1564delT, 1565delT, 1566delA,
1567delT, 1568delA, 1569delG, 1570delG, 1571delA, 1572delG, 1573delC, 1574delA,
1575delT, 1576delT, 1577delT, 1578delG, 1579delT, 1580delT, 1581delA, 1582delC, 1583delT,
1584delG, 1585delA, 1586delG, 1587delC, 1588delC, 1589delA, 1590delC, 1591delA,
1592delG, 1593delA, 1594delT, 1595delA, 1596delA, 1597delT, 1598delA, 1599delC,
1600delA, 1601delA, 1602delG, 1603delA, 1604delG, 1605delC, 1606delG, 1607delT,
1608delC, 1609delC, 1610delC, 1611delC, 1612delT, 1613delC, 1614delA, 1615delC,
1616delA, 1617delA, 1618delA, 1619delT, 1620delA, 1621delA, 1622delA, 1623delT,
1624delT, 1625delA, 1626delA, 1627delA, 1628delG, 1629delC, 1630delG, 1631delT,
1632delA, 1633delA, 1634delA, 1635delA, 1636delG, 1637delG, 1638delA, 1639delG,
1640delA, 1641delC, 1642delC, 1643delT, 1644delA, 1645delC, 1646delA, 1647delT,
1648delC, 1649delA, 1650delG, 1651delG, 1652delC, 1653delC, 1654delT, 1655delT,
1656delC, 1657delA, 1658delT, 1659delC, 1660delC, 1661delT, 1662delG, 1663delA,
1664delG, 1665delG, 1666delA, 1667delT, 1668delT, 1669delT, 1670delT, 1671delA,
1672delT, 1673delC, 1674delA, 1675delA, 1676delG, 1677delA, 1678delA, 1679delA,
1680delG, 1681delC, 1682delA, 1683delG, 1684delA, 1685delT, 1686delT, 1687delT,
1688delG, 1689delG, 1690delC, 1691delA, 1692delG, 1693delT, 1694delT, 1695delC,
1696delA, 1697delA, 1698delA, 1699delA, 1700delG, 1701delA, 1702delC, 1703delT,
1704delC, 1705delC, 1706delT, 1707delG, 1708delA, 1709delA, 1710delA, 1711delT,
1712delG, 1713delA, 1714delT, 1715delA, 1716delA, 1717delA, 1718delT, 1719delC,
1720delA, 1721delG, 1722delG, 1723delG, 1724delA, 1725delA, 1726delC, 1727delT,
1728delA, 1729delA, 1730delC, 1731delC, 1732delA, 1733delA, 1734delA, 1735delC,
1736delG, 1737delG, 1738delA, 1739delG, 1740delC, 1741delA, 1742delG, 1743delA,
1744delA, 1745delT, 1746delG, 1747delG, 1748delT, 1749delC, 1750delA, 1751delA,
1752delG, 1753delT, 1754delG, 1755delA, 1756delT, 1757delG, 1758delA, 1759delA,
1760delT, 1761delA, 1762delT, 1763delT, 1764delA, 1765delC, 1766delT, 1767delA,
1768delA, 1769delT, 1770delA, 1771delG, 1772delT, 1773delG, 1774delG, 1775delT,
1776delC, 1777delA, 1778delT, 1779delG, 1780delA, 1781delG, 1782delA, 1783delA,
1784delT, 1785delA, 1786delA, 1787delA, 1788delA, 1789delC, 1790delA, 1791delA,
1792delA, 1793delA, 1794delG, 1795delG, 1796delT, 1797delG, 1798delA, 1799delT,
1800delT, 1801delC, 1802delT, 1803delA, 1804delT, 1805delT, 1806delC, 1807delA,
1808delG, 1809delA, 1810delA, 1811delT, 1812delG, 1813delA, 1814delG, 1815delA,
1816delA, 1817delA, 1818delA, 1819delA, 1820delT, 1821delC, 1822delC, 1823delT,
1824delA, 1825delA, 1826delC, 1827delC, 1828delC, 1829delA, 1830delA, 1831delT,
1832delA, 1833delG, 1834delA, 1835delA, 1836delT, 1837delC, 1838delA, 1839delC,
1840delT, 1841delC, 1842delG, 1843delA, 1844delA, 1845delA, 1846delA, 1847delA,
1848delG, 1849delA, 1850delA, 1851delT, 1852delC, 1853delT, 1854delG, 1855delC,
1856delT, 1857delT, 1858delT, 1859delC, 1860delA, 1861delA, 1862delA, 1863delA,
1864delC, 1865delG, 1866delA, 1867delA, 1868delA, 1869delG, 1870delC, 1871delT,
1872delG, 1873delA, 1874delA, 1875delC, 1876delC, 1877delT, 1878delA, 1879delT,
1880delA, 1881delA, 1882delG, 1883delC, 1884delA, 1885delG, 1886delC, 1887delA,
1888delG, 1889delT, 1890delA, 1891delT, 1892delA, 1893delA, 1894delG, 1895delC,
1896delA, 1897delA, 1898delT, 1899delA, 1900delT, 1901delG, 1902delG, 1903delA,
1904delA, 1905delC, 1906delT, 1907delC, 1908delG, 1909delA, 1910delA, 1911delT,
1912delT, 1913delA, 1914delA, 1915delA, 1916delT, 1917delA, 1918delT, 1919delC,
1920delC, 1921delA, 1922delC, 1923delA, 1924delA, 1925delT, 1926delT, 1927delC,
1928delA, 1929delA, 1930delA, 1931delA, 1932delG, 1933delC, 1934delA, 1935delC,
1936delC, 1937delT, 1938delA, 1939delA, 1940delA, 1941delA, 1942delA, 1943delG,
1944delA, 1945delA, 1946delT, 1947delA, 1948delG, 1949delG, 1950delC, 1951delT,
1952delG, 1953delA, 1954delG, 1955delG, 1956delA, 1957delG, 1958delG, 1959delA,
1960delA, 1961delG, 1962delT, 1963delC, 1964delT, 1965delT, 1966delC, 1967delT,
1968delA, 1969delC, 1970delC, 1971delA, 1972delG, 1973delG, 1974delC, 1975delA,
1976delT, 1977delA, 1978delT, 1979delT, 1980delC, 1981delA, 1982delT, 1983delG,
1984delC, 1985delG, 1986delC, 1987delT, 1988delT, 1989delG, 1990delA, 1991delA,
1992delC, 1993delT, 1994delA, 1995delG, 1996delT, 1997delA, 1998delG, 1999delT,
2000delC, 2001delA, 2002delG, 2003delT, 2004delA, 2005delG, 2006delA, 2007delA,
2008delA, 2009delT, 2010delC, 2011delT, 2012delA, 2013delA, 2014delG, 2015delC,
2016delC, 2017delC, 2018delA, 2019delC, 2020delC, 2021delT, 2022delA, 2023delA,
2024delT, 2025delT, 2026delG, 2027delT, 2028delA, 2029delC, 2030delT, 2031delG,
2032delA, 2033delA, 2034delT, 2035delT, 2036delG, 2037delC, 2038delA, 2039delA,
2040delA, 2041delT, 2042delT, 2043delG, 2044delA, 2045delT, 2046delA, 2047delG,
2048delT, 2049delT, 2050delG, 2051delT, 2052delT, 2053delC, 2054delT, 2055delA,
2056delG, 2057delC, 2058delA, 2059delG, 2060delT, 2061delG, 2062delA, 2063delA,
2064delG, 2065delA, 2066delG, 2067delA, 2068delT, 2069delA, 2070delA, 2071delA,
2072delG, 2073delA, 2074delA, 2075delA, 2076delA, 2077delA, 2078delA, 2079delA,
2080delA, 2081delG, 2082delT, 2083delA, 2084delC, 2085delA, 2086delA, 2087delC,
2088delC, 2089delA, 2090delA, 2091delA, 2092delT, 2093delG, 2094delC, 2095delC,
2096delA, 2097delG, 2098delT, 2099delC, 2100delA, 2101delG, 2102delG, 2103delC,
2104delA, 2105delC, 2106delA, 2107delG, 2108delC, 2109delA, 2110delG, 2111delA,
2112delA, 2113delA, 2114delC, 2115delC, 2116delT, 2117delA, 2118delC, 2119delA,
2120delA, 2121delC, 2122delT, 2123delC, 2124delA, 2125delT, 2126delG, 2127delG,
2128delA, 2129delA, 2130delG, 2131delG, 2132delT, 2133delA, 2134delA, 2135delA,
2136delG, 2137delA, 2138delA, 2139delC, 2140delC, 2141delT, 2142delG, 2143delC,
2144delA, 2145delA, 2146delC, 2147delT, 2148delG, 2149delG, 2150delA, 2151delG,
2152delC, 2153delC, 2154delA, 2155delA, 2156delG, 2157delA, 2158delA, 2159delG,
2160delA, 2161delG, 2162delT, 2163delA, 2164delA, 2165delC, 2166delA, 2167delA,
2168delG, 2169delC, 2170delC, 2171delA, 2172delA, 2173delA, 2174delT, 2175delG,
2176delA, 2177delA, 2178delC, 2179delA, 2180delG, 2181delA, 2182delC, 2183delA,
2184delA, 2185delG, 2186delT, 2187delA, 2188delA, 2189delA, 2190delA, 2191delG,
2192delA, 2193delC, 2194delA, 2195delT, 2196delG, 2197delA, 2198delC, 2199delA,
2200delG, 2201delT, 2202delG, 2203delA, 2204delT, 2205delA, 2206delC, 2207delT,
2208delT, 2209delT, 2210delC, 2211delC, 2212delC, 2213delA, 2214delG, 2215delA,
2216delG, 2217delC, 2218delT, 2219delG, 2220delA, 2221delA, 2222delG, 2223delT,
2224delT, 2225delA, 2226delA, 2227delC, 2228delA, 2229delA, 2230delA, 2231delT,
2232delG, 2233delC, 2234delA, 2235delC, 2236delC, 2237delT, 2238delG, 2239delG,
2240delT, 2241delT, 2242delC, 2243delT, 2244delT, 2245delT, 2246delT, 2247delA, 2248delC,
2249delT, 2250delA, 2251delA, 2252delG, 2253delT, 2254delG, 2255delT, 2256delT,
2257delC, 2258delA, 2259delA, 2260delA, 2261delT, 2262delA, 2263delC, 2264delC,
2265delA, 2266delG, 2267delT, 2268delG, 2269delA, 2270delA, 2271delC, 2272delT,
2273delT, 2274delA, 2275delA, 2276delA, 2277delG, 2278delA, 2279delA, 2280delT,
2281delT, 2282delT, 2283delG, 2284delT, 2285delC, 2286delA, 2287delA, 2288delT,
2289delC, 2290delC, 2291delT, 2292delA, 2293delG, 2294delC, 2295delC, 2296delT,
2297delT, 2298delC, 2299delC, 2300delA, 2301delA, 2302delG, 2303delA, 2304delG,
2305delA, 2306delA, 2307delG, 2308delA, 2309delA, 2310delA, 2311delA, 2312delA,
2313delG, 2314delA, 2315delA, 2316delG, 2317delA, 2318delG, 2319delA, 2320delA,
2321delA, 2322delC, 2323delT, 2324delA, 2325delG, 2326delA, 2327delA, 2328delA,
2329delC, 2330delA, 2331delG, 2332delT, 2333delT, 2334delA, 2335delA, 2336delA,
2337delG, 2338delT, 2339delG, 2340delT, 2341delC, 2342delT, 2343delA, 2344delA,
2345delT, 2346delA, 2347delA, 2348delT, 2349delG, 2350delC, 2351delT, 2352delG,
2353delA, 2354delA, 2355delG, 2356delA, 2357delC, 2358delC, 2359delC, 2360delC,
2361delA, 2362delA, 2363delA, 2364delG, 2365delA, 2366delT, 2367delC, 2368delT,
2369delC, 2370delA, 2371delT, 2372delG, 2373delT, 2374delT, 2375delA, 2376delA,
2377delG, 2378delT, 2379delG, 2380delG, 2381delA, 2382delG, 2383delA, 2384delA,
2385delA, 2386delG, 2387delG, 2388delG, 2389delT, 2390delT, 2391delT, 2392delT,
2393delG, 2394delC, 2395delA, 2396delA, 2397delA, 2398delC, 2399delT, 2400delG,
2401delA, 2402delA, 2403delA, 2404delG, 2405delA, 2406delT, 2407delC, 2408delT,
2409delG, 2410delT, 2411delA, 2412delG, 2413delA, 2414delG, 2415delA, 2416delG,
2417delT, 2418delA, 2419delG, 2420delC, 2421delA, 2422delG, 2423delT, 2424delA,
2425delT, 2426delT, 2427delT, 2428delC, 2429delA, 2430delC, 2431delT, 2432delG,
2433delG, 2434delT, 2435delA, 2436delC, 2437delC, 2438delT, 2439delG, 2440delG,
2441delT, 2442delA, 2443delC, 2444delT, 2445delG, 2446delA, 2447delT, 2448delT,
2449delA, 2450delT, 2451delG, 2452delG, 2453delC, 2454delA, 2455delC, 2456delT,
2457delC, 2458delA, 2459delG, 2460delG, 2461delA, 2462delA, 2463delA, 2464delG,
2465delT, 2466delA, 2467delT, 2468delC, 2469delT, 2470delC, 2471delG, 2472delT,
2473delT, 2474delA, 2475delC, 2476delT, 2477delG, 2478delG, 2479delA, 2480delA,
2481delG, 2482delT, 2483delT, 2484delA, 2485delG, 2486delC, 2487delA, 2488delC,
2489delT, 2490delC, 2491delT, 2492delA, 2493delG, 2494delG, 2495delG, 2496delA,
2497delA, 2498delG, 2499delG, 2500delC, 2501delA, 2502delA, 2503delA, 2504delA,
2505delA, 2506delC, 2507delA, 2508delG, 2509delA, 2510delA, 2511delC, 2512delC,
2513delA, 2514delA, 2515delA, 2516delT, 2517delA, 2518delA, 2519delA, 2520delT,
2521delG, 2522delT, 2523delG, 2524delT, 2525delG, 2526delA, 2527delG, 2528delT,
2529delC, 2530delA, 2531delG, 2532delT, 2533delG, 2534delT, 2535delG, 2536delC,
2537delA, 2538delG, 2539delC, 2540delA, 2541delT, 2542delT, 2543delT, 2544delG,
2545delA, 2546delA, 2547delA, 2548delA, 2549delC, 2550delC, 2551delC, 2552delC,
2553delA, 2554delA, 2555delG, 2556delG, 2557delG, 2558delA, 2559delC, 2560delT,
2561delA, 2562delA, 2563delT, 2564delT, 2565delC, 2566delA, 2567delT, 2568delG,
2569delG, 2570delT, 2571delT, 2572delG, 2573delT, 2574delT, 2575delC, 2576delC,
2577delA, 2578delA, 2579delA, 2580delG, 2581delA, 2582delT, 2583delA, 2584delA,
2585delT, 2586delA, 2587delG, 2588delA, 2589delA, 2590delA, 2591delT, 2592delG,
2593delA, 2594delC, 2595delA, 2596delC, 2597delA, 2598delG, 2599delA, 2600delA,
2601delG, 2602delG, 2603delC, 2604delT, 2605delT, 2606delT, 2607delA, 2608delA,
2609delG, 2610delT, 2611delA, 2612delT, 2613delC, 2614delC, 2615delA, 2616delT,
2617delT, 2618delG, 2619delG, 2620delG, 2621delA, 2622delC, 2623delA, 2624delT,
2625delG, 2626delA, 2627delA, 2628delG, 2629delT, 2630delT, 2631delA, 2632delA,
2633delC, 2634delC, 2635delA, 2636delC, 2637delA, 2638delG, 2639delT, 2640delC,
2641delG, 2642delG, 2643delG, 2644delA, 2645delA, 2646delA, 2647delC, 2648delA,
2649delA, 2650delG, 2651delC, 2652delA, 2653delT, 2654delA, 2655delG, 2656delA,
2657delA, 2658delA, 2659delT, 2660delG, 2661delG, 2662delA, 2663delA, 2664delG,
2665delA, 2666delA, 2667delA, 2668delG, 2669delT, 2670delG, 2671delA, 2672delA,
2673delC, 2674delT, 2675delT, 2676delG, 2677delA, 2678delT, 2679delG, 2680delC,
2681delT, 2682delC, 2683delA, 2684delG, 2685delT, 2686delA, 2687delT, 2688delT,
2689delT, 2690delG, 2691delC, 2692delA, 2693delG, 2694delA, 2695delA, 2696delT,
2697delA, 2698delC, 2699delA, 2700delT, 2701delT, 2702delC, 2703delA, 2704delA,
2705delG, 2706delG, 2707delT, 2708delT, 2709delT, 2710delC, 2711delA, 2712delA,
2713delA, 2714delG, 2715delC, 2716delG, 2717delC, 2718delC, 2719delA, 2720delG,
2721delT, 2722delC, 2723delA, 2724delT, 2725delT, 2726delT, 2727delG, 2728delC,
2729delT, 2730delC, 2731delT, 2732delG, 2733delT, 2734delT, 2735delT, 2736delT, 2737delC,
2738delA, 2739delA, 2740delA, 2741delT, 2742delC, 2743delC, 2744delA, 2745delG,
2746delG, 2747delA, 2748delA, 2749delA, 2750delT, 2751delG, 2752delC, 2753delA,
2754delG, 2755delA, 2756delA, 2757delG, 2758delA, 2759delG, 2760delG, 2761delA,
2762delA, 2763delT, 2764delG, 2765delT, 2766delG, 2767delC, 2768delA, 2769delA,
2770delC, 2771delA, 2772delT, 2773delT, 2774delC, 2775delT, 2776delC, 2777delT,
2778delG, 2779delC, 2780delC, 2781delC, 2782delA, 2783delC, 2784delT, 2785delC,
2786delT, 2787delG, 2788delG, 2789delG, 2790delT, 2791delC, 2792delC, 2793delT,
2794delT, 2795delA, 2796delA, 2797delA, 2798delG, 2799delA, 2800delA, 2801delA,
2802delC, 2803delA, 2804delA, 2805delA, 2806delG, 2807delT, 2808delC, 2809delC,
2810delA, 2811delA, 2812delA, 2813delA, 2814delG, 2815delT, 2816delC, 2817delA,
2818delC, 2819delT, 2820delT, 2821delT, 2822delT, 2823delG, 2824delA, 2825delA,
2826delT, 2827delG, 2828delT, 2829delG, 2830delA, 2831delA, 2832delC, 2833delA,
2834delA, 2835delA, 2836delA, 2837delG, 2838delG, 2839delA, 2840delA, 2841delG,
2842delA, 2843delA, 2844delA, 2845delA, 2846delT, 2847delC, 2848delA, 2849delA,
2850delG, 2851delG, 2852delA, 2853delA, 2854delA, 2855delG, 2856delA, 2857delA,
2858delT, 2859delG, 2860delA, 2861delG, 2862delT, 2863delC, 2864delT, 2865delA,
2866delA, 2867delT, 2868delA, 2869delT, 2870delC, 2871delA, 2872delA, 2873delG,
2874delC, 2875delC, 2876delT, 2877delG, 2878delT, 2879delA, 2880delC, 2881delA,
2882delG, 2883delA, 2884delC, 2885delA, 2886delG, 2887delT, 2888delT, 2889delA,
2890delA, 2891delT, 2892delA, 2893delT, 2894delC, 2895delA, 2896delC, 2897delT,
2898delG, 2899delC, 2900delA, 2901delG, 2902delG, 2903delC, 2904delT, 2905delT,
2906delT, 2907delC, 2908delC, 2909delT, 2910delG, 2911delT, 2912delG, 2913delG,
2914delT, 2915delT, 2916delG, 2917delG, 2918delT, 2919delC, 2920delA, 2921delG,
2922delA, 2923delA, 2924delA, 2925delG, 2926delA, 2927delT, 2928delA, 2929delA,
2930delG, 2931delC, 2932delC, 2933delA, 2934delG, 2935delT, 2936delT, 2937delG,
2938delA, 2939delT, 2940delA, 2941delA, 2942delT, 2943delG, 2944delC, 2945delC,
2946delA, 2947delA, 2948delA, 2949delT, 2950delG, 2951delT, 2952delA, 2953delG,
2954delT, 2955delA, 2956delT, 2957delC, 2958delA, 2959delA, 2960delA, 2961delG,
2962delG, 2963delA, 2964delG, 2965delG, 2966delC, 2967delT, 2968delC, 2969delT,
2970delA, 2971delG, 2972delG, 2973delT, 2974delT, 2975delT, 2976delT, 2977delG,
2978delT, 2979delC, 2980delT, 2981delA, 2982delT, 2983delC, 2984delA, 2985delT,
2986delC, 2987delT, 2988delC, 2989delA, 2990delG, 2991delT, 2992delT, 2993delC,
2994delA, 2995delG, 2996delA, 2997delG, 2998delG, 2999delC, 3000delA, 3001delA,
3002delC, 3003delG, 3004delA, 3005delA, 3006delA, 3007delC, 3008delT, 3009delG,
3010delG, 3011delA, 3012delC, 3013delT, 3014delC, 3015delA, 3016delT, 3017delT,
3018delA, 3019delC, 3020delT, 3021delC, 3022delC, 3023delA, 3024delA, 3025delA,
3026delT, 3027delA, 3028delA, 3029delA, 3030delC, 3031delA, 3032delT, 3033delG,
3034delG, 3035delA, 3036delC, 3037delT, 3038delT, 3039delT, 3040delT, 3041delA,
3042delC, 3043delA, 3044delA, 3045delA, 3046delA, 3047delC, 3048delC, 3049delC,
3050delA, 3051delT, 3052delA, 3053delT, 3054delC, 3055delG, 3056delT, 3057delA,
3058delT, 3059delA, 3060delC, 3061delC, 3062delA, 3063delC, 3064delC, 3065delA,
3066delC, 3067delT, 3068delT, 3069delT, 3070delT, 3071delT, 3072delC, 3073delC, 3074delC,
3075delA, 3076delT, 3077delC, 3078delA, 3079delA, 3080delG, 3081delT, 3082delC,
3083delA, 3084delT, 3085delT, 3086delT, 3087delG, 3088delT, 3089delT, 3090delA,
3091delA, 3092delA, 3093delA, 3094delC, 3095delT, 3096delA, 3097delA, 3098delA,
3099delT, 3100delG, 3101delT, 3102delA, 3103delA, 3104delG, 3105delA, 3106delA,
3107delA, 3108delA, 3109delA, 3110delT, 3111delC, 3112delT, 3113delG, 3114delC,
3115delT, 3116delA, 3117delG, 3118delA, 3119delG, 3120delG, 3121delA, 3122delA,
3123delA, 3124delA, 3125delC, 3126delT, 3127delT, 3128delT, 3129delG, 3030delA,
3131delG, 3132delG, 3133delA, 3134delA, 3135delC, 3136delA, 3137delT, 3138delT,
3139delC, 3140delA, 3141delA, 3142delT, 3143delG, 3144delT, 3145delC, 3146delA,
3147delC, 3148delC, 3149delT, 3150delG, 3151delA, 3152delA, 3153delA, 3154delG,
3155delA, 3156delG, 3157delA, 3158delA, 3159delA, 3160delT, 3161delG, 3162delG,
3163delG, 3164delA, 3165delA, 3166delA, 3167delT, 3168delG, 3169delA, 3170delG,
3171delA, 3172delA, 3173delC, 3174delA, 3175delT, 3176delT, 3177delC, 3178delC,
3179delA, 3180delA, 3181delG, 3182delT, 3183delA, 3184delC, 3185delA, 3186delG,
3187delT, 3188delG, 3189delA, 3190delG, 3191delC, 3192delA, 3193delC, 3194delA,
3195delA, 3196delT, 3197delT, 3198delA, 3199delG, 3200delC, 3201delC, 3202delG,
3203delT, 3204delA, 3205delA, 3206delT, 3207delA, 3208delA, 3209delC, 3210delA,
3211delT, 3212delT, 3213delA, 3214delG, 3215delA, 3216delG, 3217delA, 3218delA,
3219delA, 3220delA, 3221delT, 3222delG, 3223delT, 3224delT, 3225delT, 3226delT,
3227delT, 3228delA, 3229delA, 3230delA, 3231delG, 3232delG, 3233delA, 3234delG,
3235delC, 3236delC, 3237delA, 3238delG, 3239delC, 3240delT, 3241delC, 3242delA,
3243delA, 3244delG, 3245delC, 3246delA, 3247delA, 3248delT, 3249delA, 3250delT,
3251delT, 3252delA, 3253delA, 3254delT, 3255delG, 3256delA, 3257delA, 3258delG,
3259delT, 3260delA, 3261delG, 3262delG, 3263delT, 3264delT, 3265delC, 3266delC,
3267delA, 3268delG, 3269delT, 3270delA, 3271delC, 3272delT, 3273delA, 3274delA,
3275delT, 3276delG, 3277delA, 3278delA, 3279delG, 3280delT, 3281delG, 3282delG,
3283delG, 3284delC, 3285delT, 3286delC, 3287delC, 3288delA, 3289delG, 3290delT,
3291delA, 3292delT, 3293delT, 3294delA, 3295delA, 3296delT, 3297delG, 3298delA,
3299delA, 3300delA, 3301delT, 3302delA, 3303delG, 3304delG, 3305delT, 3306delT,
3307delC, 3308delC, 3309delA, 3310delG, 3311delT, 3312delG, 3313delA, 3314delT,
3315delG, 3316delA, 3317delA, 3318delA, 3319delA, 3320delC, 3321delA, 3322delT,
3323delT, 3324delC, 3325delA, 3326delA, 3327delG, 3328delC, 3329delA, 3330delG,
3331delA, 3332delA, 3333delC, 3334delT, 3335delA, 3336delG, 3337delG, 3338delT,
3339delA, 3340delG, 3341delA, 3342delA, 3343delA, 3344delC, 3345delA, 3346delG,
3347delA, 3348delG, 3349delG, 3350delG, 3351delC, 3352delC, 3353delA, 3354delA,
3355delA, 3356delA, 3357delT, 3358delT, 3359delG, 3360delA, 3361delA, 3362delT,
3363delG, 3364delC, 3365delT, 3366delA, 3367delT, 3368delG, 3369delC, 3370delT,
3371delT, 3372delA, 3373delG, 3374delA, 3375delT, 3376delT, 3377delA, 3378delG,
3379delG, 3380delG, 3381delG, 3382delT, 3383delT, 3384delT, 3385delT, 3386delG,
3387delC, 3388delA, 3389delA, 3390delC, 3391delC, 3392delT, 3393delG, 3394delA,
3395delG, 3396delG, 3397delT, 3398delC, 3399delT, 3400delA, 3401delT, 3402delA,
3403delA, 3404delA, 3405delC, 3406delA, 3407delA, 3408delA, 3409delG, 3410delT,
3411delC, 3412delT, 3413delT, 3414delC, 3415delC, 3416delT, 3417delG, 3418delG,
3419delA, 3420delA, 3421delG, 3422delT, 3423delA, 3424delA, 3425delT, 3426delT,
3427delG, 3428delT, 3429delA, 3430delA, 3431delG, 3432delC, 3433delA, 3434delT,
3435delC, 3436delC, 3437delT, 3438delG, 3439delA, 3440delA, 3441delA, 3442delT,
3443delA, 3444delA, 3445delA, 3446delA, 3447delA, 3448delA, 3449delG, 3450delC,
3451delA, 3452delA, 3453delG, 3454delA, 3455delA, 3456delT, 3457delA, 3458delT,
3459delG, 3460delA, 3461delA, 3462delG, 3463delA, 3464delA, 3465delG, 3466delT,
3467delA, 3468delG, 3469delT, 3470delT, 3471delC, 3472delA, 3473delG, 3474delA,
3475delC, 3476delT, 3477delG, 3478delT, 3479delT, 3480delA, 3481delA, 3482delT,
3483delA, 3484delC, 3485delA, 3486delG, 3487delA, 3488delT, 3489delT, 3490delT,
3491delC, 3492delT, 3493delC, 3494delT, 3495delC, 3496delC, 3497delA, 3498delT,
3499delA, 3500delT, 3501delC, 3502delT, 3503delG, 3504delA, 3505delT, 3506delT,
3507delT, 3508delC, 3509delA, 3510delG, 3511delA, 3512delT, 3513delA, 3514delA,
3515delC, 3516delT, 3517delT, 3518delA, 3519delG, 3520delA, 3521delA, 3522delC,
3523delA, 3524delG, 3525delC, 3526delC, 3527delT, 3528delA, 3529delT, 3530delG,
3531delG, 3532delG, 3533delA, 3534delA, 3535delG, 3536delT, 3537delA, 3538delG,
3539delT, 3540delC, 3541delA, 3542delT, 3543delG, 3544delC, 3545delA, 3546delT,
3547delC, 3548delT, 3549delC, 3550delA, 3551delG, 3552delG, 3553delT, 3554delT,
3555delT, 3556delG, 3557delT, 3558delT, 3559delC, 3560delT, 3561delG, 3562delA,
3563delG, 3564delA, 3565delC, 3566delA, 3567delC, 3568delC, 3569delT, 3570delG,
3571delA, 3572delT, 3573delG, 3574delA, 3575delC, 3576delC, 3577delT, 3578delG,
3579delT, 3580delT, 3581delA, 3582delG, 3583delA, 3584delT, 3585delG, 3586delA,
3587delT, 3588delG, 3589delG, 3590delT, 3591delG, 3592delA, 3593delA, 3594delA,
3595delT, 3596delA, 3597delA, 3598delA, 3599delG, 3600delG, 3601delA, 3602delA,
3603delG, 3604delA, 3605delT, 3606delA, 3607delC, 3608delT, 3609delA, 3610delG,
3611delT, 3612delT, 3613delT, 3614delT, 3615delG, 3616delC, 3617delT, 3618delG,
3619delA, 3620delA, 3621delA, 3622delA, 3623delT, 3624delG, 3625delA, 3626delC,
3627delA, 3628delT, 3629delT, 3630delA, 3631delA, 3632delG, 3633delG, 3634delA,
3635delA, 3636delA, 3637delG, 3638delT, 3639delT, 3640delC, 3641delT, 3642delG,
3643delC, 3644delT, 3645delG, 3646delT, 3647delT, 3648delT, 3649delT, 3650delT, 3651delA,
3652delG, 3653delC, 3654delA, 3655delA, 3656delA, 3657delA, 3658delG, 3659delC,
3660delG, 3661delT, 3662delC, 3663delC, 3664delA, 3665delG, 3666delA, 3667delG,
3668delA, 3669delG, 3670delG, 3671delA, 3672delG, 3673delA, 3674delG, 3675delC
3676delT, 3677delT, 3678delA, 3679delG, 3680delC, 3681delA, 3682delG, 3683delG,
3684delA, 3685delG, 3686delT, 3687delC, 3688delC, 3689delT, 3690delA, 3691delG,
3692delC, 3693delC, 3694delC, 3695delT, 3696delT, 3697delT, 3698delC, 3699delA,
3700delC, 3701delC, 3702delC, 3703delA, 3704delT, 3705delA, 3706delC, 3707delA,
3708delC, 3709delA, 3710delT, 3711delT, 3712delT, 3713delG, 3714delG, 3715delC,
3716delT, 3717delC, 3718delA, 3719delG, 3720delG, 3721delG, 3722delT, 3723delT,
3724delA, 3725delC, 3726delC, 3727delG, 3728delA, 3729delA, 3730delG, 3731delA,
3732delG, 3733delG, 3734delG, 3735delG, 3736delC, 3737delC, 3738delA, 3739delA,
3740delG, 3741delA, 3742delA, 3743delA, 3744delT, 3745delT, 3746delA, 3747delG,
3748delA, 3749delG, 3750delT, 3751delC, 3752delC, 3753delT, 3754delC, 3755delA,
3756delG, 3757delA, 3758delA, 3759delG, 3760delA, 3761delG, 3762delA, 3763delA,
3764delC, 3765delT, 3766delT, 3767delA, 3768delT, 3769delC, 3770delT, 3771delA,
3772delG, 3773delT, 3774delG, 3775delA, 3776delG, 3777delG, 3778delA, 3779delT,
3780delG, 3781delA, 3782delA, 3783delG, 3784delA, 3785delG, 3786delC, 3787delT,
3788delT, 3789delC, 3790delC, 3791delC, 3792delT, 3793delG, 3794delC, 3795delT,
3796delT, 3797delC, 3798delC, 3799delA, 3800delA, 3801delC, 3802delA, 3803delC,
3804delT, 3805delT, 3806delG, 3807delT, 3808delT, 3809delA, 3810delT, 3811delT, 3812delT,
3813delG, 3814delG, 3815delT, 3816delA, 3817delA, 3818delA, 3819delG, 3820delT,
3821delA, 3822delA, 3823delA, 3824delC, 3825delA, 3826delA, 3827delT, 3828delA,
3829delT, 3830delA, 3831delC, 3832delC, 3833delT, 3834delT, 3835delC, 3836delT,
3837delC, 3838delA, 3839delG, 3840delT, 3841delC, 3842delT, 3843delA, 3844delC,
3845delT, 3846delA, 3847delG, 3848delG, 3849delC, 3850delA, 3851delT, 3852delA,
3853delG, 3854delC, 3855delA, 3856delC, 3857delC, 3858delG, 3859delT, 3860delT,
3861delG, 3862delC, 3863delT, 3864delA, 3865delC, 3866delC, 3867delG, 3868delA,
3869delG, 3870delT, 3871delG, 3872delT, 3873delC, 3874delT, 3875delG, 3876delT,
3877delC, 3878delT, 3879delA, 3880delA, 3881delG, 3882delA, 3883delA, 3884delC,
3885delA, 3886delC, 3887delA, 3888delG, 3889delA, 3890delG, 3891delG, 3892delA,
3893delG, 3894delA, 3895delA, 3896delT, 3897delT, 3898delT, 3899delA, 3900delT,
3901delT, 3902delA, 3903delT, 3904delC, 3905delA, 3906delT, 3907delT, 3908delG,
3909delA, 3910delA, 3911delG, 3912delA, 3913delA, 3914delT, 3915delA, 3916delG,
3917delC, 3918delT, 3919delT, 3920delA, 3921delA, 3922delA, 3923delT, 3924delG,
3925delA, 3926delC, 3927delT, 3928delG, 3929delC, 3930delA, 3931delG, 3932delT,
3933delA, 3934delA, 3935delC, 3936delC, 3937delA, 3938delG, 3939delG, 3940delT,
3941delA, 3942delA, 3943delT, 3944delA, 3945delT, 3946delT, 3947delG, 3948delG,
3949delC, 3950delA, 3951delA, 3952delA, 3953delG, 3954delG, 3955delC, 3956delA,
3957delT, 3958delC, 3959delT, 3960delC, 3961delA, 3962delG, 3963delG, 3964delA,
3965delA, 3966delC, 3967delA, 3968delT, 3969delC, 3970delA, 3971delC, 3972delC,
3973delT, 3974delT, 3975delA, 3976delG, 3977delT, 3978delG, 3979delA, 3980delG,
3981delG, 3982delA, 3983delA, 3984delA, 3985delC, 3986delA, 3987delA, 3988delA,
3989delA, 3990delT, 3991delG, 3992delT, 3993delT, 3994delC, 3995delT, 3996delG,
3997delC, 3998delT, 3999delA, 4000delG, 4001delC, 4002delT, 4003delT, 4004delG,
4005delT, 4006delT, 4007delT, 4008delT, 4009delC, 4010delT, 4011delT, 4012delC, 4013delA,
4014delC, 4015delA, 4016delG, 4017delT, 4018delG, 4019delC, 4020delA, 4021delG,
4022delT, 4023delG, 4024delA, 4025delA, 4026delT, 4027delT, 4028delG, 4029delG,
4030delA, 4031delA, 4032delG, 4033delA, 4034delC, 4035delT, 4036delT, 4037delG,
4038delA, 4039delC, 4040delT, 4041delG, 4042delC, 4043delA, 4044delA, 4045delA,
4046delT, 4047delA, 4048delC, 4049delA, 4050delA, 4051delA, 4052delC, 4053delA,
4054delC, 4055delC, 4056delC, 4057delA, 4058delG, 4059delG, 4060delA, 4061delT,
4062delC, 4063delC, 4064delT, 4065delT, 4066delT, 4067delC, 4068delT, 4069delT,
4070delG, 4071delA, 4072delT, 4073delT, 4074delG, 4075delG, 4076delT, 4077delT,
4078delC, 4079delT, 4080delT, 4081delC, 4082delC, 4083delA, 4084delA, 4085delA,
4086delC, 4087delA, 4088delA, 4089delA, 4090delT, 4091delG, 4092delA, 4093delG,
4094delG, 4095delC, 4096delA, 4097delT, 4098delC, 4099delA, 4100delG, 4101delT,
4102delC, 4103delT, 4104delG, 4105delA, 4106delA, 4107delA, 4108delG, 4109delC,
4110delC, 4111delA, 4112delG, 4113delG, 4114delG, 4115delA, 4116delG, 4117delT,
4118delT, 4119delG, 4120delG, 4121delT, 4122delC, 4123delT, 4124delG, 4125delA,
4126delG, 4127delT, 4128delG, 4129delA, 4130delC, 4131delA, 4132delA, 4133delG,
4134delG, 4135delA, 4136delA, 4137delT, 4138delT, 4139delG, 4140delG, 4141delT,
4142delT, 4143delT, 4144delC, 4145delA, 4146delG, 4147delA, 4148delT, 4149delG,
4150delA, 4151delT, 4152delG, 4153delA, 4154delA, 4155delG, 4156delA, 4157delA,
4158delA, 4159delG, 4160delA, 4161delG, 4162delG, 4163delA, 4164delA, 4165delC,
4166delG, 4167delG, 4168delG, 4169delC, 4170delT, 4171delT, 4172delG, 4173delG,
4174delA, 4175delA, 4176delG, 4177delA, 4178delA, 4179delA, 4180delA, 4181delT,
4182delA, 4183delA, 4184delT, 4185delC, 4186delA, 4187delA, 4188delG, 4189delA,
4190delA, 4191delG, 4192delA, 4193delG, 4194delC, 4195delA, 4196delA, 4197delA,
4198delG, 4199delC, 4200delA, 4201delT, 4202delG, 4203delG, 4204delA, 4205delT,
4206delT, 4207delC, 4208delA, 4209delA, 4210delA, 4211delC, 4212delT, 4213delT,
4214delA, 4215delG, 4216delG, 4217delT, 4218delG, 4219delA, 4220delA, 4221delG,
4222delC, 4223delA, 4224delG, 4225delC, 4226delA, 4227delT, 4228delC, 4229delT,
4230delG, 4231delG, 4232delG, 4233delT, 4234delG, 4235delT, 4236delG, 4237delA,
4238delG, 4239delA, 4240delG, 4241delT, 4242delG, 4243delA, 4244delA, 4245delA,
4246delC, 4247delA, 4248delA, 4249delG, 4250delC, 4251delG, 4252delT, 4253delC,
4254delT, 4255delC, 4256delT, 4257delG, 4258delA, 4259delA, 4260delG, 4261delA,
4262delC, 4263delT, 4264delG, 4265delC, 4266delT, 4267delC, 4268delA, 4269delG,
4270delG, 4271delG, 4272delC, 4273delT, 4274delA, 4275delT, 4276delC, 4277delC,
4278delT, 4279delC, 4280delT, 4281delC, 4282delA, 4283delG, 4284delA, 4285delG,
4286delT, 4287delG, 4288delA, 4289delC, 4290delA, 4291delT, 4292delT, 4293delT,
4294delT, 4295delA, 4296delA, 4297delC, 4298delC, 4299delA, 4300delC, 4301delT,
4302delC, 4303delA, 4304delG, 4305delC, 4306delA, 4307delG, 4308delA, 4309delG,
4310delG, 4311delG, 4312delA, 4313delT, 4314delA, 4315delC, 4316delC, 4317delA,
4318delT, 4319delG, 4320delC, 4321delA, 4322delA, 4323delC, 4324delA, 4325delT,
4326delA, 4327delA, 4328delC, 4329delC, 4330delT, 4331delG, 4332delA, 4333delT,
4334delA, 4335delA, 4336delA, 4337delG, 4338delC, 4339delT, 4340delC, 4341delC,
4342delA, 4343delG, 4344delC, 4345delA, 4346delG, 4347delG, 4348delA, 4349delA,
4350delA, 4351delT, 4352delG, 4353delG, 4354delC, 4355delT, 4356delG, 4357delA,
4358delA, 4359delC, 4360delT, 4361delA, 4362delG, 4363delA, 4364delA, 4365delG,
4366delC, 4367delT, 4368delG, 4369delT, 4370delG, 4371delT, 4372delT, 4373delA,
4374delG, 4375delA, 4376delA, 4377delC, 4378delA, 4379delG, 4380delC, 4381delA,
4382delT, 4383delG, 4384delG, 4385delG, 4386delA, 4387delG, 4388delC, 4389delC,
4390delA, 4391delG, 4392delC, 4393delC, 4394delT, 4395delT, 4396delC, 4397delT,
4398delA, 4399delA, 4400delC, 4401delA, 4402delG, 4403delC, 4404delT, 4405delA,
4406delC, 4407delC, 4408delC, 4409delT, 4410delT, 4411delC, 4412delC, 4413delA,
4414delT, 4415delC, 4416delA, 4417delT, 4418delA, 4419delA, 4420delG, 4421delT,
4422delG, 4423delA, 4424delC, 4425delT, 4426delC, 4427delC, 4428delT, 4429delC,
4430delT, 4431delG, 4432delC, 4433delC, 4434delC, 4435delT, 4436delT, 4437delG,
4438delA, 4439delG, 4440delG, 4441delA, 4442delC, 4443delC, 4444delT, 4445delG,
4446delC, 4447delG, 4448delA, 4449delA, 4450delA, 4451delT, 4452delC, 4453delC,
4454delA, 4455delG, 4456delA, 4457delA, 4458delC, 4459delA, 4460delA, 4461delA,
4462delG, 4463delC, 4464delA, 4465delC, 4466delA, 4467delT, 4468delC, 4469delA,
4470delG, 4471delA, 4472delA, 4473delA, 4474delA, 4475delA, 4476delG, 4477delC,
4478delA, 4479delG, 4480delT, 4481delA, 4482delT, 4483delT, 4484delA, 4485delA,
4486delC, 4487delT, 4488delT, 4489delC, 4490delA, 4491delC, 4492delA, 4493delG,
4494delA, 4495delA, 4496delA, 4497delA, 4498delG, 4499delT, 4500delA, 4501delG,
4502delT, 4503delG, 4504delA, 4505delA, 4506delT, 4507delA, 4508delC, 4509delC,
4510delC, 4511delT, 4512delA, 4513delT, 4514delA, 4515delA, 4516delG, 4517delC,
4518delC, 4519delA, 4520delG, 4521delA, 4522delA, 4523delT, 4524delC, 4525delC,
4526delA, 4527delG, 4528delA, 4529delA, 4530delG, 4531delG, 4532delC, 4533delC,
4534delT, 4535delT, 4536delT, 4537delC, 4538delT, 4539delG, 4540delC, 4541delT,
4542delG, 4543delA, 4544delC, 4545delA, 4546delA, 4547delG, 4548delT, 4549delT,
4550delT, 4551delG, 4552delA, 4553delG, 4554delG, 4555delT, 4556delG, 4557delT,
4558delC, 4559delT, 4560delG, 4561delC, 4562delA, 4563delG, 4564delA, 4565delT,
4566delA, 4567delG, 4568delT, 4569delT, 4570delC, 4571delT, 4572delA, 4573delC,
4574delC, 4575delA, 4576delG, 4577delT, 4578delA, 4579delA, 4580delA, 4581delA,
4582delA, 4583delT, 4584delA, 4585delA, 4586delA, 4587delG, 4588delA, 4589delA,
4590delC, 4591delC, 4592delA, 4593delG, 4594delG, 4595delA, 4596delG, 4597delT,
4598delG, 4599delG, 4600delA, 4601delA, 4602delA, 4603delG, 4604delG, 4605delT,
4606delC, 4607delA, 4608delT, 4609delC, 4610delC, 4611delC, 4612delC, 4613delT,
4614delT, 4615delC, 4616delT, 4617delA, 4618delA, 4619delA, 4620delT, 4621delG,
4622delC, 4623delC, 4624delC, 4625delA, 4626delT, 4627delC, 4628delA, 4629delT,
4630delT, 4631delA, 4632delG, 4633delA, 4634delT, 4635delG, 4636delA, 4637delT,
4638delA, 4639delG, 4640delG, 4641delT, 4642delG, 4643delG, 4644delT, 4645delA,
4646delC, 4647delA, 4648delT, 4649delG, 4650delC, 4651delA, 4652delC, 4653delA,
4654delG, 4655delT, 4656delT, 4657delG, 4658delC, 4659delT, 4660delC, 4661delT,
4662delG, 4663delG, 4664delG, 4665delA, 4666delG, 4667delT, 4668delC, 4669delT,
4670delT, 4671delC, 4672delA, 4673delG, 4674delA, 4675delA, 4676delT, 4677delA,
4678delG, 4679delA, 4680delA, 4681delA, 4682delC, 4683delT, 4684delA, 4685delC,
4686delC, 4687delC, 4688delA, 4689delT, 4690delC, 4691delT, 4692delC, 4693delA,
4694delA, 4695delG, 4696delA, 4697delG, 4698delG, 4699delA, 4700delG, 4701delC,
4702delT, 4703delC, 4704delA, 4705delT, 4706delT, 4707delA, 4708delA, 4709delG,
4710delG, 4711delT, 4712delT, 4713delG, 4714delT, 4715delT, 4716delG, 4717delA,
4718delT, 4719delG, 4720delT, 4721delG, 4722delG, 4723delA, 4724delG, 4725delG,
4726delA, 4727delG, 4728delC, 4729delA, 4730delA, 4731delC, 4732delA, 4733delG,
4734delC, 4735delT, 4736delG, 4737delG, 4738delA, 4739delA, 4740delG, 4741delA,
4742delG, 4743delT, 4744delC, 4745delT, 4746delG, 4747delG, 4748delG, 4749delC,
4750delC, 4751delA, 4752delC, 4753delA, 4754delC, 4755delG, 4756delA, 4757delT,
4758delT, 4759delT, 4760delG, 4761delA, 4762delC, 4763delG, 4764delG, 4765delA,
4766delA, 4767delA, 4768delC, 4769delA, 4770delT, 4771delC, 4772delT, 4773delT,
4774delA, 4775delC, 4776delT, 4777delT, 4778delG, 4779delC, 4780delC, 4781delA,
4782delA, 4783delG, 4784delG, 4785delC, 4786delA, 4787delA, 4788delG, 4789delA,
4790delT, 4791delC, 4792delT, 4793delA, 4794delG, 4795delA, 4796delG, 4797delG,
4798delG, 4799delA, 4800delA, 4801delC, 4802delC, 4803delC, 4804delC, 4805delT,
4806delT, 4807delA, 4808delC, 4809delC, 4810delT, 4811delG, 4812delG, 4813delA,
4814delA, 4815delT, 4816delC, 4817delT, 4818delG, 4819delG, 4820delA, 4821delA,
4822delT, 4823delC, 4824delA, 4825delG, 4826delC, 4827delC, 4828delT, 4829delC,
4830delT, 4831delT, 4832delC, 4833delT, 4834delC, 4835delT, 4836delG, 4837delA,
4838delT, 4839delG, 4840delA, 4841delC, 4842delC, 4843delC, 4844delT, 4845delG,
4846delA, 4847delA, 4848delT, 4849delC, 4850delT, 4851delG, 4852delA, 4853delT,
4854delC, 4855delC, 4856delT, 4857delT, 4858delC, 4859delT, 4860delG, 4861delA,
4862delA, 4863delG, 4864delA, 4865delC, 4866delA, 4867delG, 4868delA, 4869delG,
4870delC, 4871delC, 4872delC, 4873delC, 4874delA, 4875delG, 4876delA, 4877delG,
4878delT, 4879delC, 4880delA, 4881delG, 4882delC, 4883delT, 4884delC, 4885delG,
4886delT, 4887delG, 4888delT, 4889delT, 4890delG, 4891delG, 4892delC, 4893delA,
4894delA, 4895delC, 4896delA, 4897delT, 4898delA, 4899delC, 4900delC, 4901delA,
4902delT, 4903delC, 4904delT, 4905delT, 4906delC, 4907delA, 4908delA, 4909delC,
4910delC, 4911delT, 4912delC, 4913delT, 4914delG, 4915delC, 4916delA, 4917delT,
4918delT, 4919delG, 4920delA, 4921delA, 4922delA, 4923delG, 4924delT, 4925delT,
4926delC, 4927delC, 4928delC, 4929delC, 4930delA, 4931delA, 4932delT, 4933delT,
4934delG, 4935delA, 4936delA, 4937delA, 4938delG, 4939delT, 4940delT, 4941delG,
4942delC, 4943delA, 4944delG, 4945delA, 4946delA, 4947delT, 4948delC, 4949delT,
4950delG, 4951delC, 4952delC, 4953delC, 4954delA, 4955delG, 4956delG, 4957delG,
4958delT, 4959delC, 4960delC, 4961delA, 4962delG, 4963delC, 4964delT, 4965delG,
4966delC, 4967delT, 4968delG, 4969delC, 4970delT, 4971delC, 4972delA, 4973delT,
4974delA, 4975delC, 4976delT, 4977delA, 4978delC, 4979delT, 4980delG, 4981delA,
4982delT, 4983delA, 4984delC, 4985delT, 4986delG, 4987delC, 4988delT, 4989delG,
4990delG, 4991delG, 4992delT, 4993delA, 4994delT, 4995delA, 4996delA, 4997delT,
4998delG, 4999delC, 5000delA, 5001delA, 5002delT, 5003delG, 5004delG, 5005delA,
5006delA, 5007delG, 5008delA, 5009delA, 5010delA, 5011delG, 5012delT, 5013delG,
5014delT, 5015delG, 5016delA, 5017delG, 5018delC, 5019delA, 5020delG, 5021delG,
5022delG, 5023delA, 5024delG, 5025delA, 5026delA, 5027delG, 5028delC, 5029delC,
5030delA, 5031delG, 5032delA, 5033delA, 5034delT, 5035delT, 5036delG, 5037delA,
5038delC, 5039delA, 5040delG, 5041delC, 5042delT, 5043delT, 5044delC, 5045delA,
5046delA, 5047delC, 5048delA, 5049delG, 5050delA, 5051delA, 5052delA, 5053delG,
5054delG, 5055delG, 5056delT, 5057delC, 5058delA, 5059delA, 5060delC, 5061delA,
5062delA, 5063delA, 5064delA, 5065delG, 5066delA, 5067delA, 5068delT, 5069delG,
5070delT, 5071delC, 5072delC, 5073delA, 5074delT, 5075delG, 5076delG, 5077delT,
5078delG, 5079delG, 5080delT, 5081delG, 5082delT, 5083delC, 5084delT, 5085delG,
5086delG, 5087delC, 5088delC, 5089delT, 5090delG, 5091delA, 5092delC, 5093delC,
5094delC, 5095delC, 5096delA, 5097delG, 5098delA, 5099delA, 5100delG, 5101delA,
5102delA, 5103delT, 5104delT, 5105delT, 5106delA, 5107delT, 5108delG, 5109delC,
5110delT, 5111delC, 5112delG, 5113delT, 5114delG, 5115delT, 5116delA, 5117delC,
5118delA, 5119delA, 5120delG, 5121delT, 5122delT, 5123delT, 5124delG, 5125delC,
5126delC, 5127delA, 5128delG, 5129delA, 5130delA, 5131delA, 5132delA, 5133delC,
5134delA, 5135delC, 5136delC, 5137delA, 5138delC, 5139delA, 5140delT, 5141delC,
5142delA, 5143delC, 5144delT, 5145delT, 5146delT, 5147delA, 5148delA, 5149delC,
5150delT, 5151delA, 5152delA, 5153delT, 5154delC, 5155delT, 5156delA, 5157delA,
5158delT, 5159delT, 5160delA, 5161delC, 5162delT, 5163delG, 5164delA, 5165delA,
5166delG, 5167delA, 5168delG, 5169delA, 5170delC, 5171delT, 5172delA, 5173delC,
5174delT, 5175delC, 5176delA, 5177delT, 5178delG, 5179delT, 5180delT, 5181delG,
5182delT, 5183delT, 5184delA, 5185delT, 5186delG, 5187delA, 5188delA, 5189delA,
5190delA, 5191delC, 5192delA, 5193delG, 5194delA, 5195delT, 5196delG, 5197delC,
5198delT, 5199delG, 5200delA, 5201delG, 5202delT, 5203delT, 5204delT, 5205delG,
5206delT, 5207delG, 5208delT, 5209delG, 5210delT, 5211delG, 5212delA, 5213delA,
5214delC, 5215delG, 5216delG, 5217delA, 5218delC, 5219delA, 5220delC, 5221delT,
5222delG, 5223delA, 5224delA, 5225delA, 5226delT, 5227delA, 5228delT, 5229delT,
5230delT, 5231delT, 5232delC, 5233delT, 5234delA, 5235delG, 5236delG, 5237delA,
5238delA, 5239delT, 5240delT, 5241delG, 5242delC, 5243delG, 5244delG, 5245delG,
5246delA, 5247delG, 5248delG, 5249delA, 5250delA, 5251delA, 5252delA, 5253delT,
5254delG, 5255delG, 5256delG, 5257delT, 5258delA, 5259delG, 5260delT, 5261delT,
5262delA, 5263delG, 5264delC, 5265delT, 5266delA, 5267delT, 5268delT, 5269delT,
5270delC, 5271delT, 5272delG, 5273delG, 5274delG, 5275delT, 5276delG, 5277delA,
5278delC, 5279delC, 5280delC, 5281delA, 5282delG, 5283delT, 5284delC, 5285delT,
5286delA, 5287delT, 5288delT, 5289delA, 5290delA, 5291delA, 5292delG, 5293delA,
5294delA, 5295delA, 5296delG, 5297delA, 5298delA, 5299delA, 5300delA, 5301delA,
5302delT, 5303delG, 5304delC, 5305delT, 5306delG, 5307delA, 5308delA, 5309delT,
5310delG, 5311delA, 5312delG, 5313delC, 5314delA, 5315delT, 5316delG, 5317delA,
5318delT, 5319delT, 5320delT, 5321delT, 5322delG, 5323delA, 5324delA, 5325delG,
5326delT, 5327delC, 5328delA, 5329delG, 5330delA, 5331delG, 5332delG, 5333delA,
5334delG, 5335delA, 5336delT, 5337delG, 5338delT, 5339delG, 5340delG, 5341delT,
5342delC, 5343delA, 5344delA, 5345delT, 5346delG, 5347delG, 5348delA, 5349delA,
5350delG, 5351delA, 5352delA, 5353delA, 5354delC, 5355delC, 5356delA, 5357delC,
5358delC, 5359delA, 5360delA, 5361delG, 5362delG, 5363delT, 5364delC, 5365delC,
5366delA, 5367delA, 5368delA, 5369delG, 5370delC, 5371delG, 5372delA, 5373delG,
5374delC, 5375delA, 5376delA, 5377delG, 5378delA, 5379delG, 5380delA, 5381delA,
5382delT, 5383delC, 5384delC, 5385delC, 5386delA, 5387delG, 5388delG, 5389delA,
5390delC, 5391delA, 5392delG, 5393delA, 5394delA, 5395delA, 5396delG, 5397delA,
5398delT, 5399delC, 5400delT, 5401delT, 5402delC, 5403delA, 5404delG, 5405delG,
5406delG, 5407delG, 5408delG, 5409delC, 5410delT, 5411delA, 5412delG, 5413delA,
5414delA, 5415delA, 5416delT, 5417delC, 5418delT, 5419delG, 5420delT, 5421delT,
5422delG, 5423delC, 5424delT, 5425delA, 5426delT, 5427delG, 5428delG, 5429delG,
5430delC, 5431delC, 5432delC, 5433delT, 5434delT, 5435delC, 5436delA, 5437delC,
5438delC, 5439delA, 5440delA, 5441delC, 5442delA, 5443delT, 5444delG, 5445delC,
5446delC, 5447delC, 5448delA, 5449delC, 5450delA, 5451delG, 5452delA, 5453delT,
5454delC, 5455delA, 5456delA, 5457delC, 5458delT, 5459delG, 5460delG, 5461delA,
5462delA, 5463delT, 5464delG, 5465delG, 5466delA, 5467delT, 5468delG, 5469delG,
5470delT, 5471delA, 5472delC, 5473delA, 5474delG, 5475delC, 5476delT, 5477delG,
5478delT, 5479delG, 5480delT, 5481delG, 5482delG, 5483delT, 5484delG, 5485delC,
5486delT, 5487delT, 5488delC, 5489delT, 5490delG, 5491delT, 5492delG, 5493delG,
5494delT, 5495delG, 5496delA, 5497delA, 5498delG, 5499delG, 5500delA, 5501delG,
5502delC, 5503delT, 5504delT, 5505delT, 5506delC, 5507delA, 5508delT, 5509delC,
5510delA, 5511delT, 5512delT, 5513delC, 5514delA, 5515delC, 5516delC, 5517delC,
5518delT, 5519delT, 5520delG, 5521delG, 5522delC, 5523delA, 5524delC, 5525delA,
5526delG, 5527delG, 5528delT, 5529delG, 5530delT, 5531delC, 5532delC, 5533delA,
5534delC, 5535delC, 5536delC, 5537delA, 5538delA, 5539delT, 5540delT, 5541delG,
5542delT, 5543delG, 5544delG, 5545delT, 5546delT, 5547delG, 5548delT, 5549delG,
5550delC, 5551delA, 5552delG, 5553delC, 5554delC, 5555delA, 5556delG, 5557delA,
5558delT, 5559delG, 5560delC, 5561delC, 5562delT, 5563delG, 5564delG, 5565delA,
5566delC, 5567delA, 5568delG, 5569delA, 5570delG, 5571delG, 5572delA, 5573delC,
5574delA, 5575delA, 5576delT, 5577delG, 5578delG, 5579delC, 5580delT, 5581delT,
5582delC, 5583delC, 5584delA, 5585delT, 5586delG, 5587delC, 5588delA, 5589delA,
5590delT, 5591delT, 5592delG, 5593delG, 5594delG, 5595delC, 5596delA, 5597delG,
5598delA, 5599delT, 5600delG, 5601delT, 5602delG, 5603delT, 5604delG, 5605delA,
5606delG, 5607delG, 5608delC, 5609delA, 5610delC, 5611delC, 5612delT, 5613delG,
5614delT, 5615delG, 5616delG, 5617delT, 5618delG, 5619delA, 5620delC, 5621delC,
5622delC, 5623delG, 5624delA, 5625delG, 5626delA, 5627delG, 5628delT, 5629delG,
5630delG, 5631delG, 5632delT, 5633delG, 5634delT, 5635delT, 5636delG, 5637delG,
5638delA, 5639delC, 5640delA, 5641delG, 5642delT, 5643delG, 5644delT, 5645delA,
5646delG, 5647delC, 5648delA, 5649delC, 5650delT, 5651delC, 5652delT, 5653delA,
5654delC, 5655delC, 5656delA, 5657delG, 5658delT, 5659delG, 5660delC, 5661delC,
5662delA, 5663delG, 5664delG, 5665delA, 5666delG, 5667delC, 5668delT, 5669delG,
5670delG, 5671delA, 5672delC, 5673delA, 5674delC, 5675delC, 5676delT, 5677delA,
5678delC, 5679delC.

TABLE 10
List of Two Base Deletions

4delGA, 5delAT, 6delTT, 7delTT, 8delTA, 9delAT, 10delTC, 11delCT, 12delTG, 132delGC,
133delCT, 134delTC, 135delCT, 136delTT, 137delTC, 138delCG, 139delGC, 140delCG,
141delGT, 142delTT, 143delTG, 144delGA, 145delAA, 146delAG, 147delGA, 148delAA,
149delAG, 150delGT, 151delTA, 152delAC, 153delCA, 154delAA, 155delAA, 156delAA,
157delAT, 158delTG, 159delGT, 160delTC, 161delCA, 162delAT, 163delTT, 164delTA,
165delAA, 166delAT, 167delTG, 168delGC, 169delCT, 170delTA, 171delAT, 172delTG,
173delGC, 174delCA, 175delAG, 176delGA, 177delAA, 178delAA, 179delAA, 180delAT,
181delTC, 182delCT, 183delTT, 184delTA, 185delAG, 186delGA, 187delAG, 188delGT,
189delTG, 190delGT, 191delTC, 192delCC, 193delCC, 194delCA, 195delAT, 196delTC,
197delCT, 198delTG, 199delGT, 200delTC, 201delCT, 202delTG, 203delGG, 204delGA,
205delAG, 206delGT, 207delTT, 208delTG, 209delGA, 210delAT, 211delTC, 212delCA,
213delAA, 214delAG, 215delGG, 216delGA, 217delAA, 218delAC, 219delCC, 220delCT,
221delTG, 222delGT, 223delTC, 224delCT, 225delTC, 226delCC, 227delCA, 228delAC,
229delCA, 230delAA, 231delAA, 232delAG, 233delGT, 234delTG, 235delGT, 236delTG,
237delGA, 238delAC, 239delCC, 240delCA, 241delAC, 242delCA, 243delAT, 244delTA,
245delAT, 246delTT, 247delTT, 248delTT, 249delTG, 250delGC, 251delCA, 252delAA,
253delAA, 254delAT, 255delTT, 256delTT, 257delTT, 258delTG, 259delGC, 260delCA,
261delAT, 262delTG, 263delGC, 264delCT, 265delTG, 266delGA, 267delAA, 268delAA,
269delAC, 270delCT, 271delTT, 272delTC, 273delCT, 274delTC, 275delCA, 276delAA,
277delAC, 278delCC, 279delCA, 280delAG, 281delGA, 282delAA, 283delAG, 284delGA,
285delAA, 286delAA, 287delAG, 288delGG, 289delGG, 290delGC, 291delCC, 292delCT,
293delTT, 294delTC, 295delCA, 296delAC, 297delCA, 298delAG, 299delGT, 300delTG,
301delGT, 302delTC, 303delCC, 304delCT, 305delTT, 306delTT, 307delTA, 308delAT,
309delTG, 310delGT, 311delTA, 312delAA, 313delAG, 314delGA, 315delAA, 316delAT,
317delTG, 318delGA, 319delAT, 320delTA, 321delAT, 322delTA, 323delAA, 324delAC,
325delCC, 326delCA, 327delAA, 328delAA, 329delAA, 330delAG, 331delGG, 332delGA,
333delAG, 334delGC, 335delCC, 336delCT, 337delTA, 338delAC, 339delCA, 340delAA,
341delAG, 342delGA, 343delAA, 344delAA, 345delAG, 346delGT, 347delTA, 348delAC,
349delCG, 350delGA, 351delAG, 352delGA, 353delAT, 354delTT, 355delTT, 356delTA,
357delAG, 358delGT, 359delTC, 360delCA, 361delAA, 362delAC, 363delCT, 364delTT,
365delTG, 366delGT, 367delTT, 368delTG, 369delGA, 370delAA, 371delAG, 372delGA,
373delAG, 374delGC, 375delCT, 376delTA, 377delAT, 378delTT, 379delTG, 380delGA,
381delAA, 382delAA, 383delAA, 384delAT, 385delTC, 386delCA, 387delAT, 388delTT,
389delTT, 390delTG, 391delGT, 392delTG, 393delGC, 394delCT, 395delTT, 396delTT,
397delTT, 398delTC, 399delCA, 400delAG, 401delGC, 402delCT, 403delTT, 404delTG,
405delGA, 406delAC, 407delCA, 408delAC, 409delCA, 410delAG, 411delGG, 412delGT,
413delTT, 414delTT, 415delTG, 416delGG, 417delGA, 418delAG, 419delGT, 420delTA,
421delAT, 422delTG, 423delGC, 424delCA, 425delAA, 426delAA, 427delAC, 428delCA,
429delAG, 430delGC, 431delCT, 432delTA, 433delAT, 434delTA, 435delAA, 436delAT,
437delTT, 438delTT, 439delTT, 440delTG, 441delGC, 442delCA, 443delAA, 444delAA,
445delAA, 446delAA, 447delAA, 448delAG, 449delGG, 450delGA, 451delAA, 452delAA,
453delAA, 454delAT, 455delTA, 456delAA, 457delAC, 458delCT, 459delTC, 460delCT,
461delTC, 462delCC, 463delCT, 464delTG, 465delGA, 466delAA, 467delAC, 468delCA,
469delAT, 470delTC, 471delCT, 472delTA, 473delAA, 474delAA, 475delAA, 476delAG,
477delGA, 478delAT, 479delTG, 480delGA, 481delAA, 482delAG, 483delGT, 484delTT,
485delTT, 486delTC, 487delCT, 488delTA, 489delAT, 490delTC, 491delCA, 492delAT,
493delTC, 494delCC, 495delCA, 496delAA, 497delAA, 498delAG, 499delGT, 500delTA,
501delAT, 502delTG, 503delGG, 504delGG, 505delGC, 506delCT, 507delTA, 508delAC,
509delCA, 510delAG, 511delGA, 512delAA, 513delAA, 514delAC, 515delCC, 516delCG,
517delGT, 518delTG, 519delGC, 520delCC, 521delCA, 522delAA, 523delAA, 524delAA,
525delAG, 526delGA, 527delAC, 528delCT, 529delTT, 530delTC, 531delCT, 532delTA,
533delAC, 534delCA, 535delAG, 536delGA, 537delAG, 538delGT, 539delTG, 540delGA,
541delAA, 542delAC, 543delCC, 544delCC, 545delCG, 546delGA, 547delAA, 548delAA,
549delAA, 550delAT, 551delTC, 552delCC, 553delCT, 554delTT, 555delTC, 556delCC,
557delCT, 558delTT, 559delTG, 560delGC, 561delCA, 562delAG, 563delGG, 564delGA,
565delAA, 566delAA, 567delAC, 568delCC, 569delCA, 570delAG, 571delGT, 572delTC,
573delCT, 574delTC, 575delCA, 576delAG, 577delGT, 578delTG, 579delGT, 580delTC,
581delCC, 582delCA, 583delAA, 584delAC, 585delCT, 586delTC, 587delCT, 588delTC,
589delCT, 590delTA, 591delAA, 592delAC, 593delCC, 594delCT, 595delTT, 596delTG,
597delGG, 598delGA, 599delAA, 600delAC, 601delCT, 602delTG, 603delGT, 604delTG,
605delGA, 606delAG, 607delGA, 608delAA, 609delAC, 610delCT, 611delTC, 612delCT,
613delTG, 614delGA, 615delAG, 616delGG, 617delGA, 618delAC, 619delCA, 620delAA,
621delAA, 622delAG, 623delGC, 624delCA, 625delAG, 626delGC, 627delCG, 628delGG,
629delGA, 630delAT, 631delTA, 632delAC, 633delCA, 634delAA, 635delAC, 636delCC,
637delCT, 638delTC, 639delCA, 640delAA, 641delAA, 642delAA, 643delAG, 644delGA,
645delAC, 646delCG, 647delGT, 648delTC, 649delCT, 650delTG, 651delGT, 652delTC,
653delCT, 654delTA, 655delAC, 656delCA, 657delAT, 658delTT, 659delTG, 660delGA,
661delAA, 662delAT, 663delTT, 664delTG, 665delGG, 666delGG, 667delGA, 668delAT,
669delTC, 670delCT, 671delTG, 672delGA, 673delAT, 674delTT, 675delTC, 676delCT,
677delTT, 678delTC, 679delCT, 680delTG, 681delGA, 682delAA, 683delAG, 684delGA,
685delAT, 686delTA, 687delAC, 688delCC, 689delCG, 690delGT, 691delTT, 692delTA,
693delAA, 694delAT, 695delTA, 696delAA, 697delAG, 698delGG, 699delGC, 700delCA,
701delAA, 702delAC, 703delCT, 704delTT, 705delTA, 706delAT, 707delTT, 708delTG,
709delGC, 710delCA, 711delAG, 712delGT, 713delTG, 714delGT, 715delTG, 716delGG,
717delGG, 718delGA, 719delAG, 720delGA, 721delAT, 722delTC, 723delCA, 724delAA,
725delAG, 726delGA, 727delAA, 728delAT, 729delTT, 730delTG, 731delGT, 732delTT,
733delTA, 734delAC, 735delCA, 736delAA, 737delAA, 738delAT, 739delTC, 740delCA,
741delAC, 742delCC, 743delCC, 744delCC, 745delCT, 746delTC, 747delCA, 748delAA,
749delAG, 750delGG, 751delGA, 752delAA, 753delAC, 754delCC, 755delCA, 756delAG,
757delGG, 758delGG, 759delGA, 760delAT, 761delTG, 762delGA, 763delAA, 764delAA,
765delAT, 766delTC, 767delCA, 768delAG, 769delGT, 770delTT, 771delTT, 772delTG,
773delGG, 774delGA, 775delAT, 776delTT, 777delTC, 778delCT, 779delTG, 780delGC,
781delCA, 782delAA, 783delAA, 784delAA, 785delAA, 786delAA, 787delAG, 788delGG,
789delGC, 790delCT, 791delTG, 792delGC, 793delCT, 794delTT, 795delTG, 796delGT,
797delTG, 798delGA, 799delAA, 800delAT, 801delTT, 802delTT, 803delTT, 804delTC,
805delCT, 806delTG, 807delGA, 808delAG, 809delGA, 810delAC, 811delCG, 812delGG,
813delGA, 814delAT, 815delTG, 816delGT, 817delTA, 818delAA, 819delAC, 820delCA,
821delAA, 822delAA, 823delAT, 824delTA, 825delAC, 826delCT, 827delTG, 828delGA,
829delAA, 830delAC, 831delCA, 832delAT, 833delTC, 834delCA, 835delAT, 836delTC,
837delCA, 838delAA, 839delAC, 840delCC, 841delCC, 842delCA, 843delAG, 844delGT,
845delTA, 846delAA, 847delAT, 848delTA, 849delAA, 850delAT, 851delTG, 852delGA,
853delAT, 854delTT, 855delTT, 856delTG, 857delGA, 858delAA, 859delAC, 860delCA,
861delAC, 862delCC, 863delCA, 864delAC, 865delCT, 866delTG, 867delGA, 868delAG,
869delGA, 870delAA, 871delAG, 872delGC, 873delCG, 874delGT, 875delTG, 876delGC,
877delCA, 878delAG, 879delGC, 880delCT, 881delTG, 882delGA, 883delAG, 884delGA,
885delAG, 886delGG, 887delGC, 888delCA, 889delAT, 890delTC, 891delCC, 892delCA,
893delAG, 894delGA, 895delAA, 896delAA, 897delAA, 898delAG, 899delGT, 900delTA,
901delAT, 902delTC, 903delCA, 904delAG, 905delGG, 906delGG, 907delGT, 908delTA,
909delAG, 910delGT, 911delTT, 912delTC, 913delCT, 914delTG, 915delGT, 916delTT,
917delTT, 918delTC, 919delCA, 920delAA, 921delAA, 922delAC, 923delCT, 924delTT,
925delTG, 926delGC, 927delCA, 928delAT, 929delTG, 930delGT, 931delTG, 932delGG,
933delGA, 934delAG, 935delGC, 936delCC, 937delCA, 938delAT, 939delTG, 940delGT,
941delTG, 942delGG, 943delGC, 944delCA, 945delAC, 946delCA, 947delAA, 948delAA,
949delAT, 950delTA, 951delAC, 952delCT, 953delTC, 954delCA, 955delAT, 956delTG,
957delGC, 958delCC, 959delCA, 960delAG, 961delGC, 962delCT, 963delTC, 964delCA,
965delAT, 966delTT, 967delTA, 968delAC, 969delCA, 970delAG, 971delGC, 972delCA,
973delAT, 974delTG, 975delGA, 976delAG, 977delGA, 978delAA, 979delAC, 980delCA,
981delAG, 982delGC, 983delCA, 984delAG, 985delGT, 986delTT, 987delTT, 988delTA,
989delAT, 990delTT, 991delTA, 992delAC, 993delCT, 994delTC, 995delCA, 996delAC,
997delCT, 998delTA, 999delAA, 1000delAA, 1001delAG, 1002delGA, 1003delAC,
1004delCA, 1005delAG, 1006delGA, 1007delAA, 1008delAT, 1009delTG, 1010delGA,
1011delAA, 1012delAT, 1013delTG, 1014delGT, 1015delTA, 1016delAG, 1017delGA,
1018delAA, 1019delAA, 1020delAA, 1021delAG, 1022delGG, 1023delGC, 1024delCT,
1025delTG, 1026delGA, 1027delAA, 1028delAT, 1029delTT, 1030delTC, 1031delCT,
1032delTG, 1033delGT, 1034delTA, 1035delAA, 1036delAT, 1037delTA, 1038delAA,
1039delAA, 1040delAA, 1041delAG, 1042delGC, 1043delCA, 1044delAA, 1045delAA,
1046delAC, 1047delCA, 1048delAG, 1049delGC, 1050delCC, 1051delCT, 1052delTG,
1053delGG, 1054delGC, 1055delCT, 1056delTT, 1057delTA, 1058delAG, 1059delGC,
1060delCA, 1061delAA, 1062delAG, 1063delGG, 1064delGA, 1065delAG, 1066delGC,
1067delCC, 1068delCA, 1069delAA, 1070delAC, 1071delCA, 1072delAT, 1073delTA,
1074delAA, 1075delAC, 1076delCA, 1077delAG, 1078delGA, 1079delAT, 1080delTG,
1081delGG, 1082delGG, 1083delGC, 1084delCT, 1085delTG, 1086delGG, 1087delGA,
1088delAA, 1089delAG, 1090delGT, 1091delTA, 1092delAA, 1093delAG, 1094delGG,
1095delGA, 1096delAA, 1097delAA, 1098delAC, 1099delCA, 1100delAT, 1101delTG,
1102delGT, 1103delTA, 1104delAA, 1105delAT, 1106delTG, 1107delGA, 1108delAT,
1109delTA, 1110delAG, 1111delGG, 1112delGC, 1113delCG, 1114delGG, 1115delGA,
1116delAC, 1117delCT, 1118delTC, 1119delCC, 1120delCC, 1121delCA, 1122delAG,
1123delGC, 1124delCA, 1125delAC, 1126delCA, 1127delAG, 1128delGA, 1129delAA,
1130delAA, 1131delAA, 1132delAA, 1133delAA, 1134delAA, 1135delAG, 1136delGG,
1137delGT, 1138delTA, 1139delAG, 1140delGA, 1141delAT, 1142delTC, 1143delCT,
1144delTG, 1145delGA, 1146delAA, 1147delAT, 1148delTG, 1149delGC, 1150delCT,
1151delTG, 1152delGA, 1153delAT, 1154delTC, 1155delCC, 1156delCC, 1157delCC,
1158delCT, 1159delTG, 1160delGT, 1161delTG, 1162delGT, 1163delTG, 1164delGA,
1165delAG, 1166delGA, 1167delAG, 1168delGA, 1169delAA, 1170delAA, 1171delAA,
1172delAG, 1173delGA, 1174delAA, 1175delAT, 1176delTG, 1177delGG, 1178delGA,
1179delAA, 1180delAT, 1181delTA, 1182delAA, 1183delAG, 1184delGC, 1185delCA,
1186delAG, 1187delGA, 1188delAA, 1189delAA, 1190delAC, 1191delCT, 1192delTG,
1193delGC, 1194delCC, 1195delCA, 1196delAT, 1197delTG, 1198delGC, 1199delCT,
1200delTC, 1201delCA, 1202delAG, 1203delGA, 1204delAG, 1205delGA, 1206delAA,
1207delAT, 1208delTC, 1209delCC, 1210delCT, 1211delTA, 1212delAG, 1213delGA,
1214delAG, 1215delGA, 1216delAT, 1217delTA, 1218delAC, 1219delCT, 1220delTG,
1221delGA, 1222delAA, 1223delAG, 1224delGA, 1225delAT, 1226delTG, 1227delGT,
1228delTT, 1229delTC, 1230delCC, 1331delCT, 1232delTT, 1233delTG, 1234delGG,
1235delGA, 1236delAT, 1237delTA, 1238delAA, 1239delAC, 1240delCA, 1241delAC,
1242delCT, 1243delTA, 1244delAA, 1245delAA, 1246delAT, 1247delTA, 1248delAG,
1249delGC, 1250delCA, 1251delAG, 1252delGC, 1253delCA, 1254delAT, 1255delTT,
1256delTC, 1257delCA, 1258delAG, 1259delGA, 1260delAA, 1261delAA, 1262delAG,
1263delGT, 1264delTT, 1265delTA, 1266delAA, 1267delAT, 1268delTG, 1269delGA,
1270delAG, 1271delGT, 1272delTG, 1273delGG, 1274delGT, 1275delTT, 1276delTT,
1277delTT, 1278delTC, 1279delCC, 1280delCA, 1281delAG, 1282delGA, 1283delAA,
1284delAG, 1285delGT, 1286delTG, 1287delGA, 1288delAT, 1289delTG, 1290delGA,
1291delAA, 1292delAC, 1293delCT, 1294delTG, 1295delGT, 1296delTT, 1297delTA,
1298delAG, 1299delGG, 1300delGT, 1301delTT, 1302delTC, 1303delCT, 1304delTG,
1305delGA, 1306delAT, 1307delTG, 1308delGA, 1309delAC, 1310delCT, 1311delTC,
1312delCA, 1313delAC, 1314delCA, 1315delAT, 1316delTG, 1317delGA, 1318delAT,
1319delTG, 1320delGG, 1321delGG, 1322delGG, 1323delGA, 1324delAG, 1325delGT,
1326delTC, 1327delCT, 1328delTG, 1329delGA, 1330delAA, 1331delAT, 1332delTC,
1333delCA, 1334delAA, 1335delAA, 1336delAT, 1337delTG, 1338delGC, 1339delCC,
1340delCA, 1341delAA, 1342delAA, 1343delAG, 1344delGT, 1345delTA, 1346delAG,
1347delGC, 1348delCT, 1349delTG, 1350delGA, 1351delAT, 1352delTG, 1353delGT,
1354delTA, 1355delAT, 1356delTT, 1357delTG, 1358delGG, 1359delGA, 1360delAC,
1361delCG, 1362delGT, 1363delTT, 1364delTC, 1365delCT, 1366delTA, 1367delAA,
1368delAA, 1369delAT, 1370delTG, 1371delGA, 1372delAG, 1373delGG, 1374delGT,
1375delTA, 1376delAG, 1377delGA, 1378delAT, 1379delTG, 1380delGA, 1381delAA,
1382delAT, 1383delTA, 1384delAT, 1385delTT, 1386delTC, 1387delCT, 1388delTG,
1389delGG, 1390delGT, 1391delTT, 1392delTC, 1393delCT, 1394delTT, 1395delTC,
1396delCA, 1397delAG, 1398delGA, 1399delAG, 1400delGA, 1401delAA, 1402delAA,
1403delAA, 1404delAT, 1405delTA, 1406delAG, 1407delGA, 1408delAC, 1409delCT,
1410delTT, 1411delTA, 1412delAC, 1413delCT, 1414delTG, 1415delGG, 1416delGC,
1417delCC, 1418delCA, 1419delAG, 1420delGT, 1421delTG, 1422delGA, 1423delAT,
1424delTC, 1425delCC, 1426delCT, 1427delTC, 1428delCA, 1429delAT, 1430delTG,
1431delGA, 1432delAG, 1433delGG, 1434delGC, 1435delCT, 1436delTT, 1437delTT,
1438delTA, 1439delAA, 1440delAT, 1441delTA, 1442delAT, 1443delTG, 1444delGT,
1445delTA, 1446delAA, 1447delAA, 1448delAA, 1449delAG, 1450delGT, 1451delTG,
1452delGA, 1453delAA, 1454delAA, 1455delAG, 1456delGA, 1457delAG, 1458delGT,
1459delTT, 1460delTC, 1461delCA, 1462delAC, 1463delCT, 1464delTC, 1465delCC,
1466delCA, 1467delAA, 1468delAA, 1469delAT, 1470delTC, 1471delCA, 1472delAG,
1473delGT, 1474delTA, 1475delAG, 1476delGA, 1477delAG, 1478delGA, 1479delAG,
1480delGT, 1481delTA, 1482delAA, 1483delAT, 1484delTA, 1485delAT, 1486delTT,
1487delTG, 1488delGA, 1489delAA, 1490delAG, 1491delGA, 1492delAC, 1493delCA,
1494delAA, 1495delAA, 1496delAA, 1497delAT, 1498delTA, 1499delAT, 1500delTT,
1501delTT, 1502delTG, 1503delGG, 1504delGG, 1505delGA, 1506delAA, 1507delAA,
1508delAA, 1509delAC, 1510delCC, 1511delCT, 1512delTA, 1513delAT, 1514delTC,
1515delCG, 1516delGG, 1517delGA, 1518delAA, 1519delAG, 1520delGA, 1521delAA,
1522delAG, 1523delGG, 1524delGC, 1525delCA, 1526delAA, 1527delAG, 1528delGC,
1529delCC, 1530delCT, 1531delTC, 1532delCC, 1533delCC, 1534delCC, 1535delCA,
1536delAA, 1537delAC, 1538delCT, 1539delTT, 1540delTA, 1541delAA, 1542delAG,
1543delGC, 1544delCC, 1545delCA, 1546delAT, 1547delTG, 1548delGT, 1549delTA,
1550delAA, 1551delAC, 1552delCT, 1553delTG, 1554delGA, 1555delAA, 1556delAA,
1557delAA, 1558delAT, 1559delTC, 1560delCT, 1561delTA, 1562delAA, 1563delAT,
1564delTT, 1565delTA, 1566delAT, 1567delTA, 1568delAG, 1569delGG, 1570delGA,
1571delAG, 1572delGC, 1573delCA, 1574delAT, 1575delTT, 1576delTT, 1577delTG,
1578delGT, 1579delTT, 1580delTA, 1581delAC, 1582delCT, 1583delTG, 1584delGA,
1585delAG, 1586delGC, 1587delCC, 1588delCA, 1589delAC, 1590delCA, 1591delAG,
1592delGA, 1593delAT, 1594delTA, 1595delAA, 1596delAT, 1597delTA, 1598delAC,
1599delCA, 1600delAA, 1601delAG, 1602delGA, 1603delAG, 1604delGC, 1605delCG,
1606delGT, 1607delTC, 1608delCC, 1609delCC, 1610delCC, 1611delCT, 1612delTC,
1613delCA, 1614delAC, 1615delCA, 1616delAA, 1617delAA, 1618delAT, 1619delTA,
1620delAA, 1621delAA, 1622delAT, 1623delTT, 1624delTA, 1625delAA, 1626delAA,
1627delAG, 1628delGC, 1629delCG, 1630delGT, 1631delTA, 1632delAA, 1633delAA,
1634delAA, 1635delAG, 1636delGG, 1637delGA, 1638delAG, 1639delGA, 1640delAC,
1641delCC, 1642delCT, 1643delTA, 1644delAC, 1645delCA, 1646delAT, 1647delTC,
1648delCA, 1649delAG, 1650delGG, 1651delGC, 1652delCC, 1653delCT, 1654delTT,
1655delTC, 1656delCA, 1657delAT, 1658delTC, 1659delCC, 1660delCT, 1661delTG,
1662delGA, 1663delAG, 1664delGG, 1665delGA, 1666delAT, 1667delTT, 1668delTT,
1669delTT, 1670delTA, 1671delAT, 1672delTC, 1673delCA, 1674delAA, 1675delAG,
1676delGA, 1677delAA, 1678delAA, 1679delAG, 1680delGC, 1681delCA, 1682delAG,
1683delGA, 1684delAT, 1685delTT, 1686delTT, 1687delTG, 1688delGG, 1689delGC,
1690delCA, 1691delAG, 1692delGT, 1693delTT, 1694delTC, 1695delCA, 1696delAA,
1697delAA, 1698delAA, 1699delAG, 1700delGA, 1701delAC, 1702delCT, 1703delTC,
1704delCC, 1705delCT, 1706delTG, 1707delGA, 1708delAA, 1709delAA, 1710delAT,
1711delTG, 1712delGA, 1713delAT, 1714delTA, 1715delAA, 1716delAA, 1717delAT,
1718delTC, 1719delCA, 1720delAG, 1721delGG, 1722delGG, 1723delGA, 1724delAA,
1725delAC, 1726delCT, 1727delTA, 1728delAA, 1729delAC, 1730delCC, 1731delCA,
1732delAA, 1733delAA, 1734delAC, 1735delCG, 1736delGG, 1737delGA, 1738delAG,
1739delGC, 1740delCA, 1741delAG, 1742delGA, 1743delAA, 1744delAT, 1745delTG,
1746delGG, 1747delGT, 1748delTC, 1749delCA, 1750delAA, 1751delAG, 1752delGT,
1753delTG, 1754delGA, 1755delAT, 1756delTG, 1757delGA, 1758delAA, 1759delAT,
1760delTA, 1761delAT, 1762delTT, 1763delTA, 1764delAC, 1765delCT, 1766delTA,
1767delAA, 1768delAT, 1769delTA, 1770delAG, 1771delGT, 1772delTG, 1773delGG,
1774delGT, 1775delTC, 1776delCA, 1777delAT, 1778delTG, 1779delGA, 1780delAG,
1781delGA, 1782delAA, 1783delAT, 1784delTA, 1785delAA, 1786delAA, 1787delAA,
1788delAC, 1789delCA, 1790delAA, 1791delAA, 1792delAA, 1793delAG, 1794delGG,
1795delGT, 1796delTG, 1797delGA, 1798delAT, 1799delTT, 1800delTC, 1801delCT,
1802delTA, 1803delAT, 1804delTT, 1805delTC, 1806delCA, 1807delAG, 1808delGA,
1809delAA, 1810delAT, 1811delTG, 1812delGA, 1813delAG, 1814delGA, 1815delAA,
1816delAA, 1817delAA, 1818delAA, 1819delAT, 1820delTC, 1821delCC, 1822delCT,
1823delTA, 1824delAA, 1825delAC, 1826delCC, 1827delCC, 1828delCA, 1829delAA,
1830delAT, 1831delTA, 1832delAG, 1833delGA, 1834delAA, 1835delAT, 1836delTC,
1837delCA, 1838delAC, 1839delCT, 1840delTC, 1841delCG, 1842delGA, 1843delAA,
1844delAA, 1845delAA, 1846delAA, 1847delAG, 1848delGA, 1849delAA, 1850delAT,
1851delTC, 1852delCT, 1853delTG, 1854delGC, 1855delCT, 1856delTT, 1857delTT,
1858delTC, 1859delCA, 1860delAA, 1861delAA, 1862delAA, 1863delAC, 1864delCG,
1865delGA, 1866delAA, 1867delAA, 1868delAG, 1869delGC, 1870delCT, 1871delTG,
1872delGA, 1873delAA, 1874delAC, 1875delCC, 1876delCT, 1877delTA, 1878delAT,
1879delTA, 1880delAA, 1881delAG, 1882delGC, 1883delCA, 1884delAG, 1885delGC,
1886delCA, 1887delAG, 1888delGT, 1889delTA, 1890delAT, 1891delTA, 1892delAA,
1893delAG, 1894delGC, 1895delCA, 1896delAA, 1897delAT, 1898delTA, 1899delAT,
1900delTG, 1901delGG, 1902delGA, 1903delAA, 1904delAC, 1905delCT, 1906delTC,
1907delCG, 1908delGA, 1909delAA, 1910delAT, 1911delTT, 1912delTA, 1913delAA,
1914delAA, 1915delAT, 1916delTA, 1917delAT, 1918delTC, 1919delCC, 1920delCA,
1921delAC, 1922delCA, 1923delAA, 1924delAT, 1925delTT, 1926delTC, 1927delCA,
1928delAA, 1929delAA, 1930delAA, 1931delAG, 1932delGC, 1933delCA, 1934delAC,
1935delCC, 1936delCT, 1937delTA, 1938delAA, 1939delAA, 1940delAA, 1941delAA,
1942delAG, 1943delGA, 1944delAA, 1945delAT, 1946delTA, 1947delAG, 1948delGG,
1949delGC, 1950delCT, 1951delTG, 1952delGA, 1953delAG, 1954delGG, 1955delGA,
1956delAG, 1957delGG, 1958delGA, 1959delAA, 1960delAG, 1961delGT, 1962delTC,
1963delCT, 1964delTT, 1965delTC, 1966delCT, 1967delTA, 1968delAC, 1969delCC,
1970delCA, 1971delAG, 1972delGG, 1973delGC, 1974delCA, 1975delAT, 1976delTA,
1977delAT, 1978delTT, 1979delTC, 1980delCA, 1981delAT, 1982delTG, 1983delGC,
1984delCG, 1985delGC, 1986delCT, 1987delTT, 1988delTG, 1989delGA, 1990delAA,
1991delAC, 1992delCT, 1993delTA, 1994delAG, 1995delGT, 1996delTA, 1997delAG,
1998delGT, 1999delTC, 2000delCA, 2001delAG, 2002delGT, 2003delTA, 2004delAG,
2005delGA, 2006delAA, 2007delAA, 2008delAT, 2009delTC, 2010delCT, 2011delTA,
2012delAA, 2013delAG, 2014delGC, 2015delCC, 2016delCC, 2017delCA, 2018delAC,
2019delCC, 2020delCT, 2021delTA, 2022delAA, 2023delAT, 2024delTT, 2025delTG,
2026delGT, 2027delTA, 2028delAC, 2029delCT, 2030delTG, 2031delGA, 2032delAA,
2033delAT, 2034delTT, 2035delTG, 2036delGC, 2037delCA, 2038delAA, 2039delAA,
2040delAT, 2041delTT, 2042delTG, 2043delGA, 2044delAT, 2045delTA, 2046delAG,
2047delGT, 2048delTT, 2049delTG, 2050delGT, 2051delTT, 2052delTC, 2053delCT,
2054delTA, 2055delAG, 2056delGC, 2057delCA, 2058delAG, 2059delGT, 2060delTG,
2061delGA, 2062delAA, 2063delAG, 2064delGA, 2065delAG, 2066delGA, 2067delAT,
2068delTA, 2069delAA, 2070delAA, 2071delAG, 2072delGA, 2073delAA, 2074delAA,
2075delAA, 2076delAA, 2077delAA, 2078delAA, 2079delAA, 2080delAG, 2081delGT,
2082delTA, 2083delAC, 2084delCA, 2085delAA, 2086delAC, 2087delCC, 2088delCA,
2089delAA, 2090delAA, 2091delAT, 2092delTG, 2093delGC, 2094delCC, 2095delCA,
2096delAG, 2097delGT, 2098delTC, 2099delCA, 2100delAG, 2101delGG, 2102delGC,
2103delCA, 2104delAC, 2105delCA, 2106delAG, 2107delGC, 2108delCA, 2109delAG,
2110delGA, 2111delAA, 2112delAA, 2113delAC, 2114delCC, 2115delCT, 2116delTA,
2117delAC, 2118delCA, 2119delAA, 2120delAC, 2121delCT, 2122delTC, 2123delCA,
2124delAT, 2125delTG, 2126delGG, 2127delGA, 2128delAA, 2129delAG, 2130delGG,
2131delGT, 2132delTA, 2133delAA, 2134delAA, 2135delAG, 2136delGA, 2137delAA,
2138delAC, 2139delCC, 2140delCT, 2141delTG, 2142delGC, 2143delCA, 2144delAA,
2145delAC, 2146delCT, 2147delTG, 2148delGG, 2149delGA, 2150delAG, 2151delGC,
2152delCC, 2153delCA, 2154delAA, 2155delAG, 2156delGA, 2157delAA, 2158delAG,
2159delGA, 2160delAG, 2161delGT, 2162delTA, 2163delAA, 2164delAC, 2165delCA,
2166delAA, 2167delAG, 2168delGC, 2169delCC, 2170delCA, 2171delAA, 2172delAA,
2173delAT, 2174delTG, 2175delGA, 2176delAA, 2177delAC, 2178delCA, 2179delAG,
2180delGA, 2181delAC, 2182delCA, 2183delAA, 2184delAG, 2185delGT, 2186delTA,
2187delAA, 2188delAA, 2189delAA, 2190delAG, 2191delGA, 2192delAC, 2193delCA,
2194delAT, 2195delTG, 2196delGA, 2197delAC, 2198delCA, 2199delAG, 2200delGT,
2201delTG, 2202delGA, 2203delAT, 2204delTA, 2205delAC, 2206delCT, 2207delTT,
2208delTT, 2209delTC, 2210delCC, 2211delCC, 2212delCA, 2213delAG, 2214delGA,
2215delAG, 2216delGC, 2217delCT, 2218delTG, 2219delGA, 2220delAA, 2221delAG,
2222delGT, 2223delTT, 2224delTA, 2225delAA, 2226delAC, 2227delCA, 2228delAA,
2229delAA, 2230delAT, 2231delTG, 2232delGC, 2233delCA, 2234delAC, 2235delCC,
2236delCT, 2237delTG, 2238delGG, 2239delGT, 2240delTT, 2241delTC, 2242delCT,
2243delTT, 2244delTT, 2245delTT, 2246delTA, 2247delAC, 2248delCT, 2249delTA,
2250delAA, 2251delAG, 2252delGT, 2253delTG, 2254delGT, 2255delTT, 2256delTC,
2257delCA, 2258delAA, 2259delAA, 2260delAT, 2261delTA, 2262delAC, 2263delCC,
2264delCA, 2265delAG, 2266delGT, 2267delTG, 2268delGA, 2269delAA, 2270delAC,
2271delCT, 2272delTT, 2273delTA, 2274delAA, 2275delAA, 2276delAG, 2277delGA,
2278delAA, 2279delAT, 2280delTT, 2281delTT, 2282delTG, 2283delGT, 2284delTC,
2285delCA, 2286delAA, 2287delAT, 2288delTC, 2289delCC, 2290delCT, 2291delTA,
2292delAG, 2293delGC, 2294delCC, 2295delCT, 2296delTT, 2297delTC, 2298delCC,
2299delCA, 2300delAA, 2301delAG, 2302delGA, 2303delAG, 2304delGA, 2305delAA,
2306delAG, 2307delGA, 2308delAA, 2309delAA, 2310delAA, 2311delAA, 2312delAG,
2313delGA, 2314delAA, 2315delAG, 2316delGA, 2317delAG, 2318delGA, 2319delAA,
2320delAA, 2321delAC, 2322delCT, 2323delTA, 2324delAG, 2325delGA, 2326delAA,
2327delAA, 2328delAC, 2329delCA, 2330delAG, 2331delGT, 2332delTT, 2333delTA,
2334delAA, 2335delAA, 2336delAG, 2337delGT, 2338delTG, 2339delGT, 2340delTC,
2341delCT, 2342delTA, 2343delAA, 2344delAT, 2345delTA, 2346delAA, 2347delAT,
2348delTG, 2349delGC, 2350delCT, 2351delTG, 2352delGA, 2353delAA, 2354delAG,
2355delGA, 2356delAC, 2357delCC, 2358delCC, 2359delCC, 2360delCA, 2361delAA,
2362delAA, 2363delAG, 2364delGA, 2365delAT, 2366delTC, 2367delCT, 2368delTC,
2369delCA, 2370delAT, 2371delTG, 2372delGT, 2373delTT, 2374delTA, 2375delAA,
2376delAG, 2377delGT, 2378delTG, 2379delGG, 2380delGA, 2381delAG, 2382delGA,
2383delAA, 2384delAA, 2385delAG, 2386delGG, 2387delGG, 2388delGT, 2389delTT,
2390delTT, 2391delTT, 2392delTG, 2393delGC, 2394delCA, 2395delAA, 2396delAA,
2397delAC, 2398delCT, 2399delTG, 2400delGA, 2401delAA, 2402delAA, 2403delAG,
2404delGA, 2405delAT, 2406delTC, 2407delCT, 2408delTG, 2409delGT, 2410delTA,
2411delAG, 2412delGA, 2413delAG, 2414delGA, 2415delAG, 2416delGT, 2417delTA,
2418delAG, 2419delGC, 2420delCA, 2421delAG, 2422delGT, 2423delTA, 2424delAT,
2425delTT, 2426delTT, 2427delTC, 2428delCA, 2429delAC, 2430delCT, 2431delTG,
2432delGG, 2433delGT, 2434delTA, 2435delAC, 2436delCC, 2437delCT, 2438delTG,
2439delGG, 2440delGT, 2441delTA, 2442delAC, 2443delCT, 2444delTG, 2445delGA,
2446delAT, 2447delTT, 2448delTA, 2449delAT, 2450delTG, 2451delGG, 2452delGC,
2453delCA, 2454delAC, 2455delCT, 2456delTC, 2457delCA, 2458delAG, 2459delGG,
2460delGA, 2461delAA, 2462delAA, 2463delAG, 2464delGT, 2465delTA, 2466delAT,
2467delTC, 2468delCT, 2469delTC, 2470delCG, 2471delGT, 2472delTT, 2473delTA,
2474delAC, 2475delCT, 2476delTG, 2477delGG, 2478delGA, 2479delAA, 2480delAG,
2481delGT, 2482delTT, 2483delTA, 2484delAG, 2485delGC, 2486delCA, 2487delAC,
2488delCT, 2489delTC, 2490delCT, 2491delTA, 2492delAG, 2493delGG, 2494delGG,
2495delGA, 2496delAA, 2497delAG, 2498delGG, 2499delGC, 2500delCA, 2501delAA,
2502delAA, 2503delAA, 2504delAA, 2505delAC, 2506delCA, 2507delAG, 2508delGA,
2509delAA, 2510delAC, 2511delCC, 2512delCA, 2513delAA, 2514delAA, 2515delAT,
2516delTA, 2517delAA, 2518delAA, 2519delAT, 2520delTG, 2521delGT, 2522delTG,
2523delGT, 2524delTG, 2525delGA, 2526delAG, 2527delGT, 2528delTC, 2529delCA,
2530delAG, 2531delGT, 2532delTG, 2533delGT, 2534delTG, 2535delGC, 2536delCA,
2537delAG, 2538delGC, 2539delCA, 2540delAT, 2541delTT, 2542delTT, 2543delTG,
2544delGA, 2545delAA, 2546delAA, 2547delAA, 2548delAC, 2549delCC, 2550delCC,
2551delCC, 2552delCA, 2553delAA, 2554delAG, 2555delGG, 2556delGG, 2557delGA,
2558delAC, 2559delCT, 2560delTA, 2561delAA, 2562delAT, 2563delTT, 2564delTC,
2565delCA, 2566delAT, 2567delTG, 2568delGG, 2569delGT, 2570delTT, 2571delTG,
2572delGT, 2573delTT, 2574delTC, 2575delCC, 2576delCA, 2577delAA, 2578delAA,
2579delAG, 2580delGA, 2581delAT, 2582delTA, 2583delAA, 2584delAT, 2585delTA,
2586delAG, 2587delGA, 2588delAA, 2589delAA, 2590delAT, 2591delTG, 2592delGA,
2593delAC, 2594delCA, 2595delAC, 2596delCA, 2597delAG, 2598delGA, 2599delAA,
2600delAG, 2601delGG, 2602delGC, 2603delCT, 2604delTT, 2605delTT, 2606delTA,
2607delAA, 2608delAG, 2609delGT, 2610delTA, 2611delAT, 2612delTC, 2613delCC,
2614delCA, 2615delAT, 2616delTT, 2617delTG, 2618delGG, 2619delGG, 2620delGA,
2621delAC, 2622delCA, 2623delAT, 2624delTG, 2625delGA, 2626delAA, 2627delAG,
2628delGT, 2629delTT, 2630delTA, 2631delAA, 2632delAC, 2633delCC, 2634delCA,
2635delAC, 2636delCA, 2637delAG, 2638delGT, 2639delTC, 2640delCG, 2641delGG,
2642delGG, 2643delGA, 2644delAA, 2645delAA, 2646delAC, 2647delCA, 2648delAA,
2649delAG, 2650delGC, 2651delCA, 2652delAT, 2653delTA, 2654delAG, 2655delGA,
2656delAA, 2657delAA, 2658delAT, 2659delTG, 2660delGG, 2661delGA, 2662delAA,
2663delAG, 2664delGA, 2665delAA, 2666delAA, 2667delAG, 2668delGT, 2669delTG,
2670delGA, 2671delAA, 2672delAC, 2673delCT, 2674delTT, 2675delTG, 2676delGA,
2677delAT, 2678delTG, 2679delGC, 2680delCT, 2681delTC, 2682delCA, 2683delAG,
2684delGT, 2685delTA, 2686delAT, 2687delTT, 2688delTT, 2689delTG, 2690delGC,
2691delCA, 2692delAG, 2693delGA, 2694delAA, 2695delAT, 2696delTA, 2697delAC,
2698delCA, 2699delAT, 2700delTT, 2701delTC, 2702delCA, 2703delAA, 2704delAG,
2705delGG, 2706delGT, 2707delTT, 2708delTT, 2709delTC, 2710delCA, 2711delAA,
2712delAA, 2713delAG, 2714delGC, 2715delCG, 2716delGC, 2717delCC, 2718delCA,
2719delAG, 2720delGT, 2721delTC, 2722delCA, 2723delAT, 2724delTT, 2725delTT,
2726delTG, 2727delGC, 2728delCT, 2729delTC, 2730delCT, 2731delTG, 2732delGT,
2733delTT, 2734delTT, 2735delTT, 2736delTC, 2737delCA, 2738delAA, 2739delAA,
2740delAT, 2741delTC, 2742delCC, 2743delCA, 2744delAG, 2745delGG, 2746delGA,
2747delAA, 2748delAA, 2749delAT, 2750delTG, 2751delGC, 2752delCA, 2753delAG,
2754delGA, 2755delAA, 2756delAG, 2757delGA, 2758delAG, 2759delGG, 2760delGA,
2761delAA, 2762delAT, 2763delTG, 2764delGT, 2765delTG, 2766delGC, 2767delCA,
2768delAA, 2769delAC, 2770delCA, 2771delAT, 2772delTT, 2773delTC, 2774delCT,
2775delTC, 2776delCT, 2777delTG, 2778delGC, 2779delCC, 2780delCC, 2781delCA,
2782delAC, 2783delCT, 2784delTC, 2785delCT, 2786delTG, 2787delGG, 2788delGG,
2789delGT, 2790delTC, 2791delCC, 2792delCT, 2793delTT, 2794delTA, 2795delAA,
2796delAA, 2797delAG, 2798delGA, 2799delAA, 2800delAA, 2801delAC, 2802delCA,
2803delAA, 2804delAA, 2805delAG, 2806delGT, 2807delTC, 2808delCC, 2809delCA,
2810delAA, 2811delAA, 2812delAA, 2813delAG, 2814delGT, 2815delTC, 2816delCA,
2817delAC, 2818delCT, 2819delTT, 2820delTT, 2821delTT, 2822delTG, 2823delGA,
2824delAA, 2825delAT, 2826delTG, 2827delGT, 2828delTG, 2829delGA, 2830delAA,
2831delAC, 2832delCA, 2833delAA, 2834delAA, 2835delAA, 2836delAG, 2837delGG,
2838delGA, 2839delAA, 2840delAG, 2841delGA, 2842delAA, 2843delAA, 2844delAA,
2845delAT, 2846delTC, 2847delCA, 2848delAA, 2849delAG, 2850delGG, 2851delGA,
2852delAA, 2853delAA, 2854delAG, 2855delGA, 2856delAA, 2857delAT, 2858delTG,
2859delGA, 2860delAG, 2861delGT, 2862delTC, 2863delCT, 2864delTA, 2865delAA,
2866delAT, 2867delTA, 2868delAT, 2869delTC, 2870delCA, 2871delAA, 2872delAG,
2873delGC, 2874delCC, 2875delCT, 2876delTG, 2877delGT, 2878delTA, 2879delAC,
2880delCA, 2881delAG, 2882delGA, 2883delAC, 2884delCA, 2885delAG, 2886delGT,
2887delTT, 2888delTA, 2889delAA, 2890delAT, 2891delTA, 2892delAT, 2893delTC,
2894delCA, 2895delAC, 2896delCT, 2897delTG, 2898delGC, 2899delCA, 2900delAG,
2901delGG, 2902delGC, 2903delCT, 2904delTT, 2905delTT, 2906delTC, 2907delCC,
2908delCT, 2909delTG, 2910delGT, 2911delTG, 2912delGG, 2913delGT, 2914delTT,
2915delTG, 2916delGG, 2917delGT, 2918delTC, 2919delCA, 2920delAG, 2921delGA,
2922delAA, 2923delAA, 2924delAG, 2925delGA, 2926delAT, 2927delTA, 2928delAA,
2929delAG, 2930delGC, 2931delCC, 2932delCA, 2933delAG, 2934delGT, 2935delTT,
2936delTG, 2937delGA, 2938delAT, 2939delTA, 2940delAA, 2941delAT, 2942delTG,
2943delGC, 2944delCC, 2945delCA, 2946delAA, 2947delAA, 2948delAT, 2949delTG,
2950delGT, 2951delTA, 2952delAG, 2953delGT, 2954delTA, 2955delAT, 2956delTC,
2957delCA, 2958delAA, 2959delAA, 2960delAG, 2961delGG, 2962delGA, 2963delAG,
2964delGG, 2965delGC, 2966delCT, 2967delTC, 2968delCT, 2969delTA, 2970delAG,
2971delGG, 2972delGT, 2973delTT, 2974delTT, 2975delTT, 2976delTG, 2977delGT,
2978delTC, 2979delCT, 2980delTA, 2981delAT, 2982delTC, 2983delCA, 2984delAT,
2985delTC, 2986delCT, 2987delTC, 2988delCA, 2989delAG, 2990delGT, 2991delTT,
2992delTC, 2993delCA, 2994delAG, 2995delGA, 2996delAG, 2997delGG, 2998delGC,
2999delCA, 3000delAA, 3001delAC, 3002delCG, 3003delGA, 3004delAA, 3005delAA,
3006delAC, 3007delCT, 3008delTG, 3009delGG, 3010delGA, 3011delAC, 3012delCT,
3013delTC, 3014delCA, 3015delAT, 3016delTT, 3017delTA, 3018delAC, 3019delCT,
3020delTC, 3021delCC, 3022delCA, 3023delAA, 3024delAA, 3025delAT, 3026delTA,
3027delAA, 3028delAA, 3029delAC, 3030delCA, 3031delAT, 3032delTG, 3033delGG,
3034delGA, 3035delAC, 3036delCT, 3037delTT, 3038delTT, 3039delTT, 3040delTA,
3041delAC, 3042delCA, 3043delAA, 3044delAA, 3045delAA, 3046delAC, 3047delCC,
3048delCC, 3049delCA, 3050delAT, 3051delTA, 3052delAT, 3053delTC, 3054delCG,
3055delGT, 3056delTA, 3057delAT, 3058delTA, 3059delAC, 3060delCC, 3061delCA,
3062delAC, 3063delCC, 3064delCA, 3065delAC, 3066delCT, 3067delTT, 3068delTT,
3069delTT, 3070delTT, 3071delTC, 3072delCC, 3073delCC, 3074delCA, 3075delAT,
3076delTC, 3077delCA, 3078delAA, 3079delAG, 3080delGT, 3081delTC, 3082delCA,
3083delAT, 3084delTT, 3085delTT, 3086delTG, 3087delGT, 3088delTT, 3089delTA,
3090delAA, 3091delAA, 3092delAA, 3093delAC, 3094delCT, 3095delTA, 3096delAA,
3097delAA, 3098delAT, 3099delTG, 3100delGT, 3101delTA, 3102delAA, 3103delAG,
3104delGA, 3105delAA, 3106delAA, 3107delAA, 3108delAA, 3109delAT, 3110delTC,
3111delCT, 3112delTG, 3113delGC, 3114delCT, 3115delTA, 3116delAG, 3117delGA,
3118delAG, 3119delGG, 3120delGA, 3121delAA, 3122delAA, 3123delAA, 3124delAC,
3125delCT, 3126delTT, 3127delTT, 3128delTG, 3129delGA, 3130delAG, 3131delGG,
3132delGA, 3133delAA, 3134delAC, 3135delCA, 3136delAT, 3137delTT, 3138delTC,
3139delCA, 3140delAA, 3141delAT, 3142delTG, 3143delGT, 3144delTC, 3145delCA,
3146delAC, 3147delCC, 3148delCT, 3149delTG, 3150delGA, 3151delAA, 3152delAA,
3153delAG, 3154delGA, 3155delAG, 3156delGA, 3157delAA, 3158delAA, 3159delAT,
3160delTG, 3161delGG, 3162delGG, 3163delGA, 3164delAA, 3165delAA, 3166delAT,
3167delTG, 3168delGA, 3169delAG, 3170delGA, 3171delAA, 3172delAC, 3173delCA,
3174delAT, 3175delTT, 3176delTC, 3177delCC, 3178delCA, 3179delAA, 3180delAG,
3181delGT, 3182delTA, 3183delAC, 3184delCA, 3185delAG, 3186delGT, 3187delTG,
3188delGA, 3189delAG, 3190delGC, 3191delCA, 3192delAC, 3193delCA, 3194delAA,
3195delAT, 3196delTT, 3197delTA, 3198delAG, 3199delGC, 3200delCC, 3201delCG,
3202delGT, 3203delTA, 3204delAA, 3205delAT, 3206delTA, 3207delAA, 3208delAC,
3209delCA, 3210delAT, 3211delTT, 3212delTA, 3213delAG, 3214delGA, 3215delAG,
3216delGA, 3217delAA, 3218delAA, 3219delAA, 3220delAT, 3221delTG, 3222delGT,
3223delTT, 3224delTT, 3225delTT, 3226delTT, 3227delTA, 3228delAA, 3229delAA,
3230delAG, 3231delGG, 3232delGA, 3233delAG, 3234delGC, 3235delCC, 3236delCA,
3237delAG, 3238delGC, 3239delCT, 3240delTC, 3241delCA, 3242delAA, 3243delAG,
3244delGC, 3245delCA, 3246delAA, 3247delAT, 3248delTA, 3249delAT, 3250delTT,
3251delTA, 3252delAA, 3253delAT, 3254delTG, 3255delGA, 3256delAA, 3257delAG,
3258delGT, 3259delTA, 3260delAG, 3261delGG, 3262delGT, 3263delTT, 3264delTC,
3265delCC, 3266delCA, 3267delAG, 3268delGT, 3269delTA, 3270delAC, 3271delCT,
3272delTA, 3273delAA, 3274delAT, 3275delTG, 3276delGA, 3277delAA, 3278delAG,
3279delGT, 3280delTG, 3281delGG, 3282delGG, 3283delGC, 3284delCT, 3285delTC,
3286delCC, 3287delCA, 3288delAG, 3289delGT, 3290delTA, 3291delAT, 3292delTT,
3293delTA, 3294delAA, 3295delAT, 3296delTG, 3297delGA, 3298delAA, 3299delAA,
3300delAT, 3301delTA, 3302delAG, 3303delGG, 3304delGT, 3305delTT, 3306delTC,
3307delCC, 3308delCA, 3309delAG, 3310delGT, 3311delTG, 3312delGA, 3313delAT,
3314delTG, 3315delGA, 3316delAA, 3317delAA, 3318delAA, 3319delAC, 3320delCA,
3321delAT, 3322delTT, 3323delTC, 3324delCA, 3325delAA, 3326delAG, 3327delGC,
3328delCA, 3329delAG, 3330delGA, 3331delAA, 3332delAC, 3333delCT, 3334delTA,
3335delAG, 3336delGG, 3337delGT, 3338delTA, 3339delAG, 3340delGA, 3341delAA,
3342delAA, 3343delAC, 3344delCA, 3345delAG, 3346delGA, 3347delAG, 3348delGG,
3349delGG, 3350delGC, 3351delCC, 3352delCA, 3353delAA, 3354delAA, 3355delAA,
3356delAT, 3357delTT, 3358delTG, 3359delGA, 3360delAA, 3361delAT, 3362delTG,
3363delGC, 3364delCT, 3365delTA, 3366delAT, 3367delTG, 3368delGC, 3369delCT,
3370delTT, 3371delTA, 3372delAG, 3373delGA, 3374delAT, 3375delTT, 3376delTA,
3377delAG, 3378delGG, 3379delGG, 3380delGG, 3381delGT, 3382delTT, 3383delTT,
3384delTT, 3385delTG, 3386delGC, 3387delCA, 3388delAA, 3389delAC, 3390delCC,
3391delCT, 3392delTG, 3393delGA, 3394delAG, 3395delGG, 3396delGT, 3397delTC,
3398delCT, 3399delTA, 3400delAT, 3401delTA, 3402delAA, 3403delAA, 3404delAC,
3405delCA, 3406delAA, 3407delAA, 3408delAG, 3409delGT, 3410delTC, 3411delCT,
3412delTT, 3413delTC, 3414delCC, 3415delCT, 3416delTG, 3417delGG, 3418delGA,
3419delAA, 3420delAG, 3421delGT, 3422delTA, 3423delAA, 3424delAT, 3425delTT,
3426delTG, 3427delGT, 3428delTA, 3429delAA, 3430delAG, 3431delGC, 3432delCA,
3433delAT, 3434delTC, 3435delCC, 3436delCT, 3437delTG, 3438delGA, 3439delAA,
3440delAA, 3441delAT, 3442delTA, 3443delAA, 3444delAA, 3445delAA, 3446delAA,
3447delAA, 3448delAG, 3449delGC, 3450delCA, 3451delAA, 3452delAG, 3453delGA,
3454delAA, 3455delAT, 3456delTA, 3457delAT, 3458delTG, 3459delGA, 3460delAA,
3461delAG, 3462delGA, 3463delAA, 3464delAG, 3465delGT, 3466delTA, 3467delAG,
3468delGT, 3469delTT, 3470delTC, 3471delCA, 3472delAG, 3473delGA, 3474delAC,
3475delCT, 3476delTG, 3477delGT, 3478delTT, 3479delTA, 3480delAA, 3481delAT,
3482delTA, 3483delAC, 3484delCA, 3485delAG, 3486delGA, 3487delAT, 3488delTT,
3489delTT, 3490delTC, 3491delCT, 3492delTC, 3493delCT, 3494delTC, 3495delCC,
3496delCA, 3497delAT, 3498delTA, 3499delAT, 3500delTC, 3501delCT, 3502delTG,
3503delGA, 3504delAT, 3505delTT, 3506delTT, 3507delTC, 3508delCA, 3509delAG,
3510delGA, 3511delAT, 3512delTA, 3513delAA, 3514delAC, 3515delCT, 3516delTT,
3517delTA, 3518delAG, 3519delGA, 3520delAA, 3521delAC, 3522delCA, 3523delAG,
3524delGC, 3525delCC, 3526delCT, 3527delTA, 3528delAT, 3529delTG, 3530delGG,
3531delGG, 3532delGA, 3533delAA, 3534delAG, 3535delGT, 3536delTA, 3537delAG,
3538delGT, 3539delTC, 3540delCA, 3541delAT, 3542delTG, 3543delGC, 3544delCA,
3545delAT, 3546delTC, 3547delCT, 3548delTC, 3549delCA, 3550delAG, 3551delGG,
3552delGT, 3553delTT, 3554delTT, 3555delTG, 3556delGT, 3557delTT, 3558delTC,
3559delCT, 3560delTG, 3561delGA, 3562delAG, 3563delGA, 3564delAC, 3565delCA,
3566delAC, 3567delCC, 3568delCT, 3569delTG, 3570delGA, 3571delAT, 3572delTG,
3573delGA, 3574delAC, 3575delCC, 3576delCT, 3577delTG, 3578delGT, 3579delTT,
3580delTA, 3581delAG, 3582delGA, 3583delAT, 3584delTG, 3585delGA, 3586delAT,
3587delTG, 3588delGG, 3589delGT, 3590delTG, 3591delGA, 3592delAA, 3593delAA,
3594delAT, 3595delTA, 3596delAA, 3597delAA, 3598delAG, 3599delGG, 3600delGA,
3601delAA, 3602delAG, 3603delGA, 3604delAT, 3605delTA, 3606delAC, 3607delCT,
3608delTA, 3609delAG, 3610delGT, 3611delTT, 3612delTT, 3613delTT, 3614delTG,
3615delGC, 3616delCT, 3617delTG, 3618delGA, 3619delAA, 3620delAA, 3621delAA,
3622delAT, 3623delTG, 3624delGA, 3625delAC, 3626delCA, 3627delAT, 3628delTT,
3629delTA, 3630delAA, 3631delAG, 3632delGG, 3633delGA, 3634delAA, 3635delAA,
3636delAG, 3637delGT, 3638delTT, 3639delTC, 3640delCT, 3641delTG, 3642delGC,
3643delCT, 3644delTG, 3645delGT, 3646delTT, 3647delTT, 3648delTT, 3649delTT,
3650delTA, 3651delAG, 3652delGC, 3653delCA, 3654delAA, 3655delAA, 3656delAA,
3657delAG, 3658delGC, 3659delCG, 3660delGT, 3661delTC, 3662delCC, 3663delCA,
3664delAG, 3665delGA, 3666delAG, 3667delGA, 3668delAG, 3669delGG, 3670delGA,
3671delAG, 3672delGA, 3673delAG, 3674delGC, 3675delCT, 3676delTT, 3677delTA,
3678delAG, 3679delGC, 3680delCA, 3681delAG, 3682delGG, 3683delGA, 3684delAG,
3685delGT, 3686delTC, 3687delCC, 3688delCT, 3689delTA, 3690delAG, 3691delGC,
3692delCC, 3693delCC, 3694delCT, 3695delTT, 3696delTT, 3697delTC, 3698delCA,
3699delAC, 3700delCC, 3701delCC, 3702delCA, 3703delAT, 3704delTA, 3705delAC,
3706delCA, 3707delAC, 3708delCA, 3709delAT, 3710delTT, 3711delTT, 3712delTG,
3713delGG, 3714delGC, 3715delCT, 3716delTC, 3717delCA, 3718delAG, 3719delGG,
3720delGG, 3721delGT, 3722delTT, 3723delTA, 3724delAC, 3725delCC, 3726delCG,
3727delGA, 3728delAA, 3729delAG, 3730delGA, 3731delAG, 3732delGG, 3733delGG,
3734delGG, 3735delGC, 3736delCC, 3737delCA, 3738delAA, 3739delAG, 3740delGA,
3741delAA, 3742delAA, 3743delAT, 3744delTT, 3745delTA, 3746delAG, 3747delGA,
3748delAG, 3749delGT, 3750delTC, 3751delCC, 3752delCT, 3753delTC, 3754delCA,
3755delAG, 3756delGA, 3757delAA, 3758delAG, 3759delGA, 3760delAG, 3761delGA,
3762delAA, 3763delAC, 3764delCT, 3765delTT, 3766delTA, 3767delAT, 3768delTC,
3769delCT, 3770delTA, 3771delAG, 3772delGT, 3773delTG, 3774delGA, 3775delAG,
3776delGG, 3777delGA, 3778delAT, 3779delTG, 3780delGA, 3781delAA, 3782delAG,
3783delGA, 3784delAG, 3785delGC, 3786delCT, 3787delTT, 3788delTC, 3789delCC,
3790delCC, 3791delCT, 3792delTG, 3793delGC, 3794delCT, 3795delTT, 3796delTC,
3797delCC, 3798delCA, 3799delAA, 3800delAC, 3801delCA, 3802delAC, 3803delCT,
3804delTT, 3805delTG, 3806delGT, 3807delTT, 3808delTA, 3809delAT, 3810delTT,
3811delTT, 3812delTG, 3813delGG, 3814delGT, 3815delTA, 3816delAA, 3817delAA,
3818delAG, 3819delGT, 3820delTA, 3821delAA, 3822delAA, 3823delAC, 3824delCA,
3825delAA, 3826delAT, 3827delTA, 3828delAT, 3829delTA, 3830delAC, 3831delCC,
3832delCT, 3833delTT, 3834delTC, 3835delCT, 3836delTC, 3837delCA, 3838delAG,
3839delGT, 3840delTC, 3841delCT, 3842delTA, 3843delAC, 3844delCT, 3845delTA,
3846delAG, 3847delGG, 3848delGC, 3849delCA, 3850delAT, 3851delTA, 3852delAG,
3853delGC, 3854delCA, 3855delAC, 3856delCC, 3857delCG, 3858delGT, 3859delTT,
3860delTG, 3861delGC, 3862delCT, 3863delTA, 3864delAC, 3865delCC, 3866delCG,
3867delGA, 3868delAG, 3869delGT, 3870delTG, 3871delGT, 3872delTC, 3873delCT,
3874delTG, 3875delGT, 3876delTC, 3877delCT, 3878delTA, 3879delAA, 3880delAG,
3881delGA, 3882delAA, 3883delAC, 3884delCA, 3885delAC, 3886delCA, 3887delAG,
3888delGA, 3889delAG, 3890delGG, 3891delGA, 3892delAG, 3893delGA, 3894delAA,
3895delAT, 3896delTT, 3897delTT, 3898delTA, 3899delAT, 3900delTT, 3901delTA,
3902delAT, 3903delTC, 3904delCA, 3905delAT, 3906delTT, 3907delTG, 3908delGA,
3909delAA, 3910delAG, 3911delGA, 3912delAA, 3913delAT, 3914delTA, 3915delAG,
3916delGC, 3917delCT, 3918delTT, 3919delTA, 3920delAA, 3921delAA, 3922delAT,
3923delTG, 3924delGA, 3925delAC, 3926delCT, 3927delTG, 3928delGC, 3929delCA,
3930delAG, 3931delGT, 3932delTA, 3933delAA, 3934delAC, 3935delCC, 3936delCA,
3937delAG, 3938delGG, 3939delGT, 3940delTA, 3941delAA, 3942delAT, 3943delTA,
3944delAT, 3945delTT, 3946delTG, 3947delGG, 3948delGC, 3949delCA, 3950delAA,
3951delAA, 3952delAG, 3953delGG, 3954delGC, 3955delCA, 3956delAT, 3957delTC,
3958delCT, 3959delTC, 3960delCA, 3961delAG, 3962delGG, 3963delGA, 3964delAA,
3965delAC, 3966delCA, 3967delAT, 3968delTC, 3969delCA, 3970delAC, 3971delCC,
3972delCT, 3973delTT, 3974delTA, 3975delAG, 3976delGT, 3977delTG, 3978delGA,
3979delAG, 3980delGG, 3981delGA, 3982delAA, 3983delAA, 3984delAC, 3985delCA,
3986delAA, 3987delAA, 3988delAA, 3989delAT, 3990delTG, 3991delGT, 3992delTT,
3993delTC, 3994delCT, 3995delTG, 3996delGC, 3997delCT, 3998delTA, 3999delAG,
4000delGC, 4001delCT, 4002delTT, 4003delTG, 4004delGT, 4005delTT, 4006delTT,
4007delTT, 4008delTC, 4009delCT, 4010delTT, 4011delTC, 4012delCA, 4013delAC,
4014delCA, 4015delAG, 4016delGT, 4017delTG, 4018delGC, 4019delCA, 4020delAG,
4021delGT, 4022delTG, 4023delGA, 4024delAA, 4025delAT, 4026delTT, 4027delTG,
4028delGG, 4029delGA, 4030delAA, 4031delAG, 4032delGA, 4033delAC, 4034delCT,
4035delTT, 4036delTG, 4037delGA, 4038delAC, 4039delCT, 4040delTG, 4041delGC,
4042delCA, 4043delAA, 4044delAA, 4045delAT, 4046delTA, 4047delAC, 4048delCA,
4049delAA, 4050delAA, 4051delAC, 4052delCA, 4053delAC, 4054delCC, 4055delCC,
4056delCA, 4057delAG, 4058delGG, 4059delGA, 4060delAT, 4061delTC, 4062delCC,
4063delCT, 4064delTT, 4065delTT, 4066delTC, 4067delCT, 4068delTT, 4069delTG,
4070delGA, 4071delAT, 4072delTT, 4073delTG, 4074delGG, 4075delGT, 4076delTT,
4077delTC, 4078delCT, 4079delTT, 4080delTC, 4081delCC, 4082delCA, 4083delAA,
4084delAA, 4085delAC, 4086delCA, 4087delAA, 4088delAA, 4089delAT, 4090delTG,
4091delGA, 4092delAG, 4093delGG, 4094delGC, 4095delCA, 4096delAT, 4097delTC,
4098delCA, 4099delAG, 4100delGT, 4101delTC, 4102delCT, 4103delTG, 4104delGA,
4105delAA, 4106delAA, 4107delAG, 4108delGC, 4109delCC, 4110delCA, 4111delAG,
4112delGG, 4113delGG, 4114delGA, 4115delAG, 4116delGT, 4117delTT, 4118delTG,
4119delGG, 4120delGT, 4121delTC, 4122delCT, 4123delTG, 4124delGA, 4125delAG,
4126delGT, 4127delTG, 4128delGA, 4129delAC, 4130delCA, 4131delAA, 4132delAG,
4133delGG, 4134delGA, 4135delAA, 4136delAT, 4137delTT, 4138delTG, 4139delGG,
4140delGT, 4141delTT, 4142delTT, 4143delTC, 4144delCA, 4145delAG, 4146delGA,
4147delAT, 4148delTG, 4149delGA, 4150delAT, 4151delTG, 4152delGA, 4153delAA,
4154delAG, 4155delGA, 4156delAA, 4157delAA, 4158delAG, 4159delGA, 4160delAG,
4161delGG, 4162delGA, 4163delAA, 4164delAC, 4165delCG, 4166delGG, 4167delGG,
4168delGC, 4169delCT, 4170delTT, 4171delTG, 4172delGG, 4173delGA, 4174delAA,
4175delAG, 4176delGA, 4177delAA, 4178delAA, 4179delAA, 4180delAT, 4181delTA,
4182delAA, 4183delAT, 4184delTC, 4185delCA, 4186delAA, 4187delAG, 4188delGA,
4189delAA, 4190delAG, 4191delGA, 4192delAG, 4193delGC, 4194delCA, 4195delAA,
4196delAA, 4197delAG, 4198delGC, 4199delCA, 4200delAT, 4201delTG, 4202delGG,
4203delGA, 4204delAT, 4205delTT, 4206delTC, 4207delCA, 4208delAA, 4209delAA,
4210delAC, 4211delCT, 4212delTT, 4213delTA, 4214delAG, 4215delGG, 4216delGT,
4217delTG, 4218delGA, 4219delAA, 4220delAG, 4221delGC, 4222delCA, 4223delAG,
4224delGC, 4225delCA, 4226delAT, 4227delTC, 4228delCT, 4229delTG, 4230delGG,
4231delGG, 4232delGT, 4233delTG, 4234delGT, 4235delTG, 4236delGA, 4237delAG,
4238delGA, 4239delAG, 4240delGT, 4241delTG, 4242delGA, 4243delAA, 4244delAA,
4245delAC, 4246delCA, 4247delAA, 4248delAG, 4249delGC, 4250delCG, 4251delGT,
4252delTC, 4253delCT, 4254delTC, 4255delCT, 4256delTG, 4257delGA, 4258delAA,
4259delAG, 4260delGA, 4261delAC, 4262delCT, 4263delTG, 4264delGC, 4265delCT,
4266delTC, 4267delCA, 4268delAG, 4269delGG, 4270delGG, 4271delGC, 4272delCT,
4273delTA, 4274delAT, 4275delTC, 4276delCC, 4277delCT, 4278delTC, 4279delCT,
4280delTC, 4281delCA, 4282delAG, 4283delGA, 4284delAG, 4285delGT, 4286delTG,
4287delGA, 4288delAC, 4289delCA, 4290delAT, 4291delTT, 4292delTT, 4293delTT,
4294delTA, 4295delAA, 4296delAC, 4297delCC, 4298delCA, 4299delAC, 4300delCT,
4301delTC, 4302delCA, 4303delAG, 4304delGC, 4305delCA, 4306delAG, 4307delGA,
4308delAG, 4309delGG, 4310delGG, 4311delGA, 4312delAT, 4313delTA, 4314delAC,
4315delCC, 4316delCA, 4317delAT, 4318delTG, 4319delGC, 4320delCA, 4321delAA,
4322delAC, 4323delCA, 4324delAT, 4325delTA, 4326delAA, 4327delAC, 4328delCC,
4329delCT, 4330delTG, 4331delGA, 4332delAT, 4333delTA, 4334delAA, 4335delAA,
4336delAG, 4337delGC, 4338delCT, 4339delTC, 4340delCC, 4341delCA, 4342delAG,
4343delGC, 4344delCA, 4345delAG, 4346delGG, 4347delGA, 4348delAA, 4349delAA,
4350delAT, 4351delTG, 4352delGG, 4353delGC, 4354delCT, 4355delTG, 4356delGA,
4357delAA, 4358delAC, 4359delCT, 4360delTA, 4361delAG, 4362delGA, 4363delAA,
4364delAG, 4365delGC, 4366delCT, 4367delTG, 4368delGT, 4369delTG, 4370delGT,
4371delTT, 4372delTA, 4373delAG, 4374delGA, 4375delAA, 4376delAC, 4377delCA,
4378delAG, 4379delGC, 4380delCA, 4381delAT, 4382delTG, 4383delGG, 4384delGG,
4385delGA, 4386delAG, 4387delGC, 4388delCC, 4389delCA, 4390delAG, 4391delGC,
4392delCC, 4393delCT, 4394delTT, 4395delTC, 4396delCT, 4397delTA, 4398delAA,
4399delAC, 4400delCA, 4401delAG, 4402delGC, 4403delCT, 4404delTA, 4405delAC,
4406delCC, 4407delCC, 4408delCT, 4409delTT, 4410delTC, 4411delCC, 4412delCA,
4413delAT, 4414delTC, 4415delCA, 4416delAT, 4417delTA, 4418delAA, 4419delAG,
4420delGT, 4421delTG, 4422delGA, 4423delAC, 4424delCT, 4425delTC, 4426delCC,
4427delCT, 4428delTC, 4429delCT, 4430delTG, 4431delGC, 4432delCC, 4433delCC,
4434delCT, 4435delTT, 4436delTG, 4437delGA, 4438delAG, 4439delGG, 4440delGA,
4441delAC, 4442delCC, 4443delCT, 4444delTG, 4445delGC, 4446delCG, 4447delGA,
4448delAA, 4449delAA, 4450delAT, 4451delTC, 4452delCC, 4453delCA, 4454delAG,
4455delGA, 4456delAA, 4457delAC, 4458delCA, 4459delAA, 4460delAA, 4461delAG,
4462delGC, 4463delCA, 4464delAC, 4465delCA, 4466delAT, 4467delTC, 4468delCA,
4469delAG, 4470delGA, 4471delAA, 4472delAA, 4473delAA, 4474delAA, 4475delAG,
4476delGC, 4477delCA, 4478delAG, 4479delGT, 4480delTA, 4481delAT, 4482delTT,
4483delTA, 4484delAA, 4485delAC, 4486delCT, 4487delTT, 4488delTC, 4489delCA,
4490delAC, 4491delCA, 4492delAG, 4493delGA, 4494delAA, 4495delAA, 4496delAA,
4497delAG, 4498delGT, 4499delTA, 4500delAG, 4501delGT, 4502delTG, 4503delGA,
4504delAA, 4505delAT, 4506delTA, 4507delAC, 4508delCC, 4509delCC, 4510delCT,
4511delTA, 4512delAT, 4513delTA, 4514delAA, 4515delAG, 4516delGC, 4517delCC,
4518delCA, 4519delAG, 4520delGA, 4521delAA, 4522delAT, 4523delTC, 4524delCC,
4525delCA, 4526delAG, 4527delGA, 4528delAA, 4529delAG, 4530delGG, 4531delGC,
4532delCC, 4533delCT, 4534delTT, 4535delTT, 4536delTC, 4537delCT, 4538delTG,
4539delGC, 4540delCT, 4541delTG, 4542delGA, 4543delAC, 4544delCA, 4545delAA,
4546delAG, 4547delGT, 4548delTT, 4549delTT, 4550delTG, 4551delGA, 4552delAG,
4553delGG, 4554delGT, 4555delTG, 4556delGT, 4557delTC, 4558delCT, 4559delTG,
4560delGC, 4561delCA, 4562delAG, 4563delGA, 4564delAT, 4565delTA, 4566delAG,
4567delGT, 4568delTT, 4569delTC, 4570delCT, 4571delTA, 4572delAC, 4573delCC,
4574delCA, 4575delAG, 4576delGT, 4577delTA, 4578delAA, 4579delAA, 4580delAA,
4581delAA, 4582delAT, 4583delTA, 4584delAA, 4585delAA, 4586delAG, 4587delGA,
4588delAA, 4589delAC, 4590delCC, 4591delCA, 4592delAG, 4593delGG, 4594delGA,
4595delAG, 4596delGT, 4597delTG, 4598delGG, 4599delGA, 4600delAA, 4601delAA,
4602delAG, 4603delGG, 4604delGT, 4605delTC, 4606delCA, 4607delAT, 4608delTC,
4609delCC, 4610delCC, 4611delCC, 4612delCT, 4613delTT, 4614delTC, 4615delCT,
4616delTA, 4617delAA, 4618delAA, 4619delAT, 4620delTG, 4621delGC, 4622delCC,
4623delCC, 4624delCA, 4625delAT, 4626delTC, 4627delCA, 4628delAT, 4629delTT,
4630delTA, 4631delAG, 4632delGA, 4633delAT, 4634delTG, 4635delGA, 4636delAT,
4637delTA, 4638delAG, 4639delGG, 4640delGT, 4641delTG, 4642delGG, 4643delGT,
4644delTA, 4645delAC, 4646delCA, 4647delAT, 4648delTG, 4649delGC, 4650delCA,
4651delAC, 4652delCA, 4653delAG, 4654delGT, 4655delTT, 4656delTG, 4657delGC,
4658delCT, 4659delTC, 4660delCT, 4661delTG, 4662delGG, 4663delGG, 4664delGA,
4665delAG, 4666delGT, 4667delTC, 4668delCT, 4669delTT, 4670delTC, 4671delCA,
4672delAG, 4673delGA, 4674delAA, 4675delAT, 4676delTA, 4677delAG, 4678delGA,
4679delAA, 4680delAA, 4681delAC, 4682delCT, 4683delTA, 4684delAC, 4685delCC,
4686delCC, 4687delCA, 4688delAT, 4689delTC, 4690delCT, 4691delTC, 4692delCA,
4693delAA, 4694delAG, 4695delGA, 4696delAG, 4697delGG, 4698delGA, 4699delAG,
4700delGC, 4701delCT, 4702delTC, 4703delCA, 4704delAT, 4705delTT, 4706delTA,
4707delAA, 4708delAG, 4709delGG, 4710delGT, 4711delTT, 4712delTG, 4713delGT,
4714delTT, 4715delTG, 4716delGA, 4717delAT, 4718delTG, 4719delGT, 4720delTG,
4721delGG, 4722delGA, 4723delAG, 4724delGG, 4725delGA, 4726delAG, 4727delGC,
4728delCA, 4729delAA, 4730delAC, 4731delCA, 4732delAG, 4733delGC, 4734delCT,
4735delTG, 4736delGG, 4737delGA, 4738delAA, 4739delAG, 4740delGA, 4741delAG,
4742delGT, 4743delTC, 4744delCT, 4745delTG, 4746delGG, 4747delGG, 4748delGC,
4749delCC, 4750delCA, 4751delAC, 4752delCA, 4753delAC, 4754delCG, 4755delGA,
4756delAT, 4757delTT, 4758delTT, 4759delTG, 4760delGA, 4761delAC, 4762delCG,
4763delGG, 4764delGA, 4765delAA, 4766delAA, 4767delAC, 4768delCA, 4769delAT,
4770delTC, 4771delCT, 4772delTT, 4773delTA, 4774delAC, 4775delCT, 4776delTT,
4777delTG, 4778delGC, 4779delCC, 4280delCA, 4781delAA, 4782delAG, 4783delGG,
4784delGC, 4785delCA, 4786delAA, 4787delAG, 4788delGA, 4789delAT, 4790delTC,
4791delCT, 4792delTA, 4793delAG, 4794delGA, 4795delAG, 4796delGG, 4797delGG,
4798delGA, 4799delAA, 4800delAC, 4801delCC, 4802delCC, 4803delCC, 4804delCT,
4805delTT, 4806delTA, 4807delAC, 4808delCC, 4809delCT, 4810delTG, 4811delGG,
4812delGA, 4813delAA, 4814delAT, 4815delTC, 4816delCT, 4817delTG, 4818delGG,
4819delGA, 4820delAA, 4821delAT, 4822delTC, 4823delCA, 4824delAG, 4825delGC,
4826delCC, 4827delCT, 4828delTC, 4829delCT, 4830delTT, 4831delTC, 4832delCT,
4833delTC, 4834delCT, 4835delTG, 4836delGA, 4837delAT, 4838delTG, 4839delGA,
4840delAC, 4841delCC, 4842delCC, 4843delCT, 4844delTG, 4845delGA, 4846delAA,
4847delAT, 4848delTC, 4849delCT, 4850delTG, 4851delGA, 4852delAT, 4853delTC,
4854delCC, 4855delCT, 4856delTT, 4857delTC, 4858delCT, 4859delTG, 4860delGA,
4861delAA, 4862delAG, 4863delGA, 4864delAC, 4865delCA, 4866delAG, 4867delGA,
4868delAG, 4869delGC, 4870delCC, 4871delCC, 4872delCC, 4873delCA, 4874delAG,
4875delGA, 4876delAG, 4877delGT, 4878delTC, 4879delCA, 4880delAG, 4881delGC,
4882delCT, 4883delTC, 4884delCG, 4885delGT, 4886delTG, 4887delGT, 4888delTT,
4889delTG, 4890delGG, 4891delGC, 4892delCA, 4893delAA, 4894delAC, 4895delCA,
4896delAT, 4897delTA, 4898delAC, 4899delCC, 4900delCA, 4901delAT, 4902delTC,
4903delCT, 4904delTT, 4905delTC, 4906delCA, 4907delAA, 4908delAC, 4909delCC,
4910delCT, 4911delTC, 4912delCT, 4913delTG, 4914delGC, 4915delCA, 4916delAT,
4917delTT, 4918delTG, 4919delGA, 4920delAA, 4921delAA, 4922delAG, 4923delGT,
4924delTT, 4925delTC, 4926delCC, 4927delCC, 4928delCC, 4929delCA, 4930delAA,
4931delAT, 4932delTT, 4933delTG, 4934delGA, 4935delAA, 4936delAA, 4937delAG,
4938delGT, 4939delTT, 4940delTG, 4941delGC, 4942delCA, 4943delAG, 4944delGA,
4945delAA, 4946delAT, 4947delTC, 4948delCT, 4949delTG, 4950delGC, 4951delCC,
4952delCC, 4953delCA, 4954delAG, 4955delGG, 4956delGG, 4957delGT, 4958delTC,
4959delCC, 4960delCA, 4961delAG, 4962delGC, 4963delCT, 4964delTG, 4965delGC,
4966delCT, 4967delTG, 4968delGC, 4969delCT, 4970delTC, 4971delCA, 4972delAT,
4973delTA, 4974delAC, 4975delCT, 4976delTA, 4977delAC, 4978delCT, 4979delTG,
4980delGA, 4981delAT, 4982delTA, 4983delAC, 4984delCT, 4985delTG, 4986delGC,
4987delCT, 4988delTG, 4989delGG, 4990delGG, 4991delGT, 4992delTA, 4993delAT,
4994delTA, 4995delAA, 4996delAT, 4997delTG, 4998delGC, 4999delCA, 5000delAA,
5001delAT, 5002delTG, 5003delGG, 5004delGA, 5005delAA, 5006delAG, 5007delGA,
5008delAA, 5009delAA, 5010delAG, 5011delGT, 5012delTG, 5013delGT, 5014delTG,
5015delGA, 5016delAG, 5017delGC, 5018delCA, 5019delAG, 5020delGG, 5021delGG,
5022delGA, 5023delAG, 5024delGA, 5025delAA, 5026delAG, 5027delGC, 5028delCC,
5029delCA, 5030delAG, 5031delGA, 5032delAA, 5033delAT, 5034delTT, 5035delTG,
5036delGA, 5037delAC, 5038delCA, 5039delAG, 5040delGC, 5041delCT, 5042delTT,
5043delTC, 5044delCA, 5045delAA, 5046delAC, 5047delCA, 5048delAG, 5049delGA,
5050delAA, 5051delAA, 5052delAG, 5053delGG, 5054delGG, 5055delGT, 5056delTC,
5057delCA, 5058delAA, 5059delAC, 5060delCA, 5061delAA, 5062delAA, 5063delAA,
5064delAG, 5065delGA, 5066delAA, 5067delAT, 5068delTG, 5069delGT, 5070delTC,
5071delCC, 5072delCA, 5073delAT, 5074delTG, 5075delGG, 5076delGT, 5077delTG,
5078delGG, 5079delGT, 5080delTG, 5081delGT, 5082delTC, 5083delCT, 5084delTG,
5085delGG, 5086delGC, 5087delCC, 5088delCT, 5089delTG, 5090delGA, 5091delAC,
5092delCC, 5093delCC, 5094delCC, 5095delCA, 5096delAG, 5097delGA, 5098delAA,
5099delAG, 5100delGA, 5101delAA, 5102delAT, 5103delTT, 5104delTT, 5105delTA,
5106delAT, 5107delTG, 5108delGC, 5109delCT, 5110delTC, 5111delCG, 5112delGT,
5113delTG, 5114delGT, 5115delTA, 5116delAC, 5117delCA, 5118delAA, 5119delAG,
5120delGT, 5121delTT, 5122delTT, 5123delTG, 5124delGC, 5125delCC, 5126delCA,
5127delAG, 5128delGA, 5129delAA, 5130delAA, 5131delAA, 5132delAC, 5133delCA,
5134delAC, 5135delCC, 5136delCA, 5137delAC, 5138delCA, 5139delAT, 5140delTC,
5141delCA, 5142delAC, 5143delCT, 5144delTT, 5145delTT, 5146delTA, 5147delAA,
5148delAC, 5149delCT, 5150delTA, 5151delAA, 5152delAT, 5153delTC, 5154delCT,
5155delTA, 5156delAA, 5157delAT, 5158delTT, 5159delTA, 5160delAC, 5161delCT,
5162delTG, 5163delGA, 5164delAA, 5165delAG, 5166delGA, 5167delAG, 5168delGA,
5169delAC, 5170delCT, 5171delTA, 5172delAC, 5173delCT, 5174delTC, 5175delCA,
5176delAT, 5177delTG, 5178delGT, 5179delTT, 5180delTG, 5181delGT, 5182delTT,
5183delTA, 5184delAT, 5185delTG, 5186delGA, 5187delAA, 5188delAA, 5189delAA,
5190delAC, 5191delCA, 5192delAG, 5193delGA, 5194delAT, 5195delTG, 5196delGC,
5197delCT, 5198delTG, 5199delGA, 5200delAG, 5201delGT, 5202delTT, 5203delTT,
5204delTG, 5205delGT, 5206delTG, 5207delGT, 5208delTG, 5209delGT, 5210delTG,
5211delGA, 5212delAA, 5213delAC, 5214delCG, 5215delGG, 5216delGA, 5217delAC,
5218delCA, 5219delAC, 5220delCT, 5221delTG, 5222delGA, 5223delAA, 5224delAA,
5225delAT, 5226delTA, 5227delAT, 5228delTT, 5229delTT, 5230delTT, 5231delTC,
5232delCT, 5233delTA, 5234delAG, 5235delGG, 5236delGA, 5237delAA, 5238delAT,
5239delTT, 5240delTG, 5241delGC, 5242delCG, 5243delGG, 5244delGG, 5245delGA,
5246delAG, 5247delGG, 5248delGA, 5249delAA, 5250delAA, 5251delAA, 5252delAT,
5253delTG, 5254delGG, 5255delGG, 5256delGT, 5257delTA, 5258delAG, 5259delGT,
5260delTT, 5261delTA, 5262delAG, 5263delGC, 5264delCT, 5265delTA, 5266delAT,
5267delTT, 5268delTT, 5269delTC, 5270delCT, 5271delTG, 5272delGG, 5273delGG,
5274delGT, 5275delTG, 5276delGA, 5277delAC, 5278delCC, 5279delCC, 5280delCA,
5281delAG, 5282delGT, 5283delTC, 5284delCT, 5285delTA, 5286delAT, 5287delTT,
5288delTA, 5289delAA, 5290delAA, 5291delAG, 5292delGA, 5293delAA, 5294delAA,
5295delAG, 5296delGA, 5297delAA, 5298delAA, 5299delAA, 5300delAA, 5301delAT,
5302delTG, 5303delGC, 5304delCT, 5305delTG, 5306delGA, 5307delAA, 5308delAT,
5309delTG, 5310delGA, 5311delAG, 5312delGC, 5313delCA, 5314delAT, 5315delTG,
5316delGA, 5317delAT, 5318delTT, 5319delTT, 5320delTT, 5321delTG, 5322delGA,
5323delAA, 5324delAG, 5325delGT, 5126delTC, 5327delCA, 5328delAG, 5329delGA,
5330delAG, 5331delGG, 5332delGA, 5333delAG, 5334delGA, 5335delAT, 5336delTG,
5337delGT, 5338delTG, 5339delGG, 5340delGT, 5341delTC, 5342delCA, 5343delAA,
5344delAT, 5345delTG, 5346delGG, 5347delGA, 5348delAA, 5349delAG, 5350delGA,
5351delAA, 5352delAA, 5353delAC, 5354delCC, 5355delCA, 5356delAC, 5357delCC,
5358delCA, 5359delAA, 5360delAG, 5361delGG, 5362delGT, 5363delTC, 5364delCC,
5365delCA, 5366delAA, 5367delAA, 5368delAG, 5369delGC, 5370delCG, 5371delGA,
5372delAG, 5373delGC, 5374delCA, 5375delAA, 5376delAG, 5377delGA, 5378delAG,
5379delGA, 5380delAA, 5381delAT, 5382delTC, 5383delCC, 5384delCC, 5385delCA,
5386delAG, 5387delGG, 5388delGA, 5389delAC, 5390delCA, 5391delAG, 5392delGA,
5393delAA, 5394delAA, 5395delAG, 5396delGA, 5397delAT, 5398delTC, 5399delCT,
5400delTT, 5401delTC, 5402delCA, 5403delAG, 5404delGG, 5405delGG, 5406delGG,
5407delGG, 5408delGC, 5409delCT, 5410delTA, 5411delAG, 5412delGA, 5413delAA,
5414delAA, 5415delAT, 5416delTC, 5417delCT, 5418delTG, 5419delGT, 5420delTT,
5421delTG, 5422delGC, 5423delCT, 5424delTA, 5425delAT, 5426delTG, 5427delGG,
5428delGG, 5429delGC, 5430delCC, 5431delCC, 5432delCT, 5433delTT, 5434delTC,
5435delCA, 5436delAC, 5437delCC, 5438delCA, 5439delAA, 5440delAC, 5441delCA,
5442delAT, 5443delTG, 5444delGC, 5445delCC, 5446delCC, 5447delCA, 5448delAC,
5449delCA, 5450delAG, 5451delGA, 5452delAT, 5453delTC, 5454delCA, 5455delAA,
5456delAC, 5457delCT, 5458delTG, 5459delGG, 5460delGA, 5461delAA, 5462delAT,
5463delTG, 5464delGG, 5465delGA, 5466delAT, 5467delTG, 5468delGG, 5469delGT,
5470delTA, 5471delAC, 5472delCA, 5473delAG, 5474delGC, 5475delCT, 5476delTG,
5477delGT, 5478delTG, 5479delGT, 5480delTG, 5481delGG, 5482delGT, 5483delTG,
5484delGC, 5485delCT, 5486delTT, 5487delTC, 5488delCT, 5489delTG, 5490delGT,
5491delTG, 5492delGG, 5493delGT, 5494delTG, 5495delGA, 5496delAA, 5497delAG,
5498delGG, 5499delGA, 5500delAG, 5501delGC, 5502delCT, 5503delTT, 5504delTT,
5505delTC, 5506delCA, 5507delAT, 5508delTC, 5509delCA, 5510delAT, 5511delTT,
5512delTC, 5513delCA, 5514delAC, 5515delCC, 5516delCC, 5517delCT, 5518delTT,
5519delTG, 5520delGG, 5521delGC, 5522delCA, 5523delAC, 5524delCA, 5525delAG,
5526delGG, 5527delGT, 5528delTG, 5529delGT, 5530delTC, 5531delCC, 5532delCA,
5533delAC, 5534delCC, 5535delCC, 5536delCA, 5537delAA, 5538delAT, 5539delTT,
5540delTG, 5541delGT, 5542delTG, 5543delGG, 5544delGT, 5545delTT, 5546delTG,
5547delGT, 5548delTG, 5549delGC, 5550delCA, 5551delAG, 5552delGC, 5553delCC,
5554delCA, 5555delAG, 5556delGA, 5557delAT, 5558delTG, 5559delGC, 5560delCC,
5561delCT, 5562delTG, 5563delGG, 5564delGA, 5565delAC, 5566delCA, 5567delAG,
5568delGA, 5569delAG, 5570delGG, 5571delGA, 5572delAC, 5573delCA, 5574delAA,
5575delAT, 5576delTG, 5577delGG, 5578delGC, 5579delCT, 5580delTT, 5581delTC,
5582delCC, 5583delCA, 5584delAT, 5585delTG, 5586delGC, 5587delCA, 5588delAA,
5589delAT, 5590delTT, 5591delTG, 5592delGG, 5593delGG, 5594delGC, 5595delCA,
5596delAG, 5597delGA, 5598delAT, 5599delTG, 5600delGT, 5601delTG, 5602delGT,
5603delTG, 5635delTG, 5654delCC, 5660delCC, 5708delCT.

TABLE 11
List of One Base Insertions
(N = A, T, G, or C)

122insN, 123insN, 124insN, 125insN, 126insN, 127insN, 128insN, 129insN, 130insN, 131insN,
132insN, 133insN, 134insN, 135insN, 136insN, 137insN, 138insN, 139insN, 140insN, 141insN,
142insN, 143insN, 144insN, 145insN, 146insN, 147insN, 148insN, 149insN, 150insN, 151insN,
152insN, 153insN, 154insN, 155insN, 156insN, 157insN, 158insN, 159insN, 160insN, 161insN,
162insN, 163insN, 164insN, 165insN, 166insN, 167insN, 168insN, 169insN, 170insN, 171insN,
172insN, 173insN, 174insN, 175insN, 176insN, 177insN, 178insN, 179insN, 180insN, 181insN,
182insN, 183insN, 184insN, 185insN, 186insN, 187insN, 188insN, 189insN, 190insN, 191insN,
192insN, 193insN, 194insN, 195insN, 196insN, 197insN, 198insN, 199insN, 200insN, 201insN,
202insN, 203insN, 204insN, 205insN, 206insN, 207insN, 208insN, 209insN, 210insN, 211insN,
212insN, 213insN, 214insN, 215insN, 216insN, 217insN, 218insN, 219insN, 220insN, 221insN,
222insN, 223insN, 224insN, 225insN, 226insN, 227insN, 228insN, 229insN, 230insN, 231insN,
232insN, 233insN, 234insN, 235insN, 236insN, 237insN, 238insN, 239insN, 240insN, 241insN,
242insN, 243insN, 244insN, 245insN, 246insN, 247insN, 248insN, 249insN, 250insN, 251insN,
252insN, 253insN, 254insN, 255insN, 256insN, 257insN, 258insN, 259insN, 260insN, 261insN,
262insN, 263insN, 264insN, 265insN, 266insN, 267insN, 268insN, 269insN, 270insN, 271insN,
272insN, 273insN, 274insN, 275insN, 276insN, 277insN, 278insN, 279insN, 280insN, 281insN,
282insN, 283insN, 284insN, 285insN, 286insN, 287insN, 288insN, 289insN, 290insN, 291insN,
292insN, 293insN, 294insN, 295insN, 296insN, 297insN, 298insN, 299insN, 300insN, 301insN,
302insN, 303insN, 304insN, 305insN, 306insN, 307insN, 308insN, 309insN, 310insN, 311insN,
312insN, 313insN, 314insN, 315insN, 316insN, 317insN, 318insN, 319insN, 320insN, 321insN,
322insN, 323insN, 324insN, 325insN, 326insN, 327insN, 328insN, 329insN, 330insN, 331insN,
332insN, 333insN, 334insN, 335insN, 336insN, 337insN, 338insN, 339insN, 340insN, 341insN,
342insN, 343insN, 344insN, 345insN, 346insN, 347insN, 348insN, 349insN, 350insN, 351insN,
352insN, 353insN, 354insN, 355insN, 356insN, 357insN, 358insN, 359insN, 360insN, 361insN,
362insN, 363insN, 364insN, 365insN, 366insN, 367insN, 368insN, 369insN, 370insN, 371insN,
372insN, 373insN, 374insN, 375insN, 376insN, 377insN, 378insN, 379insN, 380insN, 381insN,
382insN, 383insN, 384insN, 385insN, 386insN, 387insN, 388insN, 389insN, 390insN, 391insN,
392insN, 393insN, 394insN, 395insN, 396insN, 397insN, 398insN, 399insN, 400insN, 401insN,
402insN, 403insN, 404insN, 405insN, 406insN, 407insN, 408insN, 409insN, 410insN, 411insN,
412insN, 413insN, 414insN, 415insN, 416insN, 417insN, 418insN, 419insN, 420insN, 421insN,
422insN, 423insN, 424insN, 425insN, 426insN, 427insN, 428insN, 429insN, 430insN, 431insN,
432insN, 433insN, 434insN, 435insN, 436insN, 437insN, 438insN, 439insN, 440insN, 441insN,
442insN, 443insN, 444insN, 445insN, 446insN, 447insN, 448insN, 449insN, 450insN, 451insN,
452insN, 453insN, 454insN, 455insN, 456insN, 457insN, 458insN, 459insN, 460insN, 461insN,
462insN, 463insN, 464insN, 465insN, 466insN, 467insN, 468insN, 469insN, 470insN, 471insN,
472insN, 473insN, 474insN, 475insN, 476insN, 477insN, 478insN, 479insN, 480insN, 481insN,
482insN, 483insN, 484insN, 485insN, 486insN, 487insN, 488insN, 489insN, 490insN, 491insN,
492insN, 493insN, 494insN, 495insN, 496insN, 497insN, 498insN, 499insN, 500insN, 501insN,
502insN, 503insN, 504insN, 505insN, 506insN, 507insN, 508insN, 509insN, 510insN, 511insN,
512insN, 513insN, 514insN, 515insN, 516insN, 517insN, 518insN, 519insN, 520insN, 521insN,
522insN, 523insN, 524insN, 525insN, 526insN, 527insN, 528insN, 529insN, 530insN, 531insN,
532insN, 533insN, 534insN, 535insN, 536insN, 537insN, 538insN, 539insN, 540insN, 541insN,
542insN, 543insN, 544insN, 545insN, 546insN, 547insN, 548insN, 549insN, 550insN, 551insN,
552insN, 553insN, 554insN, 555insN, 556insN, 557insN, 558insN, 559insN, 560insN, 561insN,
562insN, 563insN, 564insN, 565insN, 566insN, 567insN, 568insN, 569insN, 570insN, 571insN,
572insN, 573insN, 574insN, 575insN, 576insN, 577insN, 578insN, 579insN, 580insN, 581insN,
582insN, 583insN, 584insN, 585insN, 586insN, 587insN, 588insN, 589insN, 590insN, 591insN,
592insN, 593insN, 594insN, 595insN, 596insN, 597insN, 598insN, 599insN, 600insN, 601insN,
602insN, 603insN, 604insN, 605insN, 606insN, 607insN, 608insN, 609insN, 610insN, 611insN,
612insN, 613insN, 614insN, 615insN, 616insN, 617insN, 618insN, 619insN, 620insN, 621insN,
622insN, 623insN, 624insN, 625insN, 626insN, 627insN, 628insN, 629insN, 630insN, 631insN,
632insN, 633insN, 634insN, 635insN, 636insN, 637insN, 638insN, 639insN, 640insN, 641insN,
642insN, 643insN, 644insN, 645insN, 646insN, 647insN, 648insN, 649insN, 650insN, 651insN,
652insN, 653insN, 654insN, 655insN, 656insN, 657insN, 658insN, 659insN, 660insN, 661insN,
662insN, 663insN, 664insN, 665insN, 666insN, 667insN, 668insN, 669insN, 670insN, 671insN,
672insN, 673insN, 674insN, 675insN, 676insN, 677insN, 678insN, 679insN, 680insN, 681insN,
682insN, 683insN, 684insN, 685insN, 686insN, 687insN, 688insN, 689insN, 690insN, 691insN,
692insN, 693insN, 694insN, 695insN, 696insN, 697insN, 698insN, 699insN, 700insN, 701insN,
702insN, 703insN, 704insN, 705insN, 706insN, 707insN, 708insN, 709insN, 710insN, 711insN,
712insN, 713insN, 714insN, 715insN, 716insN, 717insN, 718insN, 719insN, 720insN, 721insN,
722insN, 723insN, 724insN, 725insN, 726insN, 727insN, 728insN, 729insN, 730insN, 731insN,
732insN, 733insN, 734insN, 735insN, 736insN, 737insN, 738insN, 739insN, 740insN, 741insN,
742insN, 743insN, 744insN, 745insN, 746insN, 747insN, 748insN, 749insN, 750insN, 751insN,
752insN, 753insN, 754insN, 755insN, 756insN, 757insN, 758insN, 759insN, 760insN, 761insN,
762insN, 763insN, 764insN, 765insN, 766insN, 767insN, 768insN, 769insN, 770insN, 771insN,
772insN, 773insN, 774insN, 775insN, 776insN, 777insN, 778insN, 779insN, 780insN, 781insN,
782insN, 783insN, 784insN, 785insN, 786insN, 787insN, 788insN, 789insN, 790insN, 791insN,
792insN, 793insN, 794insN, 795insN, 796insN, 797insN, 798insN, 799insN, 800insN, 801insN,
802insN, 803insN, 804insN, 805insN, 806insN, 807insN, 808insN, 809insN, 810insN, 811insN,
812insN, 813insN, 814insN, 815insN, 816insN, 817insN, 818insN, 819insN, 820insN, 821insN,
822insN, 823insN, 824insN, 825insN, 826insN, 827insN, 828insN, 829insN, 830insN, 831insN,
832insN, 833insN, 834insN, 835insN, 836insN, 837insN, 838insN, 839insN, 840insN, 841insN,
842insN, 843insN, 844insN, 845insN, 846insN, 847insN, 848insN, 849insN, 850insN, 851insN,
852insN, 853insN, 854insN, 855insN, 856insN, 857insN, 858insN, 859insN, 860insN, 861insN,
862insN, 863insN, 864insN, 865insN, 866insN, 867insN, 868insN, 869insN, 870insN, 871insN,
872insN, 873insN, 874insN, 875insN, 876insN, 877insN, 878insN, 879insN, 880insN, 881insN,
882insN, 883insN, 884insN, 885insN, 886insN, 887insN, 888insN, 889insN, 890insN, 891insN,
892insN, 893insN, 894insN, 895insN, 896insN, 897insN, 898insN, 899insN, 900insN, 901insN,
902insN, 903insN, 904insN, 905insN, 906insN, 907insN, 908insN, 909insN, 910insN, 911insN,
912insN, 913insN, 914insN, 915insN, 916insN, 917insN, 918insN, 919insN, 920insN, 921insN,
922insN, 923insN, 924insN, 925insN, 926insN, 927insN, 928insN, 929insN, 930insN, 931insN,
932insN, 933insN, 934insN, 935insN, 936insN, 937insN, 938insN, 939insN, 940insN, 941insN,
942insN, 943insN, 944insN, 945insN, 946insN, 947insN, 948insN, 949insN, 950insN, 951insN,
952insN, 953insN, 954insN, 955insN, 956insN, 957insN, 958insN, 959insN, 960insN, 961insN,
962insN, 963insN, 964insN, 965insN, 966insN, 967insN, 968insN, 969insN, 970insN, 971insN,
972insN, 973insN, 974insN, 975insN, 976insN, 977insN, 978insN, 979insN, 980insN, 981insN,
982insN, 983insN, 984insN, 985insN, 986insN, 987insN, 988insN, 989insN, 990insN, 991insN,
992insN, 993insN, 994insN, 995insN, 996insN, 997insN, 998insN, 999insN, 1000insN,
1001insN, 1002insN, 1003insN, 1004insN, 1005insN, 1006insN, 1007insN, 1008insN,
1009insN, 1010insN, 1011insN, 1012insN, 1013insN, 1014insN, 1015insN, 1016insN,
1017insN, 1018insN, 1019insN, 1020insN, 1021insN, 1022insN, 1023insN, 1024insN,
1025insN, 1026insN, 1027insN, 1028insN, 1029insN, 1030insN, 1031insN, 1032insN,
1033insN, 1034insN, 1035insN, 1036insN, 1037insN, 1038insN, 1039insN, 1040insN,
1041insN, 1042insN, 1043insN, 1044insN, 1045insN, 1046insN, 1047insN, 1048insN,
1049insN, 1050insN, 1051insN, 1052insN, 1053insN, 1054insN, 1055insN, 1056insN,
1057insN, 1058insN, 1059insN, 1060insN, 1061insN, 1062insN, 1063insN, 1064insN,
1065insN, 1066insN, 1067insN, 1068insN, 1069insN, 1070insN, 1071insN, 1072insN,
1073insN, 1074insN, 1075insN, 1076insN, 1077insN, 1078insN, 1079insN, 1080insN,
1081insN, 1082insN, 1083insN, 1084insN, 1085insN, 1086insN, 1087insN, 1088insN,
1089insN, 1090insN, 1091insN, 1092insN, 1093insN, 1094insN, 1095insN, 1096insN,
1097insN, 1098insN, 1099insN, 1100insN, 1101insN, 1102insN, 1103insN, 1104insN,
1105insN, 1106insN, 1107insN, 1108insN, 1109insN, 1110insN, 1111insN, 1112insN,
1113insN, 1114insN, 1115insN, 1116insN, 1117insN, 1118insN, 1119insN, 1120insN,
1121insN, 1122insN, 1123insN, 1124insN, 1125insN, 1126insN, 1127insN, 1128insN,
1129insN, 1130insN, 1131insN, 1132insN, 1133insN, 1134insN, 1135insN, 1136insN,
1137insN, 1138insN, 1139insN, 1140insN, 1141insN, 1142insN, 1143insN, 1144insN,
1145insN, 1146insN, 1147insN, 1148insN, 1149insN, 1150insN, 1151insN, 1152insN,
1153insN, 1154insN, 1155insN, 1156insN, 1157insN, 1158insN, 1159insN, 1160insN,
1161insN, 1162insN, 1163insN, 1164insN, 1165insN, 1166insN, 1167insN, 1168insN,
1169insN, 1170insN, 1171insN, 1172insN, 1173insN, 1174insN, 1175insN, 1176insN,
1177insN, 1178insN, 1179insN, 1180insN, 1181insN, 1182insN, 1183insN, 1184insN,
1185insN, 1186insN, 1187insN, 1188insN, 1189insN, 1190insN, 1191insN, 1192insN,
1193insN, 1194insN, 1195insN, 1196insN, 1197insN, 1198insN, 1199insN, 1200insN,
1201insN, 1202insN, 1203insN, 1204insN, 1205insN, 1206insN, 1207insN, 1208insN,
1209insN, 1210insN, 1211insN, 1212insN, 1213insN, 1214insN, 1215insN, 1216insN,
1217insN, 1218insN, 1219insN, 1220insN, 1221insN, 1222insN, 1223insN, 1224insN,
1225insN, 1226insN, 1227insN, 1228insN, 1229insN, 1230insN, 1231insN, 1232insN,
1233insN, 1234insN, 1235insN, 1236insN, 1237insN, 1238insN, 1239insN, 1240insN,
1241insN, 1242insN, 1243insN, 1244insN, 1245insN, 1246insN, 1247insN, 1248insN,
1249insN, 1250insN, 1251insN, 1252insN, 1253insN, 1254insN, 1255insN, 1256insN,
1257insN, 1258insN, 1259insN, 1260insN, 1261insN, 1262insN, 1263insN, 1264insN,
1265insN, 1266insN, 1267insN, 1268insN, 1269insN, 1270insN, 1271insN, 1272insN,
1273insN, 1274insN, 1275insN, 1276insN, 1277insN, 1278insN, 1279insN, 1280insN,
1281insN, 1282insN, 1283insN, 1284insN, 1285insN, 1286insN, 1287insN, 1288insN,
1289insN, 1290insN, 1291insN, 1292insN, 1293insN, 1294insN, 1295insN, 1296insN,
1297insN, 1298insN, 1299insN, 1300insN, 1301insN, 1302insN, 1303insN, 1304insN,
1305insN, 1306insN, 1307insN, 1308insN, 1309insN, 1310insN, 1311insN, 1312insN,
1313insN, 1314insN, 1315insN, 1316insN, 1317insN, 1318insN, 1319insN, 1320insN,
1321insN, 1322insN, 1323insN, 1324insN, 1325insN, 1326insN, 1327insN, 1328insN,
1329insN, 1330insN, 1331insN, 1332insN, 1333insN, 1334insN, 1335insN, 1336insN,
1337insN, 1338insN, 1339insN, 1340insN, 1341insN, 1342insN, 1343insN, 1344insN,
1345insN, 1346insN, 1347insN, 1348insN, 1349insN, 1350insN, 1351insN, 1352insN,
1353insN, 1354insN, 1355insN, 1356insN, 1357insN, 1358insN, 1359insN, 1360insN,
1361insN, 1362insN, 1363insN, 1364insN, 1365insN, 1366insN, 1367insN, 1368insN,
1369insN, 1370insN, 1371insN, 1372insN, 1373insN, 1374insN, 1375insN, 1376insN,
1377insN, 1378insN, 1379insN, 1380insN, 1381insN, 1382insN, 1383insN, 1384insN,
1385insN, 1386insN, 1387insN, 1388insN, 1389insN, 1390insN, 1391insN, 1392insN,
1393insN, 1394insN, 1395insN, 1396insN, 1397insN, 1398insN, 1399insN, 1400insN,
1401insN, 1402insN, 1403insN, 1404insN, 1405insN, 1406insN, 1407insN, 1408insN,
1409insN, 1410insN, 1411insN, 1412insN, 1413insN, 1414insN, 1415insN, 1416insN,
1417insN, 1418insN, 1419insN, 1420insN, 1421insN, 1422insN, 1423insN, 1424insN,
1425insN, 1426insN, 1427insN, 1428insN, 1429insN, 1430insN, 1431insN, 1432insN,
1433insN, 1434insN, 1435insN, 1436insN, 1437insN, 1438insN, 1439insN, 1440insN,
1441insN, 1442insN, 1443insN, 1444insN, 1445insN, 1446insN, 1447insN, 1448insN,
1449insN, 1450insN, 1451insN, 1452insN, 1453insN, 1454insN, 1455insN, 1456insN,
1457insN, 1458insN, 1459insN, 1460insN, 1461insN, 1462insN, 1463insN, 1464insN,
1465insN, 1466insN, 1467insN, 1468insN, 1469insN, 1470insN, 1471insN, 1472insN,
1473insN, 1474insN, 1475insN, 1476insN, 1477insN, 1478insN, 1479insN, 1480insN,
1481insN, 1482insN, 1483insN, 1484insN, 1485insN, 1486insN, 1487insN, 1488insN,
1489insN, 1490insN, 1491insN, 1492insN, 1493insN, 1494insN, 1495insN, 1496insN,
1497insN, 1498insN, 1499insN, 1500insN, 1501insN, 1502insN, 1503insN, 1504insN,
1505insN, 1506insN, 1507insN, 1508insN, 1509insN, 1510insN, 1511insN, 1512insN,
1513insN, 1514insN, 1515insN, 1516insN, 1517insN, 1518insN, 1519insN, 1520insN,
1521insN, 1522insN, 1523insN, 1524insN, 1525insN, 1526insN, 1527insN, 1528insN,
1529insN, 1530insN, 1531insN, 1532insN, 1533insN, 1534insN, 1535insN, 1536insN,
1537insN, 1538insN, 1539insN, 1540insN, 1541insN, 1542insN, 1543insN, 1544insN,
1545insN, 1546insN, 1547insN, 1548insN, 1549insN, 1550insN, 1551insN, 1552insN,
1553insN, 1554insN, 1555insN, 1556insN, 1557insN, 1558insN, 1559insN, 1560insN,
1561insN, 1562insN, 1563insN, 1564insN, 1565insN, 1566insN, 1567insN, 1568insN,
1569insN, 1570insN, 1571insN, 1572insN, 1573insN, 1574insN, 1575insN, 1576insN,
1577insN, 1578insN, 1579insN, 1580insN, 1581insN, 1582insN, 1583insN, 1584insN,
1585insN, 1586insN, 1587insN, 1588insN, 1589insN, 1590insN, 1591insN, 1592insN,
1593insN, 1594insN, 1595insN, 1596insN, 1597insN, 1598insN, 1599insN, 1600insN,
1601insN, 1602insN, 1603insN, 1604insN, 1605insN, 1606insN, 1607insN, 1608insN,
1609insN, 1610insN, 1611insN, 1612insN, 1613insN, 1614insN, 1615insN, 1616insN,
1617insN, 1618insN, 1619insN, 1620insN, 1621insN, 1622insN, 1623insN, 1624insN,
1625insN, 1626insN, 1627insN, 1628insN, 1629insN, 1630insN, 1631insN, 1632insN,
1633insN, 1634insN, 1635insN, 1636insN, 1637insN, 1638insN, 1639insN, 1640insN,
1641insN, 1642insN, 1643insN, 1644insN, 1645insN, 1646insN, 1647insN, 1648insN,
1649insN, 1650insN, 1651insN, 1652insN, 1653insN, 1654insN, 1655insN, 1656insN,
1657insN, 1658insN, 1659insN, 1660insN, 1661insN, 1662insN, 1663insN, 1664insN,
1665insN, 1666insN, 1667insN, 1668insN, 1669insN, 1670insN, 1671insN, 1672insN,
1673insN, 1674insN, 1675insN, 1676insN, 1677insN, 1678insN, 1679insN, 1680insN,
1681insN, 1682insN, 1683insN, 1684insN, 1685insN, 1686insN, 1687insN, 1688insN,
1689insN, 1690insN, 1691insN, 1692insN, 1693insN, 1694insN, 1695insN, 1696insN,
1697insN, 1698insN, 1699insN, 1700insN, 1701insN, 1702insN, 1703insN, 1704insN,
1705insN, 1706insN, 1707insN, 1708insN, 1709insN, 1710insN, 1711insN, 1712insN,
1713insN, 1714insN, 1715insN, 1716insN, 1717insN, 1718insN, 1719insN, 1720insN,
1721insN, 1722insN, 1723insN, 1724insN, 1725insN, 1726insN, 1727insN, 1728insN,
1729insN, 1730insN, 1731insN, 1732insN, 1733insN, 1734insN, 1735insN, 1736insN,
1737insN, 1738insN, 1739insN, 1740insN, 1741insN, 1742insN, 1743insN, 1744insN,
1745insN, 1746insN, 1747insN, 1748insN, 1749insN, 1750insN, 1751insN, 1752insN,
1753insN, 1754insN, 1755insN, 1756insN, 1757insN, 1758insN, 1759insN, 1760insN,
1761insN, 1762insN, 1763insN, 1764insN, 1765insN, 1766insN, 1767insN, 1768insN,
1769insN, 1770insN, 1771insN, 1772insN, 1773insN, 1774insN, 1775insN, 1776insN,
1777insN, 1778insN, 1779insN, 1780insN, 1781insN, 1782insN, 1783insN, 1784insN,
1785insN, 1786insN, 1787insN, 1788insN, 1789insN, 1790insN, 1791insN, 1792insN,
1793insN, 1794insN, 1795insN, 1796insN, 1797insN, 1798insN, 1799insN, 1800insN,
1801insN, 1802insN, 1803insN, 1804insN, 1805insN, 1806insN, 1807insN, 1808insN,
1809insN, 1810insN, 1811insN, 1812insN, 1813insN, 1814insN, 1815insN, 1816insN,
1817insN, 1818insN, 1819insN, 1820insN, 1821insN, 1822insN, 1823insN, 1824insN,
1825insN, 1826insN, 1827insN, 1828insN, 1829insN, 1830insN, 1831insN, 1832insN,
1833insN, 1834insN, 1835insN, 1836insN, 1837insN, 1838insN, 1839insN, 1840insN,
1841insN, 1842insN, 1843insN, 1844insN, 1845insN, 1846insN, 1847insN, 1848insN,
1849insN, 1850insN, 1851insN, 1852insN, 1853insN, 1854insN, 1855insN, 1856insN,
1857insN, 1858insN, 1859insN, 1860insN, 1861insN, 1862insN, 1863insN, 1864insN,
1865insN, 1866insN, 1867insN, 1868insN, 1869insN, 1870insN, 1871insN, 1872insN,
1873insN, 1874insN, 1875insN, 1876insN, 1877insN, 1878insN, 1879insN, 1880insN,
1881insN, 1882insN, 1883insN, 1884insN, 1885insN, 1886insN, 1887insN, 1888insN,
1889insN, 1890insN, 1891insN, 1892insN, 1893insN, 1894insN, 1895insN, 1896insN,
1897insN, 1898insN, 1899insN, 1900insN, 1901insN, 1902insN, 1903insN, 1904insN,
1905insN, 1906insN, 1907insN, 1908insN, 1909insN, 1910insN, 1911insN, 1912insN,
1913insN, 1914insN, 1915insN, 1916insN, 1917insN, 1918insN, 1919insN, 1920insN,
1921insN, 1922insN, 1923insN, 1924insN, 1925insN, 1926insN, 1927insN, 1928insN,
1929insN, 1930insN, 1931insN, 1932insN, 1933insN, 1934insN, 1935insN, 1936insN,
1937insN, 1938insN, 1939insN, 1940insN, 1941insN, 1942insN, 1943insN, 1944insN,
1945insN, 1946insN, 1947insN, 1948insN, 1949insN, 1950insN, 1951insN, 1952insN,
1953insN, 1954insN, 1955insN, 1956insN, 1957insN, 1958insN, 1959insN, 1960insN,
1961insN, 1962insN, 1963insN, 1964insN, 1965insN, 1966insN, 1967insN, 1968insN,
1969insN, 1970insN, 1971insN, 1972insN, 1973insN, 1974insN, 1975insN, 1976insN,
1977insN, 1978insN, 1979insN, 1980insN, 1981insN, 1982insN, 1983insN, 1984insN,
1985insN, 1986insN, 1987insN, 1988insN, 1989insN, 1990insN, 1991insN, 1992insN,
1993insN, 1994insN, 1995insN, 1996insN, 1997insN, 1998insN, 1999insN, 2000insN,
2001insN, 2002insN, 2003insN, 2004insN, 2005insN, 2006insN, 2007insN, 2008insN,
2009insN, 2010insN, 2011insN, 2012insN, 2013insN, 2014insN, 2015insN, 2016insN,
2017insN, 2018insN, 2019insN, 2020insN, 2021insN, 2022insN, 2023insN, 2024insN,
2025insN, 2026insN, 2027insN, 2028insN, 2029insN, 2030insN, 2031insN, 2032insN,
2033insN, 2034insN, 2035insN, 2036insN, 2037insN, 2038insN, 2039insN, 2040insN,
2041insN, 2042insN, 2043insN, 2044insN, 2045insN, 2046insN, 2047insN, 2048insN,
2049insN, 2050insN, 2051insN, 2052insN, 2053insN, 2054insN, 2055insN, 2056insN,
2057insN, 2058insN, 2059insN, 2060insN, 2061insN, 2062insN, 2063insN, 2064insN,
2065insN, 2066insN, 2067insN, 2068insN, 2069insN, 2070insN, 2071insN, 2072insN,
2073insN, 2074insN, 2075insN, 2076insN, 2077insN, 2078insN, 2079insN, 2080insN,
2081insN, 2082insN, 2083insN, 2084insN, 2085insN, 2086insN, 2087insN, 2088insN,
2089insN, 2090insN, 2091insN, 2092insN, 2093insN, 2094insN, 2095insN, 2096insN,
2097insN, 2098insN, 2099insN, 2100insN, 2101insN, 2102insN, 2103insN, 2104insN,
2105insN, 2106insN, 2107insN, 2108insN, 2109insN, 2110insN, 2111insN, 2112insN,
2113insN, 2114insN, 2115insN, 2116insN, 2117insN, 2118insN, 2119insN, 2120insN,
2121insN, 2122insN, 2123insN, 2124insN, 2125insN, 2126insN, 2127insN, 2128insN,
2129insN, 2130insN, 2131insN, 2132insN, 2133insN, 2134insN, 2135insN, 2136insN,
2137insN, 2138insN, 2139insN, 2140insN, 2141insN, 2142insN, 2143insN, 2144insN,
2145insN, 2146insN, 2147insN, 2148insN, 2149insN, 2150insN, 2151insN, 2152insN,
2153insN, 2154insN, 2155insN, 2156insN, 2157insN, 2158insN, 2159insN, 2160insN,
2161insN, 2162insN, 2163insN, 2164insN, 2165insN, 2166insN, 2167insN, 2168insN,
2169insN, 2170insN, 2171insN, 2172insN, 2173insN, 2174insN, 2175insN, 2176insN,
2177insN, 2178insN, 2179insN, 2180insN, 2181insN, 2182insN, 2183insN, 2184insN,
2185insN, 2186insN, 2187insN, 2188insN, 2189insN, 2190insN, 2191insN, 2192insN,
2193insN, 2194insN, 2195insN, 2196insN, 2197insN, 2198insN, 2199insN, 2200insN,
2201insN, 2202insN, 2203insN, 2204insN, 2205insN, 2206insN, 2207insN, 2208insN,
2209insN, 2210insN, 2211insN, 2212insN, 2213insN, 2214insN, 2215insN, 2216insN,
2217insN, 2218insN, 2219insN, 2220insN, 2221insN, 2222insN, 2223insN, 2224insN,
2225insN, 2226insN, 2227insN, 2228insN, 2229insN, 2230insN, 2231insN, 2232insN,
2233insN, 2234insN, 2235insN, 2236insN, 2237insN, 2238insN, 2239insN, 2240insN,
2241insN, 2242insN, 2243insN, 2244insN, 2245insN, 2246insN, 2247insN, 2248insN,
2249insN, 2250insN, 2251insN, 2252insN, 2253insN, 2254insN, 2255insN, 2256insN,
2257insN, 2258insN, 2259insN, 2260insN, 2261insN, 2262insN, 2263insN, 2264insN,
2265insN, 2266insN, 2267insN, 2268insN, 2269insN, 2270insN, 2271insN, 2272insN,
2273insN, 2274insN, 2275insN, 2276insN, 2277insN, 2278insN, 2279insN, 2280insN,
2281insN, 2282insN, 2283insN, 2284insN, 2285insN, 2286insN, 2287insN, 2288insN,
2289insN, 2290insN, 2291insN, 2292insN, 2293insN, 2294insN, 2295insN, 2296insN,
2297insN, 2298insN, 2299insN, 2300insN, 2301insN, 2302insN, 2303insN, 2304insN,
2305insN, 2306insN, 2307insN, 2308insN, 2309insN, 2310insN, 2311insN, 2312insN,
2313insN, 2314insN, 2315insN, 2316insN, 2317insN, 2318insN, 2319insN, 2320insN,
2321insN, 2322insN, 2323insN, 2324insN, 2325insN, 2326insN, 2327insN, 2328insN,
2329insN, 2330insN, 2331insN, 2332insN, 2333insN, 2334insN, 2335insN, 2336insN,
2337insN, 2338insN, 2339insN, 2340insN, 2341insN, 2342insN, 2343insN, 2344insN,
2345insN, 2346insN, 2347insN, 2348insN, 2349insN, 2350insN, 2351insN, 2352insN,
2353insN, 2354insN, 2355insN, 2356insN, 2357insN, 2358insN, 2359insN, 2360insN,
2361insN, 2362insN, 2363insN, 2364insN, 2365insN, 2366insN, 2367insN, 2368insN,
2369insN, 2370insN, 2371insN, 2372insN, 2373insN, 2374insN, 2375insN, 2376insN,
2377insN, 2378insN, 2379insN, 2380insN, 2381insN, 2382insN, 2383insN, 2384insN,
2385insN, 2386insN, 2387insN, 2388insN, 2389insN, 2390insN, 2391insN, 2392insN,
2393insN, 2394insN, 2395insN, 2396insN, 2397insN, 2398insN, 2399insN, 2400insN,
2401insN, 2402insN, 2403insN, 2404insN, 2405insN, 2406insN, 2407insN, 2408insN,
2409insN, 2410insN, 2411insN, 2412insN, 2413insN, 2414insN, 2415insN, 2416insN,
2417insN, 2418insN, 2419insN, 2420insN, 2421insN, 2422insN, 2423insN, 2424insN,
2425insN, 2426insN, 2427insN, 2428insN, 2429insN, 2430insN, 2431insN, 2432insN,
2433insN, 2434insN, 2435insN, 2436insN, 2437insN, 2438insN, 2439insN, 2440insN,
2441insN, 2442insN, 2443insN, 2444insN, 2445insN, 2446insN, 2447insN, 2448insN,
2449insN, 2450insN, 2451insN, 2452insN, 2453insN, 2454insN, 2455insN, 2456insN,
2457insN, 2458insN, 2459insN, 2460insN, 2461insN, 2462insN, 2463insN, 2464insN,
2465insN, 2466insN, 2467insN, 2468insN, 2469insN, 2470insN, 2471insN, 2472insN,
2473insN, 2474insN, 2475insN, 2476insN, 2477insN, 2478insN, 2479insN, 2480insN,
2481insN, 2482insN, 2483insN, 2484insN, 2485insN, 2486insN, 2487insN, 2488insN,
2489insN, 2490insN, 2491insN, 2492insN, 2493insN, 2494insN, 2495insN, 2496insN,
2497insN, 2498insN, 2499insN, 2500insN, 2501insN, 2502insN, 2503insN, 2504insN,
2505insN, 2506insN, 2507insN, 2508insN, 2509insN, 2510insN, 2511insN, 2512insN,
2513insN, 2514insN, 2515insN, 2516insN, 2517insN, 2518insN, 2519insN, 2520insN,
2521insN, 2522insN, 2523insN, 2524insN, 2525insN, 2526insN, 2527insN, 2528insN,
2529insN, 2530insN, 2531insN, 2532insN, 2533insN, 2534insN, 2535insN, 2536insN,
2537insN, 2538insN, 2539insN, 2540insN, 2541insN, 2542insN, 2543insN, 2544insN,
2545insN, 2546insN, 2547insN, 2548insN, 2549insN, 2550insN, 2551insN, 2552insN,
2553insN, 2554insN, 2555insN, 2556insN, 2557insN, 2558insN, 2559insN, 2560insN,
2561insN, 2562insN, 2563insN, 2564insN, 2565insN, 2566insN, 2567insN, 2568insN,
2569insN, 2570insN, 2571insN, 2572insN, 2573insN, 2574insN, 2575insN, 2576insN,
2577insN, 2578insN, 2579insN, 2580insN, 2581insN, 2582insN, 2583insN, 2584insN,
2585insN, 2586insN, 2587insN, 2588insN, 2589insN, 2590insN, 2591insN, 2592insN,
2593insN, 2594insN, 2595insN, 2596insN, 2597insN, 2598insN, 2599insN, 2600insN,
2601insN, 2602insN, 2603insN, 2604insN, 2605insN, 2606insN, 2607insN, 2608insN,
2609insN, 2610insN, 2611insN, 2612insN, 2613insN, 2614insN, 2615insN, 2616insN,
2617insN, 2618insN, 2619insN, 2620insN, 2621insN, 2622insN, 2623insN, 2624insN,
2625insN, 2626insN, 2627insN, 2628insN, 2629insN, 2630insN, 2631insN, 2632insN,
2633insN, 2634insN, 2635insN, 2636insN, 2637insN, 2638insN, 2639insN, 2640insN,
2641insN, 2642insN, 2643insN, 2644insN, 2645insN, 2646insN, 2647insN, 2648insN,
2649insN, 2650insN, 2651insN, 2652insN, 2653insN, 2654insN, 2655insN, 2656insN,
2657insN, 2658insN, 2659insN, 2660insN, 2661insN, 2662insN, 2663insN, 2664insN,
2665insN, 2666insN, 2667insN, 2668insN, 2669insN, 2670insN, 2671insN, 2672insN,
2673insN, 2674insN, 2675insN, 2676insN, 2677insN, 2678insN, 2679insN, 2680insN,
2681insN, 2682insN, 2683insN, 2684insN, 2685insN, 2686insN, 2687insN, 2688insN,
2689insN, 2690insN, 2691insN, 2692insN, 2693insN, 2694insN, 2695insN, 2696insN,
2697insN, 2698insN, 2699insN, 2700insN, 2701insN, 2702insN, 2703insN, 2704insN,
2705insN, 2706insN, 2707insN, 2708insN, 2709insN, 2710insN, 2711insN, 2712insN,
2713insN, 2714insN, 2715insN, 2716insN, 2717insN, 2718insN, 2719insN, 2720insN,
2721insN, 2722insN, 2723insN, 2724insN, 2725insN, 2726insN, 2727insN, 2728insN,
2729insN, 2730insN, 2731insN, 2732insN, 2733insN, 2734insN, 2735insN, 2736insN,
2737insN, 2738insN, 2739insN, 2740insN, 2741insN, 2742insN, 2743insN, 2744insN,
2745insN, 2746insN, 2747insN, 2748insN, 2749insN, 2750insN, 2751insN, 2752insN,
2753insN, 2754insN, 2755insN, 2756insN, 2757insN, 2758insN, 2759insN, 2760insN,
2761insN, 2762insN, 2763insN, 2764insN, 2765insN, 2766insN, 2767insN, 2768insN,
2769insN, 2770insN, 2771insN, 2772insN, 2773insN, 2774insN, 2775insN, 2776insN,
2777insN, 2778insN, 2779insN, 2780insN, 2781insN, 2782insN, 2783insN, 2784insN,
2785insN, 2786insN, 2787insN, 2788insN, 2789insN, 2790insN, 2791insN, 2792insN,
2793insN, 2794insN, 2795insN, 2796insN, 2797insN, 2798insN, 2799insN, 2800insN,
2801insN, 2802insN, 2803insN, 2804insN, 2805insN, 2806insN, 2807insN, 2808insN,
2809insN, 2810insN, 2811insN, 2812insN, 2813insN, 2814insN, 2815insN, 2816insN,
2817insN, 2818insN, 2819insN, 2820insN, 2821insN, 2822insN, 2823insN, 2824insN,
2825insN, 2826insN, 2827insN, 2828insN, 2829insN, 2830insN, 2831insN, 2832insN,
2833insN, 2834insN, 2835insN, 2836insN, 2837insN, 2838insN, 2839insN, 2840insN,
2841insN, 2842insN, 2843insN, 2844insN, 2845insN, 2846insN, 2847insN, 2848insN,
2849insN, 2850insN, 2851insN, 2852insN, 2853insN, 2854insN, 2855insN, 2856insN,
2857insN, 2858insN, 2859insN, 2860insN, 2861insN, 2862insN, 2863insN, 2864insN,
2865insN, 2866insN, 2867insN, 2868insN, 2869insN, 2870insN, 2871insN, 2872insN,
2873insN, 2874insN, 2875insN, 2876insN, 2877insN, 2878insN, 2879insN, 2880insN,
2881insN, 2882insN, 2883insN, 2884insN, 2885insN, 2886insN, 2887insN, 2888insN,
2889insN, 2890insN, 2891insN, 2892insN, 2893insN, 2894insN, 2895insN, 2896insN,
2897insN, 2898insN, 2899insN, 2900insN, 2901insN, 2902insN, 2903insN, 2904insN,
2905insN, 2906insN, 2907insN, 2908insN, 2909insN, 2910insN, 2911insN, 2912insN,
2913insN, 2914insN, 2915insN, 2916insN, 2917insN, 2918insN, 2919insN, 2920insN,
2921insN, 2922insN, 2923insN, 2924insN, 2925insN, 2926insN, 2927insN, 2928insN,
2929insN, 2930insN, 2931insN, 2932insN, 2933insN, 2934insN, 2935insN, 2936insN,
2937insN, 2938insN, 2939insN, 2940insN, 2941insN, 2942insN, 2943insN, 2944insN,
2945insN, 2946insN, 2947insN, 2948insN, 2949insN, 2950insN, 2951insN, 2952insN,
2953insN, 2954insN, 2955insN, 2956insN, 2957insN, 2958insN, 2959insN, 2960insN,
2961insN, 2962insN, 2963insN, 2964insN, 2965insN, 2966insN, 2967insN, 2968insN,
2969insN, 2970insN, 2971insN, 2972insN, 2973insN, 2974insN, 2975insN, 2976insN,
2977insN, 2978insN, 2979insN, 2980insN, 2981insN, 2982insN, 2983insN, 2984insN,
2985insN, 2986insN, 2987insN, 2988insN, 2989insN, 2990insN, 2991insN, 2992insN,
2993insN, 2994insN, 2995insN, 2996insN, 2997insN, 2998insN, 2999insN, 3000insN,
3001insN, 3002insN, 3003insN, 3004insN, 3005insN, 3006insN, 3007insN, 3008insN,
3009insN, 3010insN, 3011insN, 3012insN, 3013insN, 3014insN, 3015insN, 3016insN,
3017insN, 3018insN, 3019insN, 3020insN, 3021insN, 3022insN, 3023insN, 3024insN,
3025insN, 3026insN, 3027insN, 3028insN, 3029insN, 3030insN, 3031insN, 3032insN,
3033insN, 3034insN, 3035insN, 3036insN, 3037insN, 3038insN, 3039insN, 3040insN,
3041insN, 3042insN, 3043insN, 3044insN, 3045insN, 3046insN, 3047insN, 3048insN,
3049insN, 3050insN, 3051insN, 3052insN, 3053insN, 3054insN, 3055insN, 3056insN,
3057insN, 3058insN, 3059insN, 3060insN, 3061insN, 3062insN, 3063insN, 3064insN,
3065insN, 3066insN, 3067insN, 3068insN, 3069insN, 3070insN, 3071insN, 3072insN,
3073insN, 3074insN, 3075insN, 3076insN, 3077insN, 3078insN, 3079insN, 3080insN,
3081insN, 3082insN, 3083insN, 3084insN, 3085insN, 3086insN, 3087insN, 3088insN,
3089insN, 3090insN, 3091insN, 3092insN, 3093insN, 3094insN, 3095insN, 3096insN,
3097insN, 3098insN, 3099insN, 3100insN, 3101insN, 3102insN, 3103insN, 3104insN,
3105insN, 3106insN, 3107insN, 3108insN, 3109insN, 3110insN, 3111insN, 3112insN,
3113insN, 3114insN, 3115insN, 3116insN, 3117insN, 3118insN, 3119insN, 3120insN,
3121insN, 3122insN, 3123insN, 3124insN, 3125insN, 3126insN, 3127insN, 3128insN,
3129insN, 3130insN, 3131insN, 3132insN, 3133insN, 3134insN, 3135insN, 3136insN,
3137insN, 3138insN, 3139insN, 3140insN, 3141insN, 3142insN, 3143insN, 3144insN,
3145insN, 3146insN, 3147insN, 3148insN, 3149insN, 3150insN, 3151insN, 3152insN,
3153insN, 3154insN, 3155insN, 3156insN, 3157insN, 3158insN, 3159insN, 3160insN,
3161insN, 3162insN, 3163insN, 3164insN, 3165insN, 3166insN, 3167insN, 3168insN,
3169insN, 3170insN, 3171insN, 3172insN, 3173insN, 3174insN, 3175insN, 3176insN,
3177insN, 3178insN, 3179insN, 3180insN, 3181insN, 3182insN, 3183insN, 3184insN,
3185insN, 3186insN, 3187insN, 3188insN, 3189insN, 3190insN, 3191insN, 3192insN,
3193insN, 3194insN, 3195insN, 3196insN, 3197insN, 3198insN, 3199insN, 3200insN,
3201insN, 3202insN, 3203insN, 3204insN, 3205insN, 3206insN, 3207insN, 3208insN,
3209insN, 3210insN, 3211insN, 3212insN, 3213insN, 3214insN, 3215insN, 3216insN,
3217insN, 3218insN, 3219insN, 3220insN, 3221insN, 3222insN, 3223insN, 3224insN,
3225insN, 3226insN, 3227insN, 3228insN, 3229insN, 3230insN, 3231insN, 3232insN,
3233insN, 3234insN, 3235insN, 3236insN, 3237insN, 3238insN, 3239insN, 3240insN,
3241insN, 3242insN, 3243insN, 3244insN, 3245insN, 3246insN, 3247insN, 3248insN,
3249insN, 3250insN, 3251insN, 3252insN, 3253insN, 3254insN, 3255insN, 3256insN,
3257insN, 3258insN, 3259insN, 3260insN, 3261insN, 3262insN, 3263insN, 3264insN,
3265insN, 3266insN, 3267insN, 3268insN, 3269insN, 3270insN, 3271insN, 3272insN,
3273insN, 3274insN, 3275insN, 3276insN, 3277insN, 3278insN, 3279insN, 3280insN,
3281insN, 3282insN, 3283insN, 3284insN, 3285insN, 3286insN, 3287insN, 3288insN,
3289insN, 3290insN, 3291insN, 3292insN, 3293insN, 3294insN, 3295insN, 3296insN,
3297insN, 3298insN, 3299insN, 3300insN, 3301insN, 3302insN, 3303insN, 3304insN,
3305insN, 3306insN, 3307insN, 3308insN, 3309insN, 3310insN, 3311insN, 3312insN,
3313insN, 3314insN, 3315insN, 3316insN, 3317insN, 3318insN, 3319insN, 3320insN,
3321insN, 3322insN, 3323insN, 3324insN, 3325insN, 3326insN, 3327insN, 3328insN,
3329insN, 3330insN, 3331insN, 3332insN, 3333insN, 3334insN, 3335insN, 3336insN,
3337insN, 3338insN, 3339insN, 3340insN, 3341insN, 3342insN, 3343insN, 3344insN,
3345insN, 3346insN, 3347insN, 3348insN, 3349insN, 3350insN, 3351insN, 3352insN,
3353insN, 3354insN, 3355insN, 3356insN, 3357insN, 3358insN, 3359insN, 3360insN,
3361insN, 3362insN, 3363insN, 3364insN, 3365insN, 3366insN, 3367insN, 3368insN,
3369insN, 3370insN, 3371insN, 3372insN, 3373insN, 3374insN, 3375insN, 3376insN,
3377insN, 3378insN, 3379insN, 3380insN, 3381insN, 3382insN, 3383insN, 3384insN,
3385insN, 3386insN, 3387insN, 3388insN, 3389insN, 3390insN, 3391insN, 3392insN,
3393insN, 3394insN, 3395insN, 3396insN, 3397insN, 3398insN, 3399insN, 3400insN,
3401insN, 3402insN, 3403insN, 3404insN, 3405insN, 3406insN, 3407insN, 3408insN,
3409insN, 3410insN, 3411insN, 3412insN, 3413insN, 3414insN, 3415insN, 3416insN,
3417insN, 3418insN, 3419insN, 3420insN, 3421insN, 3422insN, 3423insN, 3424insN,
3425insN, 3426insN, 3427insN, 3428insN, 3429insN, 3430insN, 3431insN, 3432insN,
3433insN, 3434insN, 3435insN, 3436insN, 3437insN, 3438insN, 3439insN, 3440insN,
3441insN, 3442insN, 3443insN, 3444insN, 3445insN, 3446insN, 3447insN, 3448insN,
3449insN, 3450insN, 3451insN, 3452insN, 3453insN, 3454insN, 3455insN, 3456insN,
3457insN, 3458insN, 3459insN, 3460insN, 3461insN, 3462insN, 3463insN, 3464insN,
3465insN, 3466insN, 3467insN, 3468insN, 3469insN, 3470insN, 3471insN, 3472insN,
3473insN, 3474insN, 3475insN, 3476insN, 3477insN, 3478insN, 3479insN, 3480insN,
3481insN, 3482insN, 3483insN, 3484insN, 3485insN, 3486insN, 3487insN, 3488insN,
3489insN, 3490insN, 3491insN, 3492insN, 3493insN, 3494insN, 3495insN, 3496insN,
3497insN, 3498insN, 3499insN, 3500insN, 3501insN, 3502insN, 3503insN, 3504insN,
3505insN, 3506insN, 3507insN, 3508insN, 3509insN, 3510insN, 3511insN, 3512insN,
3513insN, 3514insN, 3515insN, 3516insN, 3517insN, 3518insN, 3519insN, 3520insN,
3521insN, 3522insN, 3523insN, 3524insN, 3525insN, 3526insN, 3527insN, 3528insN,
3529insN, 3530insN, 3531insN, 3532insN, 3533insN, 3534insN, 3535insN, 3536insN,
3537insN, 3538insN, 3539insN, 3540insN, 3541insN, 3542insN, 3543insN, 3544insN,
3545insN, 3546insN, 3547insN, 3548insN, 3549insN, 3550insN, 3551insN, 3552insN,
3553insN, 3554insN, 3555insN, 3556insN, 3557insN, 3558insN, 3559insN, 3560insN,
3561insN, 3562insN, 3563insN, 3564insN, 3565insN, 3566insN, 3567insN, 3568insN,
3569insN, 3570insN, 3571insN, 3572insN, 3573insN, 3574insN, 3575insN, 3576insN,
3577insN, 3578insN, 3579insN, 3580insN, 3581insN, 3582insN, 3583insN, 3584insN,
3585insN, 3586insN, 3587insN, 3588insN, 3589insN, 3590insN, 3591insN, 3592insN,
3593insN, 3594insN, 3595insN, 3596insN, 3597insN, 3598insN, 3599insN, 3600insN,
3601insN, 3602insN, 3603insN, 3604insN, 3605insN, 3606insN, 3607insN, 3608insN,
3609insN, 3610insN, 3611insN, 3612insN, 3613insN, 3614insN, 3615insN, 3616insN,
3617insN, 3618insN, 3619insN, 3620insN, 3621insN, 3622insN, 3623insN, 3624insN,
3625insN, 3626insN, 3627insN, 3628insN, 3629insN, 3630insN, 3631insN, 3632insN,
3633insN, 3634insN, 3635insN, 3636insN, 3637insN, 3638insN, 3639insN, 3640insN,
3641insN, 3642insN, 3643insN, 3644insN, 3645insN, 3646insN, 3647insN, 3648insN,
3649insN, 3650insN, 3651insN, 3652insN, 3653insN, 3654insN, 3655insN, 3656insN,
3657insN, 3658insN, 3659insN, 3660insN, 3661insN, 3662insN, 3663insN, 3664insN,
3665insN, 3666insN, 3667insN, 3668insN, 3669insN, 3670insN, 3671insN, 3672insN,
3673insN, 3674insN, 3675insN, 3676insN, 3677insN, 3678insN, 3679insN, 3680insN,
3681insN, 3682insN, 3683insN, 3684insN, 3685insN, 3686insN, 3687insN, 3688insN,
3689insN, 3690insN, 3691insN, 3692insN, 3693insN, 3694insN, 3695insN, 3696insN,
3697insN, 3698insN, 3699insN, 3700insN, 3701insN, 3702insN, 3703insN, 3704insN,
3705insN, 3706insN, 3707insN, 3708insN, 3709insN, 3710insN, 3711insN, 3712insN,
3713insN, 3714insN, 3715insN, 3716insN, 3717insN, 3718insN, 3719insN, 3720insN,
3721insN, 3722insN, 3723insN, 3724insN, 3725insN, 3726insN, 3727insN, 3728insN,
3729insN, 3730insN, 3731insN, 3732insN, 3733insN, 3734insN, 3735insN, 3736insN,
3737insN, 3738insN, 3739insN, 3740insN, 3741insN, 3742insN, 3743insN, 3744insN,
3745insN, 3746insN, 3747insN, 3748insN, 3749insN, 3750insN, 3751insN, 3752insN,
3753insN, 3754insN, 3755insN, 3756insN, 3757insN, 3758insN, 3759insN, 3760insN,
3761insN, 3762insN, 3763insN, 3764insN, 3765insN, 3766insN, 3767insN, 3768insN,
3769insN, 3770insN, 3771insN, 3772insN, 3773insN, 3774insN, 3775insN, 3776insN,
3777insN, 3778insN, 3779insN, 3780insN, 3781insN, 3782insN, 3783insN, 3784insN,
3785insN, 3786insN, 3787insN, 3788insN, 3789insN, 3790insN, 3791insN, 3792insN,
3793insN, 3794insN, 3795insN, 3796insN, 3797insN, 3798insN, 3799insN, 3800insN,
3801insN, 3802insN, 3803insN, 3804insN, 3805insN, 3806insN, 3807insN, 3808insN,
3809insN, 3810insN, 3811insN, 3812insN, 3813insN, 3814insN, 3815insN, 3816insN,
3817insN, 3818insN, 3819insN, 3820insN, 3821insN, 3822insN, 3823insN, 3824insN,
3825insN, 3826insN, 3827insN, 3828insN, 3829insN, 3830insN, 3831insN, 3832insN,
3833insN, 3834insN, 3835insN, 3836insN, 3837insN, 3838insN, 3839insN, 3840insN,
3841insN, 3842insN, 3843insN, 3844insN, 3845insN, 3846insN, 3847insN, 3848insN,
3849insN, 3850insN, 3851insN, 3852insN, 3853insN, 3854insN, 3855insN, 3856insN,
3857insN, 3858insN, 3859insN, 3860insN, 3861insN, 3862insN, 3863insN, 3864insN,
3865insN, 3866insN, 3867insN, 3868insN, 3869insN, 3870insN, 3871insN, 3872insN,
3873insN, 3874insN, 3875insN, 3876insN, 3877insN, 3878insN, 3879insN, 3880insN,
3881insN, 3882insN, 3883insN, 3884insN, 3885insN, 3886insN, 3887insN, 3888insN,
3889insN, 3890insN, 3891insN, 3892insN, 3893insN, 3894insN, 3895insN, 3896insN,
3897insN, 3898insN, 3899insN, 3900insN, 3901insN, 3902insN, 3903insN, 3904insN,
3905insN, 3906insN, 3907insN, 3908insN, 3909insN, 3910insN, 3911insN, 3912insN,
3913insN, 3914insN, 3915insN, 3916insN, 3917insN, 3918insN, 3919insN, 3920insN,
3921insN, 3922insN, 3923insN, 3924insN, 3925insN, 3926insN, 3927insN, 3928insN,
3929insN, 3930insN, 3931insN, 3932insN, 3933insN, 3934insN, 3935insN, 3936insN,
3937insN, 3938insN, 3939insN, 3940insN, 3941insN, 3942insN, 3943insN, 3944insN,
3945insN, 3946insN, 3947insN, 3948insN, 3949insN, 3950insN, 3951insN, 3952insN,
3953insN, 3954insN, 3955insN, 3956insN, 3957insN, 3958insN, 3959insN, 3960insN,
3961insN, 3962insN, 3963insN, 3964insN, 3965insN, 3966insN, 3967insN, 3968insN,
3969insN, 3970insN, 3971insN, 3972insN, 3973insN, 3974insN, 3975insN, 3976insN,
3977insN, 3978insN, 3979insN, 3980insN, 3981insN, 3982insN, 3983insN, 3984insN,
3985insN, 3986insN, 3987insN, 3988insN, 3989insN, 3990insN, 3991insN, 3992insN,
3993insN, 3994insN, 3995insN, 3996insN, 3997insN, 3998insN, 3999insN, 4000insN,
4001insN, 4002insN, 4003insN, 4004insN, 4005insN, 4006insN, 4007insN, 4008insN,
4009insN, 4010insN, 4011insN, 4012insN, 4013insN, 4014insN, 4015insN, 4016insN,
4017insN, 4018insN, 4019insN, 4020insN, 4021insN, 4022insN, 4023insN, 4024insN,
4025insN, 4026insN, 4027insN, 4028insN, 4029insN, 4030insN, 4031insN, 4032insN,
4033insN, 4034insN, 4035insN, 4036insN, 4037insN, 4038insN, 4039insN, 4040insN,
4041insN, 4042insN, 4043insN, 4044insN, 4045insN, 4046insN, 4047insN, 4048insN,
4049insN, 4050insN, 4051insN, 4052insN, 4053insN, 4054insN, 4055insN, 4056insN,
4057insN, 4058insN, 4059insN, 4060insN, 4061insN, 4062insN, 4063insN, 4064insN,
4065insN, 4066insN, 4067insN, 4068insN, 4069insN, 4070insN, 4071insN, 4072insN,
4073insN, 4074insN, 4075insN, 4076insN, 4077insN, 4078insN, 4079insN, 4080insN,
4081insN, 4082insN, 4083insN, 4084insN, 4085insN, 4086insN, 4087insN, 4088insN,
4089insN, 4090insN, 4091insN, 4092insN, 4093insN, 4094insN, 4095insN, 4096insN,
4097insN, 4098insN, 4099insN, 4100insN, 4101insN, 4102insN, 4103insN, 4104insN,
4105insN, 4106insN, 4107insN, 4108insN, 4109insN, 4110insN, 4111insN, 4112insN,
4113insN, 4114insN, 4115insN, 4116insN, 4117insN, 4118insN, 4119insN, 4120insN,
4121insN, 4122insN, 4123insN, 4124insN, 4125insN, 4126insN, 4127insN, 4128insN,
4129insN, 4130insN, 4131insN, 4132insN, 4133insN, 4134insN, 4135insN, 4136insN,
4137insN, 4138insN, 4139insN, 4140insN, 4141insN, 4142insN, 4143insN, 4144insN,
4145insN, 4146insN, 4147insN, 4148insN, 4149insN, 4150insN, 4151insN, 4152insN,
4153insN, 4154insN, 4155insN, 4156insN, 4157insN, 4158insN, 4159insN, 4160insN,
4161insN, 4162insN, 4163insN, 4164insN, 4165insN, 4166insN, 4167insN, 4168insN,
4169insN, 4170insN, 4171insN, 4172insN, 4173insN, 4174insN, 4175insN, 4176insN,
4177insN, 4178insN, 4179insN, 4180insN, 4181insN, 4182insN, 4183insN, 4184insN,
4185insN, 4186insN, 4187insN, 4188insN, 4189insN, 4190insN, 4191insN, 4192insN,
4193insN, 4194insN, 4195insN, 4196insN, 4197insN, 4198insN, 4199insN, 4200insN,
4201insN, 4202insN, 4203insN, 4204insN, 4205insN, 4206insN, 4207insN, 4208insN,
4209insN, 4210insN, 4211insN, 4212insN, 4213insN, 4214insN, 4215insN, 4216insN,
4217insN, 4218insN, 4219insN, 4220insN, 4221insN, 4222insN, 4223insN, 4224insN,
4225insN, 4226insN, 4227insN, 4228insN, 4229insN, 4230insN, 4231insN, 4232insN,
4233insN, 4234insN, 4235insN, 4236insN, 4237insN, 4238insN, 4239insN, 4240insN,
4241insN, 4242insN, 4243insN, 4244insN, 4245insN, 4246insN, 4247insN, 4248insN,
4249insN, 4250insN, 4251insN, 4252insN, 4253insN, 4254insN, 4255insN, 4256insN,
4257insN, 4258insN, 4259insN, 4260insN, 4261insN, 4262insN, 4263insN, 4264insN,
4265insN, 4266insN, 4267insN, 4268insN, 4269insN, 4270insN, 4271insN, 4272insN,
4273insN, 4274insN, 4275insN, 4276insN, 4277insN, 4278insN, 4279insN, 4280insN,
4281insN, 4282insN, 4283insN, 4284insN, 4285insN, 4286insN, 4287insN, 4288insN,
4289insN, 4290insN, 4291insN, 4292insN, 4293insN, 4294insN, 4295insN, 4296insN,
4297insN, 4298insN, 4299insN, 4300insN, 4301insN, 4302insN, 4303insN, 4304insN,
4305insN, 4306insN, 4307insN, 4308insN, 4309insN, 4310insN, 4311insN, 4312insN,
4313insN, 4314insN, 4315insN, 4316insN, 4317insN, 4318insN, 4319insN, 4320insN,
4321insN, 4322insN, 4323insN, 4324insN, 4325insN, 4326insN, 4327insN, 4328insN,
4329insN, 4330insN, 4331insN, 4332insN, 4333insN, 4334insN, 4335insN, 4336insN,
4337insN, 4338insN, 4339insN, 4340insN, 4341insN, 4342insN, 4343insN, 4344insN,
4345insN, 4346insN, 4347insN, 4348insN, 4349insN, 4350insN, 4351insN, 4352insN,
4353insN, 4354insN, 4355insN, 4356insN, 4357insN, 4358insN, 4359insN, 4360insN,
4361insN, 4362insN, 4363insN, 4364insN, 4365insN, 4366insN, 4367insN, 4368insN,
4369insN, 4370insN, 4371insN, 4372insN, 4373insN, 4374insN, 4375insN, 4376insN,
4377insN, 4378insN, 4379insN, 4380insN, 4381insN, 4382insN, 4383insN, 4384insN,
4385insN, 4386insN, 4387insN, 4388insN, 4389insN, 4390insN, 4391insN, 4392insN,
4393insN, 4394insN, 4395insN, 4396insN, 4397insN, 4398insN, 4399insN, 4400insN,
4401insN, 4402insN, 4403insN, 4404insN, 4405insN, 4406insN, 4407insN, 4408insN,
4409insN, 4410insN, 4411insN, 4412insN, 4413insN, 4414insN, 4415insN, 4416insN,
4417insN, 4418insN, 4419insN, 4420insN, 4421insN, 4422insN, 4423insN, 4424insN,
4425insN, 4426insN, 4427insN, 4428insN, 4429insN, 4430insN, 4431insN, 4432insN,
4433insN, 4434insN, 4435insN, 4436insN, 4437insN, 4438insN, 4439insN, 4440insN,
4441insN, 4442insN, 4443insN, 4444insN, 4445insN, 4446insN, 4447insN, 4448insN,
4449insN, 4450insN, 4451insN, 4452insN, 4453insN, 4454insN, 4455insN, 4456insN,
4457insN, 4458insN, 4459insN, 4460insN, 4461insN, 4462insN, 4463insN, 4464insN,
4465insN, 4466insN, 4467insN, 4468insN, 4469insN, 4470insN, 4471insN, 4472insN,
4473insN, 4474insN, 4475insN, 4476insN, 4477insN, 4478insN, 4479insN, 4480insN,
4481insN, 4482insN, 4483insN, 4484insN, 4485insN, 4486insN, 4487insN, 4488insN,
4489insN, 4490insN, 4491insN, 4492insN, 4493insN, 4494insN, 4495insN, 4496insN,
4497insN, 4498insN, 4499insN, 4500insN, 4501insN, 4502insN, 4503insN, 4504insN,
4505insN, 4506insN, 4507insN, 4508insN, 4509insN, 4510insN, 4511insN, 4512insN,
4513insN, 4514insN, 4515insN, 4516insN, 4517insN, 4518insN, 4519insN, 4520insN,
4521insN, 4522insN, 4523insN, 4524insN, 4525insN, 4526insN, 4527insN, 4528insN,
4529insN, 4530insN, 4531insN, 4532insN, 4533insN, 4534insN, 4535insN, 4536insN,
4537insN, 4538insN, 4539insN, 4540insN, 4541insN, 4542insN, 4543insN, 4544insN,
4545insN, 4546insN, 4547insN, 4548insN, 4549insN, 4550insN, 4551insN, 4552insN,
4553insN, 4554insN, 4555insN, 4556insN, 4557insN, 4558insN, 4559insN, 4560insN,
4561insN, 4562insN, 4563insN, 4564insN, 4565insN, 4566insN, 4567insN, 4568insN,
4569insN, 4570insN, 4571insN, 4572insN, 4573insN, 4574insN, 4575insN, 4576insN,
4577insN, 4578insN, 4579insN, 4580insN, 4581insN, 4582insN, 4583insN, 4584insN,
4585insN, 4586insN, 4587insN, 4588insN, 4589insN, 4590insN, 4591insN, 4592insN,
4593insN, 4594insN, 4595insN, 4596insN, 4597insN, 4598insN, 4599insN, 4600insN,
4601insN, 4602insN, 4603insN, 4604insN, 4605insN, 4606insN, 4607insN, 4608insN,
4609insN, 4610insN, 4611insN, 4612insN, 4613insN, 4614insN, 4615insN, 4616insN,
4617insN, 4618insN, 4619insN, 4620insN, 4621insN, 4622insN, 4623insN, 4624insN,
4625insN, 4626insN, 4627insN, 4628insN, 4629insN, 4630insN, 4631insN, 4632insN,
4633insN, 4634insN, 4635insN, 4636insN, 4637insN, 4638insN, 4639insN, 4640insN,
4641insN, 4642insN, 4643insN, 4644insN, 4645insN, 4646insN, 4647insN, 4648insN,
4649insN, 4650insN, 4651insN, 4652insN, 4653insN, 4654insN, 4655insN, 4656insN,
4657insN, 4658insN, 4659insN, 4660insN, 4661insN, 4662insN, 4663insN, 4664insN,
4665insN, 4666insN, 4667insN, 4668insN, 4669insN, 4670insN, 4671insN, 4672insN,
4673insN, 4674insN, 4675insN, 4676insN, 4677insN, 4678insN, 4679insN, 4680insN,
4681insN, 4682insN, 4683insN, 4684insN, 4685insN, 4686insN, 4687insN, 4688insN,
4689insN, 4690insN, 4691insN, 4692insN, 4693insN, 4694insN, 4695insN, 4696insN,
4697insN, 4698insN, 4699insN, 4700insN, 4701insN, 4702insN, 4703insN, 4704insN,
4705insN, 4706insN, 4707insN, 4708insN, 4709insN, 4710insN, 4711insN, 4712insN,
4713insN, 4714insN, 4715insN, 4716insN, 4717insN, 4718insN, 4719insN, 4720insN,
4721insN, 4722insN, 4723insN, 4724insN, 4725insN, 4726insN, 4727insN, 4728insN,
4729insN, 4730insN, 4731insN, 4732insN, 4733insN, 4734insN, 4735insN, 4736insN,
4737insN, 4738insN, 4739insN, 4740insN, 4741insN, 4742insN, 4743insN, 4744insN,
4745insN, 4746insN, 4747insN, 4748insN, 4749insN, 4750insN, 4751insN, 4752insN,
4753insN, 4754insN, 4755insN, 4756insN, 4757insN, 4758insN, 4759insN, 4760insN,
4761insN, 4762insN, 4763insN, 4764insN, 4765insN, 4766insN, 4767insN, 4768insN,
4769insN, 4770insN, 4771insN, 4772insN, 4773insN, 4774insN, 4775insN, 4776insN,
4777insN, 4778insN, 4779insN, 4780insN, 4781insN, 4782insN, 4783insN, 4784insN,
4785insN, 4786insN, 4787insN, 4788insN, 4789insN, 4790insN, 4791insN, 4792insN,
4793insN, 4794insN, 4795insN, 4796insN, 4797insN, 4798insN, 4799insN, 4800insN,
4801insN, 4802insN, 4803insN, 4804insN, 4805insN, 4806insN, 4807insN, 4808insN,
4809insN, 4810insN, 4811insN, 4812insN, 4813insN, 4814insN, 4815insN, 4816insN,
4817insN, 4818insN, 4819insN, 4820insN, 4821insN, 4822insN, 4823insN, 4824insN,
4825insN, 4826insN, 4827insN, 4828insN, 4829insN, 4830insN, 4831insN, 4832insN,
4833insN, 4834insN, 4835insN, 4836insN, 4837insN, 4838insN, 4839insN, 4840insN,
4841insN, 4842insN, 4843insN, 4844insN, 4845insN, 4846insN, 4847insN, 4848insN,
4849insN, 4850insN, 4851insN, 4852insN, 4853insN, 4854insN, 4855insN, 4856insN,
4857insN, 4858insN, 4859insN, 4860insN, 4861insN, 4862insN, 4863insN, 4864insN,
4865insN, 4866insN, 4867insN, 4868insN, 4869insN, 4870insN, 4871insN, 4872insN,
4873insN, 4874insN, 4875insN, 4876insN, 4877insN, 4878insN, 4879insN, 4880insN,
4881insN, 4882insN, 4883insN, 4884insN, 4885insN, 4886insN, 4887insN, 4888insN,
4889insN, 4890insN, 4891insN, 4892insN, 4893insN, 4894insN, 4895insN, 4896insN,
4897insN, 4898insN, 4899insN, 4900insN, 4901insN, 4902insN, 4903insN, 4904insN,
4905insN, 4906insN, 4907insN, 4908insN, 4909insN, 4910insN, 4911insN, 4912insN,
4913insN, 4914insN, 4915insN, 4916insN, 4917insN, 4918insN, 4919insN, 4920insN,
4921insN, 4922insN, 4923insN, 4924insN, 4925insN, 4926insN, 4927insN, 4928insN,
4929insN, 4930insN, 4931insN, 4932insN, 4933insN, 4934insN, 4935insN, 4936insN,
4937insN, 4938insN, 4939insN, 4940insN, 4941insN, 4942insN, 4943insN, 4944insN,
4945insN, 4946insN, 4947insN, 4948insN, 4949insN, 4950insN, 4951insN, 4952insN,
4953insN, 4954insN, 4955insN, 4956insN, 4957insN, 4958insN, 4959insN, 4960insN,
4961insN, 4962insN, 4963insN, 4964insN, 4965insN, 4966insN, 4967insN, 4968insN,
4969insN, 4970insN, 4971insN, 4972insN, 4973insN, 4974insN, 4975insN, 4976insN,
4977insN, 4978insN, 4979insN, 4980insN, 4981insN, 4982insN, 4983insN, 4984insN,
4985insN, 4986insN, 4987insN, 4988insN, 4989insN, 4990insN, 4991insN, 4992insN,
4993insN, 4994insN, 4995insN, 4996insN, 4997insN, 4998insN, 4999insN, 5000insN,
5001insN, 5002insN, 5003insN, 5004insN, 5005insN, 5006insN, 5007insN, 5008insN,
5009insN, 5010insN, 5011insN, 5012insN, 5013insN, 5014insN, 5015insN, 5016insN,
5017insN, 5018insN, 5019insN, 5020insN, 5021insN, 5022insN, 5023insN, 5024insN,
5025insN, 5026insN, 5027insN, 5028insN, 5029insN, 5030insN, 5031insN, 5032insN,
5033insN, 5034insN, 5035insN, 5036insN, 5037insN, 5038insN, 5039insN, 5040insN,
5041insN, 5042insN, 5043insN, 5044insN, 5045insN, 5046insN, 5047insN, 5048insN,
5049insN, 5050insN, 5051insN, 5052insN, 5053insN, 5054insN, 5055insN, 5056insN,
5057insN, 5058insN, 5059insN, 5060insN, 5061insN, 5062insN, 5063insN, 5064insN,
5065insN, 5066insN, 5067insN, 5068insN, 5069insN, 5070insN, 5071insN, 5072insN,
5073insN, 5074insN, 5075insN, 5076insN, 5077insN, 5078insN, 5079insN, 5080insN,
5081insN, 5082insN, 5083insN, 5084insN, 5085insN, 5086insN, 5087insN, 5088insN,
5089insN, 5090insN, 5091insN, 5092insN, 5093insN, 5094insN, 5095insN, 5096insN,
5097insN, 5098insN, 5099insN, 5100insN, 5101insN, 5102insN, 5103insN, 5104insN,
5105insN, 5106insN, 5107insN, 5108insN, 5109insN, 5110insN, 5111insN, 5112insN,
5113insN, 5114insN, 5115insN, 5116insN, 5117insN, 5118insN, 5119insN, 5120insN,
5121insN, 5122insN, 5123insN, 5124insN, 5125insN, 5126insN, 5127insN, 5128insN,
5129insN, 5130insN, 5131insN, 5132insN, 5133insN, 5134insN, 5135insN, 5136insN,
5137insN, 5138insN, 5139insN, 5140insN, 5141insN, 5142insN, 5143insN, 5144insN,
5145insN, 5146insN, 5147insN, 5148insN, 5149insN, 5150insN, 5151insN, 5152insN,
5153insN, 5154insN, 5155insN, 5156insN, 5157insN, 5158insN, 5159insN, 5160insN,
5161insN, 5162insN, 5163insN, 5164insN, 5165insN, 5166insN, 5167insN, 5168insN,
5169insN, 5170insN, 5171insN, 5172insN, 5173insN, 5174insN, 5175insN, 5176insN,
5177insN, 5178insN, 5179insN, 5180insN, 5181insN, 5182insN, 5183insN, 5184insN,
5185insN, 5186insN, 5187insN, 5188insN, 5189insN, 5190insN, 5191insN, 5192insN,
5193insN, 5194insN, 5195insN, 5196insN, 5197insN, 5198insN, 5199insN, 5200insN,
5201insN, 5202insN, 5203insN, 5204insN, 5205insN, 5206insN, 5207insN, 5208insN,
5209insN, 5210insN, 5211insN, 5212insN, 5213insN, 5214insN, 5215insN, 5216insN,
5217insN, 5218insN, 5219insN, 5220insN, 5221insN, 5222insN, 5223insN, 5224insN,
5225insN, 5226insN, 5227insN, 5228insN, 5229insN, 5230insN, 5231insN, 5232insN,
5233insN, 5234insN, 5235insN, 5236insN, 5237insN, 5238insN, 5239insN, 5240insN,
5241insN, 5242insN, 5243insN, 5244insN, 5245insN, 5246insN, 5247insN, 5248insN,
5249insN, 5250insN, 5251insN, 5252insN, 5253insN, 5254insN, 5255insN, 5256insN,
5257insN, 5258insN, 5259insN, 5260insN, 5261insN, 5262insN, 5263insN, 5264insN,
5265insN, 5266insN, 5267insN, 5268insN, 5269insN, 5270insN, 5271insN, 5272insN,
5273insN, 5274insN, 5275insN, 5276insN, 5277insN, 5278insN, 5279insN, 5280insN,
5281insN, 5282insN, 5283insN, 5284insN, 5285insN, 5286insN, 5287insN, 5288insN,
5289insN, 5290insN, 5291insN, 5292insN, 5293insN, 5294insN, 5295insN, 5296insN,
5297insN, 5298insN, 5299insN, 5300insN, 5301insN, 5302insN, 5303insN, 5304insN,
5305insN, 5306insN, 5307insN, 5308insN, 5309insN, 5310insN, 5311insN, 5312insN,
5313insN, 5314insN, 5315insN, 5316insN, 5317insN, 5318insN, 5319insN, 5320insN,
5321insN, 5322insN, 5323insN, 5324insN, 5325insN, 5326insN, 5327insN, 5328insN,
5329insN, 5330insN, 5331insN, 5332insN, 5333insN, 5334insN, 5335insN, 5336insN,
5337insN, 5338insN, 5339insN, 5340insN, 5341insN, 5342insN, 5343insN, 5344insN,
5345insN, 5346insN, 5347insN, 5348insN, 5349insN, 5350insN, 5351insN, 5352insN,
5353insN, 5354insN, 5355insN, 5356insN, 5357insN, 5358insN, 5359insN, 5360insN,
5361insN, 5362insN, 5363insN, 5364insN, 5365insN, 5366insN, 5367insN, 5368insN,
5369insN, 5370insN, 5371insN, 5372insN, 5373insN, 5374insN, 5375insN, 5376insN,
5377insN, 5378insN, 5379insN, 5380insN, 5381insN, 5382insN, 5383insN, 5384insN,
5385insN, 5386insN, 5387insN, 5388insN, 5389insN, 5390insN, 5391insN, 5392insN,
5393insN, 5394insN, 5395insN, 5396insN, 5397insN, 5398insN, 5399insN, 5400insN,
5401insN, 5402insN, 5403insN, 5404insN, 5405insN, 5406insN, 5407insN, 5408insN,
5409insN, 5410insN, 5411insN, 5412insN, 5413insN, 5414insN, 5415insN, 5416insN,
5417insN, 5418insN, 5419insN, 5420insN, 5421insN, 5422insN, 5423insN, 5424insN,
5425insN, 5426insN, 5427insN, 5428insN, 5429insN, 5430insN, 5431insN, 5432insN,
5433insN, 5434insN, 5435insN, 5436insN, 5437insN, 5438insN, 5439insN, 5440insN,
5441insN, 5442insN, 5443insN, 5444insN, 5445insN, 5446insN, 5447insN, 5448insN,
5449insN, 5450insN, 5451insN, 5452insN, 5453insN, 5454insN, 5455insN, 5456insN,
5457insN, 5458insN, 5459insN, 5460insN, 5461insN, 5462insN, 5463insN, 5464insN,
5465insN, 5466insN, 5467insN, 5468insN, 5469insN, 5470insN, 5471insN, 5472insN,
5473insN, 5474insN, 5475insN, 5476insN, 5477insN, 5478insN, 5479insN, 5480insN,
5481insN, 5482insN, 5483insN, 5484insN, 5485insN, 5486insN, 5487insN, 5488insN,
5489insN, 5490insN, 5491insN, 5492insN, 5493insN, 5494insN, 5495insN, 5496insN,
5497insN, 5498insN, 5499insN, 5500insN, 5501insN, 5502insN, 5503insN, 5504insN,
5505insN, 5506insN, 5507insN, 5508insN, 5509insN, 5510insN, 5511insN, 5512insN,
5513insN, 5514insN, 5515insN, 5516insN, 5517insN, 5518insN, 5519insN, 5520insN,
5521insN, 5522insN, 5523insN, 5524insN, 5525insN, 5526insN, 5527insN, 5528insN,
5529insN, 5530insN, 5531insN, 5532insN, 5533insN, 5534insN, 5535insN, 5536insN,
5537insN, 5538insN, 5539insN, 5540insN, 5541insN, 5542insN, 5543insN, 5544insN,
5545insN, 5546insN, 5547insN, 5548insN, 5549insN, 5550insN, 5551insN, 5552insN,
5553insN, 5554insN, 5555insN, 5556insN, 5557insN, 5558insN, 5559insN, 5560insN,
5561insN, 5562insN, 5563insN, 5564insN, 5565insN, 5566insN, 5567insN, 5568insN,
5569insN, 5570insN, 5571insN, 5572insN, 5573insN, 5574insN, 5575insN, 5576insN,
5577insN, 5578insN, 5579insN, 5580insN, 5581insN, 5582insN, 5583insN, 5584insN,
5585insN, 5586insN, 5587insN, 5588insN, 5589insN, 5590insN, 5591insN, 5592insN,
5593insN, 5594insN, 5595insN, 5596insN, 5597insN, 5598insN, 5599insN, 5600insN,
5601insN, 5602insN, 5628insN, 5629insN, 5652insN, 5653insN, 5658insN, 5659insN,
5676insN, 5677insN, 5706insN, 5707insN.

TABLE 12
List of Two Base Insertions
(NN = AA, AT, AG, AC, TA, TT, TG, TC, GA, GT, GG, GC, CA, CT, CG, or CC)

122insNN, 123insNN, 124insNN, 125insNN, 126insNN, 127insNN, 128insNN, 129insNN,
130insNN, 131insNN, 132insNN, 133insNN, 134insNN, 135insNN, 136insNN, 137insNN,
138insNN, 139insNN, 140insNN, 141insNN, 142insNN, 143insNN, 144insNN, 145insNN,
146insNN, 147insNN, 148insNN, 149insNN, 150insNN, 151insNN, 152insNN, 153insNN,
154insNN, 155insNN, 156insNN, 157insNN, 158insNN, 159insNN, 160insNN, 161insNN,
162insNN, 163insNN, 164insNN, 165insNN, 166insNN, 167insNN, 168insNN, 169insNN,
170insNN, 171insNN, 172insNN, 173insNN, 174insNN, 175insNN, 176insNN, 177insNN,
178insNN, 179insNN, 180insNN, 181insNN, 182insNN, 183insNN, 184insNN, 185insNN,
186insNN, 187insNN, 188insNN, 189insNN, 190insNN, 191insNN, 192insNN, 193insNN,
194insNN, 195insNN, 196insNN, 197insNN, 198insNN, 199insNN, 200insNN, 201insNN,
202insNN, 203insNN, 204insNN, 205insNN, 206insNN, 207insNN, 208insNN, 209insNN,
210insNN, 211insNN, 212insNN, 213insNN, 214insNN, 215insNN, 216insNN, 217insNN,
218insNN, 219insNN, 220insNN, 221insNN, 222insNN, 223insNN, 224insNN, 225insNN,
226insNN, 227insNN, 228insNN, 229insNN, 230insNN, 231insNN, 232insNN, 233insNN,
234insNN, 235insNN, 236insNN, 237insNN, 238insNN, 239insNN, 240insNN, 241insNN,
242insNN, 243insNN, 244insNN, 245insNN, 246insNN, 247insNN, 248insNN, 249insNN,
250insNN, 251insNN, 252insNN, 253insNN, 254insNN, 255insNN, 256insNN, 257insNN,
258insNN, 259insNN, 260insNN, 261insNN, 262insNN, 263insNN, 264insNN, 265insNN,
266insNN, 267insNN, 268insNN, 269insNN, 270insNN, 271insNN, 272insNN, 273insNN,
274insNN, 275insNN, 276insNN, 277insNN, 278insNN, 279insNN, 280insNN, 281insNN,
282insNN, 283insNN, 284insNN, 285insNN, 286insNN, 287insNN, 288insNN, 289insNN,
290insNN, 291insNN, 292insNN, 293insNN, 294insNN, 295insNN, 296insNN, 297insNN,
298insNN, 299insNN, 300insNN, 301insNN, 302insNN, 303insNN, 304insNN, 305insNN,
306insNN, 307insNN, 308insNN, 309insNN, 310insNN, 311insNN, 312insNN, 313insNN,
314insNN, 315insNN, 316insNN, 317insNN, 318insNN, 319insNN, 320insNN, 321insNN,
322insNN, 323insNN, 324insNN, 325insNN, 326insNN, 327insNN, 328insNN, 329insNN,
330insNN, 331insNN, 332insNN, 333insNN, 334insNN, 335insNN, 336insNN, 337insNN,
338insNN, 339insNN, 340insNN, 341insNN, 342insNN, 343insNN, 344insNN, 345insNN,
346insNN, 347insNN, 348insNN, 349insNN, 350insNN, 351insNN, 352insNN, 353insNN,
354insNN, 355insNN, 356insNN, 357insNN, 358insNN, 359insNN, 360insNN, 361insNN,
362insNN, 363insNN, 364insNN, 365insNN, 366insNN, 367insNN, 368insNN, 369insNN,
370insNN, 371insNN, 372insNN, 373insNN, 374insNN, 375insNN, 376insNN, 377insNN,
378insNN, 379insNN, 380insNN, 381insNN, 382insNN, 383insNN, 384insNN, 385insNN,
386insNN, 387insNN, 388insNN, 389insNN, 390insNN, 391insNN, 392insNN, 393insNN,
394insNN, 395insNN, 396insNN, 397insNN, 398insNN, 399insNN, 400insNN, 401insNN,
402insNN, 403insNN, 404insNN, 405insNN, 406insNN, 407insNN, 408insNN, 409insNN,
410insNN, 411insNN, 412insNN, 413insNN, 414insNN, 415insNN, 416insNN, 417insNN,
418insNN, 419insNN, 420insNN, 421insNN, 422insNN, 423insNN, 424insNN, 425insNN,
426insNN, 427insNN, 428insNN, 429insNN, 430insNN, 431insNN, 432insNN, 433insNN,
434insNN, 435insNN, 436insNN, 437insNN, 438insNN, 439insNN, 440insNN, 441insNN,
442insNN, 443insNN, 444insNN, 445insNN, 446insNN, 447insNN, 448insNN, 449insNN,
450insNN, 451insNN, 452insNN, 453insNN, 454insNN, 455insNN, 456insNN, 457insNN,
458insNN, 459insNN, 460insNN, 461insNN, 462insNN, 463insNN, 464insNN, 465insNN,
466insNN, 467insNN, 468insNN, 469insNN, 470insNN, 471insNN, 472insNN, 473insNN,
474insNN, 475insNN, 476insNN, 477insNN, 478insNN, 479insNN, 480insNN, 481insNN,
482insNN, 483insNN, 484insNN, 485insNN, 486insNN, 487insNN, 488insNN, 489insNN,
490insNN, 491insNN, 492insNN, 493insNN, 494insNN, 495insNN, 496insNN, 497insNN,
498insNN, 499insNN, 500insNN, 501insNN, 502insNN, 503insNN, 504insNN, 505insNN,
506insNN, 507insNN, 508insNN, 509insNN, 510insNN, 511insNN, 512insNN, 513insNN,
514insNN, 515insNN, 516insNN, 517insNN, 518insNN, 519insNN, 520insNN, 521insNN,
522insNN, 523insNN, 524insNN, 525insNN, 526insNN, 527insNN, 528insNN, 529insNN,
530insNN, 531insNN, 532insNN, 533insNN, 534insNN, 535insNN, 536insNN, 537insNN,
538insNN, 539insNN, 540insNN, 541insNN, 542insNN, 543insNN, 544insNN, 545insNN,
546insNN, 547insNN, 548insNN, 549insNN, 550insNN, 551insNN, 552insNN, 553insNN,
554insNN, 555insNN, 556insNN, 557insNN, 558insNN, 559insNN, 560insNN, 561insNN,
562insNN, 563insNN, 564insNN, 565insNN, 566insNN, 567insNN, 568insNN, 569insNN,
570insNN, 571insNN, 572insNN, 573insNN, 574insNN, 575insNN, 576insNN, 577insNN,
578insNN, 579insNN, 580insNN, 581insNN, 582insNN, 583insNN, 584insNN, 585insNN,
586insNN, 587insNN, 588insNN, 589insNN, 590insNN, 591insNN, 592insNN, 593insNN,
594insNN, 595insNN, 596insNN, 597insNN, 598insNN, 599insNN, 600insNN, 601insNN,
602insNN, 603insNN, 604insNN, 605insNN, 606insNN, 607insNN, 608insNN, 609insNN,
610insNN, 611insNN, 612insNN, 613insNN, 614insNN, 615insNN, 616insNN, 617insNN,
618insNN, 619insNN, 620insNN, 621insNN, 622insNN, 623insNN, 624insNN, 625insNN,
626insNN, 627insNN, 628insNN, 629insNN, 630insNN, 631insNN, 632insNN, 633insNN,
634insNN, 635insNN, 636insNN, 637insNN, 638insNN, 639insNN, 640insNN, 641insNN,
642insNN, 643insNN, 644insNN, 645insNN, 646insNN, 647insNN, 648insNN, 649insNN,
650insNN, 651insNN, 652insNN, 653insNN, 654insNN, 655insNN, 656insNN, 657insNN,
658insNN, 659insNN, 660insNN, 661insNN, 662insNN, 663insNN, 664insNN, 665insNN,
666insNN, 667insNN, 668insNN, 669insNN, 670insNN, 671insNN, 672insNN, 673insNN,
674insNN, 675insNN, 676insNN, 677insNN, 678insNN, 679insNN, 680insNN, 681insNN,
682insNN, 683insNN, 684insNN, 685insNN, 686insNN, 687insNN, 688insNN, 689insNN,
690insNN, 691insNN, 692insNN, 693insNN, 694insNN, 695insNN, 696insNN, 697insNN,
698insNN, 699insNN, 700insNN, 701insNN, 702insNN, 703insNN, 704insNN, 705insNN,
706insNN, 707insNN, 708insNN, 709insNN, 710insNN, 711insNN, 712insNN, 713insNN,
714insNN, 715insNN, 716insNN, 717insNN, 718insNN, 719insNN, 720insNN, 721insNN,
722insNN, 723insNN, 724insNN, 725insNN, 726insNN, 727insNN, 728insNN, 729insNN,
730insNN, 731insNN, 732insNN, 733insNN, 734insNN, 735insNN, 736insNN, 737insNN,
738insNN, 739insNN, 740insNN, 741insNN, 742insNN, 743insNN, 744insNN, 745insNN,
746insNN, 747insNN, 748insNN, 749insNN, 750insNN, 751insNN, 752insNN, 753insNN,
754insNN, 755insNN, 756insNN, 757insNN, 758insNN, 759insNN, 760insNN, 761insNN,
762insNN, 763insNN, 764insNN, 765insNN, 766insNN, 767insNN, 768insNN, 769insNN,
770insNN, 771insNN, 772insNN, 773insNN, 774insNN, 775insNN, 776insNN, 777insNN,
778insNN, 779insNN, 780insNN, 781insNN, 782insNN, 783insNN, 784insNN, 785insNN,
786insNN, 787insNN, 788insNN, 789insNN, 790insNN, 791insNN, 792insNN, 793insNN,
794insNN, 795insNN, 796insNN, 797insNN, 798insNN, 799insNN, 800insNN, 801insNN,
802insNN, 803insNN, 804insNN, 805insNN, 806insNN, 807insNN, 808insNN, 809insNN,
810insNN, 811insNN, 812insNN, 813insNN, 814insNN, 815insNN, 816insNN, 817insNN,
818insNN, 819insNN, 820insNN, 821insNN, 822insNN, 823insNN, 824insNN, 825insNN,
826insNN, 827insNN, 828insNN, 829insNN, 830insNN, 831insNN, 832insNN, 833insNN,
834insNN, 835insNN, 836insNN, 837insNN, 838insNN, 839insNN, 840insNN, 841insNN,
842insNN, 843insNN, 844insNN, 845insNN, 846insNN, 847insNN, 848insNN, 849insNN,
850insNN, 851insNN, 852insNN, 853insNN, 854insNN, 855insNN, 856insNN, 857insNN,
858insNN, 859insNN, 860insNN, 861insNN, 862insNN, 863insNN, 864insNN, 865insNN,
866insNN, 867insNN, 868insNN, 869insNN, 870insNN, 871insNN, 872insNN, 873insNN,
874insNN, 875insNN, 876insNN, 877insNN, 878insNN, 879insNN, 880insNN, 881insNN,
882insNN, 883insNN, 884insNN, 885insNN, 886insNN, 887insNN, 888insNN, 889insNN,
890insNN, 891insNN, 892insNN, 893insNN, 894insNN, 895insNN, 896insNN, 897insNN,
898insNN, 899insNN, 900insNN, 901insNN, 902insNN, 903insNN, 904insNN, 905insNN,
906insNN, 907insNN, 908insNN, 909insNN, 910insNN, 911insNN, 912insNN, 913insNN,
914insNN, 915insNN, 916insNN, 917insNN, 918insNN, 919insNN, 920insNN, 921insNN,
922insNN, 923insNN, 924insNN, 925insNN, 926insNN, 927insNN, 928insNN, 929insNN,
930insNN, 931insNN, 932insNN, 933insNN, 934insNN, 935insNN, 936insNN, 937insNN,
938insNN, 939insNN, 940insNN, 941insNN, 942insNN, 943insNN, 944insNN, 945insNN,
946insNN, 947insNN, 948insNN, 949insNN, 950insNN, 951insNN, 952insNN, 953insNN,
954insNN, 955insNN, 956insNN, 957insNN, 958insNN, 959insNN, 960insNN, 961insNN,
962insNN, 963insNN, 964insNN, 965insNN, 966insNN, 967insNN, 968insNN, 969insNN,
970insNN, 971insNN, 972insNN, 973insNN, 974insNN, 975insNN, 976insNN, 977insNN,
978insNN, 979insNN, 980insNN, 981insNN, 982insNN, 983insNN, 984insNN, 985insNN,
986insNN, 987insNN, 988insNN, 989insNN, 990insNN, 991insNN, 992insNN, 993insNN,
994insNN, 995insNN, 996insNN, 997insNN, 998insNN, 999insNN, 1000insNN, 1001insNN,
1002insNN, 1003insNN, 1004insNN, 1005insNN, 1006insNN, 1007insNN, 1008insNN,
1009insNN, 1010insNN, 1011insNN, 1012insNN, 1013insNN, 1014insNN, 1015insNN,
1016insNN, 1017insNN, 1018insNN, 1019insNN, 1020insNN, 1021insNN, 1022insNN,
1023insNN, 1024insNN, 1025insNN, 1026insNN, 1027insNN, 1028insNN, 1029insNN,
1030insNN, 1031insNN, 1032insNN, 1033insNN, 1034insNN, 1035insNN, 1036insNN,
1037insNN, 1038insNN, 1039insNN, 1040insNN, 1041insNN, 1042insNN, 1043insNN,
1044insNN, 1045insNN, 1046insNN, 1047insNN, 1048insNN, 1049insNN, 1050insNN,
1051insNN, 1052insNN, 1053insNN, 1054insNN, 1055insNN, 1056insNN, 1057insNN,
1058insNN, 1059insNN, 1060insNN, 1061insNN, 1062insNN, 1063insNN, 1064insNN,
1065insNN, 1066insNN, 1067insNN, 1068insNN, 1069insNN, 1070insNN, 1071insNN,
1072insNN, 1073insNN, 1074insNN, 1075insNN, 1076insNN, 1077insNN, 1078insNN,
1079insNN, 1080insNN, 1081insNN, 1082insNN, 1083insNN, 1084insNN, 1085insNN,
1086insNN, 1087insNN, 1088insNN, 1089insNN, 1090insNN, 1091insNN, 1092insNN,
1093insNN, 1094insNN, 1095insNN, 1096insNN, 1097insNN, 1098insNN, 1099insNN,
1100insNN, 1101insNN, 1102insNN, 1103insNN, 1104insNN, 1105insNN, 1106insNN,
1107insNN, 1108insNN, 1109insNN, 1110insNN, 1111insNN, 1112insNN, 1113insNN,
1114insNN, 1115insNN, 1116insNN, 1117insNN, 1118insNN, 1119insNN, 1120insNN,
1121insNN, 1122insNN, 1123insNN, 1124insNN, 1125insNN, 1126insNN, 1127insNN,
1128insNN, 1129insNN, 1130insNN, 1131insNN, 1132insNN, 1133insNN, 1134insNN,
1135insNN, 1136insNN, 1137insNN, 1138insNN, 1139insNN, 1140insNN, 1141insNN,
1142insNN, 1143insNN, 1144insNN, 1145insNN, 1146insNN, 1147insNN, 1148insNN,
1149insNN, 1150insNN, 1151insNN, 1152insNN, 1153insNN, 1154insNN, 1155insNN,
1156insNN, 1157insNN, 1158insNN, 1159insNN, 1160insNN, 1161insNN, 1162insNN,
1163insNN, 1164insNN, 1165insNN, 1166insNN, 1167insNN, 1168insNN, 1169insNN,
1170insNN, 1171insNN, 1172insNN, 1173insNN, 1174insNN, 1175insNN, 1176insNN,
1177insNN, 1178insNN, 1179insNN, 1180insNN, 1181insNN, 1182insNN, 1183insNN,
1184insNN, 1185insNN, 1186insNN, 1187insNN, 1188insNN, 1189insNN, 1190insNN,
1191insNN, 1192insNN, 1193insNN, 1194insNN, 1195insNN, 1196insNN, 1197insNN,
1198insNN, 1199insNN, 1200insNN, 1201insNN, 1202insNN, 1203insNN, 1204insNN,
1205insNN, 1206insNN, 1207insNN, 1208insNN, 1209insNN, 1210insNN, 1211insNN,
1212insNN, 1213insNN, 1214insNN, 1215insNN, 1216insNN, 1217insNN, 1218insNN,
1219insNN, 1220insNN, 1221insNN, 1222insNN, 1223insNN, 1224insNN, 1225insNN,
1226insNN, 1227insNN, 1228insNN, 1229insNN, 1230insNN, 1231insNN, 1232insNN,
1233insNN, 1234insNN, 1235insNN, 1236insNN, 1237insNN, 1238insNN, 1239insNN,
1240insNN, 1241insNN, 1242insNN, 1243insNN, 1244insNN, 1245insNN, 1246insNN,
1247insNN, 1248insNN, 1249insNN, 1250insNN, 1251insNN, 1252insNN, 1253insNN,
1254insNN, 1255insNN, 1256insNN, 1257insNN, 1258insNN, 1259insNN, 1260insNN,
1261insNN, 1262insNN, 1263insNN, 1264insNN, 1265insNN, 1266insNN, 1267insNN,
1268insNN, 1269insNN, 1270insNN, 1271insNN, 1272insNN, 1273insNN, 1274insNN,
1275insNN, 1276insNN, 1277insNN, 1278insNN, 1279insNN, 1280insNN, 1281insNN,
1282insNN, 1283insNN, 1284insNN, 1285insNN, 1286insNN, 1287insNN, 1288insNN,
1289insNN, 1290insNN, 1291insNN, 1292insNN, 1293insNN, 1294insNN, 1295insNN,
1296insNN, 1297insNN, 1298insNN, 1299insNN, 1300insNN, 1301insNN, 1302insNN,
1303insNN, 1304insNN, 1305insNN, 1306insNN, 1307insNN, 1308insNN, 1309insNN,
1310insNN, 1311insNN, 1312insNN, 1313insNN, 1314insNN, 1315insNN, 1316insNN,
1317insNN, 1318insNN, 1319insNN, 1320insNN, 1321insNN, 1322insNN, 1323insNN,
1324insNN, 1325insNN, 1326insNN, 1327insNN, 1328insNN, 1329insNN, 1330insNN,
1331insNN, 1332insNN, 1333insNN, 1334insNN, 1335insNN, 1336insNN, 1337insNN,
1338insNN, 1339insNN, 1340insNN, 1341insNN, 1342insNN, 1343insNN, 1344insNN,
1345insNN, 1346insNN, 1347insNN, 1348insNN, 1349insNN, 1350insNN, 1351insNN,
1352insNN, 1353insNN, 1354insNN, 1355insNN, 1356insNN, 1357insNN, 1358insNN,
1359insNN, 1360insNN, 1361insNN, 1362insNN, 1363insNN, 1364insNN, 1365insNN,
1366insNN, 1367insNN, 1368insNN, 1369insNN, 1370insNN, 1371insNN, 1372insNN,
1373insNN, 1374insNN, 1375insNN, 1376insNN, 1377insNN, 1378insNN, 1379insNN,
1380insNN, 1381insNN, 1382insNN, 1383insNN, 1384insNN, 1385insNN, 1386insNN,
1387insNN, 1388insNN, 1389insNN, 1390insNN, 1391insNN, 1392insNN, 1393insNN,
1394insNN, 1395insNN, 1396insNN, 1397insNN, 1398insNN, 1399insNN, 1400insNN,
1401insNN, 1402insNN, 1403insNN, 1404insNN, 1405insNN, 1406insNN, 1407insNN,
1408insNN, 1409insNN, 1410insNN, 1411insNN, 1412insNN, 1413insNN, 1414insNN,
1415insNN, 1416insNN, 1417insNN, 1418insNN, 1419insNN, 1420insNN, 1421insNN,
1422insNN, 1423insNN, 1424insNN, 1425insNN, 1426insNN, 1427insNN, 1428insNN,
1429insNN, 1430insNN, 1431insNN, 1432insNN, 1433insNN, 1434insNN, 1435insNN,
1436insNN, 1437insNN, 1438insNN, 1439insNN, 1440insNN, 1441insNN, 1442insNN,
1443insNN, 1444insNN, 1445insNN, 1446insNN, 1447insNN, 1448insNN, 1449insNN,
1450insNN, 1451insNN, 1452insNN, 1453insNN, 1454insNN, 1455insNN, 1456insNN,
1457insNN, 1458insNN, 1459insNN, 1460insNN, 1461insNN, 1462insNN, 1463insNN,
1464insNN, 1465insNN, 1466insNN, 1467insNN, 1468insNN, 1469insNN, 1470insNN,
1471insNN, 1472insNN, 1473insNN, 1474insNN, 1475insNN, 1476insNN, 1477insNN,
1478insNN, 1479insNN, 1480insNN, 1481insNN, 1482insNN, 1483insNN, 1484insNN,
1485insNN, 1486insNN, 1487insNN, 1488insNN, 1489insNN, 1490insNN, 1491insNN,
1492insNN, 1493insNN, 1494insNN, 1495insNN, 1496insNN, 1497insNN, 1498insNN,
1499insNN, 1500insNN, 1501insNN, 1502insNN, 1503insNN, 1504insNN, 1505insNN,
1506insNN, 1507insNN, 1508insNN, 1509insNN, 1510insNN, 1511insNN, 1512insNN,
1513insNN, 1514insNN, 1515insNN, 1516insNN, 1517insNN, 1518insNN, 1519insNN,
1520insNN, 1521insNN, 1522insNN, 1523insNN, 1524insNN, 1525insNN, 1526insNN,
1527insNN, 1528insNN, 1529insNN, 1530insNN, 1531insNN, 1532insNN, 1533insNN,
1534insNN, 1535insNN, 1536insNN, 1537insNN, 1538insNN, 1539insNN, 1540insNN,
1541insNN, 1542insNN, 1543insNN, 1544insNN, 1545insNN, 1546insNN, 1547insNN,
1548insNN, 1549insNN, 1550insNN, 1551insNN, 1552insNN, 1553insNN, 1554insNN,
1555insNN, 1556insNN, 1557insNN, 1558insNN, 1559insNN, 1560insNN, 1561insNN,
1562insNN, 1563insNN, 1564insNN, 1565insNN, 1566insNN, 1567insNN, 1568insNN,
1569insNN, 1570insNN, 1571insNN, 1572insNN, 1573insNN, 1574insNN, 1575insNN,
1576insNN, 1577insNN, 1578insNN, 1579insNN, 1580insNN, 1581insNN, 1582insNN,
1583insNN, 1584insNN, 1585insNN, 1586insNN, 1587insNN, 1588insNN, 1589insNN,
1590insNN, 1591insNN, 1592insNN, 1593insNN, 1594insNN, 1595insNN, 1596insNN,
1597insNN, 1598insNN, 1599insNN, 1600insNN, 1601insNN, 1602insNN, 1603insNN,
1604insNN, 1605insNN, 1606insNN, 1607insNN, 1608insNN, 1609insNN, 1610insNN,
1611insNN, 1612insNN, 1613insNN, 1614insNN, 1615insNN, 1616insNN, 1617insNN,
1618insNN, 1619insNN, 1620insNN, 1621insNN, 1622insNN, 1623insNN, 1624insNN,
1625insNN, 1626insNN, 1627insNN, 1628insNN, 1629insNN, 1630insNN, 1631insNN,
1632insNN, 1633insNN, 1634insNN, 1635insNN, 1636insNN, 1637insNN, 1638insNN,
1639insNN, 1640insNN, 1641insNN, 1642insNN, 1643insNN, 1644insNN, 1645insNN,
1646insNN, 1647insNN, 1648insNN, 1649insNN, 1650insNN, 1651insNN, 1652insNN,
1653insNN, 1654insNN, 1655insNN, 1656insNN, 1657insNN, 1658insNN, 1659insNN,
1660insNN, 1661insNN, 1662insNN, 1663insNN, 1664insNN, 1665insNN, 1666insNN,
1667insNN, 1668insNN, 1669insNN, 1670insNN, 1671insNN, 1672insNN, 1673insNN,
1674insNN, 1675insNN, 1676insNN, 1677insNN, 1678insNN, 1679insNN, 1680insNN,
1681insNN, 1682insNN, 1683insNN, 1684insNN, 1685insNN, 1686insNN, 1687insNN,
1688insNN, 1689insNN, 1690insNN, 1691insNN, 1692insNN, 1693insNN, 1694insNN,
1695insNN, 1696insNN, 1697insNN, 1698insNN, 1699insNN, 1700insNN, 1701insNN,
1702insNN, 1703insNN, 1704insNN, 1705insNN, 1706insNN, 1707insNN, 1708insNN,
1709insNN, 1710insNN, 1711insNN, 1712insNN, 1713insNN, 1714insNN, 1715insNN,
1716insNN, 1717insNN, 1718insNN, 1719insNN, 1720insNN, 1721insNN, 1722insNN,
1723insNN, 1724insNN, 1725insNN, 1726insNN, 1727insNN, 1728insNN, 1729insNN,
1730insNN, 1731insNN, 1732insNN, 1733insNN, 1734insNN, 1735insNN, 1736insNN,
1737insNN, 1738insNN, 1739insNN, 1740insNN, 1741insNN, 1742insNN, 1743insNN,
1744insNN, 1745insNN, 1746insNN, 1747insNN, 1748insNN, 1749insNN, 1750insNN,
1751insNN, 1752insNN, 1753insNN, 1754insNN, 1755insNN, 1756insNN, 1757insNN,
1758insNN, 1759insNN, 1760insNN, 1761insNN, 1762insNN, 1763insNN, 1764insNN,
1765insNN, 1766insNN, 1767insNN, 1768insNN, 1769insNN, 1770insNN, 1771insNN,
1772insNN, 1773insNN, 1774insNN, 1775insNN, 1776insNN, 1777insNN, 1778insNN,
1779insNN, 1780insNN, 1781insNN, 1782insNN, 1783insNN, 1784insNN, 1785insNN,
1786insNN, 1787insNN, 1788insNN, 1789insNN, 1790insNN, 1791insNN, 1792insNN,
1793insNN, 1794insNN, 1795insNN, 1796insNN, 1797insNN, 1798insNN, 1799insNN,
1800insNN, 1801insNN, 1802insNN, 1803insNN, 1804insNN, 1805insNN, 1806insNN,
1807insNN, 1808insNN, 1809insNN, 1810insNN, 1811insNN, 1812insNN, 1813insNN,
1814insNN, 1815insNN, 1816insNN, 1817insNN, 1818insNN, 1819insNN, 1820insNN,
1821insNN, 1822insNN, 1823insNN, 1824insNN, 1825insNN, 1826insNN, 1827insNN,
1828insNN, 1829insNN, 1830insNN, 1831insNN, 1832insNN, 1833insNN, 1834insNN,
1835insNN, 1836insNN, 1837insNN, 1838insNN, 1839insNN, 1840insNN, 1841insNN,
1842insNN, 1843insNN, 1844insNN, 1845insNN, 1846insNN, 1847insNN, 1848insNN,
1849insNN, 1850insNN, 1851insNN, 1852insNN, 1853insNN, 1854insNN, 1855insNN,
1856insNN, 1857insNN, 1858insNN, 1859insNN, 1860insNN, 1861insNN, 1862insNN,
1863insNN, 1864insNN, 1865insNN, 1866insNN, 1867insNN, 1868insNN, 1869insNN,
1870insNN, 1871insNN, 1872insNN, 1873insNN, 1874insNN, 1875insNN, 1876insNN,
1877insNN, 1878insNN, 1879insNN, 1880insNN, 1881insNN, 1882insNN, 1883insNN,
1884insNN, 1885insNN, 1886insNN, 1887insNN, 1888insNN, 1889insNN, 1890insNN,
1891insNN, 1892insNN, 1893insNN, 1894insNN, 1895insNN, 1896insNN, 1897insNN,
1898insNN, 1899insNN, 1900insNN, 1901insNN, 1902insNN, 1903insNN, 1904insNN,
1905insNN, 1906insNN, 1907insNN, 1908insNN, 1909insNN, 1910insNN, 1911insNN,
1912insNN, 1913insNN, 1914insNN, 1915insNN, 1916insNN, 1917insNN, 1918insNN,
1919insNN, 1920insNN, 1921insNN, 1922insNN, 1923insNN, 1924insNN, 1925insNN,
1926insNN, 1927insNN, 1928insNN, 1929insNN, 1930insNN, 1931insNN, 1932insNN,
1933insNN, 1934insNN, 1935insNN, 1936insNN, 1937insNN, 1938insNN, 1939insNN,
1940insNN, 1941insNN, 1942insNN, 1943insNN, 1944insNN, 1945insNN, 1946insNN,
1947insNN, 1948insNN, 1949insNN, 1950insNN, 1951insNN, 1952insNN, 1953insNN,
1954insNN, 1955insNN, 1956insNN, 1957insNN, 1958insNN, 1959insNN, 1960insNN,
1961insNN, 1962insNN, 1963insNN, 1964insNN, 1965insNN, 1966insNN, 1967insNN,
1968insNN, 1969insNN, 1970insNN, 1971insNN, 1972insNN, 1973insNN, 1974insNN,
1975insNN, 1976insNN, 1977insNN, 1978insNN, 1979insNN, 1980insNN, 1981insNN,
1982insNN, 1983insNN, 1984insNN, 1985insNN, 1986insNN, 1987insNN, 1988insNN,
1989insNN, 1990insNN, 1991insNN, 1992insNN, 1993insNN, 1994insNN, 1995insNN,
1996insNN, 1997insNN, 1998insNN, 1999insNN, 2000insNN, 2001insNN, 2002insNN,
2003insNN, 2004insNN, 2005insNN, 2006insNN, 2007insNN, 2008insNN, 2009insNN,
2010insNN, 2011insNN, 2012insNN, 2013insNN, 2014insNN, 2015insNN, 2016insNN,
2017insNN, 2018insNN, 2019insNN, 2020insNN, 2021insNN, 2022insNN, 2023insNN,
2024insNN, 2025insNN, 2026insNN, 2027insNN, 2028insNN, 2029insNN, 2030insNN,
2031insNN, 2032insNN, 2033insNN, 2034insNN, 2035insNN, 2036insNN, 2037insNN,
2038insNN, 2039insNN, 2040insNN, 2041insNN, 2042insNN, 2043insNN, 2044insNN,
2045insNN, 2046insNN, 2047insNN, 2048insNN, 2049insNN, 2050insNN, 2051insNN,
2052insNN, 2053insNN, 2054insNN, 2055insNN, 2056insNN, 2057insNN, 2058insNN,
2059insNN, 2060insNN, 2061insNN, 2062insNN, 2063insNN, 2064insNN, 2065insNN,
2066insNN, 2067insNN, 2068insNN, 2069insNN, 2070insNN, 2071insNN, 2072insNN,
2073insNN, 2074insNN, 2075insNN, 2076insNN, 2077insNN, 2078insNN, 2079insNN,
2080insNN, 2081insNN, 2082insNN, 2083insNN, 2084insNN, 2085insNN, 2086insNN,
2087insNN, 2088insNN, 2089insNN, 2090insNN, 2091insNN, 2092insNN, 2093insNN,
2094insNN, 2095insNN, 2096insNN, 2097insNN, 2098insNN, 2099insNN, 2100insNN,
2101insNN, 2102insNN, 2103insNN, 2104insNN, 2105insNN, 2106insNN, 2107insNN,
2108insNN, 2109insNN, 2110insNN, 2111insNN, 2112insNN, 2113insNN, 2114insNN,
2115insNN, 2116insNN, 2117insNN, 2118insNN, 2119insNN, 2120insNN, 2121insNN,
2122insNN, 2123insNN, 2124insNN, 2125insNN, 2126insNN, 2127insNN, 2128insNN,
2129insNN, 2130insNN, 2131insNN, 2132insNN, 2133insNN, 2134insNN, 2135insNN,
2136insNN, 2137insNN, 2138insNN, 2139insNN, 2140insNN, 2141insNN, 2142insNN,
2143insNN, 2144insNN, 2145insNN, 2146insNN, 2147insNN, 2148insNN, 2149insNN,
2150insNN, 2151insNN, 2152insNN, 2153insNN, 2154insNN, 2155insNN, 2156insNN,
2157insNN, 2158insNN, 2159insNN, 2160insNN, 2161insNN, 2162insNN, 2163insNN,
2164insNN, 2165insNN, 2166insNN, 2167insNN, 2168insNN, 2169insNN, 2170insNN,
2171insNN, 2172insNN, 2173insNN, 2174insNN, 2175insNN, 2176insNN, 2177insNN,
2178insNN, 2179insNN, 2180insNN, 2181insNN, 2182insNN, 2183insNN, 2184insNN,
2185insNN, 2186insNN, 2187insNN, 2188insNN, 2189insNN, 2190insNN, 2191insNN,
2192insNN, 2193insNN, 2194insNN, 2195insNN, 2196insNN, 2197insNN, 2198insNN,
2199insNN, 2200insNN, 2201insNN, 2202insNN, 2203insNN, 2204insNN, 2205insNN,
2206insNN, 2207insNN, 2208insNN, 2209insNN, 2210insNN, 2211insNN, 2212insNN,
2213insNN, 2214insNN, 2215insNN, 2216insNN, 2217insNN, 2218insNN, 2219insNN,
2220insNN, 2221insNN, 2222insNN, 2223insNN, 2224insNN, 2225insNN, 2226insNN,
2227insNN, 2228insNN, 2229insNN, 2230insNN, 2231insNN, 2232insNN, 2233insNN,
2234insNN, 2235insNN, 2236insNN, 2237insNN, 2238insNN, 2239insNN, 2240insNN,
2241insNN, 2242insNN, 2243insNN, 2244insNN, 2245insNN, 2246insNN, 2247insNN,
2248insNN, 2249insNN, 2250insNN, 2251insNN, 2252insNN, 2253insNN, 2254insNN,
2255insNN, 2256insNN, 2257insNN, 2258insNN, 2259insNN, 2260insNN, 2261insNN,
2262insNN, 2263insNN, 2264insNN, 2265insNN, 2266insNN, 2267insNN, 2268insNN,
2269insNN, 2270insNN, 2271insNN, 2272insNN, 2273insNN, 2274insNN, 2275insNN,
2276insNN, 2277insNN, 2278insNN, 2279insNN, 2280insNN, 2281insNN, 2282insNN,
2283insNN, 2284insNN, 2285insNN, 2286insNN, 2287insNN, 2288insNN, 2289insNN,
2290insNN, 2291insNN, 2292insNN, 2293insNN, 2294insNN, 2295insNN, 2296insNN,
2297insNN, 2298insNN, 2299insNN, 2300insNN, 2301insNN, 2302insNN, 2303insNN,
2304insNN, 2305insNN, 2306insNN, 2307insNN, 2308insNN, 2309insNN, 2310insNN,
2311insNN, 2312insNN, 2313insNN, 2314insNN, 2315insNN, 2316insNN, 2317insNN,
2318insNN, 2319insNN, 2320insNN, 2321insNN, 2322insNN, 2323insNN, 2324insNN,
2325insNN, 2326insNN, 2327insNN, 2328insNN, 2329insNN, 2330insNN, 2331insNN,
2332insNN, 2333insNN, 2334insNN, 2335insNN, 2336insNN, 2337insNN, 2338insNN,
2339insNN, 2340insNN, 2341insNN, 2342insNN, 2343insNN, 2344insNN, 2345insNN,
2346insNN, 2347insNN, 2348insNN, 2349insNN, 2350insNN, 2351insNN, 2352insNN,
2353insNN, 2354insNN, 2355insNN, 2356insNN, 2357insNN, 2358insNN, 2359insNN,
2360insNN, 2361insNN, 2362insNN, 2363insNN, 2364insNN, 2365insNN, 2366insNN,
2367insNN, 2368insNN, 2369insNN, 2370insNN, 2371insNN, 2372insNN, 2373insNN,
2374insNN, 2375insNN, 2376insNN, 2377insNN, 2378insNN, 2379insNN, 2380insNN,
2381insNN, 2382insNN, 2383insNN, 2384insNN, 2385insNN, 2386insNN, 2387insNN,
2388insNN, 2389insNN, 2390insNN, 2391insNN, 2392insNN, 2393insNN, 2394insNN,
2395insNN, 2396insNN, 2397insNN, 2398insNN, 2399insNN, 2400insNN, 2401insNN,
2402insNN, 2403insNN, 2404insNN, 2405insNN, 2406insNN, 2407insNN, 2408insNN,
2409insNN, 2410insNN, 2411insNN, 2412insNN, 2413insNN, 2414insNN, 2415insNN,
2416insNN, 2417insNN, 2418insNN, 2419insNN, 2420insNN, 2421insNN, 2422insNN,
2423insNN, 2424insNN, 2425insNN, 2426insNN, 2427insNN, 2428insNN, 2429insNN,
2430insNN, 2431insNN, 2432insNN, 2433insNN, 2434insNN, 2435insNN, 2436insNN,
2437insNN, 2438insNN, 2439insNN, 2440insNN, 2441insNN, 2442insNN, 2443insNN,
2444insNN, 2445insNN, 2446insNN, 2447insNN, 2448insNN, 2449insNN, 2450insNN,
2451insNN, 2452insNN, 2453insNN, 2454insNN, 2455insNN, 2456insNN, 2457insNN,
2458insNN, 2459insNN, 2460insNN, 2461insNN, 2462insNN, 2463insNN, 2464insNN,
2465insNN, 2466insNN, 2467insNN, 2468insNN, 2469insNN, 2470insNN, 2471insNN,
2472insNN, 2473insNN, 2474insNN, 2475insNN, 2476insNN, 2477insNN, 2478insNN,
2479insNN, 2480insNN, 2481insNN, 2482insNN, 2483insNN, 2484insNN, 2485insNN,
2486insNN, 2487insNN, 2488insNN, 2489insNN, 2490insNN, 2491insNN, 2492insNN,
2493insNN, 2494insNN, 2495insNN, 2496insNN, 2497insNN, 2498insNN, 2499insNN,
2500insNN, 2501insNN, 2502insNN, 2503insNN, 2504insNN, 2505insNN, 2506insNN,
2507insNN, 2508insNN, 2509insNN, 2510insNN, 2511insNN, 2512insNN, 2513insNN,
2514insNN, 2515insNN, 2516insNN, 2517insNN, 2518insNN, 2519insNN, 2520insNN,
2521insNN, 2522insNN, 2523insNN, 2524insNN, 2525insNN, 2526insNN, 2527insNN,
2528insNN, 2529insNN, 2530insNN, 2531insNN, 2532insNN, 2533insNN, 2534insNN,
2535insNN, 2536insNN, 2537insNN, 2538insNN, 2539insNN, 2540insNN, 2541insNN,
2542insNN, 2543insNN, 2544insNN, 2545insNN, 2546insNN, 2547insNN, 2548insNN,
2549insNN, 2550insNN, 2551insNN, 2552insNN, 2553insNN, 2554insNN, 2555insNN,
2556insNN, 2557insNN, 2558insNN, 2559insNN, 2560insNN, 2561insNN, 2562insNN,
2563insNN, 2564insNN, 2565insNN, 2566insNN, 2567insNN, 2568insNN, 2569insNN,
2570insNN, 2571insNN, 2572insNN, 2573insNN, 2574insNN, 2575insNN, 2576insNN,
2577insNN, 2578insNN, 2579insNN, 2580insNN, 2581insNN, 2582insNN, 2583insNN,
2584insNN, 2585insNN, 2586insNN, 2587insNN, 2588insNN, 2589insNN, 2590insNN,
2591insNN, 2592insNN, 2593insNN, 2594insNN, 2595insNN, 2596insNN, 2597insNN,
2598insNN, 2599insNN, 2600insNN, 2601insNN, 2602insNN, 2603insNN, 2604insNN,
2605insNN, 2606insNN, 2607insNN, 2608insNN, 2609insNN, 2610insNN, 2611insNN,
2612insNN, 2613insNN, 2614insNN, 2615insNN, 2616insNN, 2617insNN, 2618insNN,
2619insNN, 2620insNN, 2621insNN, 2622insNN, 2623insNN, 2624insNN, 2625insNN,
2626insNN, 2627insNN, 2628insNN, 2629insNN, 2630insNN, 2631insNN, 2632insNN,
2633insNN, 2634insNN, 2635insNN, 2636insNN, 2637insNN, 2638insNN, 2639insNN,
2640insNN, 2641insNN, 2642insNN, 2643insNN, 2644insNN, 2645insNN, 2646insNN,
2647insNN, 2648insNN, 2649insNN, 2650insNN, 2651insNN, 2652insNN, 2653insNN,
2654insNN, 2655insNN, 2656insNN, 2657insNN, 2658insNN, 2659insNN, 2660insNN,
2661insNN, 2662insNN, 2663insNN, 2664insNN, 2665insNN, 2666insNN, 2667insNN,
2668insNN, 2669insNN, 2670insNN, 2671insNN, 2672insNN, 2673insNN, 2674insNN,
2675insNN, 2676insNN, 2677insNN, 2678insNN, 2679insNN, 2680insNN, 2681insNN,
2682insNN, 2683insNN, 2684insNN, 2685insNN, 2686insNN, 2687insNN, 2688insNN,
2689insNN, 2690insNN, 2691insNN, 2692insNN, 2693insNN, 2694insNN, 2695insNN,
2696insNN, 2697insNN, 2698insNN, 2699insNN, 2700insNN, 2701insNN, 2702insNN,
2703insNN, 2704insNN, 2705insNN, 2706insNN, 2707insNN, 2708insNN, 2709insNN,
2710insNN, 2711insNN, 2712insNN, 2713insNN, 2714insNN, 2715insNN, 2716insNN,
2717insNN, 2718insNN, 2719insNN, 2720insNN, 2721insNN, 2722insNN, 2723insNN,
2724insNN, 2725insNN, 2726insNN, 2727insNN, 2728insNN, 2729insNN, 2730insNN,
2731insNN, 2732insNN, 2733insNN, 2734insNN, 2735insNN, 2736insNN, 2737insNN,
2738insNN, 2739insNN, 2740insNN, 2741insNN, 2742insNN, 2743insNN, 2744insNN,
2745insNN, 2746insNN, 2747insNN, 2748insNN, 2749insNN, 2750insNN, 2751insNN,
2752insNN, 2753insNN, 2754insNN, 2755insNN, 2756insNN, 2757insNN, 2758insNN,
2759insNN, 2760insNN, 2761insNN, 2762insNN, 2763insNN, 2764insNN, 2765insNN,
2766insNN, 2767insNN, 2768insNN, 2769insNN, 2770insNN, 2771insNN, 2772insNN,
2773insNN, 2774insNN, 2775insNN, 2776insNN, 2777insNN, 2778insNN, 2779insNN,
2780insNN, 2781insNN, 2782insNN, 2783insNN, 2784insNN, 2785insNN, 2786insNN,
2787insNN, 2788insNN, 2789insNN, 2790insNN, 2791insNN, 2792insNN, 2793insNN,
2794insNN, 2795insNN, 2796insNN, 2797insNN, 2798insNN, 2799insNN, 2800insNN,
2801insNN, 2802insNN, 2803insNN, 2804insNN, 2805insNN, 2806insNN, 2807insNN,
2808insNN, 2809insNN, 2810insNN, 2811insNN, 2812insNN, 2813insNN, 2814insNN,
2815insNN, 2816insNN, 2817insNN, 2818insNN, 2819insNN, 2820insNN, 2821insNN,
2822insNN, 2823insNN, 2824insNN, 2825insNN, 2826insNN, 2827insNN, 2828insNN,
2829insNN, 2830insNN, 2831insNN, 2832insNN, 2833insNN, 2834insNN, 2835insNN,
2836insNN, 2837insNN, 2838insNN, 2839insNN, 2840insNN, 2841insNN, 2842insNN,
2843insNN, 2844insNN, 2845insNN, 2846insNN, 2847insNN, 2848insNN, 2849insNN,
2850insNN, 2851insNN, 2852insNN, 2853insNN, 2854insNN, 2855insNN, 2856insNN,
2857insNN, 2858insNN, 2859insNN, 2860insNN, 2861insNN, 2862insNN, 2863insNN,
2864insNN, 2865insNN, 2866insNN, 2867insNN, 2868insNN, 2869insNN, 2870insNN,
2871insNN, 2872insNN, 2873insNN, 2874insNN, 2875insNN, 2876insNN, 2877insNN,
2878insNN, 2879insNN, 2880insNN, 2881insNN, 2882insNN, 2883insNN, 2884insNN,
2885insNN, 2886insNN, 2887insNN, 2888insNN, 2889insNN, 2890insNN, 2891insNN,
2892insNN, 2893insNN, 2894insNN, 2895insNN, 2896insNN, 2897insNN, 2898insNN,
2899insNN, 2900insNN, 2901insNN, 2902insNN, 2903insNN, 2904insNN, 2905insNN,
2906insNN, 2907insNN, 2908insNN, 2909insNN, 2910insNN, 2911insNN, 2912insNN,
2913insNN, 2914insNN, 2915insNN, 2916insNN, 2917insNN, 2918insNN, 2919insNN,
2920insNN, 2921insNN, 2922insNN, 2923insNN, 2924insNN, 2925insNN, 2926insNN,
2927insNN, 2928insNN, 2929insNN, 2930insNN, 2931insNN, 2932insNN, 2933insNN,
2934insNN, 2935insNN, 2936insNN, 2937insNN, 2938insNN, 2939insNN, 2940insNN,
2941insNN, 2942insNN, 2943insNN, 2944insNN, 2945insNN, 2946insNN, 2947insNN,
2948insNN, 2949insNN, 2950insNN, 2951insNN, 2952insNN, 2953insNN, 2954insNN,
2955insNN, 2956insNN, 2957insNN, 2958insNN, 2959insNN, 2960insNN, 2961insNN,
2962insNN, 2963insNN, 2964insNN, 2965insNN, 2966insNN, 2967insNN, 2968insNN,
2969insNN, 2970insNN, 2971insNN, 2972insNN, 2973insNN, 2974insNN, 2975insNN,
2976insNN, 2977insNN, 2978insNN, 2979insNN, 2980insNN, 2981insNN, 2982insNN,
2983insNN, 2984insNN, 2985insNN, 2986insNN, 2987insNN, 2988insNN, 2989insNN,
2990insNN, 2991insNN, 2992insNN, 2993insNN, 2994insNN, 2995insNN, 2996insNN,
2997insNN, 2998insNN, 2999insNN, 3000insNN, 3001insNN, 3002insNN, 3003insNN,
3004insNN, 3005insNN, 3006insNN, 3007insNN, 3008insNN, 3009insNN, 3010insNN,
3011insNN, 3012insNN, 3013insNN, 3014insNN, 3015insNN, 3016insNN, 3017insNN,
3018insNN, 3019insNN, 3020insNN, 3021insNN, 3022insNN, 3023insNN, 3024insNN,
3025insNN, 3026insNN, 3027insNN, 3028insNN, 3029insNN, 3030insNN, 3031insNN,
3032insNN, 3033insNN, 3034insNN, 3035insNN, 3036insNN, 3037insNN, 3038insNN,
3039insNN, 3040insNN, 3041insNN, 3042insNN, 3043insNN, 3044insNN, 3045insNN,
3046insNN, 3047insNN, 3048insNN, 3049insNN, 3050insNN, 3051insNN, 3052insNN,
3053insNN, 3054insNN, 3055insNN, 3056insNN, 3057insNN, 3058insNN, 3059insNN,
3060insNN, 3061insNN, 3062insNN, 3063insNN, 3064insNN, 3065insNN, 3066insNN,
3067insNN, 3068insNN, 3069insNN, 3070insNN, 3071insNN, 3072insNN, 3073insNN,
3074insNN, 3075insNN, 3076insNN, 3077insNN, 3078insNN, 3079insNN, 3080insNN,
3081insNN, 3082insNN, 3083insNN, 3084insNN, 3085insNN, 3086insNN, 3087insNN,
3088insNN, 3089insNN, 3090insNN, 3091insNN, 3092insNN, 3093insNN, 3094insNN,
3095insNN, 3096insNN, 3097insNN, 3098insNN, 3099insNN, 3100insNN, 3101insNN,
3102insNN, 3103insNN, 3104insNN, 3105insNN, 3106insNN, 3107insNN, 3108insNN,
3109insNN, 3110insNN, 3111insNN, 3112insNN, 3113insNN, 3114insNN, 3115insNN,
3116insNN, 3117insNN, 3118insNN, 3119insNN, 3120insNN, 3121insNN, 3122insNN,
3123insNN, 3124insNN, 3125insNN, 3126insNN, 3127insNN, 3128insNN, 3129insNN,
3130insNN, 3131insNN, 3132insNN, 3133insNN, 3134insNN, 3135insNN, 3136insNN,
3137insNN, 3138insNN, 3139insNN, 3140insNN, 3141insNN, 3142insNN, 3143insNN,
3144insNN, 3145insNN, 3146insNN, 3147insNN, 3148insNN, 3149insNN, 3150insNN,
3151insNN, 3152insNN, 3153insNN, 3154insNN, 3155insNN, 3156insNN, 3157insNN,
3158insNN, 3159insNN, 3160insNN, 3161insNN, 3162insNN, 3163insNN, 3164insNN,
3165insNN, 3166insNN, 3167insNN, 3168insNN, 3169insNN, 3170insNN, 3171insNN,
3172insNN, 3173insNN, 3174insNN, 3175insNN, 3176insNN, 3177insNN, 3178insNN,
3179insNN, 3180insNN, 3181insNN, 3182insNN, 3183insNN, 3184insNN, 3185insNN,
3186insNN, 3187insNN, 3188insNN, 3189insNN, 3190insNN, 3191insNN, 3192insNN,
3193insNN, 3194insNN, 3195insNN, 3196insNN, 3197insNN, 3198insNN, 3199insNN,
3200insNN, 3201insNN, 3202insNN, 3203insNN, 3204insNN, 3205insNN, 3206insNN,
3207insNN, 3208insNN, 3209insNN, 3210insNN, 3211insNN, 3212insNN, 3213insNN,
3214insNN, 3215insNN, 3216insNN, 3217insNN, 3218insNN, 3219insNN, 3220insNN,
3221insNN, 3222insNN, 3223insNN, 3224insNN, 3225insNN, 3226insNN, 3227insNN,
3228insNN, 3229insNN, 3230insNN, 3231insNN, 3232insNN, 3233insNN, 3234insNN,
3235insNN, 3236insNN, 3237insNN, 3238insNN, 3239insNN, 3240insNN, 3241insNN,
3242insNN, 3243insNN, 3244insNN, 3245insNN, 3246insNN, 3247insNN, 3248insNN,
3249insNN, 3250insNN, 3251insNN, 3252insNN, 3253insNN, 3254insNN, 3255insNN,
3256insNN, 3257insNN, 3258insNN, 3259insNN, 3260insNN, 3261insNN, 3262insNN,
3263insNN, 3264insNN, 3265insNN, 3266insNN, 3267insNN, 3268insNN, 3269insNN,
3270insNN, 3271insNN, 3272insNN, 3273insNN, 3274insNN, 3275insNN, 3276insNN,
3277insNN, 3278insNN, 3279insNN, 3280insNN, 3281insNN, 3282insNN, 3283insNN,
3284insNN, 3285insNN, 3286insNN, 3287insNN, 3288insNN, 3289insNN, 3290insNN,
3291insNN, 3292insNN, 3293insNN, 3294insNN, 3295insNN, 3296insNN, 3297insNN,
3298insNN, 3299insNN, 3300insNN, 3301insNN, 3302insNN, 3303insNN, 3304insNN,
3305insNN, 3306insNN, 3307insNN, 3308insNN, 3309insNN, 3310insNN, 3311insNN,
3312insNN, 3313insNN, 3314insNN, 3315insNN, 3316insNN, 3317insNN, 3318insNN,
3319insNN, 3320insNN, 3321insNN, 3322insNN, 3323insNN, 3324insNN, 3325insNN,
3326insNN, 3327insNN, 3328insNN, 3329insNN, 3330insNN, 3331insNN, 3332insNN,
3333insNN, 3334insNN, 3335insNN, 3336insNN, 3337insNN, 3338insNN, 3339insNN,
3340insNN, 3341insNN, 3342insNN, 3343insNN, 3344insNN, 3345insNN, 3346insNN,
3347insNN, 3348insNN, 3349insNN, 3350insNN, 3351insNN, 3352insNN, 3353insNN,
3354insNN, 3355insNN, 3356insNN, 3357insNN, 3358insNN, 3359insNN, 3360insNN,
3361insNN, 3362insNN, 3363insNN, 3364insNN, 3365insNN, 3366insNN, 3367insNN,
3368insNN, 3369insNN, 3370insNN, 3371insNN, 3372insNN, 3373insNN, 3374insNN,
3375insNN, 3376insNN, 3377insNN, 3378insNN, 3379insNN, 3380insNN, 3381insNN,
3382insNN, 3383insNN, 3384insNN, 3385insNN, 3386insNN, 3387insNN, 3388insNN,
3389insNN, 3390insNN, 3391insNN, 3392insNN, 3393insNN, 3394insNN, 3395insNN,
3396insNN, 3397insNN, 3398insNN, 3399insNN, 3400insNN, 3401insNN, 3402insNN,
3403insNN, 3404insNN, 3405insNN, 3406insNN, 3407insNN, 3408insNN, 3409insNN,
3410insNN, 3411insNN, 3412insNN, 3413insNN, 3414insNN, 3415insNN, 3416insNN,
3417insNN, 3418insNN, 3419insNN, 3420insNN, 3421insNN, 3422insNN, 3423insNN,
3424insNN, 3425insNN, 3426insNN, 3427insNN, 3428insNN, 3429insNN, 3430insNN,
3431insNN, 3432insNN, 3433insNN, 3434insNN, 3435insNN, 3436insNN, 3437insNN,
3438insNN, 3439insNN, 3440insNN, 3441insNN, 3442insNN, 3443insNN, 3444insNN,
3445insNN, 3446insNN, 3447insNN, 3448insNN, 3449insNN, 3450insNN, 3451insNN,
3452insNN, 3453insNN, 3454insNN, 3455insNN, 3456insNN, 3457insNN, 3458insNN,
3459insNN, 3460insNN, 3461insNN, 3462insNN, 3463insNN, 3464insNN, 3465insNN,
3466insNN, 3467insNN, 3468insNN, 3469insNN, 3470insNN, 3471insNN, 3472insNN,
3473insNN, 3474insNN, 3475insNN, 3476insNN, 3477insNN, 3478insNN, 3479insNN,
3480insNN, 3481insNN, 3482insNN, 3483insNN, 3484insNN, 3485insNN, 3486insNN,
3487insNN, 3488insNN, 3489insNN, 3490insNN, 3491insNN, 3492insNN, 3493insNN,
3494insNN, 3495insNN, 3496insNN, 3497insNN, 3498insNN, 3499insNN, 3500insNN,
3501insNN, 3502insNN, 3503insNN, 3504insNN, 3505insNN, 3506insNN, 3507insNN,
3508insNN, 3509insNN, 3510insNN, 3511insNN, 3512insNN, 3513insNN, 3514insNN,
3515insNN, 3516insNN, 3517insNN, 3518insNN, 3519insNN, 3520insNN, 3521insNN,
3522insNN, 3523insNN, 3524insNN, 3525insNN, 3526insNN, 3527insNN, 3528insNN,
3529insNN, 3530insNN, 3531insNN, 3532insNN, 3533insNN, 3534insNN, 3535insNN,
3536insNN, 3537insNN, 3538insNN, 3539insNN, 3540insNN, 3541insNN, 3542insNN,
3543insNN, 3544insNN, 3545insNN, 3546insNN, 3547insNN, 3548insNN, 3549insNN,
3550insNN, 3551insNN, 3552insNN, 3553insNN, 3554insNN, 3555insNN, 3556insNN,
3557insNN, 3558insNN, 3559insNN, 3560insNN, 3561insNN, 3562insNN, 3563insNN,
3564insNN, 3565insNN, 3566insNN, 3567insNN, 3568insNN, 3569insNN, 3570insNN,
3571insNN, 3572insNN, 3573insNN, 3574insNN, 3575insNN, 3576insNN, 3577insNN,
3578insNN, 3579insNN, 3580insNN, 3581insNN, 3582insNN, 3583insNN, 3584insNN,
3585insNN, 3586insNN, 3587insNN, 3588insNN, 3589insNN, 3590insNN, 3591insNN,
3592insNN, 3593insNN, 3594insNN, 3595insNN, 3596insNN, 3597insNN, 3598insNN,
3599insNN, 3600insNN, 3601insNN, 3602insNN, 3603insNN, 3604insNN, 3605insNN,
3606insNN, 3607insNN, 3608insNN, 3609insNN, 3610insNN, 3611insNN, 3612insNN,
3613insNN, 3614insNN, 3615insNN, 3616insNN, 3617insNN, 3618insNN, 3619insNN,
3620insNN, 3621insNN, 3622insNN, 3623insNN, 3624insNN, 3625insNN, 3626insNN,
3627insNN, 3628insNN, 3629insNN, 3630insNN, 3631insNN, 3632insNN, 3633insNN,
3634insNN, 3635insNN, 3636insNN, 3637insNN, 3638insNN, 3639insNN, 3640insNN,
3641insNN, 3642insNN, 3643insNN, 3644insNN, 3645insNN, 3646insNN, 3647insNN,
3648insNN, 3649insNN, 3650insNN, 3651insNN, 3652insNN, 3653insNN, 3654insNN,
3655insNN, 3656insNN, 3657insNN, 3658insNN, 3659insNN, 3660insNN, 3661insNN,
3662insNN, 3663insNN, 3664insNN, 3665insNN, 3666insNN, 3667insNN, 3668insNN,
3669insNN, 3670insNN, 3671insNN, 3672insNN, 3673insNN, 3674insNN, 3675insNN,
3676insNN, 3677insNN, 3678insNN, 3679insNN, 3680insNN, 3681insNN, 3682insNN,
3683insNN, 3684insNN, 3685insNN, 3686insNN, 3687insNN, 3688insNN, 3689insNN,
3690insNN, 3691insNN, 3692insNN, 3693insNN, 3694insNN, 3695insNN, 3696insNN,
3697insNN, 3698insNN, 3699insNN, 3700insNN, 3701insNN, 3702insNN, 3703insNN,
3704insNN, 3705insNN, 3706insNN, 3707insNN, 3708insNN, 3709insNN, 3710insNN,
3711insNN, 3712insNN, 3713insNN, 3714insNN, 3715insNN, 3716insNN, 3717insNN,
3718insNN, 3719insNN, 3720insNN, 3721insNN, 3722insNN, 3723insNN, 3724insNN,
3725insNN, 3726insNN, 3727insNN, 3728insNN, 3729insNN, 3730insNN, 3731insNN,
3732insNN, 3733insNN, 3734insNN, 3735insNN, 3736insNN, 3737insNN, 3738insNN,
3739insNN, 3740insNN, 3741insNN, 3742insNN, 3743insNN, 3744insNN, 3745insNN,
3746insNN, 3747insNN, 3748insNN, 3749insNN, 3750insNN, 3751insNN, 3752insNN,
3753insNN, 3754insNN, 3755insNN, 3756insNN, 3757insNN, 3758insNN, 3759insNN,
3760insNN, 3761insNN, 3762insNN, 3763insNN, 3764insNN, 3765insNN, 3766insNN,
3767insNN, 3768insNN, 3769insNN, 3770insNN, 3771insNN, 3772insNN, 3773insNN,
3774insNN, 3775insNN, 3776insNN, 3777insNN, 3778insNN, 3779insNN, 3780insNN,
3781insNN, 3782insNN, 3783insNN, 3784insNN, 3785insNN, 3786insNN, 3787insNN,
3788insNN, 3789insNN, 3790insNN, 3791insNN, 3792insNN, 3793insNN, 3794insNN,
3795insNN, 3796insNN, 3797insNN, 3798insNN, 3799insNN, 3800insNN, 3801insNN,
3802insNN, 3803insNN, 3804insNN, 3805insNN, 3806insNN, 3807insNN, 3808insNN,
3809insNN, 3810insNN, 3811insNN, 3812insNN, 3813insNN, 3814insNN, 3815insNN,
3816insNN, 3817insNN, 3818insNN, 3819insNN, 3820insNN, 3821insNN, 3822insNN,
3823insNN, 3824insNN, 3825insNN, 3826insNN, 3827insNN, 3828insNN, 3829insNN,
3830insNN, 3831insNN, 3832insNN, 3833insNN, 3834insNN, 3835insNN, 3836insNN,
3837insNN, 3838insNN, 3839insNN, 3840insNN, 3841insNN, 3842insNN, 3843insNN,
3844insNN, 3845insNN, 3846insNN, 3847insNN, 3848insNN, 3849insNN, 3850insNN,
3851insNN, 3852insNN, 3853insNN, 3854insNN, 3855insNN, 3856insNN, 3857insNN,
3858insNN, 3859insNN, 3860insNN, 3861insNN, 3862insNN, 3863insNN, 3864insNN,
3865insNN, 3866insNN, 3867insNN, 3868insNN, 3869insNN, 3870insNN, 3871insNN,
3872insNN, 3873insNN, 3874insNN, 3875insNN, 3876insNN, 3877insNN, 3878insNN,
3879insNN, 3880insNN, 3881insNN, 3882insNN, 3883insNN, 3884insNN, 3885insNN,
3886insNN, 3887insNN, 3888insNN, 3889insNN, 3890insNN, 3891insNN, 3892insNN,
3893insNN, 3894insNN, 3895insNN, 3896insNN, 3897insNN, 3898insNN, 3899insNN,
3900insNN, 3901insNN, 3902insNN, 3903insNN, 3904insNN, 3905insNN, 3906insNN,
3907insNN, 3908insNN, 3909insNN, 3910insNN, 3911insNN, 3912insNN, 3913insNN,
3914insNN, 3915insNN, 3916insNN, 3917insNN, 3918insNN, 3919insNN, 3920insNN,
3921insNN, 3922insNN, 3923insNN, 3924insNN, 3925insNN, 3926insNN, 3927insNN,
3928insNN, 3929insNN, 3930insNN, 3931insNN, 3932insNN, 3933insNN, 3934insNN,
3935insNN, 3936insNN, 3937insNN, 3938insNN, 3939insNN, 3940insNN, 3941insNN,
3942insNN, 3943insNN, 3944insNN, 3945insNN, 3946insNN, 3947insNN, 3948insNN,
3949insNN, 3950insNN, 3951insNN, 3952insNN, 3953insNN, 3954insNN, 3955insNN,
3956insNN, 3957insNN, 3958insNN, 3959insNN, 3960insNN, 3961insNN, 3962insNN,
3963insNN, 3964insNN, 3965insNN, 3966insNN, 3967insNN, 3968insNN, 3969insNN,
3970insNN, 3971insNN, 3972insNN, 3973insNN, 3974insNN, 3975insNN, 3976insNN,
3977insNN, 3978insNN, 3979insNN, 3980insNN, 3981insNN, 3982insNN, 3983insNN,
3984insNN, 3985insNN, 3986insNN, 3987insNN, 3988insNN, 3989insNN, 3990insNN,
3991insNN, 3992insNN, 3993insNN, 3994insNN, 3995insNN, 3996insNN, 3997insNN,
3998insNN, 3999insNN, 4000insNN, 4001insNN, 4002insNN, 4003insNN, 4004insNN,
4005insNN, 4006insNN, 4007insNN, 4008insNN, 4009insNN, 4010insNN, 4011insNN,
4012insNN, 4013insNN, 4014insNN, 4015insNN, 4016insNN, 4017insNN, 4018insNN,
4019insNN, 4020insNN, 4021insNN, 4022insNN, 4023insNN, 4024insNN, 4025insNN,
4026insNN, 4027insNN, 4028insNN, 4029insNN, 4030insNN, 4031insNN, 4032insNN,
4033insNN, 4034insNN, 4035insNN, 4036insNN, 4037insNN, 4038insNN, 4039insNN,
4040insNN, 4041insNN, 4042insNN, 4043insNN, 4044insNN, 4045insNN, 4046insNN,
4047insNN, 4048insNN, 4049insNN, 4050insNN, 4051insNN, 4052insNN, 4053insNN,
4054insNN, 4055insNN, 4056insNN, 4057insNN, 4058insNN, 4059insNN, 4060insNN,
4061insNN, 4062insNN, 4063insNN, 4064insNN, 4065insNN, 4066insNN, 4067insNN,
4068insNN, 4069insNN, 4070insNN, 4071insNN, 4072insNN, 4073insNN, 4074insNN,
4075insNN, 4076insNN, 4077insNN, 4078insNN, 4079insNN, 4080insNN, 4081insNN,
4082insNN, 4083insNN, 4084insNN, 4085insNN, 4086insNN, 4087insNN, 4088insNN,
4089insNN, 4090insNN, 4091insNN, 4092insNN, 4093insNN, 4094insNN, 4095insNN,
4096insNN, 4097insNN, 4098insNN, 4099insNN, 4100insNN, 4101insNN, 4102insNN,
4103insNN, 4104insNN, 4105insNN, 4106insNN, 4107insNN, 4108insNN, 4109insNN,
4110insNN, 4111insNN, 4112insNN, 4113insNN, 4114insNN, 4115insNN, 4116insNN,
4117insNN, 4118insNN, 4119insNN, 4120insNN, 4121insNN, 4122insNN, 4123insNN,
4124insNN, 4125insNN, 4126insNN, 4127insNN, 4128insNN, 4129insNN, 4130insNN,
4131insNN, 4132insNN, 4133insNN, 4134insNN, 4135insNN, 4136insNN, 4137insNN,
4138insNN, 4139insNN, 4140insNN, 4141insNN, 4142insNN, 4143insNN, 4144insNN,
4145insNN, 4146insNN, 4147insNN, 4148insNN, 4149insNN, 4150insNN, 4151insNN,
4152insNN, 4153insNN, 4154insNN, 4155insNN, 4156insNN, 4157insNN, 4158insNN,
4159insNN, 4160insNN, 4161insNN, 4162insNN, 4163insNN, 4164insNN, 4165insNN,
4166insNN, 4167insNN, 4168insNN, 4169insNN, 4170insNN, 4171insNN, 4172insNN,
4173insNN, 4174insNN, 4175insNN, 4176insNN, 4177insNN, 4178insNN, 4179insNN,
4180insNN, 4181insNN, 4182insNN, 4183insNN, 4184insNN, 4185insNN, 4186insNN,
4187insNN, 4188insNN, 4189insNN, 4190insNN, 4191insNN, 4192insNN, 4193insNN,
4194insNN, 4195insNN, 4196insNN, 4197insNN, 4198insNN, 4199insNN, 4200insNN,
4201insNN, 4202insNN, 4203insNN, 4204insNN, 4205insNN, 4206insNN, 4207insNN,
4208insNN, 4209insNN, 4210insNN, 4211insNN, 4212insNN, 4213insNN, 4214insNN,
4215insNN, 4216insNN, 4217insNN, 4218insNN, 4219insNN, 4220insNN, 4221insNN,
4222insNN, 4223insNN, 4224insNN, 4225insNN, 4226insNN, 4227insNN, 4228insNN,
4229insNN, 4230insNN, 4231insNN, 4232insNN, 4233insNN, 4234insNN, 4235insNN,
4236insNN, 4237insNN, 4238insNN, 4239insNN, 4240insNN, 4241insNN, 4242insNN,
4243insNN, 4244insNN, 4245insNN, 4246insNN, 4247insNN, 4248insNN, 4249insNN,
4250insNN, 4251insNN, 4252insNN, 4253insNN, 4254insNN, 4255insNN, 4256insNN,
4257insNN, 4258insNN, 4259insNN, 4260insNN, 4261insNN, 4262insNN, 4263insNN,
4264insNN, 4265insNN, 4266insNN, 4267insNN, 4268insNN, 4269insNN, 4270insNN,
4271insNN, 4272insNN, 4273insNN, 4274insNN, 4275insNN, 4276insNN, 4277insNN,
4278insNN, 4279insNN, 4280insNN, 4281insNN, 4282insNN, 4283insNN, 4284insNN,
4285insNN, 4286insNN, 4287insNN, 4288insNN, 4289insNN, 4290insNN, 4291insNN,
4292insNN, 4293insNN, 4294insNN, 4295insNN, 4296insNN, 4297insNN, 4298insNN,
4299insNN, 4300insNN, 4301insNN, 4302insNN, 4303insNN, 4304insNN, 4305insNN,
4306insNN, 4307insNN, 4308insNN, 4309insNN, 4310insNN, 4311insNN, 4312insNN,
4313insNN, 4314insNN, 4315insNN, 4316insNN, 4317insNN, 4318insNN, 4319insNN,
4320insNN, 4321insNN, 4322insNN, 4323insNN, 4324insNN, 4325insNN, 4326insNN,
4327insNN, 4328insNN, 4329insNN, 4330insNN, 4331insNN, 4332insNN, 4333insNN,
4334insNN, 4335insNN, 4336insNN, 4337insNN, 4338insNN, 4339insNN, 4340insNN,
4341insNN, 4342insNN, 4343insNN, 4344insNN, 4345insNN, 4346insNN, 4347insNN,
4348insNN, 4349insNN, 4350insNN, 4351insNN, 4352insNN, 4353insNN, 4354insNN,
4355insNN, 4356insNN, 4357insNN, 4358insNN, 4359insNN, 4360insNN, 4361insNN,
4362insNN, 4363insNN, 4364insNN, 4365insNN, 4366insNN, 4367insNN, 4368insNN,
4369insNN, 4370insNN, 4371insNN, 4372insNN, 4373insNN, 4374insNN, 4375insNN,
4376insNN, 4377insNN, 4378insNN, 4379insNN, 4380insNN, 4381insNN, 4382insNN,
4383insNN, 4384insNN, 4385insNN, 4386insNN, 4387insNN, 4388insNN, 4389insNN,
4390insNN, 4391insNN, 4392insNN, 4393insNN, 4394insNN, 4395insNN, 4396insNN,
4397insNN, 4398insNN, 4399insNN, 4400insNN, 4401insNN, 4402insNN, 4403insNN,
4404insNN, 4405insNN, 4406insNN, 4407insNN, 4408insNN, 4409insNN, 4410insNN,
4411insNN, 4412insNN, 4413insNN, 4414insNN, 4415insNN, 4416insNN, 4417insNN,
4418insNN, 4419insNN, 4420insNN, 4421insNN, 4422insNN, 4423insNN, 4424insNN,
4425insNN, 4426insNN, 4427insNN, 4428insNN, 4429insNN, 4430insNN, 4431insNN,
4432insNN, 4433insNN, 4434insNN, 4435insNN, 4436insNN, 4437insNN, 4438insNN,
4439insNN, 4440insNN, 4441insNN, 4442insNN, 4443insNN, 4444insNN, 4445insNN,
4446insNN, 4447insNN, 4448insNN, 4449insNN, 4450insNN, 4451insNN, 4452insNN,
4453insNN, 4454insNN, 4455insNN, 4456insNN, 4457insNN, 4458insNN, 4459insNN,
4460insNN, 4461insNN, 4462insNN, 4463insNN, 4464insNN, 4465insNN, 4466insNN,
4467insNN, 4468insNN, 4469insNN, 4470insNN, 4471insNN, 4472insNN, 4473insNN,
4474insNN, 4475insNN, 4476insNN, 4477insNN, 4478insNN, 4479insNN, 4480insNN,
4481insNN, 4482insNN, 4483insNN, 4484insNN, 4485insNN, 4486insNN, 4487insNN,
4488insNN, 4489insNN, 4490insNN, 4491insNN, 4492insNN, 4493insNN, 4494insNN,
4495insNN, 4496insNN, 4497insNN, 4498insNN, 4499insNN, 4500insNN, 4501insNN,
4502insNN, 4503insNN, 4504insNN, 4505insNN, 4506insNN, 4507insNN, 4508insNN,
4509insNN, 4510insNN, 4511insNN, 4512insNN, 4513insNN, 4514insNN, 4515insNN,
4516insNN, 4517insNN, 4518insNN, 4519insNN, 4520insNN, 4521insNN, 4522insNN,
4523insNN, 4524insNN, 4525insNN, 4526insNN, 4527insNN, 4528insNN, 4529insNN,
4530insNN, 4531insNN, 4532insNN, 4533insNN, 4534insNN, 4535insNN, 4536insNN,
4537insNN, 4538insNN, 4539insNN, 4540insNN, 4541insNN, 4542insNN, 4543insNN,
4544insNN, 4545insNN, 4546insNN, 4547insNN, 4548insNN, 4549insNN, 4550insNN,
4551insNN, 4552insNN, 4553insNN, 4554insNN, 4555insNN, 4556insNN, 4557insNN,
4558insNN, 4559insNN, 4560insNN, 4561insNN, 4562insNN, 4563insNN, 4564insNN,
4565insNN, 4566insNN, 4567insNN, 4568insNN, 4569insNN, 4570insNN, 4571insNN,
4572insNN, 4573insNN, 4574insNN, 4575insNN, 4576insNN, 4577insNN, 4578insNN,
4579insNN, 4580insNN, 4581insNN, 4582insNN, 4583insNN, 4584insNN, 4585insNN,
4586insNN, 4587insNN, 4588insNN, 4589insNN, 4590insNN, 4591insNN, 4592insNN,
4593insNN, 4594insNN, 4595insNN, 4596insNN, 4597insNN, 4598insNN, 4599insNN,
4600insNN, 4601insNN, 4602insNN, 4603insNN, 4604insNN, 4605insNN, 4606insNN,
4607insNN, 4608insNN, 4609insNN, 4610insNN, 4611insNN, 4612insNN, 4613insNN,
4614insNN, 4615insNN, 4616insNN, 4617insNN, 4618insNN, 4619insNN, 4620insNN,
4621insNN, 4622insNN, 4623insNN, 4624insNN, 4625insNN, 4626insNN, 4627insNN,
4628insNN, 4629insNN, 4630insNN, 4631insNN, 4632insNN, 4633insNN, 4634insNN,
4635insNN, 4636insNN, 4637insNN, 4638insNN, 4639insNN, 4640insNN, 4641insNN,
4642insNN, 4643insNN, 4644insNN, 4645insNN, 4646insNN, 4647insNN, 4648insNN,
4649insNN, 4650insNN, 4651insNN, 4652insNN, 4653insNN, 4654insNN, 4655insNN,
4656insNN, 4657insNN, 4658insNN, 4659insNN, 4660insNN, 4661insNN, 4662insNN,
4663insNN, 4664insNN, 4665insNN, 4666insNN, 4667insNN, 4668insNN, 4669insNN,
4670insNN, 4671insNN, 4672insNN, 4673insNN, 4674insNN, 4675insNN, 4676insNN,
4677insNN, 4678insNN, 4679insNN, 4680insNN, 4681insNN, 4682insNN, 4683insNN,
4684insNN, 4685insNN, 4686insNN, 4687insNN, 4688insNN, 4689insNN, 4690insNN,
4691insNN, 4692insNN, 4693insNN, 4694insNN, 4695insNN, 4696insNN, 4697insNN,
4698insNN, 4699insNN, 4700insNN, 4701insNN, 4702insNN, 4703insNN, 4704insNN,
4705insNN, 4706insNN, 4707insNN, 4708insNN, 4709insNN, 4710insNN, 4711insNN,
4712insNN, 4713insNN, 4714insNN, 4715insNN, 4716insNN, 4717insNN, 4718insNN,
4719insNN, 4720insNN, 4721insNN, 4722insNN, 4723insNN, 4724insNN, 4725insNN,
4726insNN, 4727insNN, 4728insNN, 4729insNN, 4730insNN, 4731insNN, 4732insNN,
4733insNN, 4734insNN, 4735insNN, 4736insNN, 4737insNN, 4738insNN, 4739insNN,
4740insNN, 4741insNN, 4742insNN, 4743insNN, 4744insNN, 4745insNN, 4746insNN,
4747insNN, 4748insNN, 4749insNN, 4750insNN, 4751insNN, 4752insNN, 4753insNN,
4754insNN, 4755insNN, 4756insNN, 4757insNN, 4758insNN, 4759insNN, 4760insNN,
4761insNN, 4762insNN, 4763insNN, 4764insNN, 4765insNN, 4766insNN, 4767insNN,
4768insNN, 4769insNN, 4770insNN, 4771insNN, 4772insNN, 4773insNN, 4774insNN,
4775insNN, 4776insNN, 4777insNN, 4778insNN, 4779insNN, 4780insNN, 4781insNN,
4782insNN, 4783insNN, 4784insNN, 4785insNN, 4786insNN, 4787insNN, 4788insNN,
4789insNN, 4790insNN, 4791insNN, 4792insNN, 4793insNN, 4794insNN, 4795insNN,
4796insNN, 4797insNN, 4798insNN, 4799insNN, 4800insNN, 4801insNN, 4802insNN,
4803insNN, 4804insNN, 4805insNN, 4806insNN, 4807insNN, 4808insNN, 4809insNN,
4810insNN, 4811insNN, 4812insNN, 4813insNN, 4814insNN, 4815insNN, 4816insNN,
4817insNN, 4818insNN, 4819insNN, 4820insNN, 4821insNN, 4822insNN, 4823insNN,
4824insNN, 4825insNN, 4826insNN, 4827insNN, 4828insNN, 4829insNN, 4830insNN,
4831insNN, 4832insNN, 4833insNN, 4834insNN, 4835insNN, 4836insNN, 4837insNN,
4838insNN, 4839insNN, 4840insNN, 4841insNN, 4842insNN, 4843insNN, 4844insNN,
4845insNN, 4846insNN, 4847insNN, 4848insNN, 4849insNN, 4850insNN, 4851insNN,
4852insNN, 4853insNN, 4854insNN, 4855insNN, 4856insNN, 4857insNN, 4858insNN,
4859insNN, 4860insNN, 4861insNN, 4862insNN, 4863insNN, 4864insNN, 4865insNN,
4866insNN, 4867insNN, 4868insNN, 4869insNN, 4870insNN, 4871insNN, 4872insNN,
4873insNN, 4874insNN, 4875insNN, 4876insNN, 4877insNN, 4878insNN, 4879insNN,
4880insNN, 4881insNN, 4882insNN, 4883insNN, 4884insNN, 4885insNN, 4886insNN,
4887insNN, 4888insNN, 4889insNN, 4890insNN, 4891insNN, 4892insNN, 4893insNN,
4894insNN, 4895insNN, 4896insNN, 4897insNN, 4898insNN, 4899insNN, 4900insNN,
4901insNN, 4902insNN, 4903insNN, 4904insNN, 4905insNN, 4906insNN, 4907insNN,
4908insNN, 4909insNN, 4910insNN, 4911insNN, 4912insNN, 4913insNN, 4914insNN,
4915insNN, 4916insNN, 4917insNN, 4918insNN, 4919insNN, 4920insNN, 4921insNN,
4922insNN, 4923insNN, 4924insNN, 4925insNN, 4926insNN, 4927insNN, 4928insNN,
4929insNN, 4930insNN, 4931insNN, 4932insNN, 4933insNN, 4934insNN, 4935insNN,
4936insNN, 4937insNN, 4938insNN, 4939insNN, 4940insNN, 4941insNN, 4942insNN,
4943insNN, 4944insNN, 4945insNN, 4946insNN, 4947insNN, 4948insNN, 4949insNN,
4950insNN, 4951insNN, 4952insNN, 4953insNN, 4954insNN, 4955insNN, 4956insNN,
4957insNN, 4958insNN, 4959insNN, 4960insNN, 4961insNN, 4962insNN, 4963insNN,
4964insNN, 4965insNN, 4966insNN, 4967insNN, 4968insNN, 4969insNN, 4970insNN,
4971insNN, 4972insNN, 4973insNN, 4974insNN, 4975insNN, 4976insNN, 4977insNN,
4978insNN, 4979insNN, 4980insNN, 4981insNN, 4982insNN, 4983insNN, 4984insNN,
4985insNN, 4986insNN, 4987insNN, 4988insNN, 4989insNN, 4990insNN, 4991insNN,
4992insNN, 4993insNN, 4994insNN, 4995insNN, 4996insNN, 4997insNN, 4998insNN,
4999insNN, 5000insNN, 5001insNN, 5002insNN, 5003insNN, 5004insNN, 5005insNN,
5006insNN, 5007insNN, 5008insNN, 5009insNN, 5010insNN, 5011insNN, 5012insNN,
5013insNN, 5014insNN, 5015insNN, 5016insNN, 5017insNN, 5018insNN, 5019insNN,
5020insNN, 5021insNN, 5022insNN, 5023insNN, 5024insNN, 5025insNN, 5026insNN,
5027insNN, 5028insNN, 5029insNN, 5030insNN, 5031insNN, 5032insNN, 5033insNN,
5034insNN, 5035insNN, 5036insNN, 5037insNN, 5038insNN, 5039insNN, 5040insNN,
5041insNN, 5042insNN, 5043insNN, 5044insNN, 5045insNN, 5046insNN, 5047insNN,
5048insNN, 5049insNN, 5050insNN, 5051insNN, 5052insNN, 5053insNN, 5054insNN,
5055insNN, 5056insNN, 5057insNN, 5058insNN, 5059insNN, 5060insNN, 5061insNN,
5062insNN, 5063insNN, 5064insNN, 5065insNN, 5066insNN, 5067insNN, 5068insNN,
5069insNN, 5070insNN, 5071insNN, 5072insNN, 5073insNN, 5074insNN, 5075insNN,
5076insNN, 5077insNN.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All references mentioned herein are incorporated by reference.

We claim:

1. An isolated mutant BRCA1 gene or polynucleotide fragment thereof containing a mutation site, or a polynucleotide complementary to said gene or said fragment, having an in-frame stop codon before codon 1863, with the proviso that the mutation site not be one defined by TABLE 1.

2. An isolated mutant BRCA1 gene, a polynucleotide fragment of said mutant BRCA1 gene, or a complementary polynucleotide to said mutant BRCA1 gene or said fragment, according to claim 1, containing a truncating mutation and forming a stop codon as defined in TABLES 3-7, wherein the mutant BRCA1 gene codes for a truncated BRCA1 protein which lacks full biological activity of wild-type BRCA1 protein.

3. An isolated mutant BRCA1 gene, a polynucleotide fragment of said mutant BRCA1 gene or a complementary polynucleotide to said mutant BRCA1 gene or said fragment according to claim 1, containing a truncating mutation and having the sequence 5′ R1-R2-R3 3′; where
R1 is a wild type BRCA1 DNA sequence from nucleotide number 120 to X, R2 is TAA, TAG or TGA, and R3 is a wild type BRCA1 DNA sequence from nucleotide number X+4 to 5711 and where X=123 to 5710; or
R1 is a wild type BRCA1 DNA sequence from nucleotide number 120 to X, R2 is zero nucleotides, and R3 is the wild type BRCA1 DNA sequence from nucleotides X+Y+1 to 5711, where Y is an integer of 3n+1 or 3n+2 where n=0 to 1861 and where X=123 to 5707; or
R1 is a wild type BRCA1 DNA sequence from nucleotide number 120 to X, R2 is Y nucleotides of any sequence, and R3 contains the wild type BRCA1 DNA sequence of nucleotide number X+1 to 5711, where Y is 3n+1 or 3n+2 where n is an integer of zero or greater, and where X=123 to 5707;
wherein the BRCA1 gene codes for a truncated BRCA1 protein which lacks full biological activity of wild-type BRCA1 protein, with the proviso that the mutation not be one defined by TABLE 1.

4. An isolated mutant BRCA1 gene or a polynucleotide fragment thereof, or a polynucleotide complementary to said mutant BRCA1 gene or said fragment according to claim 1, containing a truncating mutation site and capable of specifically hybridizing to an oligonucleotide probe being at least 12 nucleotides in length and having the sequence 5′ R1-R2-R3 3′; where either
R1 contains at its 3′ end three nucleotides complementary to codon X-1 of the wild-type BRCA1 gene; R2 is complementary to TAG, TAA or TGA, and R3 contains at its 5′ end three nucleotides complementary to codon X+1 of the wild type BRCA1 gene, where X=2 to 1862; or
R1 contains at its 3′ end three nucleotides complementary to nucleotide numbers X-2 to X of the wild-type BRCA1 gene, R2 is an oligonucleotide having Y nucleotides of any sequence, R3 contains at its 5′ end three nucleotides complementary to nucleotide numbers X+1 to X+3 of the wild type BRCA1 gene, where Y is an integer greater than zero and is not 3 or a multiple of 3, where X=122 to 5707; or
R1 contains at its 3′ end three nucleotides complementary to nucleotide numbers X-2 to X of the wild-type BRCA1 gene, R2=zero, R3 contains at its 5′ end three nucleotides complementary to nucleotide numbers X+1+Y of the wild type BRCA1 DNA sequence, where Y=1 to 5582 but is not 3 or a multiple of 3 and where X=122 to 5706;
wherein the oligonucleotide probe is unable to specifically hybridize to the wild-type BRCA1 gene; and with the proviso that the mutation not be one defined by TABLE 1.

5. A mutant BRCA1 gene or a polynucleotide fragment thereof, or a polynucleotide complementary to said mutant BRCA1 gene or said fragment according to claim 1, containing a premature stop codon and incapable of expressing a complete BRCA1 protein;
wherein said mutant BRCA1 gene contains a mutation resulting from a substituted nucleotide in the naturally occurring (wild-type) sequence so that an in-frame stop codon is formed at any of codon numbers 2-1863; or
inserted or deleted 3n+1 or 3n+2 nucleotides, where n is an integer of 0 or greater, causing a frame shift mutation in the naturally occurring (wild-type) sequence so that an in-frame stop codon is formed at any of codon numbers 2-1863;
wherein a mutant BRCA1 protein expressed from said mutant BRCA1 gene lacks full biological activity of naturally occurring (wild type) BRCA1 protein; and
with the proviso that the mutation is not one of the mutations listed in TABLE 1.

6. A method for detecting a mutation in a sample containing a mutant BRCA1 gene according to claim 1, comprising:
a) amplifying at least a fragment of sample BRCA1 gene;
b) dermining the sequence of said at least a fragment of the sample BRCA1 gene; and
c) comparing the sequence obtained with a wild-type BRCA1 sequence;
wherein the presence of the sequence of said mutation in said sample BRCA1 gene indicates the presence of a mutation in the sample.

7. A method for detecting a mutation in a sample containing a BRCA1 gene comprising:
a) amplifying at least a fragment of sample BRCA1 gene;
b) determining the sequence of said at least a fragment of sample BRCA1 gene; and
c) comparing the sequence obtained with one or more sequences of mutant BRCA1 genes according to claim 1;
wherein the presence of a sequence of said mutant BRCA1 gene indicates the presence of a mutation in the sample.

8. A method for detecting a mutation in a BRCA1 gene comprising:
a) obtaining a BRCA1 protein expressed by a BRCA1 gene according to claim 1; and
b) determining the relative molecular weight of said BRCA1 protein compared to wild type BRCA1 protein;
c) wherein the presence of said BRCA1 protein having a molecular weight less than that of wild-type BRCA1 protein indicates the presence of a mutation in the BRCA1 gene.

9. An oligonucleotide capable of specifically hybridizing to either:
a) a DNA containing a mutant BRCA1 as defined in claim 1, or a DNA having a sequence complementary thereto; or
b) a DNA containing a wild-type BRCA1 sequence at a mutation site other than defined by TABLE 1, or a DNA having a sequence complementary thereto;
but not both a) and b).

10. A plurality of oligonucleotides comprising at least one oligonucleotide according to claim 9 and at least one additional oligonucleotide capable of specifically hybridizing to the wild-type BRCA1 gene or its complement.

11. A chip array having “n” elements for performing allele specific sequence-based techniques using the oligonucleotide probes comprising, a solid phase chip and a plurality of oligonucleotides of claim 10 having “n” different nucleotide sequences, wherein “n” is an integer greater than one;
wherein said oligonucleotides are bound to said solid phase chip in a manner which permits said oligonucleotides to effectively hybridize to complementary oligonucleotides or polynucleotides;
wherein oligonucleotides having different nucleotide sequences are bound to said solid phase chip at different locations so that a particular location on said solid phase chip exclusively binds oligonucleotides having a specific nucleotide sequence; and
wherein at least one oligonucleotide is capable of specifically hybridizing to a mutant BRCA1 gene having a truncating mutation as defined by TABLES 3-7 or a DNA complementary thereto, with the proviso that the mutation not be one defined by TABLE 1, wherein the BRCA1 gene codes for a truncated BRCA1 protein which lacks full biological activity of wild-type BRCA1 protein.

12. A method for detecting the presence or absence of a BRCA1 gene mutation in a sample DNA comprising, specifically hybridizing sample DNA with an oligonucleotide according to claim 9 under stringent conditions and determining whether said oligonucleotide specifically hybridizes to said sample DNA.

13. A method for determining therapy for an individual having a tumor comprising:
a) obtaining a DNA containing biological sample from the individual having a tumor;
b) determining whether the DNA contains a mutant BRCA1 gene according to claim 1, or a DNA complementary thereto; and
c) determining appropriate therapy based on the presence or absence of said mutant BRCA1 gene;
wherein the BRCA1 gene codes for a truncated BRCA1 protein which lacks full biological activity of wild-type BRCA1 protein.

14. A method for determining whether to begin diagnostic or prophylactic treatment for an individual comprising:
a) obtaining a DNA containing biological sample from the individual;
b) determining whether the DNA contains a mutant BRCA1 gene according to claim 1; and
c) determining appropriate diagnostic or prophylactic treatment based on the presence or absence of said mutant BRCA1 gene;
wherein the mutant BRCA1 gene codes for a truncated BRCA1 protein which lacks full biological activity of wild-type BRCA1 protein.

15. A method for treating a condition associated with a mutant BRCA1 gene comprising administering biologically active BRCA1 protein to a patient with a condition associated with a mutant BRCA1 gene wherein said patient contains cells having a mutant BRCA1 gene according to claim 1, wherein the BRCA1 gene codes for a truncated BRCA1 protein which lacks full biological activity of wild-type BRCA1 protein.

16. A method for preventing a condition associated with a mutant BRCA1 gene comprising administering biologically active BRCA1 protein to a patient with a cancer wherein said patient contains cells having a truncating BRCA1 gene mutation according to claim 1, wherein the BRCA1 gene codes for a truncated BRCA1 protein which lacks full biological activity of wild-type BRCA1 protein.

17. A method for determining appropriate gene therapy for an individual comprising detecting the presence of a mutant BRCA1 gene according to claim 1, in cells from the individual and administering a DNA containing biologically active BRCA1 gene to the individual, wherein the mutant BRCA1 gene codes for a truncated BRCA1 protein which lacks full biological activity of wild-type BRCA1 protein.

18. A method for detecting a mutation in a sample containing a mutant BRCA1 gene according to claim 2, comprising:
a) amplifying at least a fragment of sample BRCA1 gene;
b) determining the sequence of said at least a fragment of sample BRCA1 gene; and
c) comparing the sequence obtained with a wild-type BRCA1 sequence;
wherein the presence of the sequence of said mutant BRCA1 gene indicates the presence of a mutation in the sample.

19. A method for detecting a mutation in a sample containing a BRCA1 gene comprising:
a) amplifying at least a fragment of sample BRCA1 gene;
b) determining the sequence of said at least a fragment of sample BRCA1 gene; and
c) comparing the sequence obtained with one or more of mutant BRCA1 genes according to claim 2;
wherein the presence of a sequence of said mutant BRCA1 gene indicates the presence of a mutation in the sample.

20. A method for detecting a mutation in a BRCA1 gene comprising:
a) obtaining a BRCA1 protein expressed by a BRCA1 gene according to claim 2; and
b) determining the relative molecular weight of said BRCA1 protein compared to wild type BRCA1 protein;
wherein the presence of said BRCA1 protein having a molecular weight less than that of wild-type BRCA1 protein indicates the presence of a mutation in the BRCA1 gene.

21. An oligonucleotide capable of specifically hybridizing to either:
a) a DNA containing a mutant BRCA1 as defined in claim 2, or a DNA having a sequence complementary thereto; or
b) a DNA containing a wild-type BRCA1 sequence at a mutation site other than defined by TABLE 1, or a DNA having a sequence complementary thereto;
but not both a) and b).

22. A plurality of oligonucleotides comprising at least one oligonucleotide according to claim 21 and at least one additional oligonucleotide capable of specifically hybridizing to the wild-type BRCA1 gene or its complement.

23. A chip array having “n” elements for performing allele specific sequence-based techniques using the oligonucleotide probes comprising a solid phase chip and a plurality of oligonucleotides of claim 22 having “n” different nucleotide sequences, wherein “n” is an integer greater than two;
wherein said oligonucleotides are bound to said solid phase chip in a manner which permits said oligonucleotides to effectively hybridize to complementary oligonucleotides or polynucleotides;
wherein oligonucleotides having different nucleotide sequences are bound to said solid phase chip at different locations so that a particular location on said solid phase chip exclusively binds oligonucleotides having a specific nucleotide sequence; and
wherein at least one oligonucleotide is capable of specifically hybridizing to a mutant BRCA1 gene having a truncating mutation as defined by TABLES 3-7 or a DNA complementary thereto, with the proviso that the mutation not be one defined by TABLE 1, wherein the BRCA1 gene codes for a truncated BRCA1 protein which lacks full biological activity of wild-type BRCA1 protein.

24. A method for detecting the presence or absence of a BRCA1 gene mutation in a sample DNA comprising specifically hybridizing sample DNA with an oligonucleotide according to claim 21 under stringent conditions and determining whether said oligonucleotide specifically hybridizes to said sample DNA.

25. A method for determining therapy for an individual having a tumor comprising obtaining a DNA containing biological sample from the individual having a tumor, determining whether the DNA contains a mutant BRCA1 gene according to claim 2, or a DNA complementary thereto, and determining appropriate therapy based on the presence or absence of said mutant BRCA1 gene, wherein the BRCA1 gene codes for a truncated BRCA1 protein which lacks full biological activity of wild-type BRCA1 protein.

26. A method for determining whether to begin diagnostic or prophylactic treatment for an individual comprising:
a) taking a DNA containing biological sample from the individual;
b) determining whether the DNA contains a mutant BRCA1 gene according to claim 2; and
c) determining appropriate diagnostic or prophylactic treatment based on the presence or absence of said mutant BRCA1 gene;
wherein the mutant BRCA1 gene codes for a truncated BRCA1 protein which lacks full biological activity of wild-type BRCA1 protein.

27. A method for treating a condition associated with a mutant BRCA1 gene comprising, administering biologically active BRCA1 protein to a patient with a condition associated with a mutant BRCA1 gene wherein said patient contains cells having a mutant BRCA1 gene according to claim 2, wherein the BRCA1 gene codes for a truncated BRCA1 protein which lacks full biological activity of wild-type BRCA1 protein.

28. A method for preventing a condition associated with a mutant BRCA1 gene comprising, administering biologically active BRCA1 protein to a patient with a cancer wherein said patient contains cells having a truncating BRCA1 gene mutation according to claim 2, wherein the BRCA1 gene codes for a truncated BRCA1 protein which lacks full biological activity of wild-type BRCA1 protein.

29. A method for determining appropriate gene therapy for an individual comprising, detecting the presence of a mutant BRCA1 gene according to claim 2, in cells from the individual and administering a DNA containing biologically active BRCA1 gene to the individual, wherein the mutant BRCA1 gene codes for a truncated BRCA1 protein which lacks full biological activity of wild-type BRCA1 protein.

30. A method for detecting a mutation in a sample containing a mutant BRCA1 gene according to claim 3, comprising:
a) amplifying at least a fragment of sample BRCA1 gene;
b) determining the sequence of said at least a fragment of sample BRCA1 gene; and
c) comparing the sequence obtained with a wild-type BRCA1 sequence;
wherein the presence of the sequence of said mutant BRCA1 gene indicates the presence of a mutation in the sample.

31. A method for detecting a mutation in a sample containing a BRCA1 gene comprising:
a) amplifying at least a fragment of sample BRCA1 gene;
b) determining the sequence of said at least a fragment of sample BRCA1 gene; and
c) comparing the sequence obtained with one or more of mutant BRCA1 genes according to claim 3;
wherein the presence of a sequence of said mutant BRCA1 gene indicates the presence of a mutation in the sample.

32. A method for detecting a mutation in a BRCA1 gene comprising, obtaining a BRCA1 protein expressed by a BRCA1 gene according to claim 3, and determining the relative molecular weight of said BRCA1 protein compared to wild type BRCA1 protein, wherein the presence of said BRCA1 protein having a molecular weight less than that of wild-type BRCA1 protein indicates the presence of a mutation in the BRCA1 gene.

33. An oligonucleotide capable of specifically hybridizing to either:
a) a DNA containing a mutant BRCA1 as defined in claim 3, or a DNA having a sequence complementary thereto; or
b) a DNA containing a wild-type BRCA1 sequence at a mutation site other than defined by TABLE 1, or a DNA having a sequence complementary thereto;
but not both a) and b).

34. A plurality of oligonucleotides comprising at least one oligonucleotide according to claim 33 and at least one additional oligonucleotide capable of specifically hybridizing to the wild-type BRCA1 gene or its complement.

35. A chip array having “n” elements for performing allele specific sequence-based techniques using oligonucleotide probes comprising, a solid phase chip and a plurality of oligonucleotides of claim 34 having “n” different nucleotide sequences, wherein “n” is an integer greater than two;
wherein said oligonucleotides are bound to said solid phase chip in a manner which permits said oligonucleotides to effectively hybridize to complementary oligonucleotides or polynucleotides;
wherein oligonucleotides having different nucleotide sequences are bound to said solid phase chip at different locations so that a particular location on said solid phase chip exclusively binds oligonucleotides having a specific nucleotide sequence; and
wherein at least one oligonucleotide is capable of specifically hybridizing to a mutant BRCA1 gene having a truncating mutation as defined by TABLES 3-7 or a DNA complementary thereto, with the proviso that the mutation not be one defined by TABLE 1, wherein the BRCA1 gene codes for a truncated BRCA1 protein which lacks full biological activity of wild-type BRCA1 protein.

36. A method for detecting the presence or absence of a BRCA1 gene mutation in a sample DNA comprising, specifically hybridizing sample DNA with an oligonucleotide according to claim 33 under stringent conditions, and determining whether said oligonucleotide specifically hybridizes to said sample DNA.

37. A method for determining therapy for an individual having a tumor comprising;
a) obtaining a DNA containing biological sample from the individual having a tumor;
b) determining whether the DNA contains a mutant BRCA1 gene according to claim 3, or a DNA complementary thereto; and
c) determining appropriate therapy based on the presence or absence of said mutant BRCA1 gene;
wherein the BRCA1 gene codes for a truncated BRCA1 protein which lacks fill biological activity of wild-type BRCA1 protein.

38. A method for determining whether to begin diagnostic or prophylactic treatment for an individual comprising:
a) obtaining a DNA containing biological sample from the individual;
b) determining whether the DNA contains a mutant BRCA1 gene according to claim 3; and
c) determining appropriate diagnostic or prophylactic treatment based on the presence or absence of said mutant BRCA1 gene;
wherein the mutant BRCA1 gene codes for a truncated BRCA1 protein which lacks fill biological activity of wild-type BRCA1 protein.

39. A method for treating a condition associated with a mutant BRCA1 gene comprising,, administering biologically active BRCA1 protein to a patient with a condition associated with a mutant BRCA1 gene wherein said patient contains cells having a mutant BRCA1 gene according to claim 3, wherein the BRCA1 gene codes for a truncated BRCA1 protein which lacks full biological activity of wild-type BRCA1 protein.

40. A method for preventing a condition associated with a mutant BRCA1 gene comprising, administering biologically active BRCA1 protein to a patient with a cancer wherein said patient contains cells having a truncating BRCA1 gene mutation according to claim 3, wherein the BRCA1 gene codes for a truncated BRCA1 protein which lacks full biological activity of wild-type BRCA1 protein.

41. A method for determining appropriate gene therapy for an individual comprising, detecting the presence of a mutant BRCA1 gene according to claim 3 in cells from the individual and administering a DNA containing biologically active BRCA1 gene to the individual, wherein the mutant BRCA1 gene codes for a truncated BRCA1 protein which lacks full biological activity of wild-type BRCA1 protein.

42. A method for detecting a mutation in a sample containing a mutant BRCA1 gene according to claim 4, comprising:
a) amplifying at least a fragment of sample BRCA1 gene;
b) determining the sequence of said at least a fragment of sample BRCA1 gene; and
c) comparing the sequence obtained with a wild-type BRCA1 sequence;
wherein the presence of the sequence of said mutant BRCA1 gene indicates the presence of a mutation in the sample.

43. A method for detecting a mutation in a sample containing a BRCA1 gene comprising:
a) amplifying at least a fragment of sample BRCA1 gene;
b) determining the sequence of said at least a fragment of sample BRCA1 gene; and
c) comparing the sequence obtained with one or more of mutant BRCA1 genes according to claim 4;
wherein the presence of a sequence of said mutant BRCA1 gene indicates the presence of a mutation in the sample.

44. A method for detecting a mutation in a BRCA1 gene comprising, obtaining a BRCA1 protein expressed by a BRCA1 gene according to claim 4, and determining the relative molecular weight of said BRCA1 protein compared to wild type BRCA1 protein, wherein the presence of said BRCA1 protein having a molecular weight less than that of wild-type BRCA1 protein indicates the presence of a mutation in the BRCA1 gene.

45. An oligonucleotide capable of specifically hybridizing to either:
a) a DNA containing a mutant BRCA1 as defined in claim 4, or a DNA having a sequence complementary thereto; or
b) a DNA containing a wild-type BRCA1 sequence at a mutation site other than defined by TABLE 1, or a DNA having a sequence complementary thereto;
but not both a) and b).

46. A plurality of oligonucleotides comprising at least one oligonucleotide according to claim 45 and at least one additional oligonucleotide capable of specifically hybridizing to the wild-type BRCA1 gene or its complement.

47. A chip array having “n” elements for performing allele specific sequence-based techniques using the oligonucleotide probes comprising, a solid phase chip and a plurality of oligonucleotides of claim 46 having “n” different nucleotide sequences, wherein “n” is an integer greater than two;
wherein said oligonucleotides are bound to said solid phase chip in a manner which permits said oligonucleotides to effectively hybridize to complementary oligonucleotides or polynucleotides;
wherein oligonucleotides having different nucleotide sequences are bound to said solid phase chip at different locations so that a particular location on said solid phase chip exclusively binds oligonucleotides having a specific nucleotide sequence; and
wherein at least one oligonucleotide is capable of specifically hybridizing to a mutant BRCA1 gene having a truncating mutation as defined by TABLES 3-7 or a DNA complementary thereto, with the proviso that the mutation not be one defined by TABLE 1, wherein the BRCA1 gene codes for a truncated BRCA1 protein which lacks full biological activity of wild-type BRCA1 protein.

48. A method for detecting the presence or absence of a BRCA1 gene mutation in a sample DNA comprising, specifically hybridizing sample DNA with an oligonucleotide according to claim 45 under stringent conditions, determining whether said oligonucleotide specifically hybridizes to said sample DNA.

49. A method for determining therapy for an individual having a tumor comprising;
a) obtaining a DNA containing biological sample from the individual having a tumor;
b) determining whether the DNA contains a mutant BRCA1 gene according to claim 4, or a DNA complementary thereto; and
c) determining appropriate therapy based on the presence or absence of said mutant BRCA1 gene;
wherein the BRCA1 gene codes for a truncated BRCA1 protein which lacks full biological activity of wild-type BRCA1 protein.

50. A method for determining whether to begin diagnostic or prophylactic treatment for an individual comprising:
a) taking a DNA containing biological sample from the individual;
b) determining whether the DNA contains a mutant BRCA1 gene according to claim 4; and
c) determining appropriate diagnostic or prophylactic treatment based on the presence or absence of said mutant BRCA1 gene;
wherein the mutant BRCA1 gene codes for a truncated BRCA1 protein which lacks full biological activity of wild-type BRCA1 protein.

51. A method for treating a condition associated with a mutant BRCA1 gene comprising, administering biologically active BRCA1 protein to a patient with a condition associated with a mutant BRCA1 gene wherein said patient contains cells having a mutant BRCA1 gene according to claim 4, wherein the BRCA1 gene codes for a truncated BRCA1 protein which lacks full biological activity of wild-type BRCA1 protein.

52. A method for preventing a condition associated with a mutant BRCA1 gene comprising, administering biologically active BRCA1 protein to a patient with a cancer, wherein said patient contains cells having a truncating BRCA1 gene mutation according to claim 4, wherein the BRCA1 gene codes for a truncated BRCA1 protein which lacks full biological activity of wild-type BRCA1 protein.

53. A method for determining appropriate gene therapy for an individual comprising, detecting the presence of a mutant BRCA1 gene according to claim 4 in cells from the individual, and administering a DNA containing biologically active BRCA1 gene to the individual, wherein the mutant BRCA1 gene codes for a truncated BRCA1 protein which lacks full biological activity of wild-type BRCA1 protein.

54. A method for detecting a mutation in a sample containing a mutant BRCA1 gene according to claim 5, comprising, amplifying at least a fragment of said BRCA1 gene, determining the sequence of said at least a fragment of said BRCA1 gene, and comparing the sequence obtained with a wild-type BRCA1 sequence, wherein the presence of the sequence of said mutant BRCA1 gene indicates the presence of a mutation in the sample.

55. A method for detecting a mutation in a sample containing a BRCA1 gene comprising, amplifying at least a fragment of said BRCA1 gene, determining the sequence of said at least a fragment of said BRCA1 gene, and comparing the sequence obtained with one or more of mutant BRCA1 genes according to claim 5, wherein the presence of a sequence of said mutant BRCA1 gene indicates the presence of a mutation in the sample.

56. A method for detecting a mutation in a BRCA1 gene comprising, obtaining a BRCA1 protein expressed by a BRCA1 gene according to claim 5, and determining the relative molecular weight of said BRCA1 protein compared to wild type BRCA1 protein, wherein the presence of said BRCA1 protein having a molecular weight less than that of wild-type BRCA1 protein indicates the presence of a mutation in the BRCA1 gene.

57. An oligonucleotide capable of specifically hybridizing to either:
a) a DNA containing a mutant BRCA1 as defined in claim 5, or a DNA having a sequence complementary thereto; or
b) a DNA containing a wild-type BRCA1 sequence at a mutation site other than defined by TABLE 1, or a DNA having a sequence complementary thereto;
but not both a) and b).

58. A plurality of oligonucleotides comprising at least one oligonucleotide according to claim 57 and at least one additional oligonucleotide capable of specifically hybridizing to the wild-type BRCA1 gene or its complement.

59. A chip array having “n” elements for performing allele specific sequence-based techniques using the oligonucleotide probes comprising, a solid phase chip and a plurality of oligonucleotides of claim 58 having “n” different nucleotide sequences, wherein “n” is an integer greater than two;
wherein said oligonucleotides are bound to said solid phase chip in a manner which permits said oligonucleotides to effectively hybridize to complementary oligonucleotides or polynucleotides;
wherein oligonucleotides having different nucleotide sequences are bound to said solid phase chip at different locations so that a particular location on said solid phase chip exclusively binds oligonucleotides having a specific nucleotide sequence; and
wherein at least one oligonucleotide is capable of specifically hybridizing to a mutant BRCA1 gene having a truncating mutation as defined by TABLES 3-7 or a DNA complementary thereto, with the proviso that the mutation not be one defined by TABLE 1, wherein the BRCA1 gene codes for a truncated BRCA1 protein which lacks full biological activity of wild-type BRCA1 protein.

60. A method for detecting the presence or absence of a BRCA1 gene mutation in a sample DNA comprising, specifically hybridizing sample DNA with an oligonucleotide according to claim 57 under stringent conditions, and determining whether said oligonucleotide specifically hybridizes to said sample DNA.

61. A method for determining therapy for an individual having a tumor comprising;
a) obtianing a DNA containing biological sample from the individual having a tumor;
b) determining whether the DNA contains a mutant BRCA1 gene according to claim 5, or a DNA complementary thereto; and
c) determining appropriate therapy based on the presence or absence of said mutant BRCA1 gene;
wherein the BRCA1 gene codes for a truncated BRCA1 protein which lacks full biological activity of wild-type BRCA1 protein.

62. A method for determining whether to begin diagnostic or prophylactic treatment for an individual comprising, taking a DNA containing biological sample from the individual, determining whether the DNA contains a mutant BRCA1 gene according to claim 5, and determining appropriate diagnostic or prophylactic treatment based on the presence or absence of said mutant BRCA1 gene, wherein the mutant BRCA1 gene codes for a truncated BRCA1 protein which lacks full biological activity of wild-type BRCA1 protein.

63. A method for treating a condition associated with a mutant BRCA1 gene comprising, administering biologically active BRCA1 protein to a patient with a condition associated with a mutant BRCA1 gene, wherein said patient contains cells having a mutant BRCA1 gene according to claim 5, wherein the BRCA1 gene codes for a truncated BRCA1 protein which lacks full biological activity of wild-type BRCA1 protein.

64. A method for preventing a condition associated with a mutant BRCA1 gene comprising, administering biologically active BRCA1 protein to a patient with a cancer, wherein said patient contains cells having a truncating BRCA1 gene mutation according to claim 5, wherein the BRCA1 gene codes for a truncated BRCA1 protein which lacks full biological activity of wild-type BRCA1 protein.

65. A method for determining appropriate gene therapy for an individual comprising, detecting the presence of a mutant BRCA1 gene according to claim 5 in cells from the individual, and administering a DNA containing biologically active BRCA1 gene to the individual, wherein the mutant BRCA1 gene codes for a truncated BRCA1 protein which lacks full biological activity of wild-type BRCA1 protein.

66. An isolated mutant BRCA1 gene according to claim 1, capable of expressing a truncated BRCA1 protein of less than 1854 amino acids.


SEQUENCE LISTING (10 sequences)

SEQ ID NO: 1 - DNA, 5711 bp; single-stranded, linear
agctcgctgagacttcctggaccccgcaccaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagtgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcactggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctctgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaaggagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagagaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcctctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagggtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 2 - protein, 1863 aa
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFALFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKGASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQRGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQGPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 3 - DNA, 17 bp; single-stranded, linear
aatcttagagtgtccca

SEQ ID NO: 4 - DNA, 17 bp; single-stranded, linear
atcttagtgtcccacct

SEQ ID NO: 5 - DNA, 17 bp; single-stranded, linear
cagaaaaaaaggtagat

SEQ ID NO: 6 - DNA, 17 bp; single-stranded, linear
cagaaaaaaaaggtaga

SEQ ID NO: 7 - DNA, 17 bp; single-stranded, linear
agagaatcccaggacag

SEQ ID NO: 8 - DNA, 17 bp; single-stranded, linear
agagaatccccaggaca

SEQ ID NO: 9 - DNA, 17 bp; single-stranded, linear
aggacctgcgaaatcca

SEQ ID NO: 10 - DNA, 17 bp; single-stranded, linear
aggacctgtgaaatcca